(19) United States
(12) Patent Application Publication
(10) Pub. No.: US 2026/0285871 A1
(43) Pub. Date: Sep. 24, 2026

(54) Polymorphic Compounds and Uses Thereof

(71) Applicant: TYRA BIOSCIENCES, INC., Carlsbad, CA (US)

(72) Inventors: Piyush PATEL, Garnet Valley, PA (US); Robert L. HUDKINS, Carlsbad, CA (US); Sathyanarayana Reddy Perumalla, Wilmington, DE (US)

(21) Appl. No.: 18/874,355

(22) PCT Filed: Jun. 29, 2023
(86) PCT No.: PCT/US2023/069334

§ 371 Date: Dec. 12, 2024

Related U.S. Application Data
(60) Provisional application No. 63/356,755, filed on Jun. 29, 2022.

(51) Int. Cl. C07D 487/10

(52) U.S. Cl. CPC C07D 487/10

(57) ABSTRACT

The present invention provides salt forms, and compositions and methods thereof, useful for treating various conditions, such as cancer, by the administration of small molecule therapeutics acting as kinase inhibitors.

### CROSS REFERENCE TO RELATED APPLICATIONS

This application claims the benefit of U.S. Provisional Application No. 63/356,755, filed Jun. 29, 2022, the entirety of which is incorporated by reference herein.

### TECHNICAL FIELD

The disclosure pertains to indazole compounds that are useful in treating cancer, pharmaceutical compositions that include one or more such indazole compounds, and methods of using such indazole compounds in treating cancer.

### BACKGROUND

Kinase inhibitors have been used to block the activity of kinases and thereby treat cancer (e.g., by inhibiting mitotic processes). These kinase inhibitors are often small molecules that target kinases to block the development, growth or spread of cancer.

However, although various inhibitors of kinases are known, there remains a need for selective inhibitors to be used for the treatment of diseases such as hyper-proliferative diseases, which offer one or more advantages over current compounds. Those advantages include: improved activity and/or efficacy; beneficial kinase selectivity profile according to the respective therapeutic need; improved side effect profile, such as fewer undesired side effects, lower intensity of side effects, or reduced (cyto)toxicity; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing, e.g., by shorter synthetic routes or easier purification.

Additional small molecule kinase compounds are needed.

### SUMMARY OF THE INVENTION

It has now been found that compounds of the present invention, and compositions thereof, are useful for treating, preventing, and/or reducing a risk of a disease, disorder, or condition in a kinase-mediated disorder. Such compounds are represented by the chemical structure below, denoted as compound A:

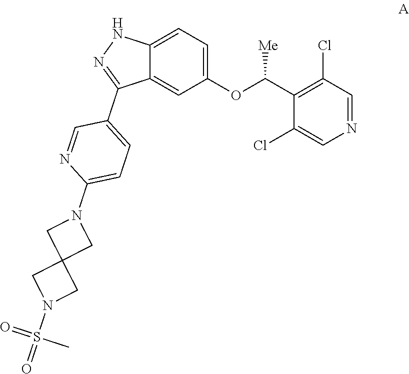 [SMILES: C[C@@H](Oc1ccc2[nH]nc(-c3ccc(N4CC5(C4)CN(S(C)(=O)=O)C5)nc3)c2c1)c1c(Cl)cncc1Cl]

or a pharmaceutically acceptable salt thereof.

Compounds of the present invention, and pharmaceutically acceptable compositions thereof, are useful for treating a variety of diseases, disorders or conditions, associated with kinase-mediation. Such diseases, disorders, or conditions include those described herein.

### BRIEF DESCRIPTION OF THE FIGURES

FIG. 1 depicts an XRPD pattern of Compound 1, Form I.

FIG. 2 depicts an XRPD pattern of Compound 2, Form I.

FIG. 3 depicts a DSC thermogram and TGA trace of Compound 2, Form I.

FIG. 4 depicts an XRPD pattern of Compound 2, Form II.

FIG. 5 depicts an XRPD pattern of Compound 2, Form III.

FIG. 6 depicts a DSC thermogram and TGA trace of Compound 2, Form III.

FIG. 7 depicts an XRPD pattern of Compound 2, Form IV.

FIG. 8 depicts an XRPD pattern of Compound 2, Form V.

FIG. 9 depicts a DSC thermogram and TGA trace of Compound 2, Form V.

FIG. 10 depicts an XRPD pattern of Compound 2, Form VI.

FIG. 11 depicts an XRPD pattern of Compound 2, Form VII.

FIG. 12 depicts a DSC thermogram and TGA trace of Compound 2, Form VII.

FIG. 13 depicts an XRPD pattern of Compound 2, Form VIII.

FIG. 14 depicts an XRPD pattern of Compound 2, Form IX.

FIG. 15 depicts an XRPD pattern of Compound 2, Form X.

FIG. 16 depicts a DSC thermogram and TGA trace of Compound 2, Form X.

FIG. 17 depicts an XRPD pattern of Compound 2, Form XI.

FIG. 18 depicts a DSC thermogram and TGA trace of Compound 2, Form XI.

FIG. 19 depicts an XRPD pattern of Compound 2, Form XII.

FIG. 20 depicts a DSC thermogram and TGA trace of Compound 2, Form XII.

FIG. 21 depicts an XRPD pattern of Compound 2, Form XIII.

FIG. 22 depicts a DSC thermogram and TGA trace of Compound 2, Form XIII.

FIG. 23 depicts an XRPD pattern of Compound 3, Form I.

FIG. 24 depicts an XRPD pattern of Compound 4, Form I.

FIG. 25 depicts an XRPD pattern of Compound 5, Form I.

FIG. 26 depicts an XRPD pattern of Compound 6, Form I.

FIG. 27 depicts an XRPD pattern of Compound 7, Form I.

FIG. 28 depicts an XRPD pattern of Compound 8, Form I.

FIG. 29 depicts an XRPD pattern of Compound 10, Form I.

FIG. 30 depicts an XRPD pattern of Compound 11, Form I.

FIG. 31 depicts an XRPD pattern of Compound 12, Form I.

FIG. 32 depicts an XRPD pattern of Compound 15.

FIG. 33 depicts a DSC thermogram and TGA trace of Compound 15.

FIG. 34 depicts an XRPD pattern of Compound 16.

FIG. 35 depicts a DSC thermogram and TGA trace of Compound 16.

FIG. 36 depicts an XRPD pattern of Compound 17.

FIG. 37 depicts a DSC thermogram and TGA trace of Compound 17.

FIG. 38 depicts an XRPD pattern of Compound 18.

FIG. 39 depicts a DSC thermogram and TGA trace of Compound 18.

FIG. 40 depicts an XRPD pattern of Compound 19.

FIG. 41 depicts a DSC thermogram and TGA trace of Compound 19.

FIG. 42 depicts an XRPD pattern of Compound 20.

FIG. 43 depicts a DSC thermogram and TGA trace of Compound 20.

FIG. 44 depicts an XRPD pattern of Compound 21.

FIG. 45 depicts a DSC thermogram and TGA trace of Compound 21.

FIG. 46 depicts an XRPD pattern of Compound 22.

FIG. 47 depicts a DSC thermogram and TGA trace of Compound 22.

FIG. 48 depicts an XRPD pattern of Compound 23, Form I.

FIG. 49 depicts a DSC thermogram and TGA trace of Compound 23, Form I.

FIG. 50 depicts an XRPD pattern of Compound 24, Form I.

FIG. 51 depicts a DSC thermogram and TGA trace of Compound 24, Form I.

FIG. 52 depicts an XRPD pattern of Compound 25, Form I.

FIG. 53 depicts a DSC thermogram and TGA trace of Compound 25, Form I.

FIG. 54 depicts an XRPD pattern of Compound 26, Form I.

FIG. 55 depicts a DSC thermogram and TGA trace of Compound 26, Form I.

FIG. 56 depicts an XRPD pattern of Compound 1, Form II.

FIG. 57 depicts a DSC thermogram and TGA trace of Compound 1, Form II.

FIG. 58 depicts an XRPD pattern of Compound 3, Form II.

FIG. 59 depicts a DSC thermogram and TGA trace of Compound 3, Form II.

FIG. 60 depicts an XRPD pattern of Compound 5, Form II.

FIG. 61 depicts a DSC thermogram and TGA trace of Compound 5, Form II.

FIG. 62 depicts an XRPD pattern of Compound 12, Form II.

FIG. 63 depicts a DSC thermogram and TGA trace of Compound 12, Form II.

### DETAILED DESCRIPTION OF THE INVENTION

### General Description of Certain Aspects of the Invention

International Patent Application No. PCT/US2021/065679, filed Dec. 30, 2021, the entirety of which is hereby incorporated herein by reference, describes certain kinase-inhibitors, including the following compound A:

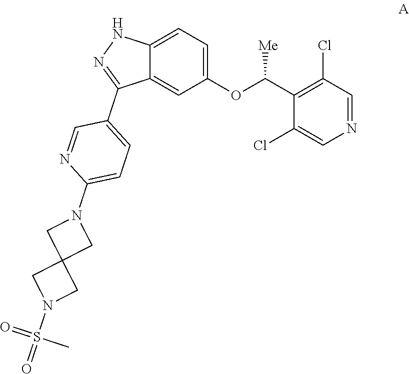 [SMILES: C[C@@H](Oc1ccc2[nH]nc(-c3ccc(N4CC5(C4)CN(S(C)(=O)=O)C5)nc3)c2c1)c1c(Cl)cncc1Cl]

Compound A, 5-[(1R)-1-(3,5-dichloro-4-pyridyl) ethoxy]-3-[6-(2-methylsulfonyl-2,6-diazaspiro[3.3]heptan-6-yl)-3-pyridyl]-1H-indazole, is designated as Example 46 in International Patent Application No. PCT/US2021/065679.

Solid forms of compound A (e.g., as a freebase thereof or salt thereof) as described herein exhibit improved characteristics such as, for example, improved aqueous solubility, stability and ease of formulation. Accordingly, the present invention provides salt forms and free base forms of compound A.

As used herein, the term “about” when used to refer to peaks from XRPD data is taken to mean 2θ±0.2.

As used herein, the term “substantially similar” when referring to an X-ray powder diffraction pattern in a figure refers to an X-ray diffraction pattern possessing similarities sufficient for those skilled in the art to understand that the same form of the same compound is represented by the figure.

As used herein, the HPLC methods for determining organic impurities are directed to ultra-high performance liquid chromatography (UPLC) methods developed and validated for the determination of assay and impurities of the compounds described herein. Gradient UPLC analysis is performed using a Waters Cortex C18, 100× 2.1 mm, 1.6 μm particle size (or equivalent) column, 0.1% trifluoroacetic in water (A) and 0.1% trifluoroacetic acid in acetonitrile (B) mobile phase and UV detection. Samples are reconstituted in methanol to a target compound concentration of 1.0 mg/mL.

Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

### Salt Forms of Compound A

In some embodiments, an acid and compound A are combined to form one of compounds 1 through 12 and 23 through 26, described below. It is contemplated that compounds 1 through 12 and 23 through 26 can exist in a variety of physical forms. For example, compounds 1 through 12 and 23 through 26 can be in solution, suspension, or in solid form. In certain embodiments, compounds 1 through 12 and 23 through 26 are in solid form. When compounds 1 through 12 and 23 through 26 are in solid form, said compounds may be amorphous, crystalline, or a mixture thereof. Exemplary such solid forms of compounds 1 through 12 and 23 through 26 are described in more detail below.

### Compound 1 (Hydrochloride Salts of Compound A)

According to one embodiment, the present invention provides a hydrochloride salt of compound A, represented by compound 1:

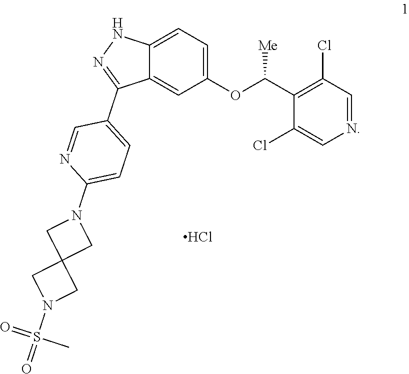 [SMILES: C[C@@H](Oc1ccc2[nH]nc(-c3ccc(N4CC5(C4)CN(S(C)(=O)=O)C5)nc3)c2c1)c1c(Cl)cncc1Cl ; Cl]

It will be appreciated by one of ordinary skill in the art that the hydrochloric acid and compound A are ionically bonded to form compound 1. It is contemplated that compound 1 can exist in a variety of physical forms. For example, compound 1 can be in solution, suspension, or in solid form. In certain embodiments, compound 1 is in solid form. When compound 1 is in solid form, said compound may be amorphous, crystalline, or a mixture thereof. Exemplary solid forms are described in more detail below.

In some embodiments, the present invention provides compound 1 substantially free of impurities. As used herein, the term “substantially free of impurities” means that the compound contains no significant amount of extraneous matter. Such extraneous matter may include excess hydrochloric acid, excess synthetic reagents, excess compound A, residual solvents, or any other impurities that may result from the preparation of, and/or isolation of, compound 1. In certain embodiments, at least about 95% by weight of compound 1 is present in a composition. In still other embodiments of the invention, at least about 99% by weight of compound 1 is present in a composition.

According to one embodiment, compound 1 is present in a composition in an amount of at least about 97, 97.5, 98.0, 98.5, 99, 99.5, 99.8 weight percent where the percentages are based on the total weight of the composition. According to another embodiment, compound 1 contains no more than about 3.0 area percent HPLC of total organic impurities. The area percent by HPLC of total organic impurities can be assessed by those skilled in the art, using conventional HPLC methods, including for example, a photodiode array detector. In certain embodiments, no more than about 1.5 area percent HPLC total organic impurities relative to the total area of the HPLC chromatogram. In other embodiments, compound 1 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 1 is also meant to include all tautomeric forms and isomeric forms of compound 1.

It has been found that compound 1 can exist in a variety of solid forms. Exemplary such forms include polymorphs such as those described herein.

In certain embodiments, compound 1 is a crystalline solid. In other embodiments, compound 1 is a crystalline solid substantially free of amorphous compound 1. As used herein, the term “substantially free of amorphous compound 1” means that the compound contains no significant amount of amorphous compound 1. In certain embodiments, at least about 95% by weight of crystalline compound 1 is present. In still other embodiments of the invention, at least about 99% by weight of crystalline compound 1 is present.

It has been found that compound 1 can exist in at least two polymorphic forms.

In some embodiments, the present invention provides a polymorphic form of Compound 1 referred to herein as Form I. In some embodiments, Form I of compound 1 is substantially free of other polymorphic forms.

In certain embodiments, the present invention provides a polymorphic form of compound 1 referred to herein as Form II. In some embodiments, Form II of compound 1 is substantially free of other polymorphic forms.

In some embodiments, compound 1 is amorphous. In some embodiments, compound 1 is amorphous, and is substantially free of crystalline compound 1.

### Form I of Compound 1

In some embodiments, Form I of compound 1 has at least 1, 2, 3, 4 or 5 spectral peak(s) selected from the peaks listed in Table 1 below.

TABLE 1
XRPD Peak Positions for Form I of Compound 1

| Pos. [°2θ]¹ | Int. I. (cps) |
|---|---|
| 5.7 | 755(22) |
| 6.9 | 8142(41) |
| 8.0 | 2212(37) |
| 9.7 | 405(26) |
| 11.3 | 839(33) |
| 12.1 | 5541(56) |
| 12.5 | 7326(64) |
| 14.1 | 2819(380) |
| 14.4 | 2541(349) |
| 14.6 | 2308(322) |
| 14.8 | 4143(558) |
| 15.8 | 735(64) |
| 17.2 | 7155(190) |
| 19.9 | 57429(453) |
| 20.6 | 1087(105) |
| 21.9 | 43194(384) |
| 22.7 | 4138(167) |
| 24.1 | 2206(188) |
| 24.5 | 8768(217) |
| 25.2 | 9361(798) |
| 25.7 | 13503(768) |
| 27.1 | 1559(171) |
| 27.4 | 1485(193) |
| 28.2 | 7364(113) |
| 28.7 | 474(177) |
| 29.0 | 940(134) |
| 29.5 | 585(44) |
| 30.3 | 2820(133) |
| 32.5 | 2300(262) |
| 32.9 | 4461(256) |
| 33.8 | 4766(188) |
| 34.1 | 1047(158) |
| 34.6 | 1533(116) |
| 35.0 | 801(129) |
| 36.3 | 4470(62) |
| 37.0 | 211(31) |
| 38.8 | 352(48) |
| 39.2 | 171(33) |
| 39.9 | 713(52) |
| 40.5 | 949(47) |
| 41.4 | 1053(78) |
| 41.9 | 1141(91) |
| 43.6 | 353(64) |
| 44.2 | 1006(210) |
| 44.7 | 1156(268) |
| 45.2 | 1002(204) |
| 47.5 | 354(36) |
| 48.6 | 157(30) |
| 49.2 | 1938(79) |
| 50.2 | 212(28) |
| 56.2 | 1609(85) |

¹In this and all subsequent tables, the position 2θ is within ±0.2.

In some embodiments, Form I of compound 1 is characterized in that it has one or more peaks in its X-ray powder diffraction pattern selected from those at about 19.9, about 21.9 and about 25.7 degrees 2-theta. In some embodiments, Form I of compound 1 is characterized in that it has two or more peaks in its X-ray powder diffraction pattern selected from those at about 19.9, about 21.9 and about 25.7 degrees 2-theta. In some embodiments, Form I of compound 1 is characterized in that it has all three peaks in its X-ray powder diffraction pattern selected from those at about 19.9, about 21.9 and about 25.7 degrees 2-theta.

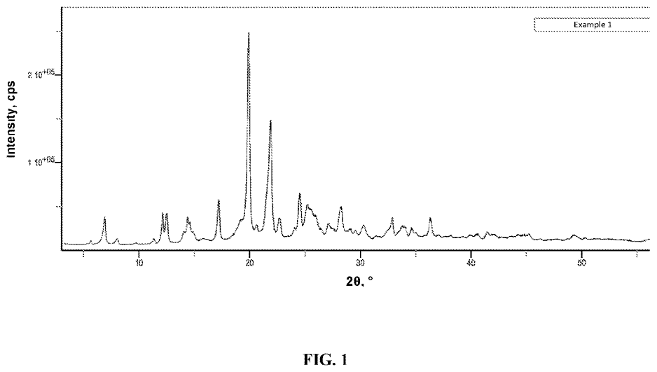

In certain embodiments, the X-ray powder diffraction pattern is substantially similar to the XRPD provided in FIG. 1.

Methods for preparing Form I of compound 1 are described infra.

### Form II of Compound 1

In some embodiments, Form II of compound 1 has at least 1, 2, 3, 4 or 5 spectral peak(s) selected from the peaks listed in Table 2 below.

TABLE 2
XRPD Peak Positions for Form II of Compound 1

| Pos. [°2θ] | Rel. Int. [%] |
|---|---|
| 5.2 | 1.94 |
| 6.0 | 6.17 |
| 7.3 | 19.96 |
| 8.5 | 2.70 |
| 10.1 | 4.27 |
| 11.7 | 11.14 |
| 12.5 | 17.94 |
| 12.9 | 61.38 |
| 14.4 | 14.18 |
| 14.8 | 55.41 |
| 15.4 | 20.16 |
| 16.3 | 5.08 |
| 17.6 | 40.45 |
| 19.6 | 23.90 |
| 20.3 | 100.00 |
| 22.2 | 56.42 |
| 23.0 | 22.46 |
| 24.5 | 12.18 |
| 25.0 | 47.66 |
| 25.6 | 45.13 |
| 25.9 | 32.96 |
| 26.4 | 29.31 |
| 27.5 | 20.12 |
| 28.6 | 18.85 |
| 30.0 | 10.49 |
| 30.7 | 12.02 |
| 31.7 | 2.87 |
| 33.2 | 5.69 |
| 34.5 | 10.31 |
| 35.1 | 7.15 |
| 36.6 | 5.74 |

In some embodiments, Form II of compound 1 is characterized in that it has one or more peaks in its X-ray powder diffraction pattern selected from those at about 12.9, about 20.3 and about 22.2 degrees 2-theta. In some embodiments, Form II of compound 1 is characterized in that it has two or more peaks in its X-ray powder diffraction pattern selected from those at about 12.9, about 20.3 and about 22.2 degrees 2-theta. In some embodiments, Form II of compound 1 is characterized in that it has all three peaks in its X-ray powder diffraction pattern selected from those at about 12.9, about 20.3 and about 22.2 degrees 2-theta.

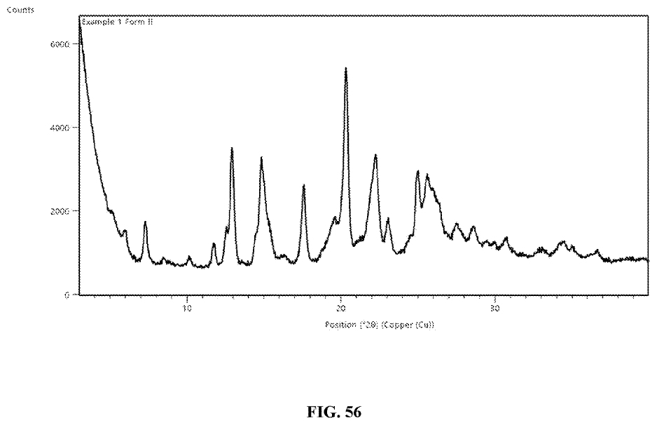

In certain embodiments, the X-ray powder diffraction pattern is substantially similar to the XRPD provided in FIG. 56.

Methods for preparing Form II of compound 1 are described infra.

In some embodiments, the present invention provides compound 1, wherein said compound is crystalline.

In some embodiments, the present invention provides compound 1, wherein said compound is a crystalline solid substantially free of amorphous compound 1.

In some embodiments, the present invention provides compound 1, wherein said compound is substantially free of impurities.

In some embodiments, the present invention provides a composition comprising Form I or Form II of compound 1 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I or Form II of compound 1 or composition thereof.

In some embodiments, the present invention provides a method of treating cancer in a patient by blocking the activity of kinases, comprising administering to said patient Form I or Form II of compound 1 or composition thereof wherein Form I or Form II of compound 1 is a crystal form as described herein. In some embodiments, the present invention provides a method of treating one or more of the diseases, disorders, or conditions described herein.

### Compound 2 (Besylate Salts of Compound A)

According to one embodiment, the present invention provides a besylate salt of compound A, represented by compound 2:

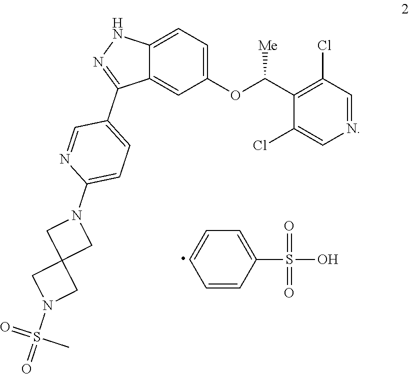 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=S(=O)(O)c1ccccc1]

It will be appreciated by one of ordinary skill in the art that the benzenesulfonic acid and compound A are ionically bonded to form compound 2. It is contemplated that compound 2 can exist in a variety of physical forms. For example, compound 2 can be in solution, suspension, or in solid form. In certain embodiments, compound 2 is in solid form. When compound 2 is in solid form, said compound may be amorphous, crystalline, or a mixture thereof. Exemplary solid forms are described in more detail below.

In some embodiments, the present invention provides compound 2 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 2. In certain embodiments, at least about 95% by weight of compound 2 is present in a composition. In still other embodiments of the invention, at least about 99% by weight of compound 2 is present in a composition.

According to one embodiment, compound 2 is present in a composition in an amount of at least about 97, 97.5, 98.0, 98.5, 99, 99.5, 99.8 weight percent where the percentages are based on the total weight of the composition. According to another embodiment, compound 2 contains no more than about 3.0 area percent HPLC of total organic impurities and, in certain embodiments, no more than about 1.5 area percent HPLC total organic impurities relative to the total area of the HPLC chromatogram. In other embodiments, compound 2 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 2 is also meant to include all tautomeric forms and isomeric forms of compound 2.

It has been found that compound 2 can exist in a variety of solid forms. Exemplary such forms include polymorphs such as those described herein.

In certain embodiments, compound 2 is a crystalline solid. In other embodiments, compound 2 is a crystalline solid substantially free of amorphous compound 2. As used herein, the term “substantially free of amorphous compound 2” means that the compound contains no significant amount of amorphous compound 2. In certain embodiments, at least about 95% by weight of crystalline compound 2 is present. In still other embodiments of the invention, at least about 99% by weight of crystalline compound 2 is present.

It has been found that compound 2 can exist in at least thirteen polymorphic forms.

In some embodiments, the present invention provides a polymorphic form of Compound 2 referred to herein as Form I. In some embodiments, Form I of compound 2 is substantially free of other polymorphic forms.

In certain embodiments, the present invention provides a polymorphic form of compound 2 referred to herein as Form II. In some embodiments, Form II of compound 2 is substantially free of other polymorphic forms.

In certain embodiments, the present invention provides a polymorphic form of compound 2 referred to herein as Form III. In some embodiments, Form III of compound 2 is substantially free of other polymorphic forms.

In certain embodiments, the present invention provides a polymorphic form of compound 2 referred to herein as Form IV. In some embodiments, Form IV of compound 2 is substantially free of other polymorphic forms.

In certain embodiments, the present invention provides a polymorphic form of compound 2 referred to herein as Form V. In some embodiments, Form V of compound 2 is substantially free of other polymorphic forms.

In certain embodiments, the present invention provides a polymorphic form of compound 2 referred to herein as Form VI. In some embodiments, Form VI of compound 2 is substantially free of other polymorphic forms.

In certain embodiments, the present invention provides a polymorphic form of compound 2 referred to herein as Form VII. In some embodiments, Form VII of compound 2 is substantially free of other polymorphic forms.

In certain embodiments, the present invention provides a polymorphic form of compound 2 referred to herein as Form VIII. In some embodiments, Form VIII of compound 2 is substantially free of other polymorphic forms.

In certain embodiments, the present invention provides a polymorphic form of compound 2 referred to herein as Form IX. In some embodiments, Form IX of compound 2 is substantially free of other polymorphic forms.

In certain embodiments, the present invention provides a polymorphic form of compound 2 referred to herein as Form X. In some embodiments, Form X of compound 2 is substantially free of other polymorphic forms.

In certain embodiments, the present invention provides a polymorphic form of compound 2 referred to herein as Form XI. In some embodiments, Form XI of compound 2 is substantially free of other polymorphic forms.

In certain embodiments, the present invention provides a polymorphic form of compound 2 referred to herein as Form XII. In some embodiments, Form XII of compound 2 is substantially free of other polymorphic forms.

In certain embodiments, the present invention provides a polymorphic form of compound 2 referred to herein as Form XIII. In some embodiments, Form XIII of compound 2 is substantially free of other polymorphic forms.

In some embodiments, compound 2 is amorphous. In some embodiments, compound 2 is amorphous, and is substantially free of crystalline compound 2.

### Form I of Compound 2

In some embodiments, Form I of compound 2 has at least 1, 2, 3, 4 or 5 spectral peak(s) selected from the peaks listed in Table 3 below.

TABLE 3
XRPD Peak Positions for Form I of Compound 2

| Pos. [°2θ] | Int. I. (cps) |
|---|---|
| 4.9 | 4508(29) |
| 6.7 | 10424(54) |
| 7.6 | 1046(31) |
| 10.8 | 1587(35) |
| 11.2 | 639(30) |
| 12.3 | 1389(43) |
| 12.7 | 4231(71) |
| 13.3 | 339(31) |
| 14.5 | 4359(133) |
| 15.0 | 8811(282) |
| 15.2 | 8395(304) |
| 15.8 | 2471(80) |
| 16.4 | 2315(107) |
| 17.7 | 7387(187) |
| 18.3 | 19860(311) |
| 18.6 | 1771(182) |
| 19.5 | 7297(166) |
| 20.0 | 32751(798) |
| 20.2 | 9954(1452) |
| 20.4 | 9283(869) |
| 21.0 | 5422(192) |
| 21.6 | 16308(223) |
| 23.1 | 5128(91) |
| 23.7 | 4317(96) |
| 24.4 | 1779(71) |
| 24.8 | 6104(164) |
| 25.2 | 4179(200) |
| 25.7 | 6236(138) |
| 26.4 | 1252(111) |
| 26.6 | 1595(141) |
| 27.1 | 12460(176) |
| 28.3 | 1394(525) |
| 28.5 | 1942(590 |
| 29.3 | 3612(162) |
| 29.8 | 4004(156) |
| 30.5 | 631(94) |
| 31.3 | 637(101) |
| 32.8 | 2547(88) |
| 33.5 | 533(61) |
| 33.9 | 3214(91) |
| 34.7 | 1408(64) |
| 35.1 | 660(64) |
| 35.9 | 1637(62) |
| 36.6 | 1378(72) |
| 39.2 | 960(98) |
| 40.5 | 715(66) |
| 41.1 | 2482(98) |
| 42.6 | 817(58) |
| 44.3 | 441(79) |
| 46.3 | 623(56) |
| 47.7 | 228(32) |
| 48.1 | 1604(47) |
| 49.5 | 1109(58) |
| 51.1 | 946(64) |
| 56.2 | 451(65) |

In some embodiments, Form I of compound 2 is characterized in that it has one or more peaks in its X-ray powder diffraction pattern selected from those at about 18.3, about 20.0 and about 21.6 degrees 2-theta. In some embodiments, Form I of compound 2 is characterized in that it has two or more peaks in its X-ray powder diffraction pattern selected from those at about 18.3, about 20.0 and about 21.6 degrees 2-theta. In some embodiments, Form I of compound 2 is characterized in that it has all three peaks in its X-ray powder diffraction pattern selected from those at about 18.3, about 20.0 and about 21.6 degrees 2-theta.

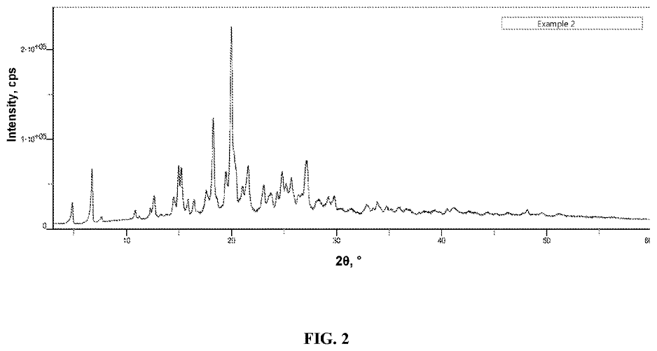

In certain embodiments, the X-ray powder diffraction pattern is substantially similar to the XRPD provided in FIG. 2.

Methods for preparing Form I of compound 2 are described infra.

### Form II of Compound 2

In some embodiments, Form II of compound 2 has at least 1, 2, 3, 4 or 5 spectral peak(s) selected from the peaks listed in Table 4 below.

TABLE 4
XRPD Peak Positions for Form II of Compound 2

| Pos. [°2θ] | Int. I. (cps) |
|---|---|
| 5.9 | 39498(195) |
| 8.5 | 181(16) |
| 9.0 | 353(25) |
| 9.7 | 251(22) |
| 10.3 | 270(26) |
| 11.4 | 975(41) |
| 11.9 | 5702(71) |
| 12.3 | 3042(74) |
| 13.4 | 5035(44) |
| 14.1 | 592(51) |
| 15.0 | 4888(73) |
| 15.8 | 4747(49) |
| 16.3 | 238(30) |
| 16.5 | 658(35) |
| 17.0 | 2625(120) |
| 17.2 | 10641(150) |
| 18.0 | 9441(134) |
| 18.3 | 2191(116) |
| 18.6 | 2866(88) |
| 19.2 | 1066(89) |
| 19.9 | 18727(155) |
| 20.4 | 2275(170) |
| 20.8 | 2205(182) |
| 21.7 | 10648(166) |
| 22.2 | 1366(38) |
| 22.7 | 3181(170) |
| 23.0 | 6765(203) |
| 23.3 | 3703(220) |
| 23.8 | 253(52) |
| 24.3 | 3114(58) |
| 24.8 | 2040(47) |
| 25.3 | 799(54) |
| 25.7 | 2350(107) |
| 26.0 | 585(67) |
| 26.7 | 532(559) |
| 26.8 | 837(581) |
| 27.9 | 1956(390) |
| 28.1 | 4487(581) |
| 28.5 | 2880(1108) |
| 28.6 | 2757(1380) |
| 29.0 | 189(18) |
| 29.4 | 794(73) |
| 29.7 | 5478(90) |
| 30.2 | 4964(76) |
| 30.9 | 425(38) |
| 31.4 | 266(46) |
| 31.9 | 3067(130) |
| 33.4 | 424(35) |
| 33.8 | 486(63) |
| 34.1 | 807(70) |
| 34.6 | 1041(78) |
| 35.5 | 1184(67) |
| 36.6 | 1815(85) |
| 37.6 | 5204(130) |
| 38.5 | 288(24) |
| 39.5 | 1325(64) |
| 40.0 | 231(31) |
| 40.6 | 280(42) |
| 40.9 | 256(43) |
| 42.4 | 146(72) |
| 42.6 | 1123(146) |
| 43.2 | 561(134) |
| 44.1 | 452(31) |
| 45.5 | 982(68) |
| 47.7 | 225(21) |
| 48.3 | 131(18) |
| 48.9 | 104(26) |
| 49.3 | 973(52) |
| 51.1 | 677(66) |
| 52.2 | 789(63) |
| 53.5 | 261(22) |
| 58.0 | 801(128) |
| 58.7 | 446(121) |

In some embodiments, Form II of compound 2 is characterized in that it has one or more peaks in its X-ray powder diffraction pattern selected from those at about 5.9, about 19.9 and about 21.7 degrees 2-theta. In some embodiments, Form II of compound 2 is characterized in that it has two or more peaks in its X-ray powder diffraction pattern selected from those at about 5.9, about 19.9 and about 21.7 degrees 2-theta. In some embodiments, Form II of compound 2 is characterized in that it has all three peaks in its X-ray powder diffraction pattern selected from those at about 5.9, about 19.9 and about 21.7 degrees 2-theta.

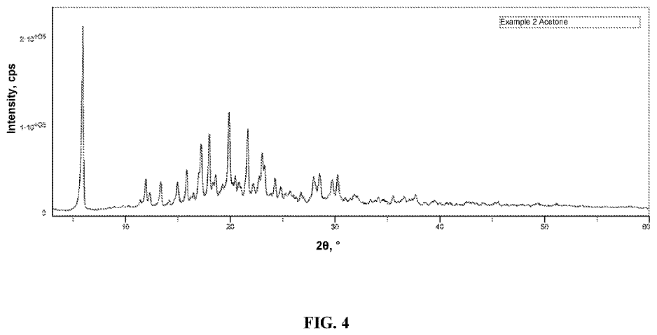

In certain embodiments, the X-ray powder diffraction pattern is substantially similar to the XRPD provided in FIG. 4.

Methods for preparing Form II of compound 2 are described infra.

### Form III of Compound 2

In some embodiments, Form III of compound 2 has at least 1, 2, 3, 4 or 5 spectral peak(s) selected from the peaks listed in Table 5 below.

TABLE 5
XRPD Peak Positions for Form III of Compound 2

| Pos. [°2θ] | Rel. Int. [%] |
|---|---|
| 5.3 | 2.25 |
| 5.9 | 1.18 |
| 6.8 | 100.00 |
| 9.7 | 0.25 |
| 11.4 | 0.46 |
| 12.4 | 0.41 |
| 13.5 | 1.06 |
| 16.5 | 3.86 |
| 18.9 | 22.55 |
| 20.3 | 34.74 |
| 21.1 | 4.51 |
| 23.1 | 2.84 |
| 25.5 | 2.39 |
| 27.1 | 8.12 |
| 29.9 | 0.92 |
| 32.4 | 0.90 |
| 34.3 | 0.71 |
| 38.0 | 0.33 |

In some embodiments, Form III of compound 2 is characterized in that it has one or more peaks in its X-ray powder diffraction pattern selected from those at about 6.8, about 18.9 and about 20.3 degrees 2-theta. In some embodiments, Form III of compound 2 is characterized in that it has two or more peaks in its X-ray powder diffraction pattern selected from those at about 6.8, about 18.9 and about 20.3 degrees 2-theta. In some embodiments, Form III of compound 2 is characterized in that it has all three peaks in its X-ray powder diffraction pattern selected from those at about 6.8, about 18.9 and about 20.3 degrees 2-theta.

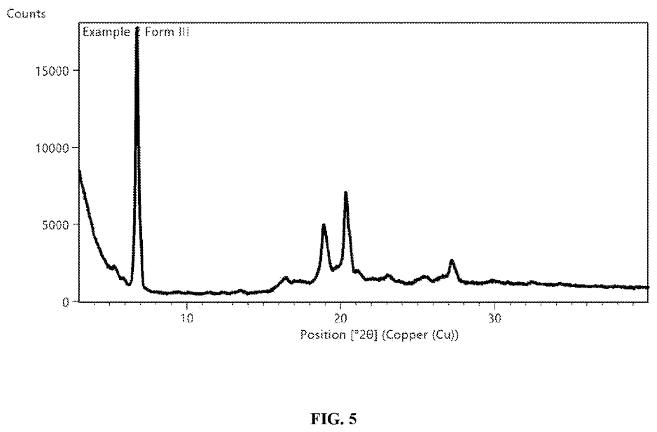

In certain embodiments, the X-ray powder diffraction pattern is substantially similar to the XRPD provided in FIG. 5.

Methods for preparing Form III of compound 2 are described infra.

### Form IV of Compound 2

In some embodiments, Form IV of compound 2 has at least 1, 2, 3, 4 or 5 spectral peak(s) selected from the peaks listed in Table 6 below.

TABLE 6
XRPD Peak Positions for Form IV of Compound 2

| Pos. [°2θ] | Rel. Int. [%] |
|---|---|
| 4.9 | 74.87 |
| 5.0 | 100.00 |
| 5.9 | 5.03 |
| 6.5 | 37.18 |
| 6.7 | 30.87 |
| 7.6 | 9.33 |
| 10.0 | 4.23 |
| 11.1 | 16.81 |
| 12.7 | 27.20 |
| 14.6 | 9.90 |
| 15.1 | 28.28 |
| 15.2 | 39.66 |
| 15.4 | 38.95 |
| 16.4 | 6.02 |
| 17.2 | 19.66 |
| 17.6 | 6.62 |
| 18.3 | 10.37 |
| 18.7 | 16.70 |
| 19.2 | 8.12 |
| 19.4 | 22.51 |
| 19.6 | 31.54 |
| 20.0 | 33.63 |
| 21.0 | 10.69 |
| 21.7 | 12.80 |
| 22.0 | 7.71 |
| 22.6 | 9.65 |
| 24.8 | 39.13 |
| 25.0 | 40.45 |
| 26.2 | 13.76 |
| 26.6 | 8.43 |
| 27.4 | 3.30 |
| 30.3 | 2.97 |
| 34.1 | 2.57 |
| 34.9 | 1.73 |

In some embodiments, Form IV of compound 2 is characterized in that it has one or more peaks in its X-ray powder diffraction pattern selected from those at about 4.9, about 5.0 and about 25.0 degrees 2-theta. In some embodiments, Form IV of compound 2 is characterized in that it has two or more peaks in its X-ray powder diffraction pattern selected from those at about 4.9, about 5.0 and about 25.0 degrees 2-theta. In some embodiments, Form IV of compound 2 is characterized in that it has all three peaks in its X-ray powder diffraction pattern selected from those at about 4.9, about 5.0 and about 25.0 degrees 2-theta.

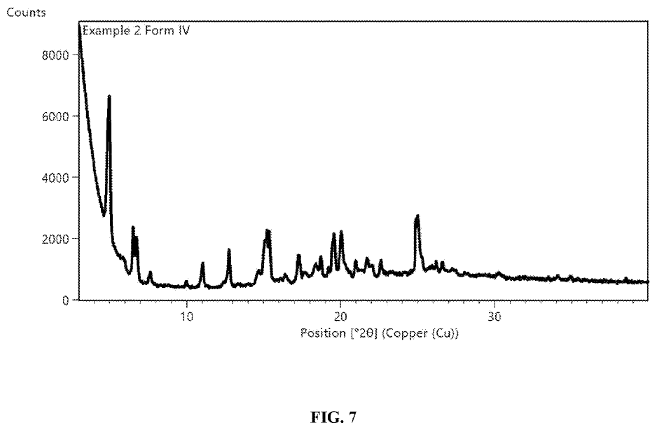

In certain embodiments, the X-ray powder diffraction pattern is substantially similar to the XRPD provided in FIG. 7.

Methods for preparing Form IV of compound 2 are described infra.

### Form V of Compound 2

In some embodiments, Form V of compound 2 has at least 1, 2, 3, 4 or 5 spectral peak(s) selected from the peaks listed in Table 7 below.

TABLE 7
XRPD Peak Positions for Form V of Compound 2

| Pos. [°2θ] | Rel. Int. [%] |
|---|---|
| 5.1 | 31.78 |
| 6.0 | 5.46 |
| 7.0 | 55.46 |
| 8.2 | 4.80 |
| 9.9 | 2.26 |
| 11.5 | 12.97 |
| 12.6 | 43.76 |
| 14.2 | 25.26 |
| 15.0 | 48.37 |
| 15.7 | 56.56 |
| 16.2 | 58.98 |
| 17.1 | 8.40 |
| 18.0 | 66.45 |
| 18.7 | 58.86 |
| 19.1 | 47.03 |
| 19.7 | 52.49 |
| 20.7 | 100.00 |
| 21.5 | 49.43 |
| 23.9 | 23.93 |
| 25.3 | 45.23 |
| 25.9 | 63.11 |
| 26.7 | 37.64 |
| 27.4 | 31.90 |
| 28.3 | 14.15 |
| 29.7 | 23.55 |
| 30.9 | 7.44 |
| 31.6 | 8.29 |
| 33.2 | 5.61 |
| 34.5 | 8.31 |
| 38.8 | 2.87 |

In some embodiments, Form V of compound 2 is characterized in that it has one or more peaks in its X-ray powder diffraction pattern selected from those at about 18.0, about 20.7 and about 25.9 degrees 2-theta. In some embodiments, Form V of compound 2 is characterized in that it has two or more peaks in its X-ray powder diffraction pattern selected from those at about 18.0, about 20.7 and about 25.9 degrees 2-theta. In some embodiments, Form V of compound 2 is characterized in that it has all three peaks in its X-ray powder diffraction pattern selected from those at about 18.0, about 20.7 and about 25.9 degrees 2-theta.

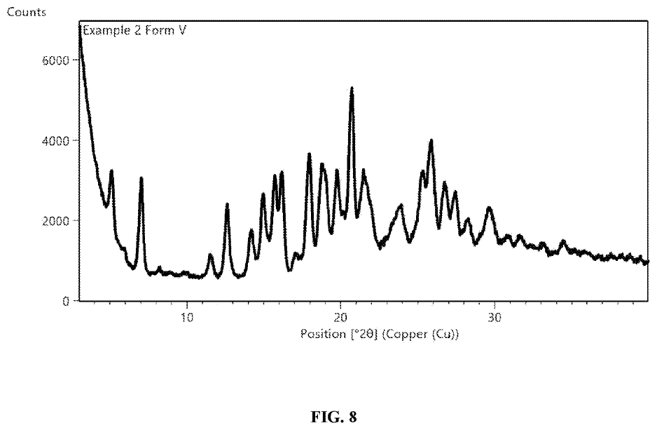

In certain embodiments, the X-ray powder diffraction pattern is substantially similar to the XRPD provided in FIG. 8.

Methods for preparing Form V of compound 2 are described infra.

### Form VI of Compound 2

In some embodiments, Form VI of compound 2 has at least 1, 2, 3, 4 or 5 spectral peak(s) selected from the peaks listed in Table 8 below.

TABLE 8
XRPD Peak Positions for Form VI of Compound 2

| Pos. [°2θ] | Rel. Int. [%] |
|---|---|
| 6.5 | 100.00 |
| 12.9 | 13.43 |
| 14.4 | 6.06 |
| 18.3 | 41.70 |
| 19.2 | 70.88 |
| 20.2 | 64.82 |
| 21.3 | 51.71 |
| 32.3 | 4.33 |

In some embodiments, Form VI of compound 2 is characterized in that it has one or more peaks in its X-ray powder diffraction pattern selected from those at about 6.5, about 19.2 and about 20.2 degrees 2-theta. In some embodiments, Form VI of compound 2 is characterized in that it has two or more peaks in its X-ray powder diffraction pattern selected from those at about 6.5, about 19.2 and about 20.2 degrees 2-theta. In some embodiments, Form VI of compound 2 is characterized in that it has all three peaks in its X-ray powder diffraction pattern selected from those at about 6.5, about 19.2 and about 20.2 degrees 2-theta.

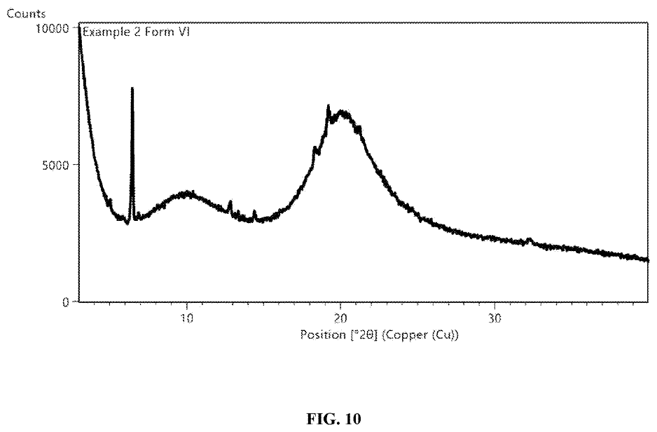

In certain embodiments, the X-ray powder diffraction pattern is substantially similar to the XRPD provided in FIG. 10.

Methods for preparing Form VI of compound 2 are described infra.

### Form VII of Compound 2

In some embodiments, Form VII of compound 2 has at least 1, 2, 3, 4 or 5 spectral peak(s) selected from the peaks listed in Table 9 below.

TABLE 9
XRPD Peak Positions for Form VII of Compound 2

| Pos. [°2θ] | Rel. Int. [%] |
|---|---|
| 5.0 | 11.63 |
| 6.0 | 3.23 |
| 6.6 | 8.48 |
| 8.2 | 0.33 |
| 9.9 | 2.94 |
| 10.4 | 32.26 |
| 11.9 | 0.21 |
| 13.0 | 1.84 |
| 13.9 | 1.84 |
| 14.4 | 1.15 |
| 14.8 | 100.00 |
| 15.2 | 7.47 |
| 16.2 | 0.92 |
| 16.3 | 1.02 |
| 17.0 | 0.39 |
| 17.7 | 0.78 |
| 19.0 | 1.42 |
| 19.5 | 13.27 |
| 20.2 | 5.25 |
| 20.8 | 5.25 |
| 22.0 | 11.31 |
| 24.7 | 9.21 |
| 24.9 | 71.35 |
| 25.6 | 3.27 |
| 26.1 | 2.85 |
| 26.6 | 4.77 |
| 28.0 | 1.15 |
| 29.7 | 6.68 |
| 29.9 | 6.08 |
| 30.3 | 2.35 |
| 31.4 | 1.56 |
| 32.6 | 3.64 |
| 33.1 | 1.41 |
| 34.0 | 0.80 |
| 34.8 | 3.22 |
| 35.4 | 1.13 |
| 35.7 | 1.14 |
| 36.2 | 1.38 |
| 37.3 | 0.81 |
| 38.6 | 1.41 |
| 39.6 | 0.50 |

In some embodiments, Form VII of compound 2 is characterized in that it has one or more peaks in its X-ray powder diffraction pattern selected from those at about 10.4, about 14.8 and about 24.9 degrees 2-theta. In some embodiments, Form VII of compound 2 is characterized in that it has two or more peaks in its X-ray powder diffraction pattern selected from those at about 10.4, about 14.8 and about 24.9 degrees 2-theta. In some embodiments, Form VII of compound 2 is characterized in that it has all three peaks in its X-ray powder diffraction pattern selected from those at about 10.4, about 14.8 and about 24.9 degrees 2-theta.

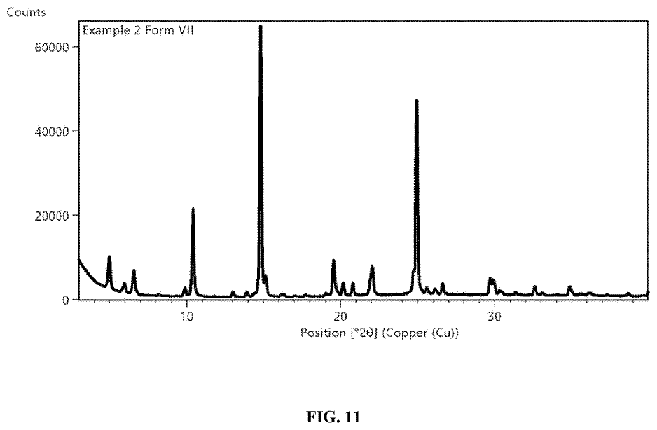

In certain embodiments, the X-ray powder diffraction pattern is substantially similar to the XRPD provided in FIG. 11.

Methods for preparing Form VII of compound 2 are described infra.

### Form VIII of Compound 2

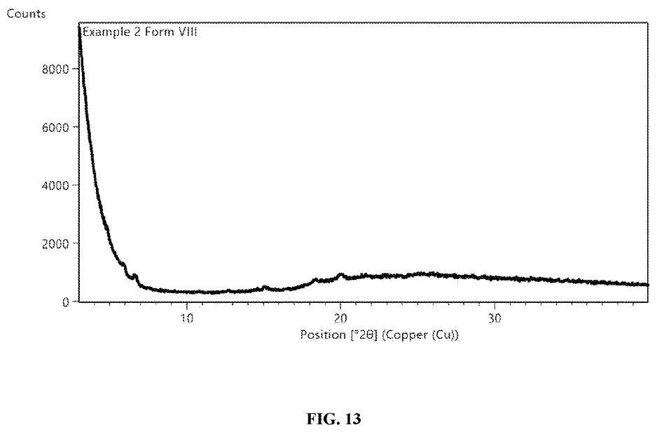

XRPD data indicated Form VIII of compound 2 is an amorphous form. In certain embodiments, the X-ray powder diffraction pattern is substantially similar to the XRPD provided in FIG. 13. It was observed that Form VIII of compound 2 transformed to Form I of compound 2 during storage at RT for about one month.

### Form IX of Compound 2

In some embodiments, Form IX of compound 2 has at least 1, 2, 3, 4 or 5 spectral peak(s) selected from the peaks listed in Table 10 below.

TABLE 10
XRPD Peak Positions for Form IX of Compound 2

| Pos. [°2θ] | Rel. Int. [%] |
|---|---|
| 6.3 | 100.00 |
| 6.7 | 24.97 |
| 12.6 | 5.78 |
| 14.6 | 5.25 |
| 15.0 | 12.61 |
| 17.8 | 10.38 |
| 18.3 | 18.10 |
| 18.9 | 45.02 |
| 19.9 | 23.15 |
| 21.1 | 9.47 |
| 25.1 | 5.04 |
| 31.7 | 3.22 |

In some embodiments, Form IX of compound 2 is characterized in that it has one or more peaks in its X-ray powder diffraction pattern selected from those at about 6.3, about 6.7 and about 18.9 degrees 2-theta. In some embodiments, Form IX of compound 2 is characterized in that it has two or more peaks in its X-ray powder diffraction pattern selected from those at about 6.3, about 6.7 and about 18.9 degrees 2-theta. In some embodiments, Form IX of compound 2 is characterized in that it has all three peaks in its X-ray powder diffraction pattern selected from those at about 6.3, about 6.7 and about 18.9 degrees 2-theta.

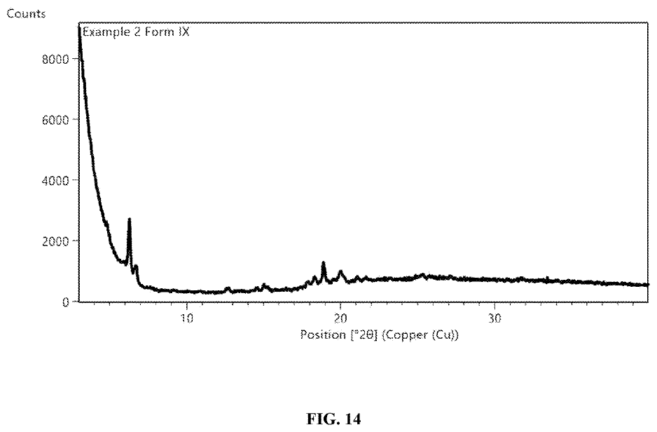

In certain embodiments, the X-ray powder diffraction pattern is substantially similar to the XRPD provided in FIG. 14.

Methods for preparing Form IX of compound 2 are described infra.

### Form X of Compound 2

In some embodiments, Form X of compound 2 has at least 1, 2, 3, 4 or 5 spectral peak(s) selected from the peaks listed in Table 11 below.

TABLE 11
XRPD Peak Positions for Form X of Compound 2

| Pos. [°2θ] | Rel. Int. [%] |
|---|---|
| 5.3 | 24.25 |
| 6.6 | 100.00 |
| 7.0 | 41.87 |
| 9.6 | 15.85 |
| 10.3 | 18.42 |
| 12.2 | 3.06 |
| 13.0 | 18.74 |
| 13.8 | 26.18 |
| 14.7 | 37.68 |
| 15.2 | 26.06 |
| 16.0 | 12.47 |
| 16.5 | 23.68 |
| 17.6 | 21.19 |
| 18.2 | 92.96 |
| 19.2 | 70.67 |
| 19.8 | 85.36 |
| 20.3 | 38.87 |
| 21.1 | 29.23 |
| 21.5 | 39.52 |
| 22.6 | 27.01 |
| 23.1 | 18.23 |
| 24.0 | 16.42 |
| 25.3 | 31.31 |
| 26.5 | 11.77 |
| 27.3 | 9.87 |
| 28.2 | 8.29 |
| 30.2 | 6.03 |
| 30.6 | 5.09 |
| 31.8 | 6.76 |
| 34.9 | 2.82 |
| 36.7 | 3.09 |

In some embodiments, Form X of compound 2 is characterized in that it has one or more peaks in its X-ray powder diffraction pattern selected from those at about 6.6, about 18.2 and about 19.8 degrees 2-theta. In some embodiments, Form X of compound 2 is characterized in that it has two or more peaks in its X-ray powder diffraction pattern selected from those at about 6.6, about 18.2 and about 19.8 degrees 2-theta. In some embodiments, Form X of compound 2 is characterized in that it has all three peaks in its X-ray powder diffraction pattern selected from those at about 6.6, about 18.2 and about 19.8 degrees 2-theta.

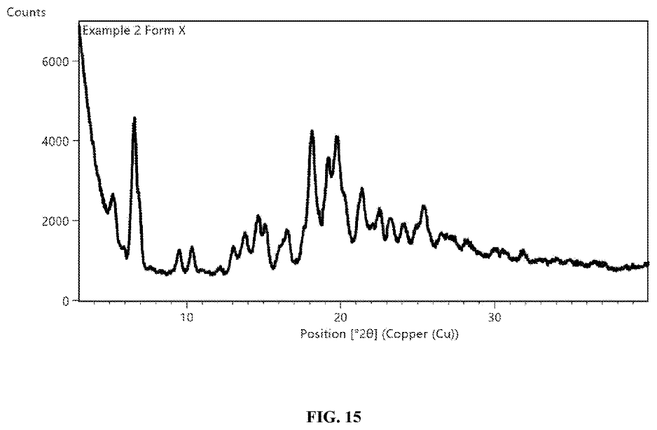

In certain embodiments, the X-ray powder diffraction pattern is substantially similar to the XRPD provided in FIG. 15.

Methods for preparing Form X of compound 2 are described infra.

### Form XI of Compound 2

In some embodiments, Form XI of compound 2 has at least 1, 2, 3, 4 or 5 spectral peak(s) selected from the peaks listed in Table 12 below.

TABLE 12
XRPD Peak Positions for Form XI of Compound 2

| Pos. [°2θ] | Rel. Int. [%] |
|---|---|
| 5.2 | 7.06 |
| 7.0 | 45.43 |
| 7.8 | 1.72 |
| 11.0 | 2.44 |
| 13.1 | 12.60 |
| 13.6 | 9.76 |
| 15.2 | 27.47 |
| 16.6 | 1.48 |
| 18.4 | 7.96 |
| 20.0 | 100.00 |
| 20.3 | 41.62 |
| 21.3 | 19.61 |
| 22.1 | 18.70 |
| 23.3 | 6.64 |
| 24.2 | 8.18 |
| 25.3 | 5.47 |
| 26.0 | 5.08 |
| 27.0 | 17.56 |
| 28.4 | 3.67 |
| 29.7 | 5.14 |
| 30.9 | 3.68 |
| 31.9 | 1.72 |
| 33.3 | 3.52 |
| 34.1 | 3.13 |
| 35.0 | 1.64 |
| 37.1 | 1.59 |

In some embodiments, Form XI of compound 2 is characterized in that it has one or more peaks in its X-ray powder diffraction pattern selected from those at about 7.0, about 20.0 and about 20.3 degrees 2-theta. In some embodiments, Form XI of compound 2 is characterized in that it has two or more peaks in its X-ray powder diffraction pattern selected from those at about 7.0, about 20.0 and about 20.3 degrees 2-theta. In some embodiments, Form XI of compound 2 is characterized in that it has all three peaks in its X-ray powder diffraction pattern selected from those at about 7.0, about 20.0 and about 20.3 degrees 2-theta.

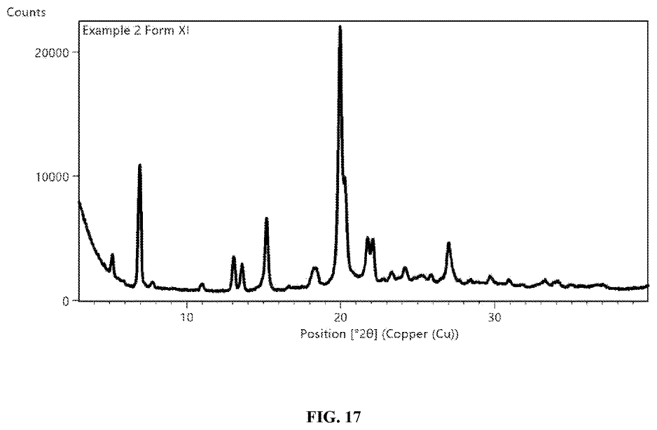

In certain embodiments, the X-ray powder diffraction pattern is substantially similar to the XRPD provided in FIG. 17.

Methods for preparing Form XI of compound 2 are described infra.

### Form XII of Compound 2

In some embodiments, Form XII of compound 2 has at least 1, 2, 3, 4 or 5 spectral peak(s) selected from the peaks listed in Table 13 below.

TABLE 13
XRPD Peak Positions for Form XII of Compound 2

| Pos. [°2θ] | Rel. Int. [%] |
|---|---|
| 5.8 | 100.00 |
| 5.9 | 69.25 |
| 8.6 | 1.17 |
| 9.0 | 0.97 |
| 10.3 | 1.23 |
| 11.5 | 9.82 |
| 12.2 | 2.11 |
| 12.5 | 5.65 |
| 13.4 | 10.91 |
| 14.2 | 6.82 |
| 15.0 | 14.07 |
| 15.3 | 6.94 |
| 15.8 | 17.30 |
| 16.0 | 11.92 |
| 16.4 | 5.13 |
| 16.7 | 11.16 |
| 16.8 | 14.16 |
| 17.1 | 18.55 |
| 17.4 | 11.14 |
| 18.1 | 22.70 |
| 18.6 | 11.84 |
| 18.9 | 7.03 |
| 19.3 | 22.76 |
| 20.1 | 17.67 |
| 20.6 | 17.20 |
| 21.4 | 9.99 |
| 21.6 | 12.63 |
| 21.8 | 13.31 |
| 22.2 | 18.68 |
| 22.4 | 14.59 |
| 23.0 | 16.52 |
| 23.3 | 17.08 |
| 23.7 | 14.68 |
| 24.4 | 6.94 |
| 25.0 | 4.77 |
| 25.6 | 5.06 |
| 26.2 | 6.37 |
| 26.5 | 3.93 |
| 27.1 | 5.46 |
| 27.8 | 8.51 |
| 28.4 | 11.99 |
| 29.1 | 8.09 |
| 29.6 | 6.24 |
| 29.9 | 2.79 |
| 30.2 | 4.58 |
| 31.0 | 4.31 |
| 31.9 | 3.42 |
| 33.0 | 1.91 |
| 34.4 | 1.83 |
| 36.5 | 1.09 |
| 37.1 | 1.94 |

In some embodiments, Form XII of compound 2 is characterized in that it has one or more peaks in its X-ray powder diffraction pattern selected from those at about 5.8, about 5.9 and about 19.3 degrees 2-theta. In some embodiments, Form XII of compound 2 is characterized in that it has two or more peaks in its X-ray powder diffraction pattern selected from those at about 5.8, about 5.9 and about 19.3 degrees 2-theta. In some embodiments, Form XII of compound 2 is characterized in that it has all three peaks in its X-ray powder diffraction pattern selected from those at about 5.8, about 5.9 and about 19.3 degrees 2-theta.

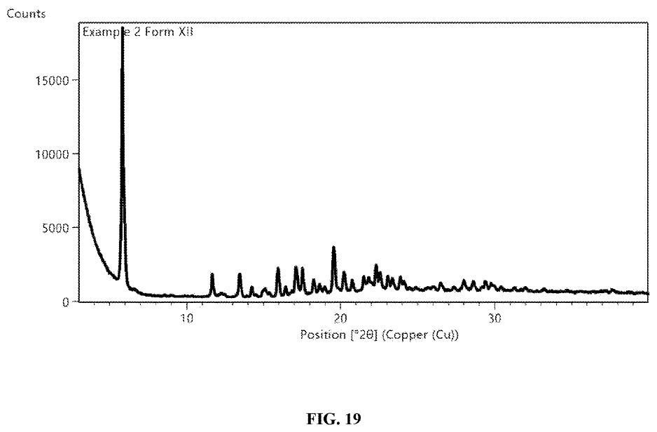

In certain embodiments, the X-ray powder diffraction pattern is substantially similar to the XRPD provided in FIG. 19.

Methods for preparing Form XII of compound 2 are described infra.

### Form XIII of Compound 2

In some embodiments, Form XIII of compound 2 has at least 1, 2, 3, 4 or 5 spectral peak(s) selected from the peaks listed in Table 14 below.

TABLE 14
XRPD Peak Positions for Form XIII of Compound 2

| Pos. [°2θ] | Rel. Int. [%] |
|---|---|
| 6.1 | 100.00 |
| 9.8 | 2.20 |
| 11.6 | 4.94 |
| 12.3 | 5.16 |
| 13.2 | 6.01 |
| 14.2 | 7.59 |
| 15.0 | 2.94 |
| 15.7 | 8.58 |
| 15.9 | 10.93 |
| 16.3 | 0.66 |
| 17.2 | 11.74 |
| 17.8 | 8.25 |
| 18.4 | 15.70 |
| 19.1 | 5.29 |
| 19.6 | 10.12 |
| 20.3 | 9.65 |
| 20.9 | 9.86 |
| 21.8 | 18.01 |
| 22.8 | 13.65 |
| 23.2 | 12.51 |
| 24.7 | 6.83 |
| 26.7 | 5.37 |
| 28.4 | 6.93 |
| 31.0 | 2.50 |

In some embodiments, Form XIII of compound 2 is characterized in that it has one or more peaks in its X-ray powder diffraction pattern selected from those at about 6.1, about 21.8 and about 22.8 degrees 2-theta. In some embodiments, Form XIII of compound 2 is characterized in that it has two or more peaks in its X-ray powder diffraction pattern selected from those at about 6.1, about 21.8 and about 22.8 degrees 2-theta. In some embodiments, Form XIII of compound 2 is characterized in that it has all three peaks in its X-ray powder diffraction pattern selected from those at about 6.1, about 21.8 and about 22.8 degrees 2-theta.

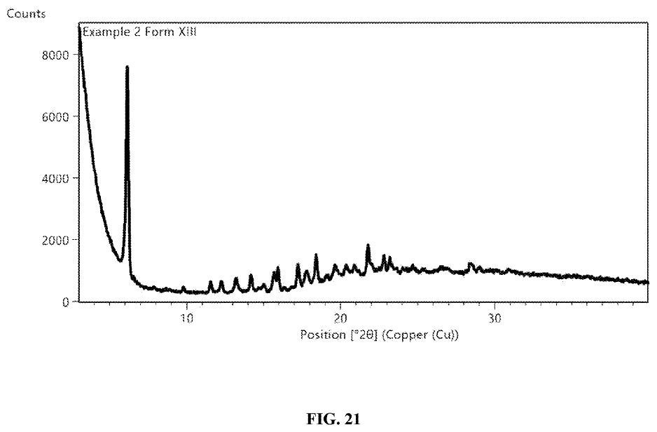

In certain embodiments, the X-ray powder diffraction pattern is substantially similar to the XRPD provided in FIG. 21.

Methods for preparing Form XIII of compound 2 are described infra.

In some embodiments, the present invention provides compound 2, wherein said compound is crystalline.

In some embodiments, the present invention provides compound 2, wherein said compound is a crystalline solid substantially free of amorphous compound 2.

In some embodiments, the present invention provides compound 2, wherein said compound is substantially free of impurities.

In some embodiments, the present invention provides a composition comprising Form I, II, III, IV, V, VI, VII, VIII, IX, X, XI, XII or XIII of compound 2 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I, II, III, IV, V, VI, VII, VIII, IX, X, XI, XII or XIII of compound 2 or composition thereof.

In some embodiments, the present invention provides a method of treating cancer in a patient by blocking the activity of kinases, comprising administering to said patient Form I, II, III, IV, V, VI, VII, VIII, IX, X, XI, XII or XIII of compound 2 or composition thereof wherein Form I, II, III, IV, V, VI, VII, VIII, IX, X, XI, XII or XIII of compound 2 is a crystal form as described herein.

### Compound 3 (Maleate Salts of Compound A)

According to one embodiment, the present invention provides a maleate salt of compound A, represented by compound 3:

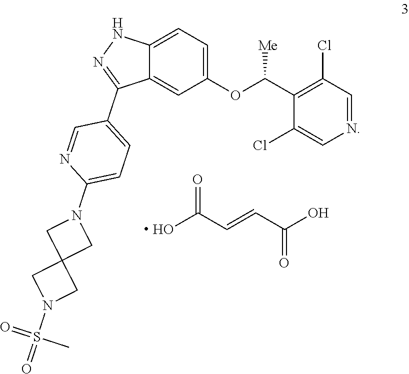

It will be appreciated by one of ordinary skill in the art that the maleic acid and compound A are ionically bonded to form compound 3. It is contemplated that compound 3 can exist in a variety of physical forms. For example, compound 3 can be in solution, suspension, or in solid form. In certain embodiments, compound 3 is in solid form. When compound 3 is in solid form, said compound may be amorphous, crystalline, or a mixture thereof. Exemplary solid forms are described in more detail below.

In some embodiments, the present invention provides compound 3 substantially free of impurities. As used herein, the term “substantially free of impurities” means that the compound contains no significant amount of extraneous matter. Such extraneous matter may include excess maleic acid, excess synthetic reagents, excess compound A, residual solvents, or any other impurities that may result from the preparation of, and/or isolation of, compound 3. In certain embodiments, at least about 95% by weight of compound 3 is present in a composition. In still other embodiments of the invention, at least about 99% by weight of compound 3 is present in a composition.

According to one embodiment, compound 3 is present in a composition in an amount of at least about 97, 97.5, 98.0, 98.5, 99, 99.5, 99.8 weight percent where the percentages are based on the total weight of the composition. According to another embodiment, compound 3 contains no more than about 3.0 area percent HPLC of total organic impurities and, in certain embodiments, no more than about 1.5 area percent HPLC total organic impurities relative to the total area of the HPLC chromatogram. In other embodiments, compound 3 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 3 is also meant to include all tautomeric forms and isomeric forms of compound 3.

It has been found that compound 3 can exist in a variety of solid forms. Exemplary such forms include polymorphs such as those described herein.

It has been found that compound 3 can exist in at least two polymorphic forms.

In some embodiments, the present invention provides a polymorphic form of Compound 3 referred to herein as Form I. In some embodiments, Form I of compound 3 is substantially free of other polymorphic forms.

In certain embodiments, the present invention provides a polymorphic form of compound 3 referred to herein as Form II. In some embodiments, Form II of compound 3 is substantially free of other polymorphic forms.

In certain embodiments, compound 3 is a crystalline solid. In other embodiments, compound 3 is a crystalline solid substantially free of amorphous compound 3. As used herein, the term “substantially free of amorphous compound 3” means that the compound contains no significant amount of amorphous compound 3. In certain embodiments, at least about 95% by weight of crystalline compound 3 is present. In still other embodiments of the invention, at least about 99% by weight of crystalline compound 3 is present.

In some embodiments, compound 3 is amorphous. In some embodiments, compound 3 is amorphous, and is substantially free of crystalline compound 3.

### Form I of Compound 3

In some embodiments, Form I of compound 3 has at least 1, 2, 3, 4 or 5 spectral peak(s) selected from the peaks listed in Table 15 below.

TABLE 15
XRPD Peak Positions for Form I of Compound 3

| Pos. [°2θ] | Int. I. (cps) |
|---|---|
| 4.9 | 284(18) |
| 6.2 | 6356(70) |
| 7.3 | 8657(47) |
| 9.9 | 682(19) |
| 12.5 | 2706(116) |
| 13.6 | 218(42) |
| 14.4 | 5298(76) |
| 14.9 | 5278(125) |
| 15.2 | 9250(169) |
| 15.8 | 910(269) |
| 16.1 | 5740(230) |
| 16.9 | 2080(50) |
| 17.1 | 3987(70) |
| 17.9 | 2194(47) |
| 18.4 | 4591(126) |
| 18.8 | 27152(1704) |
| 18.9 | 23725(1657) |
| 19.1 | 3249(523) |
| 19.8 | 2272(104) |
| 20.3 | 700(36) |
| 21.0 | 1233(26) |
| 21.4 | 1064(30) |
| 21.7 | 1599(49) |
| 21.9 | 967(93) |
| 22.1 | 617(79) |
| 22.6 | 6119(51) |
| 23.7 | 2378(81) |
| 24.1 | 11676(113) |
| 24.7 | 4255(225) |
| 25.1 | 5960(373) |
| 25.3 | 4847(267) |
| 25.5 | 19272(611) |
| 26.0 | 2754(71) |
| 26.5 | 4946(434) |
| 26.7 | 15174(569) |
| 27.0 | 1977(242) |
| 27.6 | 3722(49) |
| 29.1 | 3876(298) |
| 29.2 | 6614(309) |
| 29.7 | 3994(51) |
| 30.3 | 6155(59) |
| 30.8 | 3684(58) |
| 31.1 | 191(37) |
| 31.6 | 2371(40) |
| 32.9 | 200(27) |
| 33.2 | 763(29) |
| 33.5 | 1947(68) |
| 34.2 | 6405(95) |
| 34.6 | 2308(91) |
| 35.3 | 5280(80) |
| 36.6 | 825(74) |
| 36.9 | 1463(91) |
| 37.5 | 1832(63) |
| 38.4 | 3245(73) |
| 39.6 | 516(27) |
| 40.8 | 422(36) |
| 41.0 | 1123(55) |
| 41.7 | 120(20) |
| 42.2 | 1045(34) |
| 43.0 | 1097(38) |
| 43.9 | 1616(38) |
| 45.8 | 584(52) |
| 46.2 | 560(51) |
| 46.8 | 1434(65) |
| 47.7 | 158(22) |
| 48.2 | 145(33) |
| 48.5 | 892(39) |
| 49.0 | 837(71) |
| 49.4 | 1676(86) |
| 50.5 | 575(46) |
| 51.3 | 576(52) |
| 52.2 | 355(49) |
| 53.4 | 875(38) |
| 54.6 | 2339(82) |
| 57.5 | 914(69) |
| 58.5 | 188(29) |

In some embodiments, Form I of compound 3 is characterized in that it has one or more peaks in its X-ray powder diffraction pattern selected from those at about 18.8, about 25.5 and about 26.7 degrees 2-theta. In some embodiments, Form I of compound 3 is characterized in that it has two or more peaks in its X-ray powder diffraction pattern selected from those at about 18.8, about 25.5 and about 26.7 degrees 2-theta. In some embodiments, Form I of compound 3 is characterized in that it has all three peaks in its X-ray powder diffraction pattern selected from those at about 18.8, about 25.5 and about 26.7 degrees 2-theta.

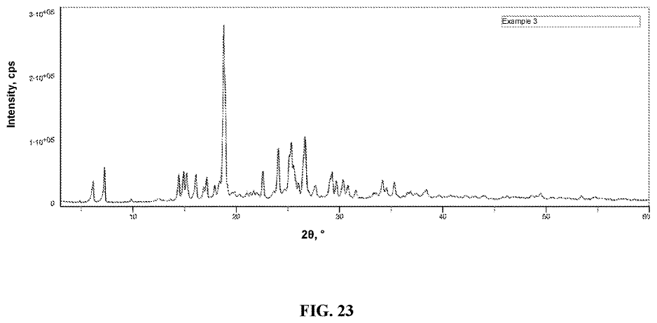

In certain embodiments, the X-ray powder diffraction pattern is substantially similar to the XRPD provided in FIG. 23.

Methods for preparing Form I of compound 3 are described infra.

### Form II of Compound 3

In some embodiments, Form II of compound 3 has at least 1, 2, 3, 4 or 5 spectral peak(s) selected from the peaks listed in Table 16 below.

TABLE 16
XRPD Peak Positions for Form II of Compound 3

| Pos. [°2θ] | Rel. Int. [%] |
|---|---|
| 5.9 | 6.62 |
| 7.2 | 3.30 |
| 7.7 | 1.16 |
| 11.5 | 8.29 |
| 13.6 | 2.43 |
| 14.5 | 5.10 |
| 15.2 | 100.00 |
| 16.7 | 10.50 |
| 18.6 | 91.20 |
| 19.4 | 12.89 |
| 20.3 | 15.85 |
| 20.9 | 38.76 |
| 21.6 | 17.12 |
| 22.2 | 12.86 |
| 22.8 | 15.59 |
| 23.6 | 4.81 |
| 24.4 | 7.97 |
| 24.9 | 8.75 |
| 25.8 | 10.48 |
| 26.5 | 5.49 |
| 27.1 | 13.06 |
| 27.7 | 21.02 |
| 28.4 | 20.89 |
| 28.7 | 5.57 |
| 29.7 | 7.00 |
| 29.9 | 6.89 |
| 30.3 | 7.08 |
| 30.5 | 8.96 |
| 30.9 | 9.98 |
| 32.8 | 22.21 |
| 34.4 | 5.09 |
| 35.1 | 4.96 |
| 35.5 | 4.46 |
| 36.4 | 10.29 |
| 37.7 | 4.34 |
| 38.2 | 13.47 |
| 39.3 | 3.41 |

In some embodiments, Form II of compound 3 is characterized in that it has one or more peaks in its X-ray powder diffraction pattern selected from those at about 15.2, about 18.6 and about 20.9 degrees 2-theta. In some embodiments, Form II of compound 3 is characterized in that it has two or more peaks in its X-ray powder diffraction pattern selected from those at about 15.2, about 18.6 and about 20.9 degrees 2-theta. In some embodiments, Form II of compound 3 is characterized in that it has all three peaks in its X-ray powder diffraction pattern selected from those at about 15.2, about 18.6 and about 20.9 degrees 2-theta.

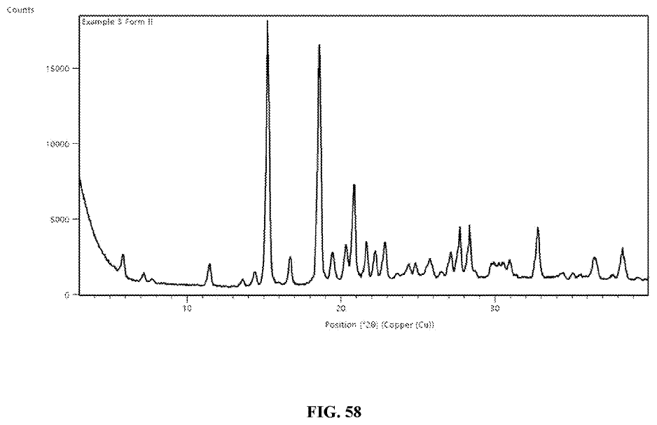

In certain embodiments, the X-ray powder diffraction pattern is substantially similar to the XRPD provided in FIG. 58.

Methods for preparing Form II of compound 3 are described infra.

In some embodiments, the present invention provides compound 3, wherein said compound is crystalline.

In some embodiments, the present invention provides compound 3, wherein said compound is a crystalline solid substantially free of amorphous compound 3.

In some embodiments, the present invention provides compound 3, wherein said compound is substantially free of impurities.

In some embodiments, the present invention provides a composition comprising Form I or Form II of compound 3 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I or Form II of compound 3 or composition thereof.

In some embodiments, the present invention provides a method of treating cancer in a patient by blocking the activity of kinases, comprising administering to said patient Form I or Form II of compound 3 or composition thereof wherein Form I or Form II of compound 3 is a crystal form as described herein.

### Compound 4 (Tosylate Salts of Compound A)

According to one embodiment, the present invention provides a tosylate salt of compound A, represented by compound 4:

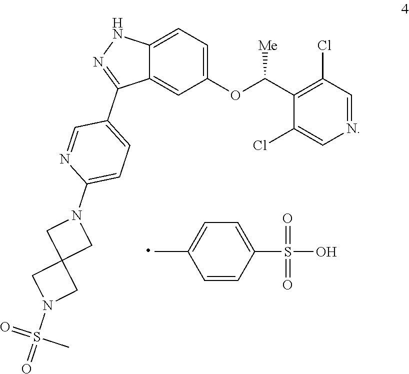 [SMILES: C[C@@H](Oc1ccc2[nH]nc(-c3ccc(N4CC5(C4)CN(S(C)(=O)=O)C5)nc3)c2c1)c1c(Cl)cncc1Cl ; Cc1ccc(S(=O)(=O)O)cc1]

By “tosylate” is meant p-toluene sulfonate, i.e., the ionic form of p-toluenesulfonic acid. It will be appreciated by one of ordinary skill in the art that the p-toluenesulfonic acid and compound A are ionically bonded to form compound 4. It is contemplated that compound 4 can exist in a variety of physical forms. For example, compound 4 can be in solution, suspension, or in solid form. In certain embodiments, compound 4 is in solid form. When compound 4 is in solid form, said compound may be amorphous, crystalline, or a mixture thereof. Exemplary solid forms are described in more detail below.

In some embodiments, the present invention provides compound 4 substantially free of impurities. As used herein, the term “substantially free of impurities” means that the compound contains no significant amount of extraneous matter. Such extraneous matter may include excess p-toluenesulfonic acid, excess synthetic reagents, excess compound A, residual solvents, or any other impurities that may result from the preparation of, and/or isolation of, compound 4. In certain embodiments, at least about 95% by weight of compound 4 is present in a composition. In still other embodiments of the invention, at least about 99% by weight of compound 4 is present in a composition.

According to one embodiment, compound 4 is present in a composition in an amount of at least about 97, 97.5, 98.0, 98.5, 99, 99.5, 99.8 weight percent where the percentages are based on the total weight of the composition. According to another embodiment, compound 4 contains no more than about 3.0 area percent HPLC of total organic impurities and, in certain embodiments, no more than about 1.5 area percent HPLC total organic impurities relative to the total area of the HPLC chromatogram. In other embodiments, compound 4 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 4 is also meant to include all tautomeric forms and isomeric forms of compound 4.

It has been found that compound 4 can exist in a variety of solid forms. Exemplary such forms include polymorphs such as those described herein.

It has been found that compound 4 can exist in at least one polymorphic form. In some embodiments, the present invention provides a polymorphic form of compound 4 referred to herein as Form I. In some embodiments, Form I of compound 4 is substantially free of other polymorphic forms.

In certain embodiments, compound 4 is a crystalline solid. In other embodiments, compound 4 is a crystalline solid substantially free of amorphous compound 4. As used herein, the term “substantially free of amorphous compound 4” means that the compound contains no significant amount of amorphous compound 4. In certain embodiments, at least about 95% by weight of crystalline compound 4 is present. In still other embodiments of the invention, at least about 99% by weight of crystalline compound 4 is present.

In some embodiments, compound 4 is amorphous. In some embodiments, compound 4 is amorphous, and is substantially free of crystalline compound 4.

### Form I of Compound 4

In some embodiments, Form I of compound 1 has at least 1, 2, 3, 4 or 5 spectral peak(s) selected from the peaks listed in Table 17 below.

TABLE 17
XRPD Peak Positions for Form I of Compound 4

| Pos. [°2θ] | Int. I. (cps) |
|---|---|
| 4.8 | 4690(227) |
| 6.2 | 54311(253) |
| 8.2 | 450(54) |
| 14.3 | 18673(927) |
| 17.8 | 35277(922) |
| 19.5 | 72093(411) |
| 25.7 | 38317(380) |
| 27.8 | 24015(382) |
| 31.3 | 899(132) |
| 51.4 | 2203(116) |

In some embodiments, Form I of compound 4 is characterized in that it has one or more peaks in its X-ray powder diffraction pattern selected from those at about 17.8, about 19.5 and about 25.7 degrees 2-theta. In some embodiments, Form I of compound 4 is characterized in that it has two or more peaks in its X-ray powder diffraction pattern selected from those at about 17.8, about 19.5 and about 25.7 degrees 2-theta. In some embodiments, Form I of compound 4 is characterized in that it has all three peaks in its X-ray powder diffraction pattern selected from those at about 17.8, about 19.5 and about 25.7 degrees 2-theta.

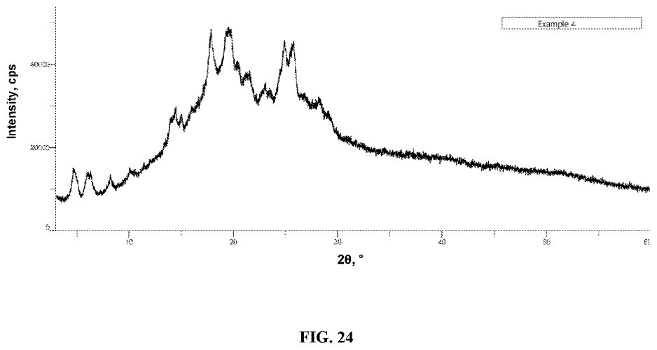

In certain embodiments, the X-ray powder diffraction pattern is substantially similar to the XRPD provided in FIG. 24.

Methods for preparing Form I compound 4 are described infra.

In some embodiments, the present invention provides compound 4, wherein said compound is crystalline.

In some embodiments, the present invention provides compound 4, wherein said compound is a crystalline solid substantially free of amorphous compound 4.

In some embodiments, the present invention provides compound 4, wherein said compound is substantially free of impurities.

In some embodiments, the present invention provides a composition comprising Form I of compound 4 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 4 or composition thereof.

In some embodiments, the present invention provides a method of treating cancer in a patient by blocking the activity of kinases, comprising administering to said patient Form I of compound 4 or composition thereof wherein Form I of compound 4 is a crystal form as described herein.

### Compound 5 (Sulfate Salts of Compound A)

According to one embodiment, the present invention provides a sulfate salt of compound A, represented by compound 5:

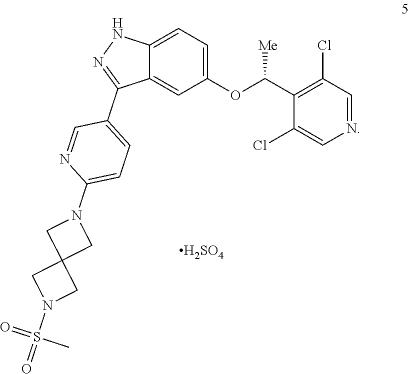 [SMILES: C[C@@H](Oc1ccc2[nH]nc(-c3ccc(N4CC5(C4)CN(S(C)(=O)=O)C5)nc3)c2c1)c1c(Cl)cncc1Cl ; "·H2SO4"]

It will be appreciated by one of ordinary skill in the art that the sulfuric acid and compound A are ionically bonded to form compound 5. It is contemplated that compound 5 can exist in a variety of physical forms. For example, compound 5 can be in solution, suspension, or in solid form. In certain embodiments, compound 5 is in solid form. When compound 5 is in solid form, said compound may be amorphous, crystalline, or a mixture thereof. Exemplary solid forms are described in more detail below.

In some embodiments, the present invention provides compound 5 substantially free of impurities. As used herein, the term “substantially free of impurities” means that the compound contains no significant amount of extraneous matter. Such extraneous matter may include excess sulfuric acid, excess synthetic reagents, excess compound A, residual solvents, or any other impurities that may result from the preparation of, and/or isolation of, compound 5. In certain embodiments, at least about 95% by weight of compound 5 is present in a composition. In still other embodiments of the invention, at least about 99% by weight of compound 5 is present in a composition.

According to one embodiment, compound 5 is present in a composition in an amount of at least about 97, 97.5, 98.0, 98.5, 99, 99.5, 99.8 weight percent where the percentages are based on the total weight of the composition. According to another embodiment, compound 5 contains no more than about 3.0 area percent HPLC of total organic impurities and, in certain embodiments, no more than about 1.5 area percent HPLC total organic impurities relative to the total area of the HPLC chromatogram. In other embodiments, compound 5 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 5 is also meant to include all tautomeric forms and isomeric forms of compound 5.

It has been found that compound 5 can exist in a variety of solid forms. Exemplary such forms include polymorphs such as those described herein.

It has been found that compound 5 can exist in at least two polymorphic forms.

In some embodiments, the present invention provides a polymorphic form of Compound 5 referred to herein as Form I. In some embodiments, Form I of compound 5 is substantially free of other polymorphic forms.

In certain embodiments, the present invention provides a polymorphic form of compound 5 referred to herein as Form II. In some embodiments, Form II of compound 5 is substantially free of other polymorphic forms.

In certain embodiments, compound 5 is a crystalline solid. In other embodiments, compound 5 is a crystalline solid substantially free of amorphous compound 5. As used herein, the term “substantially free of amorphous compound 5” means that the compound contains no significant amount of amorphous compound 5. In certain embodiments, at least about 95% by weight of crystalline compound 5 is present. In still other embodiments of the invention, at least about 99% by weight of crystalline compound 5 is present.

In some embodiments, compound 5 is amorphous. In some embodiments, compound 5 is amorphous, and is substantially free of crystalline compound 5.

### Form I of Compound 5

In some embodiments, Form I of compound 5 has at least 1, 2, 3, 4 or 5 spectral peak(s) selected from the peaks listed in Table 18 below.

TABLE 18
XRPD Peak Positions for Form I of Compound 5

| Pos. [°2θ] | Int. I. (cps) |
|---|---|
| 5.5 | 6513(57) |
| 7.3 | 251(21) |
| 12.0 | 90(30) |
| 13.4 | 4945(41) |
| 14.2 | 8543(67) |
| 14.7 | 8586(65) |
| 15.7 | 1640(36) |
| 16.4 | 7024(73) |
| 16.8 | 4885(126) |
| 17.2 | 3627(121) |
| 19.1 | 5061(104) |
| 20.0 | 27700(225) |
| 20.6 | 32930(226) |
| 21.6 | 2912(81) |
| 22.0 | 719(65) |
| 22.7 | 6423(189) |
| 23.3 | 2953(126) |
| 24.5 | 10203(133) |
| 25.2 | 7983(221) |
| 25.5 | 4630(388) |
| 25.9 | 9491(299) |
| 26.4 | 626(82) |
| 26.9 | 1085(79) |
| 27.3 | 5244(95) |
| 28.0 | 903(70) |
| 28.4 | 2010(107) |
| 28.9 | 1910(87) |
| 31.1 | 1903(131) |
| 32.3 | 767(94) |
| 33.2 | 1653(608) |
| 33.6 | 2411(606) |
| 34.4 | 1219(60) |
| 36.2 | 1405(75) |
| 36.8 | 934(57) |
| 37.8 | 982(178) |
| 38.4 | 2898(222) |
| 38.9 | 899(242) |
| 40.0 | 879(98) |
| 41.0 | 1197(73) |
| 42.3 | 356(76) |
| 44.3 | 633(45) |
| 45.0 | 1050(52) |
| 46.1 | 1698(307) |
| 46.6 | 904(311) |
| 47.7 | 213(18) |
| 50.4 | 642(138) |
| 51.1 | 2936(155) |
| 53.2 | 1117(66) |
| 55.4 | 391(49) |
| 58.4 | 646(55) |
| 59.1 | 143(43) |

In some embodiments, Form I of compound 5 is characterized in that it has one or more peaks in its X-ray powder diffraction pattern selected from those at about 20.0, about 20.6 and about 24.5 degrees 2-theta. In some embodiments, Form I of compound 5 is characterized in that it has two or more peaks in its X-ray powder diffraction pattern selected from those at about 20.0, about 20.6 and about 24.5 degrees 2-theta. In some embodiments, Form I of compound 5 is characterized in that it has all three peaks in its X-ray powder diffraction pattern selected from those at about 20.0, about 20.6 and about 24.5 degrees 2-theta.

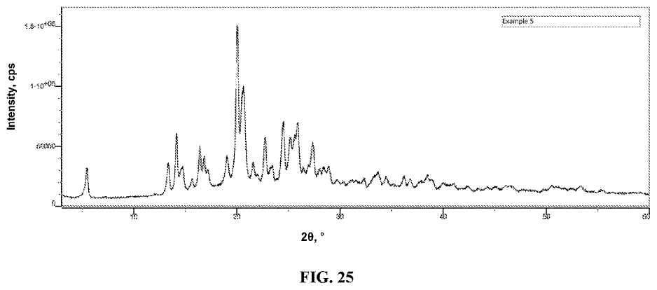

In certain embodiments, the X-ray powder diffraction pattern is substantially similar to the XRPD provided in FIG. 25.

Methods for preparing Form I compound 5 are described infra.

### Form II of Compound 5

In some embodiments, Form II of compound 5 has at least 1, 2, 3, 4 or 5 spectral peak(s) selected from the peaks listed in Table 19 below.

TABLE 19
XRPD Peak Positions for Form II of Compound 5

| Pos. [°2θ] | Rel. Int. %] |
|---|---|
| 5.8 | 12.69 |
| 8.7 | 0.80 |
| 12.4 | 0.91 |
| 13.7 | 17.45 |
| 14.5 | 28.06 |
| 15.1 | 21.74 |
| 16.0 | 8.07 |
| 16.7 | 28.78 |
| 17.2 | 22.90 |
| 17.5 | 15.24 |
| 18.9 | 15.59 |
| 19.4 | 26.63 |
| 20.4 | 100.00 |
| 21.1 | 71.79 |
| 22.0 | 22.25 |
| 23.1 | 31.80 |
| 23.9 | 16.43 |
| 24.8 | 36.99 |
| 25.5 | 37.70 |
| 26.2 | 38.46 |
| 27.8 | 27.73 |
| 28.3 | 16.40 |
| 29.3 | 18.26 |
| 30.1 | 9.80 |
| 31.4 | 8.83 |
| 32.6 | 8.74 |
| 34.0 | 12.25 |
| 34.8 | 8.63 |
| 36.6 | 7.80 |
| 37.2 | 6.67 |
| 38.7 | 7.13 |

In some embodiments, Form II of compound 5 is characterized in that it has one or more peaks in its X-ray powder diffraction pattern selected from those at about 20.4, about 21.1 and about 26.2 degrees 2-theta. In some embodiments, Form II of compound 5 is characterized in that it has two or more peaks in its X-ray powder diffraction pattern selected from those at about 20.4, about 21.1 and about 26.2 degrees 2-theta. In some embodiments, Form II of compound 5 is characterized in that it has all three peaks in its X-ray powder diffraction pattern selected from those at about 20.4, about 21.1 and about 26.2 degrees 2-theta.

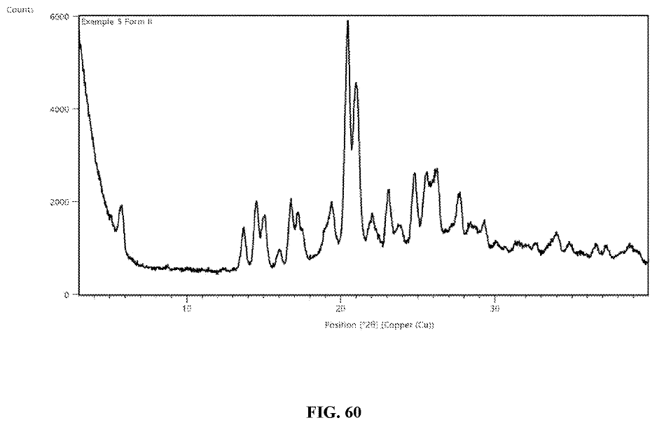

In certain embodiments, the X-ray powder diffraction pattern is substantially similar to the XRPD provided in FIG. 60.

Methods for preparing Form II of compound 5 are described infra.

In some embodiments, the present invention provides compound 5, wherein said compound is crystalline.

In some embodiments, the present invention provides compound 5, wherein said compound is a crystalline solid substantially free of amorphous compound 5.

In some embodiments, the present invention provides compound 5, wherein said compound is substantially free of impurities.

In some embodiments, the present invention provides a composition comprising Form I or Form II of compound 5 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I or Form II of compound 5 or composition thereof.

In some embodiments, the present invention provides a method of treating cancer in a patient by blocking the activity of kinases, comprising administering to said patient Form I or Form II of compound 5 or composition thereof wherein Form I or Form II of compound 5 is a crystal form as described herein.

### Compound 6 (2-Hydroxyethane Sulfonate Salts of Compound A)

According to one embodiment, the present invention provides a 2-hydroxyethane sulfonate salt of compound A, represented by compound 6:

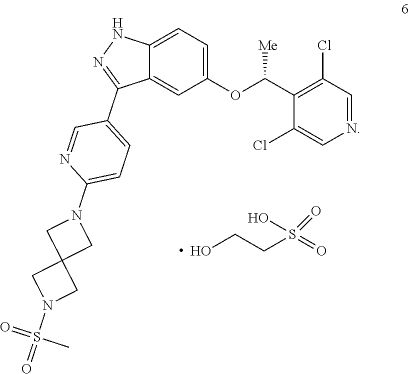 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=S(=O)(O)CCO]

It will be appreciated by one of ordinary skill in the art that the 2-hydroxyethane sulfonic acid and compound A are ionically bonded to form compound 6. It is contemplated that compound 6 can exist in a variety of physical forms. For example, compound 6 can be in solution, suspension, or in solid form. In certain embodiments, compound 6 is in solid form. When compound 6 is in solid form, said compound may be amorphous, crystalline, or a mixture thereof. Exemplary solid forms are described in more detail below.

In some embodiments, the present invention provides compound 6 substantially free of impurities. As used herein, the term “substantially free of impurities” means that the compound contains no significant amount of extraneous matter. Such extraneous matter may include excess 2-hydroxyethane sulfonic acid, excess synthetic reagents, excess compound A, residual solvents, or any other impurities that may result from the preparation of, and/or isolation of, compound 6. In certain embodiments, at least about 95% by weight of compound 6 is present in a composition. In still other embodiments of the invention, at least about 99% by weight of compound 6 is present in a composition.

According to one embodiment, compound 6 is present in a composition in an amount of at least about 97, 97.5, 98.0, 98.5, 99, 99.5, 99.8 weight percent where the percentages are based on the total weight of the composition. According to another embodiment, compound 6 contains no more than about 3.0 area percent HPLC of total organic impurities and, in certain embodiments, no more than about 1.5 area percent HPLC total organic impurities relative to the total area of the HPLC chromatogram. In other embodiments, compound 6 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 6 is also meant to include all tautomeric forms and isomeric forms of compound 6.

It has been found that compound 6 can exist in a variety of solid forms. Exemplary such forms include polymorphs such as those described herein.

It has been found that compound 6 can exist in at least one polymorphic form. In some embodiments, the present invention provides a polymorphic form of compound 6 referred to herein as Form I. In some embodiments, Form I of compound 6 is substantially free of other polymorphic forms.

In certain embodiments, compound 6 is a crystalline solid. In other embodiments, compound 6 is a crystalline solid substantially free of amorphous compound 6. As used herein, the term “substantially free of amorphous compound 6” means that the compound contains no significant amount of amorphous compound 6. In certain embodiments, at least about 95% by weight of crystalline compound 6 is present. In still other embodiments of the invention, at least about 99% by weight of crystalline compound 6 is present.

In some embodiments, compound 6 is amorphous. In some embodiments, compound 6 is amorphous, and is substantially free of crystalline compound 6.

### Form I of Compound 6

In some embodiments, Form I of compound 6 has at least 1, 2, 3, 4 or 5 spectral peak(s) selected from the peaks listed in Table 20 below.

TABLE 20
XRPD Peak Positions for Form I of Compound 6

| Pos. [°2θ] | Int. I. (cps) |
|---|---|
| 4.7 | 2276(155) |
| 4.9 | 1117(154) |
| 5.8 | 659(24) |
| 6.6 | 3628(31) |
| 10.0 | 472(56) |
| 11.6 | 1980(48) |
| 12.6 | 247(42) |
| 13.5 | 863(71) |
| 13.9 | 1113(80) |
| 14.7 | 2879(87) |
| 15.8 | 4362(101) |
| 16.4 | 1854(74) |
| 16.9 | 302(45) |
| 17.4 | 1343(45) |
| 18.5 | 915(107) |
| 18.8 | 1386(230) |
| 19.4 | 3378(170) |
| 20.1 | 11319(156) |
| 20.7 | 12797(258) |
| 21.7 | 1150(82) |
| 24.8 | 4223(268) |
| 25.6 | 7663(311) |
| 26.0 | 1192(167) |
| 26.7 | 1664(165) |
| 27.0 | 2340(190) |
| 28.0 | 163(43) |
| 28.4 | 1210(139) |
| 28.9 | 342(98) |
| 33.2 | 471(64) |
| 34.1 | 1628(83) |
| 35.5 | 558(48) |
| 37.6 | 451(62) |
| 38.7 | 629(53) |
| 41.0 | 1242(73) |

In some embodiments, Form I of compound 6 is characterized in that it has one or more peaks in its X-ray powder diffraction pattern selected from those at about 20.1, about 20.7 and about 25.6 degrees 2-theta. In some embodiments, Form I of compound 6 is characterized in that it has two or more peaks in its X-ray powder diffraction pattern selected from those at about 20.1, about 20.7 and about 25.6 degrees 2-theta. In some embodiments, Form I of compound 6 is characterized in that it has all three peaks in its X-ray powder diffraction pattern selected from those at about 20.1, about 20.7 and about 25.6 degrees 2-theta.

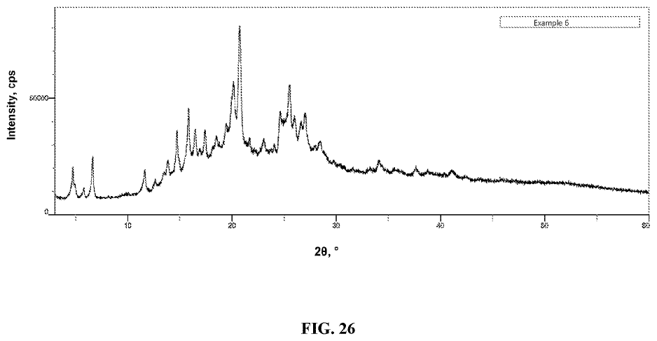

In certain embodiments, the X-ray powder diffraction pattern is substantially similar to the XRPD provided in FIG. 26.

Methods for preparing Form I compound 6 are described infra.

In some embodiments, the present invention provides compound 6, wherein said compound is crystalline.

In some embodiments, the present invention provides compound 6, wherein said compound is a crystalline solid substantially free of amorphous compound 6.

In some embodiments, the present invention provides compound 6, wherein said compound is substantially free of impurities.

In some embodiments, the present invention provides a composition comprising Form I of compound 6 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 6 or composition thereof.

In some embodiments, the present invention provides a method of treating cancer in a patient by blocking the activity of kinases, comprising administering to said patient Form I of compound 6 or composition thereof wherein Form I of compound 6 is a crystal form as described herein.

### Compound 7 (Esylate Salts of Compound A)

According to one embodiment, the present invention provides an esylate salt of compound A, represented by compound 7:

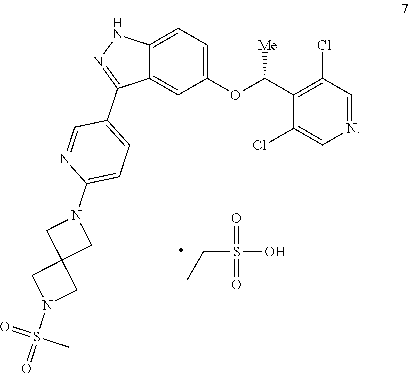

It will be appreciated by one of ordinary skill in the art that the ethane sulfonic acid and compound A are ionically bonded to form compound 7. It is contemplated that compound 7 can exist in a variety of physical forms. For example, compound 7 can be in solution, suspension, or in solid form. In certain embodiments, compound 7 is in solid form. When compound 7 is in solid form, said compound may be amorphous, crystalline, or a mixture thereof. Exemplary solid forms are described in more detail below.

In some embodiments, the present invention provides compound 7 substantially free of impurities. As used herein, the term “substantially free of impurities” means that the compound contains no significant amount of extraneous matter. Such extraneous matter may include excess ethane sulfonic acid, excess synthetic reagents, excess compound A, residual solvents, or any other impurities that may result from the preparation of, and/or isolation of, compound 7. In certain embodiments, at least about 95% by weight of compound 7 is present in a composition. In still other embodiments of the invention, at least about 99% by weight of compound 7 is present in a composition.

According to one embodiment, compound 7 is present in a composition in an amount of at least about 97, 97.5, 98.0, 98.5, 99, 99.5, 99.8 weight percent where the percentages are based on the total weight of the composition. According to another embodiment, compound 7 contains no more than about 3.0 area percent HPLC of total organic impurities and, in certain embodiments, no more than about 1.5 area percent HPLC total organic impurities relative to the total area of the HPLC chromatogram. In other embodiments, compound 7 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 7 is also meant to include all tautomeric forms and isomeric forms of compound 7.

It has been found that compound 7 can exist in a variety of solid forms. Exemplary such forms include polymorphs such as those described herein.

It has been found that compound 7 can exist in at least one polymorphic form. In some embodiments, the present invention provides a polymorphic form of compound 7 referred to herein as Form I. In some embodiments, Form I of compound 7 is substantially free of other polymorphic forms.

In certain embodiments, compound 7 is a crystalline solid. In other embodiments, compound 7 is a crystalline solid substantially free of amorphous compound 7. As used herein, the term “substantially free of amorphous compound 7” means that the compound contains no significant amount of amorphous compound 7. In certain embodiments, at least about 95% by weight of crystalline compound 7 is present. In still other embodiments of the invention, at least about 99% by weight of crystalline compound 7 is present.

In some embodiments, compound 7 is amorphous. In some embodiments, compound 7 is amorphous, and is substantially free of crystalline compound 7.

### Form I of Compound 7

In some embodiments, Form I of compound 7 has at least 1, 2, 3, 4 or 5 spectral peak(s) selected from the peaks listed in Table 21 below.

TABLE 21
XRPD Peak Positions for Form I of Compound 7

| Pos. [°2θ] | Int. I. (cps) |
|---|---|
| 4.8 | 183(32) |
| 5.2 | 868(44) |
| 6.6 | 3587(45) |
| 10.0 | 1023(40) |
| 12.3 | 173(29) |
| 13.2 | 1038(66) |
| 14.4 | 5426(82) |
| 15.8 | 414(76) |
| 16.2 | 1847(112) |
| 17.5 | 1265(130) |
| 17.8 | 1029(218) |
| 18.1 | 1564(215) |
| 18.7 | 6577(152) |
| 19.2 | 3040(100) |
| 20.1 | 1607(61) |
| 20.6 | 716(65) |
| 21.7 | 1380(85) |
| 23.6 | 996(77) |
| 24.6 | 1820(233) |
| 25.4 | 11781(378) |
| 26.4 | 5524(239) |
| 27.0 | 4023(165) |
| 29.2 | 777(69) |
| 32.0 | 635(38) |
| 33.9 | 259(41) |
| 34.5 | 1089(76) |
| 36.0 | 154(35) |
| 40.6 | 143(34) |
| 41.6 | 636(59) |
| 44.3 | 171(26) |
| 46.1 | 156(37) |
| 47.7 | 146(30) |

In some embodiments, Form I of compound 7 is characterized in that it has one or more peaks in its X-ray powder diffraction pattern selected from those at about 18.7, about 25.4 and about 26.4 degrees 2-theta. In some embodiments, Form I of compound 7 is characterized in that it has two or more peaks in its X-ray powder diffraction pattern selected from those at about 18.7, about 25.4 and about 26.4 degrees 2-theta. In some embodiments, Form I of compound 7 is characterized in that it has all three peaks in its X-ray powder diffraction pattern selected from those at about 18.7, about 25.4 and about 26.4 degrees 2-theta.

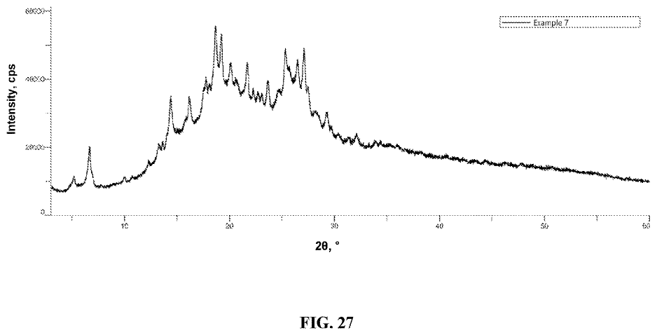

In certain embodiments, the X-ray powder diffraction pattern is substantially similar to the XRPD provided in FIG. 27.

Methods for preparing Form I compound 7 are described infra.

In some embodiments, the present invention provides compound 7, wherein said compound is crystalline.

In some embodiments, the present invention provides compound 7, wherein said compound is a crystalline solid substantially free of amorphous compound 7.

In some embodiments, the present invention provides compound 7, wherein said compound is substantially free of impurities.

In some embodiments, the present invention provides a composition comprising Form I of compound 7 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 7 or composition thereof.

In some embodiments, the present invention provides a method of treating cancer in a patient by blocking the activity of kinases, comprising administering to said patient Form I of compound 7 or composition thereof wherein Form I of compound 7 is a crystal form as described herein.

### Compound 8 (Mesylate Salts of Compound A)

According to one embodiment, the present invention provides a mesylate salt of compound A, represented by compound 8:

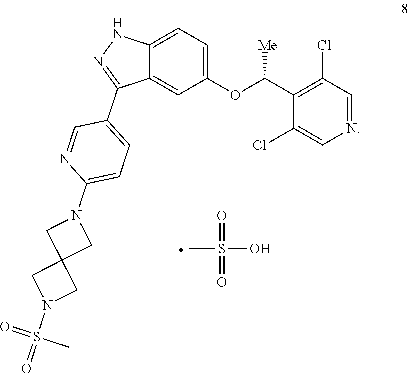

It will be appreciated by one of ordinary skill in the art that the methane sulfonic acid and compound A are ionically bonded to form compound 8. It is contemplated that compound 8 can exist in a variety of physical forms. For example, compound 8 can be in solution, suspension, or in solid form. In certain embodiments, compound 8 is in solid form. When compound 8 is in solid form, said compound may be amorphous, crystalline, or a mixture thereof. Exemplary solid forms are described in more detail below.

In some embodiments, the present invention provides compound 8 substantially free of impurities. As used herein, the term “substantially free of impurities” means that the compound contains no significant amount of extraneous matter. Such extraneous matter may include excess methane sulfonic acid, excess synthetic reagents, excess compound A, residual solvents, or any other impurities that may result from the preparation of, and/or isolation of, compound 8. In certain embodiments, at least about 95% by weight of compound 8 is present in a composition. In still other embodiments of the invention, at least about 99% by weight of compound 8 is present in a composition.

According to one embodiment, compound 8 is present in a composition in an amount of at least about 97, 97.5, 98.0, 98.5, 99, 99.5, 99.8 weight percent where the percentages are based on the total weight of the composition. According to another embodiment, compound 8 contains no more than about 3.0 area percent HPLC of total organic impurities and, in certain embodiments, no more than about 1.5 area percent HPLC total organic impurities relative to the total area of the HPLC chromatogram. In other embodiments, compound 8 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 8 is also meant to include all tautomeric forms and isomeric forms of compound 8.

It has been found that compound 8 can exist in a variety of solid forms. Exemplary such forms include polymorphs such as those described herein.

It has been found that compound 8 can exist in at least one polymorphic form. In some embodiments, the present invention provides a polymorphic form of compound 8 referred to herein as Form I. In some embodiments, Form I of compound 8 is substantially free of other polymorphic forms.

In certain embodiments, compound 8 is a crystalline solid. In other embodiments, compound 8 is a crystalline solid substantially free of amorphous compound 8. As used herein, the term “substantially free of amorphous compound 8” means that the compound contains no significant amount of amorphous compound 8. In certain embodiments, at least about 95% by weight of crystalline compound 8 is present. In still other embodiments of the invention, at least about 99% by weight of crystalline compound 8 is present.

In some embodiments, compound 8 is amorphous. In some embodiments, compound 8 is amorphous, and is substantially free of crystalline compound 8.

### Form I of Compound 8

In some embodiments, Form I of compound 8 has at least 1, 2, 3, 4 or 5 spectral peak(s) selected from the peaks listed in Table 22 below.

TABLE 22
XRPD Peak Positions for Form I of Compound 8

| Pos. [°2θ] | Int. I. (cps) |
|---|---|
| 5.0 | 2567(46) |
| 6.9 | 3400(45) |
| 10.0 | 518(56) |
| 11.7 | 484(47) |
| 14.2 | 2223(146) |
| 16.1 | 1174(149) |
| 20.0 | 13723(880) |
| 21.2 | 4446(856) |
| 25.0 | 5231(219) |
| 25.6 | 6827(209) |
| 26.8 | 3362(89) |
| 27.5 | 494(86) |
| 28.3 | 1948(85) |
| 32.0 | 387(44) |
| 33.2 | 313(43) |
| 34.3 | 758(64) |

In some embodiments, Form I of compound 8 is characterized in that it has one or more peaks in its X-ray powder diffraction pattern selected from those at about 20.0, about 25.0 and about 25.6 degrees 2-theta. In some embodiments, Form I of compound 8 is characterized in that it has two or more peaks in its X-ray powder diffraction pattern selected from those at about 20.0, about 25.0 and about 25.6 degrees 2-theta. In some embodiments, Form I of compound 8 is characterized in that it has all three peaks in its X-ray powder diffraction pattern selected from those at about 20.0, about 25.0 and about 25.6 degrees 2-theta.

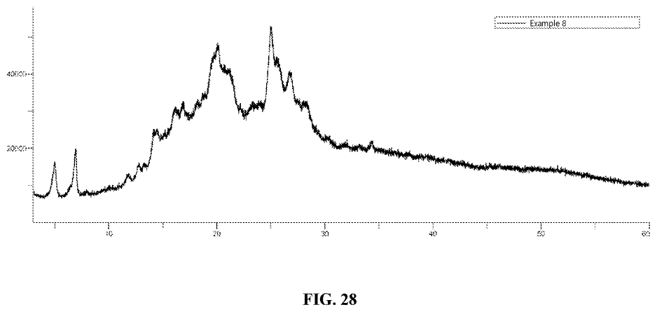

In certain embodiments, the X-ray powder diffraction pattern is substantially similar to the XRPD provided in FIG. 28.

Methods for preparing Form I compound 8 are described infra.

In some embodiments, the present invention provides compound 8, wherein said compound is crystalline.

In some embodiments, the present invention provides compound 8, wherein said compound is a crystalline solid substantially free of amorphous compound 8.

In some embodiments, the present invention provides compound 8, wherein said compound is substantially free of impurities.

In some embodiments, the present invention provides a composition comprising Form I of compound 8 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 8 or composition thereof.

In some embodiments, the present invention provides a method of treating cancer in a patient by blocking the activity of kinases, comprising administering to said patient Form I of compound 8 or composition thereof wherein Form I of compound 8 is a crystal form as described herein.

### Compound 9 (Di-Mesylate Salts of Compound A)

According to one embodiment, the present invention provides a di-mesylate salt of compound A, represented by compound 9:

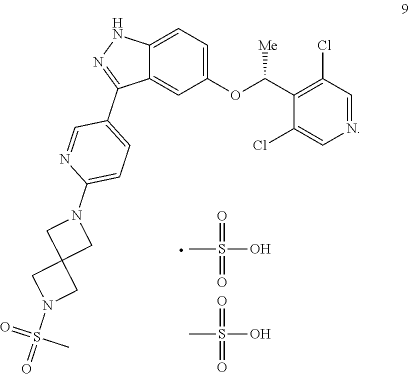

It will be appreciated by one of ordinary skill in the art that the methane sulfonic acids and compound A are ionically bonded to form compound 9. It is contemplated that compound 9 can exist in a variety of physical forms. For example, compound 9 can be in solution, suspension, or in solid form. In certain embodiments, compound 9 is in solid form. When compound 9 is in solid form, said compound may be amorphous, crystalline, or a mixture thereof. Exemplary solid forms are described in more detail below.

In some embodiments, the present invention provides compound 9 substantially free of impurities. As used herein, the term “substantially free of impurities” means that the compound contains no significant amount of extraneous matter. Such extraneous matter may include excess methane sulfonic acid, excess synthetic reagents, excess compound A, residual solvents, or any other impurities that may result from the preparation of, and/or isolation of, compound 9. In certain embodiments, at least about 95% by weight of compound 9 is present in a composition. In still other embodiments of the invention, at least about 99% by weight of compound 9 is present in a composition.

According to one embodiment, compound 9 is present in a composition in an amount of at least about 97, 97.5, 98.0, 98.5, 99, 99.5, 99.8 weight percent where the percentages are based on the total weight of the composition. According to another embodiment, compound 9 contains no more than about 3.0 area percent HPLC of total organic impurities and, in certain embodiments, no more than about 1.5 area percent HPLC total organic impurities relative to the total area of the HPLC chromatogram. In other embodiments, compound 9 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 9 is also meant to include all tautomeric forms and isomeric forms of compound 9.

It has been found that compound 9 can exist in a variety of solid forms. Exemplary such forms include polymorphs such as those described herein. In some embodiments, compound 9 is substantially free of other polymorphic forms.

In certain embodiments, compound 9 is a crystalline solid. In other embodiments, compound 9 is a crystalline solid substantially free of amorphous compound 9. As used herein, the term “substantially free of amorphous compound 9” means that the compound contains no significant amount of amorphous compound 9. In certain embodiments, at least about 95% by weight of crystalline compound 9 is present. In still other embodiments of the invention, at least about 99% by weight of crystalline compound 9 is present.

In some embodiments, compound 9 is amorphous. In some embodiments, compound 9 is amorphous, and is substantially free of crystalline compound 9.

Methods for preparing Form I compound 9 are described infra.

In some embodiments, the present invention provides compound 9, wherein said compound is crystalline.

In some embodiments, the present invention provides compound 9, wherein said compound is a crystalline solid substantially free of amorphous compound 9.

In some embodiments, the present invention provides compound 9, wherein said compound is substantially free of impurities.

In some embodiments, the present invention provides a composition comprising Form I of compound 9 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 9 or composition thereof.

In some embodiments, the present invention provides a method of treating cancer in a patient by blocking the activity of kinases, comprising administering to said patient Form I of compound 9 or composition thereof wherein Form I of compound 9 is a crystal form as described herein.

### Compound 10 (R-Camsylate Salts of Compound A)

According to one embodiment, the present invention provides an R-camsylate salt of compound A, represented by compound 10:

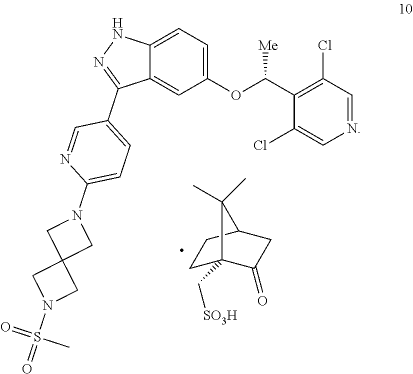

It will be appreciated by one of ordinary skill in the art that the R-camphor sulfonic acid and compound A are ionically bonded to form compound 10. It is contemplated that compound 10 can exist in a variety of physical forms. For example, compound 10 can be in solution, suspension, or in solid form. In certain embodiments, compound 10 is in solid form. When compound 10 is in solid form, said compound may be amorphous, crystalline, or a mixture thereof. Exemplary solid forms are described in more detail below.

In some embodiments, the present invention provides compound 10 substantially free of impurities. As used herein, the term “substantially free of impurities” means that the compound contains no significant amount of extraneous matter. Such extraneous matter may include excess R-camphor acid, excess synthetic reagents, excess compound A, residual solvents, or any other impurities that may result from the preparation of, and/or isolation of, compound 10. In certain embodiments, at least about 95% by weight of compound 10 is present in a composition. In still other embodiments of the invention, at least about 99% by weight of compound 10 is present in a composition.

According to one embodiment, compound 10 is present in a composition in an amount of at least about 97, 97.5, 98.0, 98.5, 99, 99.5, 99.8 weight percent where the percentages are based on the total weight of the composition. According to another embodiment, compound 10 contains no more than about 3.0 area percent HPLC of total organic impurities and, in certain embodiments, no more than about 1.5 area percent HPLC total organic impurities relative to the total area of the HPLC chromatogram. In other embodiments, compound 10 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 10 is also meant to include all tautomeric forms and isomeric forms of compound 10.

It has been found that compound 10 can exist in a variety of solid forms. Exemplary such forms include polymorphs such as those described herein.

It has been found that compound 10 can exist in at least one polymorphic form. In some embodiments, the present invention provides a polymorphic form of compound 10 referred to herein as Form I. In some embodiments, Form I of compound 10 is substantially free of other polymorphic forms.

In certain embodiments, compound 10 is a crystalline solid. In other embodiments, compound 10 is a crystalline solid substantially free of amorphous compound 10. As used herein, the term “substantially free of amorphous compound 10” means that the compound contains no significant amount of amorphous compound 10. In certain embodiments, at least about 95% by weight of crystalline compound 10 is present. In still other embodiments of the invention, at least about 99% by weight of crystalline compound 10 is present.

In some embodiments, compound 10 is amorphous. In some embodiments, compound 10 is amorphous, and is substantially free of crystalline compound 10.

### Form I of Compound 10

In some embodiments, Form I of compound 10 has at least 1, 2, 3, 4 or 5 spectral peak(s) selected from the peaks listed in Table 23 below.

TABLE 23
XRPD Peak Positions for Form I of Compound 10

| Pos. [°2θ] | Int. I. (cps) |
|---|---|
| 3.9 | 2000(60) |
| 4.4 | 3761(63) |
| 5.4 | 3038(37) |
| 6.4 | 1584(71) |
| 6.8 | 2431(102) |
| 7.3 | 710(79) |
| 12.7 | 6433(96) |
| 14.0 | 7612(247) |
| 14.6 | 12020(246) |
| 15.7 | 3748(115) |
| 16.7 | 1606(86) |
| 18.4 | 2590(55) |
| 19.5 | 2956(68) |
| 20.4 | 5956(98) |
| 22.2 | 2847(88) |
| 22.8 | 339(60) |
| 24.8 | 5782(210) |
| 25.4 | 13235(804) |
| 26.5 | 12678(768) |
| 28.1 | 7260(139) |
| 29.8 | 3756(109) |
| 32.2 | 424(47) |
| 33.4 | 1000(87) |
| 35.4 | 272(46) |
| 39.4 | 1643(91) |
| 52.0 | 1425(114) |

In some embodiments, Form I of compound 10 is characterized in that it has one or more peaks in its X-ray powder diffraction pattern selected from those at about 14.6, about 25.4 and about 26.5 degrees 2-theta. In some embodiments, Form I of compound 10 is characterized in that it has two or more peaks in its X-ray powder diffraction pattern selected from those at about 14.6, about 25.4 and about 26.5 degrees 2-theta. In some embodiments, Form I of compound 10 is characterized in that it has all three peaks in its X-ray powder diffraction pattern selected from those at about 14.6, about 25.4 and about 26.5 degrees 2-theta.

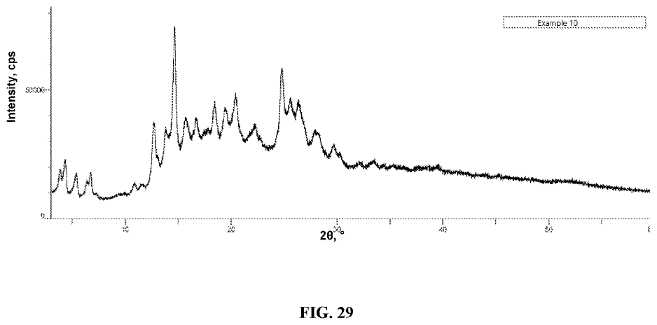

In certain embodiments, the X-ray powder diffraction pattern is substantially similar to the XRPD provided in FIG. 29.

Methods for preparing Form I of compound 10 are described infra.

In some embodiments, the present invention provides compound 10, wherein said compound is crystalline.

In some embodiments, the present invention provides compound 10, wherein said compound is a crystalline solid substantially free of amorphous compound 10.

In some embodiments, the present invention provides compound 10, wherein said compound is substantially free of impurities.

In some embodiments, the present invention provides a composition comprising Form I of compound 10 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 10 or composition thereof.

In some embodiments, the present invention provides a method of treating cancer in a patient by blocking the activity of kinases, comprising administering to said patient Form I of compound 10 or composition thereof wherein Form I of compound 10 is a crystal form as described herein.

### Compound 11 (S-Camsylate Salts of Compound A)

According to one embodiment, the present invention provides an S-camsylate salt of compound A, represented by compound 11:

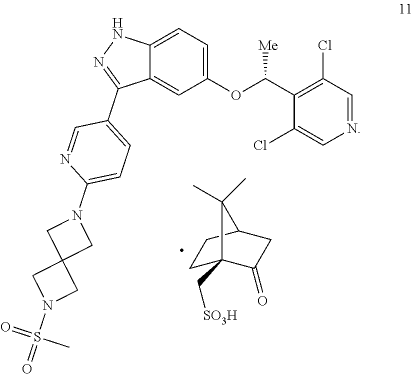

It will be appreciated by one of ordinary skill in the art that the S-camphor sulfonic acid and compound A are ionically bonded to form compound 11. It is contemplated that compound 11 can exist in a variety of physical forms. For example, compound 11 can be in solution, suspension, or in solid form. In certain embodiments, compound 11 is in solid form. When compound 11 is in solid form, said compound may be amorphous, crystalline, or a mixture thereof. Exemplary solid forms are described in more detail below.

In some embodiments, the present invention provides compound 11 substantially free of impurities. As used herein, the term “substantially free of impurities” means that the compound contains no significant amount of extraneous matter. Such extraneous matter may include excess S-camphor acid, excess synthetic reagents, excess compound A, residual solvents, or any other impurities that may result from the preparation of, and/or isolation of, compound 11. In certain embodiments, at least about 95% by weight of compound 11 is present in a composition. In still other embodiments of the invention, at least about 99% by weight of compound 11 is present in a composition.

According to one embodiment, compound 11 is present in a composition in an amount of at least about 97, 97.5, 98.0, 98.5, 99, 99.5, 99.8 weight percent where the percentages are based on the total weight of the composition. According to another embodiment, compound 11 contains no more than about 3.0 area percent HPLC of total organic impurities and, in certain embodiments, no more than about 1.5 area percent HPLC total organic impurities relative to the total area of the HPLC chromatogram. In other embodiments, compound 11 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 11 is also meant to include all tautomeric forms and isomeric forms of compound 11.

It has been found that compound 11 can exist in a variety of solid forms. Exemplary such forms include polymorphs such as those described herein.

It has been found that compound 11 can exist in at least one polymorphic form. In some embodiments, the present invention provides a polymorphic form of compound 11 referred to herein as Form I. In some embodiments, Form I of compound 11 is substantially free of other polymorphic forms.

In certain embodiments, compound 11 is a crystalline solid. In other embodiments, compound 11 is a crystalline solid substantially free of amorphous compound 11. As used herein, the term “substantially free of amorphous compound 11” means that the compound contains no significant amount of amorphous compound 11. In certain embodiments, at least about 95% by weight of crystalline compound 11 is present. In still other embodiments of the invention, at least about 99% by weight of crystalline compound 11 is present.

In some embodiments, compound 11 is amorphous. In some embodiments, compound 11 is amorphous, and is substantially free of crystalline compound 11.

### Form I of Compound 11

In some embodiments, Form I of compound 11 has at least 1, 2, 3, 4 or 5 spectral peak(s) selected from the peaks listed in Table 24 below.

TABLE 24
XRPD Peak Positions for Form I of Compound 11

| Pos. [°2θ] | Int. I. (cps) |
|---|---|
| 5.0 | 1450(99) |
| 5.3 | 6923(102) |
| 5.8 | 8981(100) |
| 8.2 | 587(129) |
| 8.9 | 1127(142) |
| 10.6 | 1755(43) |
| 11.9 | 1049(39) |
| 12.7 | 2026(73) |
| 14.0 | 1434(59) |
| 14.4 | 1199(47) |
| 15.4 | 9720(212) |
| 15.9 | 4663(196) |
| 17.5 | 12649(116) |
| 19.4 | 2088(218) |
| 20.1 | 3132(354) |
| 21.1 | 4287(280) |
| 21.3 | 5897(355) |
| 25.1 | 4377(194) |
| 26.0 | 12841(530) |
| 26.9 | 2181(449) |
| 29.3 | 958(78) |
| 32.0 | 616(314) |
| 33.0 | 662(187) |
| 34.1 | 995(125) |

In some embodiments, Form I of compound 11 is characterized in that it has one or more peaks in its X-ray powder diffraction pattern selected from those at about 15.4, about 17.5 and about 26.0 degrees 2-theta. In some embodiments, Form I of compound 11 is characterized in that it has two or more peaks in its X-ray powder diffraction pattern selected from those at about 15.4, about 17.5 and about 26.0 degrees 2-theta. In some embodiments, Form I of compound 11 is characterized in that it has all three peaks in its X-ray powder diffraction pattern selected from those at about 15.4, about 17.5 and about 26.0 degrees 2-theta.

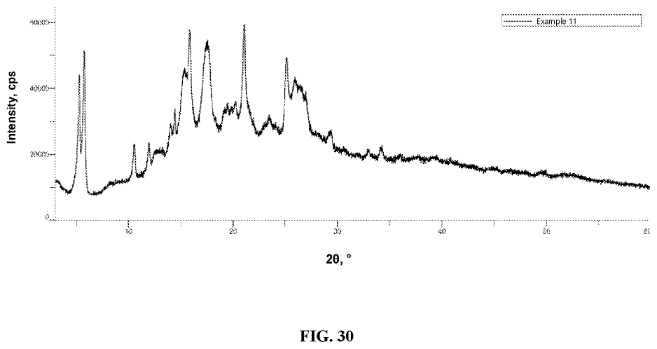

In certain embodiments, the X-ray powder diffraction pattern is substantially similar to the XRPD provided in FIG. 30.

Methods for preparing Form I of compound 11 are described infra.

In some embodiments, the present invention provides compound 11, wherein said compound is crystalline.

In some embodiments, the present invention provides compound 11, wherein said compound is a crystalline solid substantially free of amorphous compound 11.

In some embodiments, the present invention provides compound 11, wherein said compound is substantially free of impurities.

In some embodiments, the present invention provides a composition comprising Form I of compound 11 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 11 or composition thereof.

In some embodiments, the present invention provides a method of treating cancer in a patient by blocking the activity of kinases, comprising administering to said patient Form I of compound 11 or composition thereof wherein Form I of compound 11 is a crystal form as described herein.

### Compound 12 (Hydrobromide Salts of Compound A)

According to one embodiment, the present invention provides an hydrobromide salt of compound A, represented by compound 12:

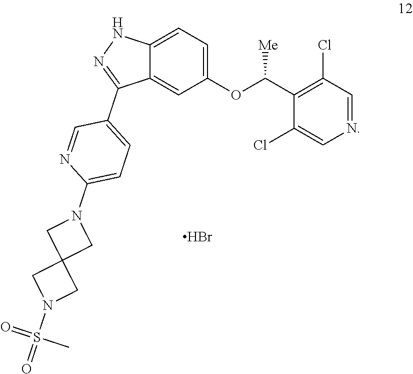 [SMILES: C[C@@H](Oc1ccc2[nH]nc(-c3ccc(N4CC5(C4)CN(S(C)(=O)=O)C5)nc3)c2c1)c1c(Cl)cncc1Cl ; Br]

It will be appreciated by one of ordinary skill in the art that the S-camphor sulfonic acid and compound A are ionically bonded to form compound 12. It is contemplated that compound 12 can exist in a variety of physical forms. For example, compound 12 can be in solution, suspension, or in solid form. In certain embodiments, compound 12 is in solid form. When compound 12 is in solid form, said compound may be amorphous, crystalline, or a mixture thereof. Exemplary solid forms are described in more detail below.

In some embodiments, the present invention provides compound 12 substantially free of impurities. As used herein, the term “substantially free of impurities” means that the compound contains no significant amount of extraneous matter. Such extraneous matter may include excess hydrobromic acid, excess synthetic reagents, excess compound A, residual solvents, or any other impurities that may result from the preparation of, and/or isolation of, compound 12. In certain embodiments, at least about 95% by weight of compound 12 is present in a composition. In still other embodiments of the invention, at least about 99% by weight of compound 12 is present in a composition.

According to one embodiment, compound 12 is present in a composition in an amount of at least about 97, 97.5, 98.0, 98.5, 99, 99.5, 99.8 weight percent where the percentages are based on the total weight of the composition. According to another embodiment, compound 12 contains no more than about 3.0 area percent HPLC of total organic impurities and, in certain embodiments, no more than about 1.5 area percent HPLC total organic impurities relative to the total area of the HPLC chromatogram. In other embodiments, compound 12 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 12 is also meant to include all tautomeric forms and isomeric forms of compound 12.

It has been found that compound 12 can exist in a variety of solid forms. Exemplary such forms include polymorphs such as those described herein.

It has been found that compound 12 can exist in at least two polymorphic forms.

In some embodiments, the present invention provides a polymorphic form of Compound 12 referred to herein as Form I. In some embodiments, Form I of compound 12 is substantially free of other polymorphic forms.

In certain embodiments, the present invention provides a polymorphic form of compound 12 referred to herein as Form II. In some embodiments, Form II of compound 12 is substantially free of other polymorphic forms.

In certain embodiments, compound 12 is a crystalline solid. In other embodiments, compound 12 is a crystalline solid substantially free of amorphous compound 12. As used herein, the term “substantially free of amorphous compound 12” means that the compound contains no significant amount of amorphous compound 12. In certain embodiments, at least about 95% by weight of crystalline compound 12 is present. In still other embodiments of the invention, at least about 99% by weight of crystalline compound 12 is present.

In some embodiments, compound 12 is amorphous. In some embodiments, compound 12 is amorphous, and is substantially free of crystalline compound 12.

### Form I of Compound 12

In some embodiments, Form I of compound 12 has at least 1, 2, 3, 4 or 5 spectral peak(s) selected from the peaks listed in Table 25 below.

TABLE 25
XRPD Peak Positions for Form I of Compound 12

| Pos. [°2θ] | Int. I. (cps) |
|---|---|
| 5.5 | 4377(33) |
| 7.1 | 762(26) |
| 7.9 | 693(26) |
| 11.0 | 1212(34) |
| 11.7 | 891(34) |
| 13.5 | 1720(42) |
| 14.2 | 9911(131) |
| 14.7 | 1914(89) |
| 15.8 | 1684(76) |
| 16.6 | 24333(135) |
| 17.0 | 6889(123) |
| 19.1 | 3988(777) |
| 19.4 | 24728(779) |
| 20.2 | 31173(146) |
| 20.8 | 1627(85) |
| 21.8 | 14984(110) |
| 23.1 | 12144(197) |
| 23.6 | 5256(102) |
| 24.2 | 2328(79) |
| 24.7 | 3305(59) |
| 25.1 | 1216(70) |
| 25.8 | 2993(116) |
| 26.0 | 3283(112) |
| 26.7 | 4839(77) |
| 27.1 | 4993(137) |
| 27.5 | 5296(131) |
| 28.3 | 5621(149) |
| 28.7 | 1333(555) |
| 28.9 | 1353(454) |
| 29.9 | 8525(103) |
| 30.5 | 627(50) |
| 31.1 | 1179(55) |
| 32.3 | 4137(255) |
| 32.7 | 1899(272) |
| 33.8 | 5014(164) |
| 34.2 | 1200(133) |
| 34.7 | 3127(94) |
| 35.2 | 1523(86) |
| 35.8 | 2997(94) |
| 36.2 | 5725(110) |
| 38.0 | 1756(137) |
| 38.4 | 662(129) |
| 38.9 | 446(66) |
| 39.4 | 3904(98) |
| 40.2 | 704(70) |
| 41.4 | 2316(62) |
| 42.1 | 668(57) |
| 42.6 | 773(66) |
| 43.5 | 666(42) |
| 44.4 | 1989(77) |
| 45.1 | 1235(86) |
| 47.7 | 1357(74) |
| 48.3 | 1146(68) |
| 50.1 | 630(50) |
| 51.3 | 4966(149) |
| 52.8 | 631(56) |
| 54.9 | 903(39) |
| 56.7 | 989(42) |
| 58.1 | 1493(56) |
| 59.1 | 156(40) |

In some embodiments, Form I of compound 12 is characterized in that it has one or more peaks in its X-ray powder diffraction pattern selected from those at about 16.6, about 19.4 and about 20.2 degrees 2-theta. In some embodiments, Form I of compound 12 is characterized in that it has two or more peaks in its X-ray powder diffraction pattern selected from those at about 16.6, about 19.4 and about 20.2 degrees 2-theta. In some embodiments, Form I of compound 12 is characterized in that it has all three peaks in its X-ray powder diffraction pattern selected from those at about 16.6, about 19.4 and about 20.2 degrees 2-theta.

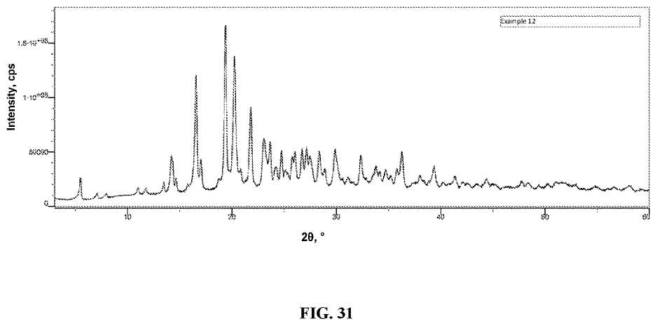

In certain embodiments, the X-ray powder diffraction pattern is substantially similar to the XRPD provided in FIG. 31.

Methods for preparing Form I of compound 12 are described infra.

### Form II of Compound 12

In some embodiments, Form II of compound 12 has at least 1, 2, 3, 4 or 5 spectral peak(s) selected from the peaks listed in Table 26 below.

TABLE 26
XRPD Peak Positions for Form II of Compound 12

| Pos. [°2θ] | Rel. Int. [%] |
|---|---|
| 5.8 | 18.67 |
| 7.4 | 1.87 |
| 8.3 | 2.05 |
| 11.3 | 7.23 |
| 12.1 | 3.97 |
| 13.8 | 22.01 |
| 14.6 | 55.91 |
| 15.0 | 22.25 |
| 16.1 | 14.58 |
| 16.9 | 95.33 |
| 17.4 | 36.88 |
| 19.6 | 68.30 |
| 20.5 | 100.00 |
| 22.0 | 38.60 |
| 23.4 | 56.80 |
| 24.5 | 25.08 |
| 25.0 | 56.87 |
| 26.0 | 48.43 |
| 26.4 | 49.57 |
| 27.0 | 38.00 |
| 27.5 | 38.71 |
| 27.9 | 37.61 |
| 28.6 | 19.31 |
| 29.2 | 22.73 |
| 30.2 | 28.81 |
| 31.4 | 13.05 |
| 32.6 | 15.94 |
| 33.8 | 15.01 |
| 34.5 | 17.10 |
| 35.1 | 14.35 |
| 36.0 | 14.08 |
| 36.5 | 16.51 |
| 38.3 | 11.73 |
| 39.6 | 13.58 |

In some embodiments, Form II of compound 12 is characterized in that it has one or more peaks in its X-ray powder diffraction pattern selected from those at about 16.9, about 19.6 and about 20.5 degrees 2-theta. In some embodiments, Form II of compound 12 is characterized in that it has two or more peaks in its X-ray powder diffraction pattern selected from those at about 16.9, about 19.6 and about 20.5 degrees 2-theta. In some embodiments, Form II of compound 12 is characterized in that it has all three peaks in its X-ray powder diffraction pattern selected from those at about 16.9, about 19.6 and about 20.5 degrees 2-theta.

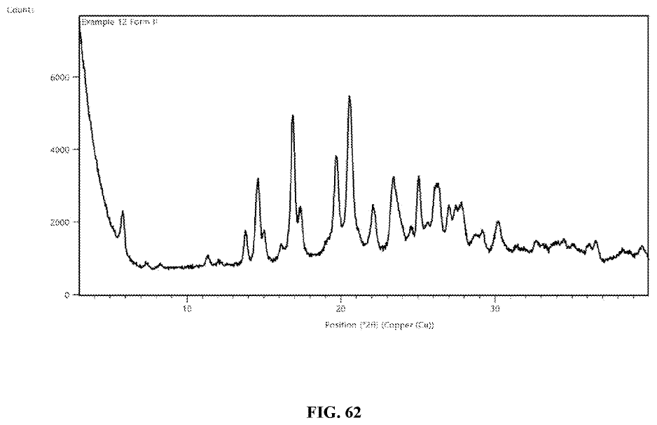

In certain embodiments, the X-ray powder diffraction pattern is substantially similar to the XRPD provided in FIG. 62.

Methods for preparing Form II of compound 12 are described infra.

In some embodiments, the present invention provides compound 12, wherein said compound is crystalline.

In some embodiments, the present invention provides compound 12, wherein said compound is a crystalline solid substantially free of amorphous compound 12.

In some embodiments, the present invention provides compound 12, wherein said compound is substantially free of impurities.

In some embodiments, the present invention provides a composition comprising Form I or Form II of compound 12 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I or Form II of compound 12 or composition thereof.

In some embodiments, the present invention provides a method of treating cancer in a patient by blocking the activity of kinases, comprising administering to said patient Form I or Form II of compound 12 or composition thereof wherein Form I or Form II of compound 12 is a crystal form as described herein.

### Compound 23 (Phosphate Salts of Compound A)

According to one embodiment, the present invention provides a phosphate salt of compound A, represented by compound 23:

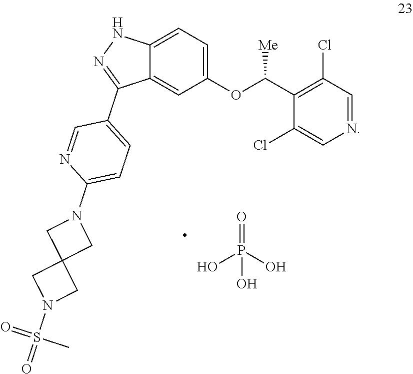

It will be appreciated by one of ordinary skill in the art that the phosphoric acid and compound A are ionically bonded to form compound 23. It is contemplated that compound 1 can exist in a variety of physical forms. For example, compound 23 can be in solution, suspension, or in solid form. In certain embodiments, compound 23 is in solid form. When compound 23 is in solid form, said compound may be amorphous, crystalline, or a mixture thereof. Exemplary solid forms are described in more detail below.

In some embodiments, the present invention provides compound 23 substantially free of impurities. As used herein, the term “substantially free of impurities” means that the compound contains no significant amount of extraneous matter. Such extraneous matter may include excess phosphoric acid, excess synthetic reagents, excess compound A, residual solvents, or any other impurities that may result from the preparation of, and/or isolation of, compound 23. In certain embodiments, at least about 95% by weight of compound 23 is present in a composition. In still other embodiments of the invention, at least about 99% by weight of compound 23 is present in a composition.

According to one embodiment, compound 23 is present in a composition in an amount of at least about 97, 97.5, 98.0, 98.5, 99, 99.5, 99.8 weight percent where the percentages are based on the total weight of the composition. According to another embodiment, compound 23 contains no more than about 3.0 area percent HPLC of total organic impurities. The area percent by HPLC of total organic impurities can be assessed by those skilled in the art, using conventional HPLC methods, including for example, a photodiode array detector. In certain embodiments, no more than about 1.5 area percent HPLC total organic impurities relative to the total area of the HPLC chromatogram. In other embodiments, compound 23 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 23 is also meant to include all tautomeric forms and isomeric forms of compound 23.

It has been found that compound 23 can exist in a variety of solid forms. Exemplary such forms include polymorphs such as those described herein.

In certain embodiments, compound 23 is a crystalline solid. In other embodiments, compound 23 is a crystalline solid substantially free of amorphous compound 23. As used herein, the term “substantially free of amorphous compound 23” means that the compound contains no significant amount of amorphous compound 23. In certain embodiments, at least about 95% by weight of crystalline compound 23 is present. In still other embodiments of the invention, at least about 99% by weight of crystalline compound 23 is present.

It has been found that compound 23 can exist in at least one polymorphic form. In some embodiments, the present invention provides a polymorphic form of compound 23 referred to herein as Form I. In some embodiments, Form I of compound 23 is substantially free of other polymorphic forms.

In some embodiments, compound 23 is amorphous. In some embodiments, compound 23 is amorphous, and is substantially free of crystalline compound 23.

### Form I of Compound 23

In some embodiments, Form I of compound 23 has at least 1, 2, 3, 4 or 5 spectral peak(s) selected from the peaks listed in Table 27 below.

TABLE 27
XRPD Peak Positions for Form I of Compound 27

| Pos. [°2θ] | Int. I. (cps) |
|---|---|
| 5.5 | 1.80 |
| 5.9 | 2.10 |
| 7.1 | 1.77 |
| 12.2 | 2.75 |
| 12.9 | 2.98 |
| 13.9 | 18.02 |
| 14.8 | 20.69 |
| 15.4 | 17.45 |
| 16.7 | 25.43 |
| 17.8 | 25.53 |
| 18.9 | 25.60 |
| 19.8 | 100.00 |
| 21.6 | 18.15 |
| 22.6 | 18.58 |
| 23.3 | 25.08 |
| 24.3 | 39.32 |
| 25.4 | 78.05 |
| 26.5 | 31.83 |
| 27.2 | 26.75 |
| 27.6 | 21.57 |
| 29.7 | 10.78 |
| 30.4 | 8.60 |
| 34.0 | 3.95 |
| 35.6 | 1.80 |
| 36.9 | 2.36 |

In some embodiments, Form I of compound 23 is characterized in that it has one or more peaks in its X-ray powder diffraction pattern selected from those at about 19.8, about 24.3 and about 25.4 degrees 2-theta. In some embodiments, Form I of compound 23 is characterized in that it has two or more peaks in its X-ray powder diffraction pattern selected from those at about 19.8, about 24.3 and about 25.4 degrees 2-theta. In some embodiments, Form I of compound 23 is characterized in that it has all three peaks in its X-ray powder diffraction pattern selected from those at about 19.8, about 24.3 and about 25.4 degrees 2-theta.

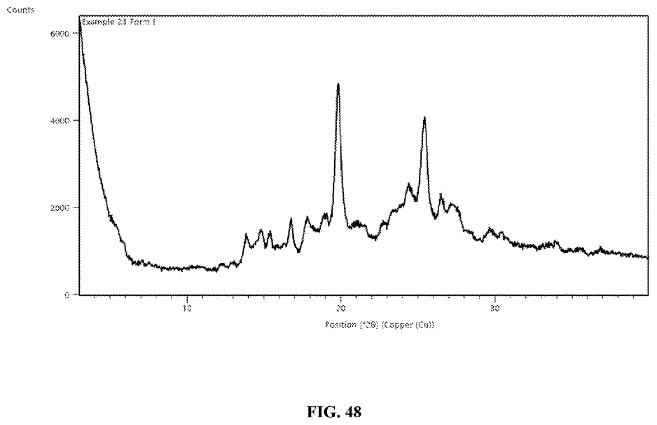

In certain embodiments, the X-ray powder diffraction pattern is substantially similar to the XRPD provided in FIG. 48.

Methods for preparing Form I of compound 23 are described infra.

In some embodiments, the present invention provides compound 23, wherein said compound is crystalline.

In some embodiments, the present invention provides compound 23, wherein said compound is a crystalline solid substantially free of amorphous compound 23.

In some embodiments, the present invention provides compound 23, wherein said compound is substantially free of impurities.

In some embodiments, the present invention provides a composition comprising Form I of compound 23 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 23 or composition thereof.

In some embodiments, the present invention provides a method of treating cancer in a patient by blocking the activity of kinases, comprising administering to said patient Form I of compound 23 or composition thereof wherein Form I of compound 23 is a crystal form as described herein.

### Compound 24 (Citrate Salts of Compound A)

According to one embodiment, the present invention provides a citrate salt of compound A, represented by compound 24:

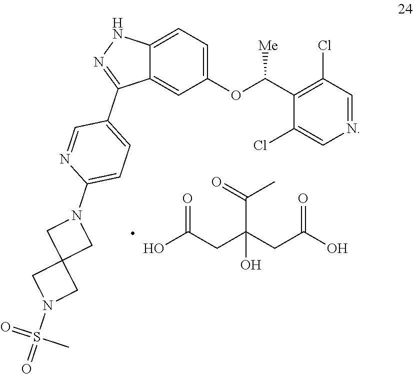

It will be appreciated by one of ordinary skill in the art that the citric acid and compound A are ionically bonded to form compound 24. It is contemplated that compound 1 can exist in a variety of physical forms. For example, compound 24 can be in solution, suspension, or in solid form. In certain embodiments, compound 24 is in solid form. When compound 24 is in solid form, said compound may be amorphous, crystalline, or a mixture thereof. Exemplary solid forms are described in more detail below.

In some embodiments, the present invention provides compound 24 substantially free of impurities. As used herein, the term “substantially free of impurities” means that the compound contains no significant amount of extraneous matter. Such extraneous matter may include excess citric acid, excess synthetic reagents, excess compound A, residual solvents, or any other impurities that may result from the preparation of, and/or isolation of, compound 24. In certain embodiments, at least about 95% by weight of compound 24 is present in a composition. In still other embodiments of the invention, at least about 99% by weight of compound 24 is present in a composition.

According to one embodiment, compound 24 is present in a composition in an amount of at least about 97, 97.5, 98.0, 98.5, 99, 99.5, 99.8 weight percent where the percentages are based on the total weight of the composition. According to another embodiment, compound 24 contains no more than about 3.0 area percent HPLC of total organic impurities. The area percent by HPLC of total organic impurities can be assessed by those skilled in the art, using conventional HPLC methods, including for example, a photodiode array detector. In certain embodiments, no more than about 1.5 area percent HPLC total organic impurities relative to the total area of the HPLC chromatogram. In other embodiments, compound 24 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 24 is also meant to include all tautomeric forms and isomeric forms of compound 24.

It has been found that compound 24 can exist in a variety of solid forms. Exemplary such forms include polymorphs such as those described herein.

In certain embodiments, compound 24 is a crystalline solid. In other embodiments, compound 24 is a crystalline solid substantially free of amorphous compound 24. As used herein, the term “substantially free of amorphous compound 24” means that the compound contains no significant amount of amorphous compound 24. In certain embodiments, at least about 95% by weight of crystalline compound 24 is present. In still other embodiments of the invention, at least about 99% by weight of crystalline compound 24 is present.

It has been found that compound 24 can exist in at least one polymorphic form. In some embodiments, the present invention provides a polymorphic form of compound 24 referred to herein as Form I. In some embodiments, Form I of compound 24 is substantially free of other polymorphic forms.

In some embodiments, compound 24 is amorphous. In some embodiments, compound 24 is amorphous, and is substantially free of crystalline compound 24.

### Form I of Compound 24

In some embodiments, Form I of compound 24 has at least 1, 2, 3, 4 or 5 spectral peak(s) selected from the peaks listed in Table 28 below.

TABLE 28
XRPD Peak Positions for Form I of Compound 24

| Pos. [°2θ] | Int. I. (cps) |
|---|---|
| 5.1 | 14.23 |
| 6.8 | 22.39 |
| 7.8 | 14.31 |
| 11.1 | 2.81 |
| 12.6 | 12.82 |
| 13.7 | 11.14 |
| 14.6 | 8.54 |
| 15.2 | 34.49 |
| 16.4 | 1.53 |
| 17.3 | 19.91 |
| 18.3 | 8.73 |
| 18.9 | 9.34 |
| 19.8 | 100.00 |
| 20.8 | 10.85 |
| 21.4 | 13.71 |
| 22.5 | 10.42 |
| 22.9 | 7.50 |
| 24.4 | 8.08 |
| 24.9 | 11.67 |
| 25.3 | 14.80 |
| 26.4 | 13.41 |
| 26.9 | 8.29 |
| 27.3 | 7.69 |
| 27.7 | 6.18 |
| 28.3 | 7.09 |
| 29.2 | 6.64 |
| 30.3 | 6.64 |
| 31.0 | 5.14 |
| 33.2 | 3.23 |
| 33.9 | 4.63 |
| 35.1 | 2.82 |
| 36.8 | 1.43 |
| 38.2 | 0.80 |

In some embodiments, Form I of compound 24 is characterized in that it has one or more peaks in its X-ray powder diffraction pattern selected from those at about 6.8, about 15.2 and about 19.8 degrees 2-theta. In some embodiments, Form I of compound 24 is characterized in that it has two or more peaks in its X-ray powder diffraction pattern selected from those at about 6.8, about 15.2 and about 19.8 degrees 2-theta. In some embodiments, Form I of compound 24 is characterized in that it has all three peaks in its X-ray powder diffraction pattern selected from those at about 6.8, about 15.2 and about 19.8 degrees 2-theta.

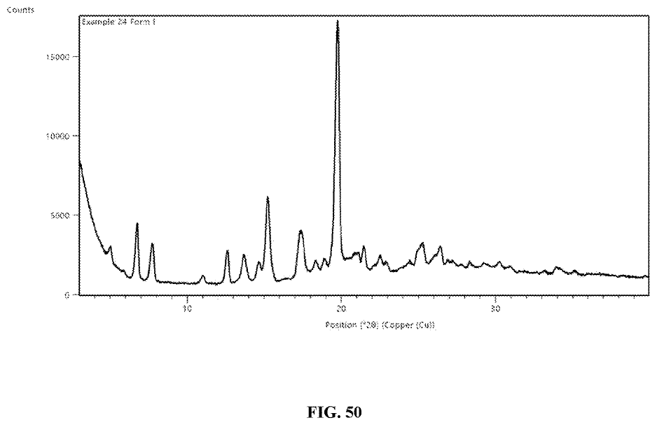

In certain embodiments, the X-ray powder diffraction pattern is substantially similar to the XRPD provided in FIG. 50.

Methods for preparing Form I of compound 24 are described infra.

In some embodiments, the present invention provides compound 24, wherein said compound is crystalline.

In some embodiments, the present invention provides compound 24, wherein said compound is a crystalline solid substantially free of amorphous compound 24.

In some embodiments, the present invention provides compound 24, wherein said compound is substantially free of impurities.

In some embodiments, the present invention provides a composition comprising Form I of compound 24 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 24 or composition thereof.

In some embodiments, the present invention provides a method of treating cancer in a patient by blocking the activity of kinases, comprising administering to said patient Form I of compound 24 or composition thereof wherein Form I of compound 24 is a crystal form as described herein.

### Compound 25 (L-Tartrate Salts of Compound A)

According to one embodiment, the present invention provides an L-tartrate salt of compound A, represented by compound 25:

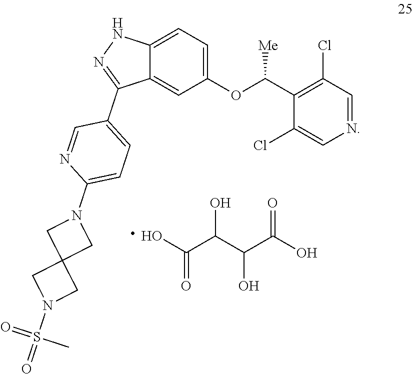

It will be appreciated by one of ordinary skill in the art that the L-tartaric acid and compound A are ionically bonded to form compound 25. It is contemplated that compound 1 can exist in a variety of physical forms. For example, compound 25 can be in solution, suspension, or in solid form. In certain embodiments, compound 25 is in solid form. When compound 25 is in solid form, said compound may be amorphous, crystalline, or a mixture thereof. Exemplary solid forms are described in more detail below.

In some embodiments, the present invention provides compound 25 substantially free of impurities. As used herein, the term “substantially free of impurities” means that the compound contains no significant amount of extraneous matter. Such extraneous matter may include excess L-tartaric acid, excess synthetic reagents, excess compound A, residual solvents, or any other impurities that may result from the preparation of, and/or isolation of, compound 25. In certain embodiments, at least about 95% by weight of compound 25 is present in a composition. In still other embodiments of the invention, at least about 99% by weight of compound 25 is present in a composition.

According to one embodiment, compound 25 is present in a composition in an amount of at least about 97, 97.5, 98.0, 98.5, 99, 99.5, 99.8 weight percent where the percentages are based on the total weight of the composition. According to another embodiment, compound 25 contains no more than about 3.0 area percent HPLC of total organic impurities. The area percent by HPLC of total organic impurities can be assessed by those skilled in the art, using conventional HPLC methods, including for example, a photodiode array detector. In certain embodiments, no more than about 1.5 area percent HPLC total organic impurities relative to the total area of the HPLC chromatogram. In other embodiments, compound 25 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 25 is also meant to include all tautomeric forms and isomeric forms of compound 25. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that compound 25 can exist in a variety of solid forms. Exemplary such forms include polymorphs such as those described herein.

In certain embodiments, compound 25 is a crystalline solid. In other embodiments, compound 25 is a crystalline solid substantially free of amorphous compound 25. As used herein, the term “substantially free of amorphous compound 25” means that the compound contains no significant amount of amorphous compound 25. In certain embodiments, at least about 95% by weight of crystalline compound 25 is present. In still other embodiments of the invention, at least about 99% by weight of crystalline compound 25 is present.

It has been found that compound 25 can exist in at least one polymorphic form. In some embodiments, the present invention provides a polymorphic form of compound 25 referred to herein as Form I. In some embodiments, Form I of compound 25 is substantially free of other polymorphic forms.

In some embodiments, compound 25 is amorphous. In some embodiments, compound 25 is amorphous, and is substantially free of crystalline compound 25.

### Form I of Compound 25

In some embodiments, Form I of compound 25 has at least 1, 2, 3, 4 or 5 spectral peak(s) selected from the peaks listed in Table 29 below.

TABLE 29
XRPD Peak Positions for Form I of Compound 25

| Pos. [°2θ] | Int. I. (cps) |
|---|---|
| 6.4 | 9.08 |
| 9.9 | 4.27 |
| 12.1 | 15.87 |
| 14.5 | 55.97 |
| 15.1 | 50.42 |
| 17.1 | 63.56 |
| 17.4 | 72.15 |
| 17.8 | 60.94 |
| 18.9 | 99.72 |
| 21.0 | 38.57 |
| 22.5 | 59.48 |
| 24.0 | 96.86 |
| 24.8 | 71.76 |
| 25.6 | 100.00 |
| 26.5 | 90.07 |
| 27.7 | 52.06 |
| 28.9 | 69.56 |
| 31.5 | 20.26 |
| 34.4 | 24.23 |
| 37.2 | 5.79 |

In some embodiments, Form I of compound 25 is characterized in that it has one or more peaks in its X-ray powder diffraction pattern selected from those at about 18.9, about 24.0 and about 25.6 degrees 2-theta. In some embodiments, Form I of compound 25 is characterized in that it has two or more peaks in its X-ray powder diffraction pattern selected from those at about 18.9, about 24.0 and about 25.6 degrees 2-theta. In some embodiments, Form I of compound 25 is characterized in that it has all three peaks in its X-ray powder diffraction pattern selected from those at about 18.9, about 24.0 and about 25.6 degrees 2-theta.

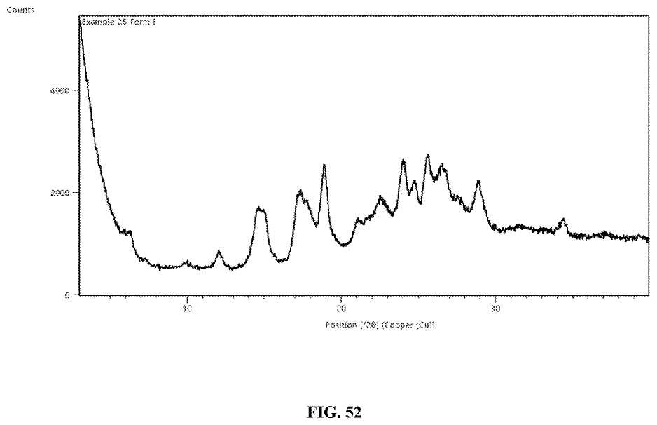

In certain embodiments, the X-ray powder diffraction pattern is substantially similar to the XRPD provided in FIG. 52.

Methods for preparing Form I of compound 25 are described infra.

In some embodiments, the present invention provides compound 25, wherein said compound is crystalline.

In some embodiments, the present invention provides compound 25, wherein said compound is a crystalline solid substantially free of amorphous compound 25.

In some embodiments, the present invention provides compound 25, wherein said compound is substantially free of impurities.

In some embodiments, the present invention provides a composition comprising Form I of compound 25 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 25 or composition thereof.

In some embodiments, the present invention provides a method of treating cancer in a patient by blocking the activity of kinases, comprising administering to said patient Form I of compound 25 or composition thereof wherein Form I of compound 25 is a crystal form as described herein.

### Compound 26 (Malonate Salts of Compound A)

According to one embodiment, the present invention provides a malonate salt of compound A, represented by compound 26:

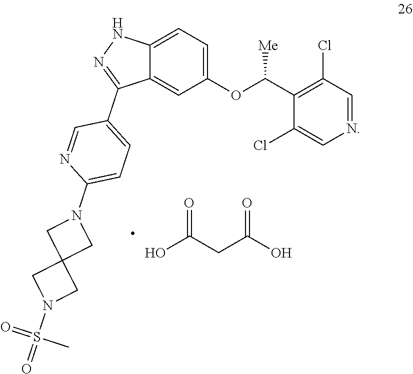

It will be appreciated by one of ordinary skill in the art that the malonic acid and compound A are ionically bonded to form compound 26. It is contemplated that compound 1 can exist in a variety of physical forms. For example, compound 26 can be in solution, suspension, or in solid form. In certain embodiments, compound 26 is in solid form. When compound 26 is in solid form, said compound may be amorphous, crystalline, or a mixture thereof. Exemplary solid forms are described in more detail below.

In some embodiments, the present invention provides compound 26 substantially free of impurities. As used herein, the term “substantially free of impurities” means that the compound contains no significant amount of extraneous matter. Such extraneous matter may include excess malonic acid, excess synthetic reagents, excess compound A, residual solvents, or any other impurities that may result from the preparation of, and/or isolation of, compound 26. In certain embodiments, at least about 95% by weight of compound 26 is present in a composition. In still other embodiments of the invention, at least about 99% by weight of compound 26 is present in a composition.

According to one embodiment, compound 26 is present in a composition in an amount of at least about 97, 97.5, 98.0, 98.5, 99, 99.5, 99.8 weight percent where the percentages are based on the total weight of the composition. According to another embodiment, compound 26 contains no more than about 3.0 area percent HPLC of total organic impurities. The area percent by HPLC of total organic impurities can be assessed by those skilled in the art, using conventional HPLC methods, including for example, a photodiode array detector. In certain embodiments, no more than about 1.5 area percent HPLC total organic impurities relative to the total area of the HPLC chromatogram. In other embodiments, compound 26 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 26 is also meant to include all tautomeric forms and isomeric forms of compound 26.

It has been found that compound 26 can exist in a variety of solid forms. Exemplary such forms include polymorphs such as those described herein.

In certain embodiments, compound 26 is a crystalline solid. In other embodiments, compound 26 is a crystalline solid substantially free of amorphous compound 26. As used herein, the term “substantially free of amorphous compound 26” means that the compound contains no significant amount of amorphous compound 26. In certain embodiments, at least about 95% by weight of crystalline compound 26 is present. In still other embodiments of the invention, at least about 99% by weight of crystalline compound 26 is present.

It has been found that compound 26 can exist in at least one polymorphic form. In some embodiments, the present invention provides a polymorphic form of compound 26 referred to herein as Form I. In some embodiments, Form I of compound 26 is substantially free of other polymorphic forms.

In some embodiments, compound 26 is amorphous. In some embodiments, compound 25 is amorphous, and is substantially free of crystalline compound 26.

### Form I of Compound 26

In some embodiments, Form I of compound 26 has at least 1, 2, 3, 4 or 5 spectral peak(s) selected from the peaks listed in Table 30 below.

TABLE 30
XRPD Peak Positions for Form I of Compound 26

| Pos. [°2θ] | Int. I. (cps) |
|---|---|
| 5.9 | 33.96 |
| 6.5 | 11.05 |
| 7.7 | 16.88 |
| 12.8 | 58.97 |
| 13.9 | 5.33 |
| 14.6 | 50.56 |
| 15.6 | 5.12 |
| 17.6 | 11.46 |
| 18.5 | 30.50 |
| 19.1 | 100.00 |
| 20.0 | 25.43 |
| 21.0 | 26.11 |
| 21.3 | 36.29 |
| 22.8 | 13.22 |
| 24.8 | 42.86 |
| 25.6 | 50.48 |
| 26.0 | 43.14 |
| 26.7 | 17.93 |
| 27.6 | 21.22 |
| 28.0 | 15.76 |
| 29.3 | 16.11 |
| 30.5 | 10.95 |
| 31.6 | 13.49 |
| 33.8 | 8.23 |
| 34.5 | 8.30 |
| 35.3 | 6.85 |
| 37.7 | 4.60 |
| 38.8 | 5.59 |

In some embodiments, Form I of compound 26 is characterized in that it has one or more peaks in its X-ray powder diffraction pattern selected from those at about 12.8, about 14.6 and about 19.1 degrees 2-theta. In some embodiments, Form I of compound 26 is characterized in that it has two or more peaks in its X-ray powder diffraction pattern selected from those at about 12.8, about 14.6 and about 19.1 degrees 2-theta. In some embodiments, Form I of compound 26 is characterized in that it has all three peaks in its X-ray powder diffraction pattern selected from those at about 12.8, about 14.6 and about 19.1 degrees 2-theta.

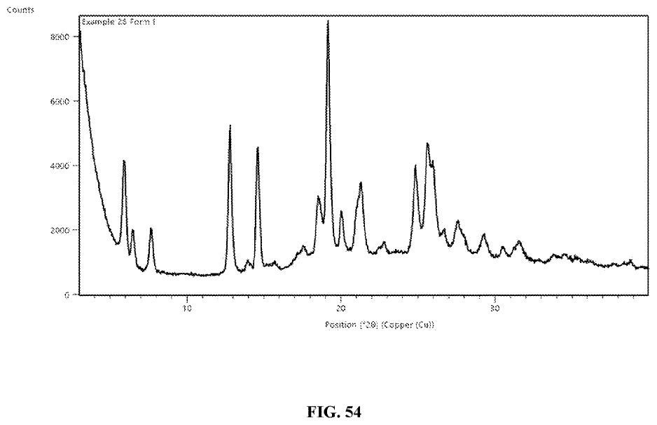

In certain embodiments, the X-ray powder diffraction pattern is substantially similar to the XRPD provided in FIG. 54.

Methods for preparing Form I of compound 26 are described infra.

In some embodiments, the present invention provides compound 26, wherein said compound is crystalline.

In some embodiments, the present invention provides compound 26, wherein said compound is a crystalline solid substantially free of amorphous compound 26.

In some embodiments, the present invention provides compound 26, wherein said compound is substantially free of impurities.

In some embodiments, the present invention provides a composition comprising Form I of compound 26 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient Form I of compound 26 or composition thereof.

In some embodiments, the present invention provides a method of treating cancer in a patient by blocking the activity of kinases, comprising administering to said patient Form I of compound 26 or composition thereof wherein Form I of compound 26 is a crystal form as described herein.

### Free Base Forms of Compounds 13 and 14

It is contemplated that compounds 13 and 14 can exist in a variety of physical forms. For example, compounds 13 and 14 can be in solution, suspension, or in solid form. In certain embodiments, compounds 13 and 14 are in solid form. When compounds 13 and 14 are in solid form, said compounds may be amorphous, crystalline, or a mixture thereof. Exemplary such solid forms of compounds 13 and 14 are described in more detail below.

Compound 13 (d₆-Compound A)

According to one embodiment, the present invention provides a freebase form of deuterated compound A, represented by compound 13:

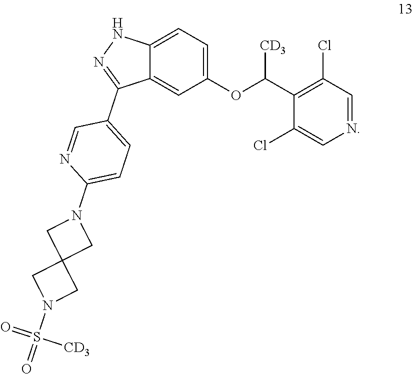 [SMILES: [2H]C([2H])([2H])C(Oc1ccc2[nH]nc(-c3ccc(N4CC5(C4)CN(S(=O)(=O)C([2H])([2H])[2H])C5)nc3)c2c1)c1c(Cl)cncc1Cl]

It is contemplated that compound 13 can exist in a variety of physical forms. For example, compound 13 can be in solution, suspension, or in solid form. In certain embodiments, compound 13 is in solid form. When compound 13 is in solid form, said compound may be amorphous, crystalline, or a mixture thereof. Exemplary solid forms are described in more detail below.

In some embodiments, the present invention provides compound 13 substantially free of impurities. As used herein, the term “substantially free of impurities” means that the compound contains no significant amount of extraneous matter. Such extraneous matter may include excess synthetic reagents, excess compound A, residual solvents, or any other impurities that may result from the preparation of, and/or isolation of, compound 13. In certain embodiments, at least about 95% by weight of compound 13 is present in a composition. In still other embodiments of the invention, at least about 99% by weight of compound 13 is present in a composition.

According to one embodiment, compound 13 is present in a composition in an amount of at least about 97, 97.5, 98.0, 98.5, 99, 99.5, 99.8 weight percent where the percentages are based on the total weight of the composition. According to another embodiment, compound 13 contains no more than about 3.0 area percent HPLC of total organic impurities and, in certain embodiments, no more than about 1.5 area percent HPLC total organic impurities relative to the total area of the HPLC chromatogram. In other embodiments, compound 13 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 13 is also meant to include all tautomeric forms and isomeric forms of compound 13.

It has been found that compound 13 can exist in a variety of solid forms. Exemplary such forms include polymorphs such as those described herein.

In certain embodiments, compound 13 is a crystalline solid. In other embodiments, compound 13 is a crystalline solid substantially free of amorphous compound 13. As used herein, the term “substantially free of amorphous compound 13” means that the compound contains no significant amount of amorphous compound 13. In certain embodiments, at least about 95% by weight of crystalline compound 13 is present. In still other embodiments of the invention, at least about 99% by weight of crystalline compound 13 is present.

It has been found that compound 13 can exist in at least one polymorphic form. In some embodiments, compound 13 is substantially free of other polymorphic forms.

In some embodiments, compound 13 is amorphous. In some embodiments, compound 13 is amorphous, and is substantially free of crystalline compound 13.

Methods for preparing compound 13 are described infra.

In some embodiments, the present invention provides compound 13, wherein said compound is crystalline.

In some embodiments, the present invention provides compound 13, wherein said compound is a crystalline solid substantially free of amorphous compound 13.

In some embodiments, the present invention provides compound 13, wherein said compound is substantially free of impurities.

In some embodiments, the present invention provides a composition comprising compound 13 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 13 or composition thereof.

In some embodiments, the present invention provides a method of treating cancer in a patient by blocking the activity of kinases, comprising administering to said patient compound 13 or composition thereof wherein compound 13 is a crystal form as described herein.

Compound 14 (d3-Compound A)

According to one embodiment, the present invention provides a freebase form of deuterated compound A, represented by compound 14:

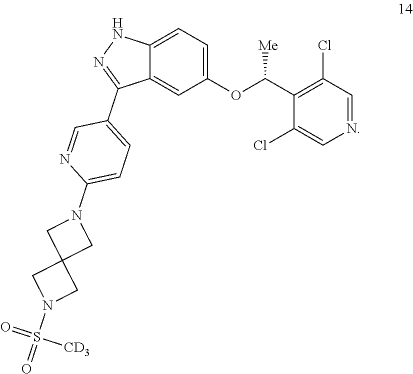 [SMILES: [2H]C([2H])([2H])S(=O)(=O)N1CC2(CN(c3ccc(-c4n[nH]c5ccc(O[C@H](C)c6c(Cl)cncc6Cl)cc45)cn3)C2)C1]

It is contemplated that compound 14 can exist in a variety of physical forms. For example, compound 14 can be in solution, suspension, or in solid form. In certain embodiments, compound 14 is in solid form. When compound 14 is in solid form, said compound may be amorphous, crystalline, or a mixture thereof. Exemplary solid forms are described in more detail below.

In some embodiments, the present invention provides compound 14 substantially free of impurities. As used herein, the term “substantially free of impurities” means that the compound contains no significant amount of extraneous matter. Such extraneous matter may include excess synthetic reagents, excess compound A, residual solvents, or any other impurities that may result from the preparation of, and/or isolation of, compound 14. In certain embodiments, at least about 95% by weight of compound 14 is present in a composition. In still other embodiments of the invention, at least about 99% by weight of compound 14 is present in a composition.

According to one embodiment, compound 14 is present in a composition in an amount of at least about 97, 97.5, 98.0, 98.5, 99, 99.5, 99.8 weight percent where the percentages are based on the total weight of the composition. According to another embodiment, compound 14 contains no more than about 3.0 area percent HPLC of total organic impurities and, in certain embodiments, no more than about 1.5 area percent HPLC total organic impurities relative to the total area of the HPLC chromatogram. In other embodiments, compound 14 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 14 is also meant to include all tautomeric forms and isomeric forms of compound 14.

It has been found that compound 14 can exist in a variety of solid forms. Exemplary such forms include polymorphs such as those described herein.

In certain embodiments, compound 14 is a crystalline solid. In other embodiments, compound 14 is a crystalline solid substantially free of amorphous compound 14. As used herein, the term “substantially free of amorphous compound 14” means that the compound contains no significant amount of amorphous compound 14. In certain embodiments, at least about 95% by weight of crystalline compound 14 is present. In still other embodiments of the invention, at least about 99% by weight of crystalline compound 14 is present.

It has been found that compound 14 can exist in at least one polymorphic form. In some embodiments, compound 14 is substantially free of other polymorphic forms.

In some embodiments, compound 14 is amorphous. In some embodiments, compound 14 is amorphous, and is substantially free of crystalline compound 14.

Methods for preparing compound 14 are described infra.

In some embodiments, the present invention provides compound 14, wherein said compound is crystalline.

In some embodiments, the present invention provides compound 14, wherein said compound is a crystalline solid substantially free of amorphous compound 14.

In some embodiments, the present invention provides compound 14, wherein said compound is substantially free of impurities.

In some embodiments, the present invention provides a composition comprising compound 14 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 14 or composition thereof.

In some embodiments, the present invention provides a method of treating cancer in a patient by blocking the activity of kinases, comprising administering to said patient compound 14 or composition thereof wherein compound 14 is a crystal form as described herein.

### Free Base Forms of Compound A

It is contemplated that compound A can exist in a variety of physical forms. For example, compound A can be in solution, suspension, or in solid form. In certain embodiments, compound A is in solid form. When compound A is in solid form, said compounds may be amorphous, crystalline, or a mixture thereof. Exemplary such solid forms of compound A are described in more detail below.

Compounds 15-22 (Free Base Forms I, II, III, IV, V, VI, VII and VIII of Compound A, respectively) It has been found that compound A can exist in at least eight polymorphic forms.

In some embodiments, the present invention provides a polymorphic form of compound 15 referred to herein as Form I of Compound A. In some embodiments, Form I of compound A is substantially free of other polymorphic forms.

In certain embodiments, the present invention provides a polymorphic form of compound 16 referred to herein as Form II of Compound A. In some embodiments, Form II of compound A is substantially free of other polymorphic forms.

In certain embodiments, the present invention provides a polymorphic form of compound 17 referred to herein as Form III of Compound A. In some embodiments, Form III of compound A is substantially free of other polymorphic forms.

In certain embodiments, the present invention provides a polymorphic form of compound 18 referred to herein as Form IV of Compound A. In some embodiments, Form IV of compound A is substantially free of other polymorphic forms.

In certain embodiments, the present invention provides a polymorphic form of compound 19 referred to herein as Form V of Compound A. In some embodiments, Form V of compound A is substantially free of other polymorphic forms.

In certain embodiments, the present invention provides a polymorphic form of compound 20 referred to herein as Form VI of Compound A. In some embodiments, Form VI of compound A is substantially free of other polymorphic forms.

In certain embodiments, the present invention provides a polymorphic form of compound 21 referred to herein as Form VII of Compound A. In some embodiments, Form VII of compound A is substantially free of other polymorphic forms.

In certain embodiments, the present invention provides a polymorphic form of compound 22 referred to herein as Form VIII of Compound A. In some embodiments, Form VIII of compound A is substantially free of other polymorphic forms.

### Compound 15-Form I of Compound A

According to one embodiment, the present invention provides free base Form I of compound A, represented by compound 15.

In some embodiments, the present invention provides compound 15 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 15. In certain embodiments, at least about 95% by weight of compound 15 is present in a composition. In still other embodiments of the invention, at least about 99% by weight of compound 15 is present in a composition.

According to one embodiment, compound 15 is present in a composition in an amount of at least about 97, 97.5, 98.0, 98.5, 99, 99.5, 99.8 weight percent where the percentages are based on the total weight of the composition. According to another embodiment, compound 15 contains no more than about 3.0 area percent HPLC of total organic impurities and, in certain embodiments, no more than about 1.5 area percent HPLC total organic impurities relative to the total area of the HPLC chromatogram. In other embodiments, compound 15 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 15 is also meant to include all tautomeric forms and isomeric forms of compound 15.

It has been found that compound 15 can exist in a variety of solid forms. Exemplary such forms include polymorphs such as those described herein.

In certain embodiments, compound 15 is crystalline solid. In other embodiments, compound 15 is a crystalline solid substantially free of amorphous compound A. As used herein, the term “substantially free of amorphous compound A” means that the compound contains no significant amount of amorphous compound A. In certain embodiments, at least about 95% by weight of crystalline compound 15 is present. In still other embodiments of the invention, at least about 99% by weight of crystalline compound 15 is present.

In some embodiments, compound 15 has at least 1, 2, 3, 4 or 5 spectral peak(s) selected from the peaks listed in Table 31 below.

TABLE 31
XRPD Peak Positions for Compound 15

| Pos. [°2θ] | Rel. Int. [%] |
|---|---|
| 10.0 | 1.71 |
| 11.6 | 16.08 |
| 12.7 | 1.91 |
| 13.4 | 59.80 |
| 13.9 | 32.74 |
| 14.4 | 27.13 |
| 15.3 | 1.94 |
| 16.0 | 32.05 |
| 17.0 | 92.98 |
| 17.8 | 71.79 |
| 18.2 | 17.05 |
| 18.5 | 42.00 |
| 19.4 | 100.00 |
| 19.9 | 34.39 |
| 20.9 | 98.58 |
| 21.7 | 12.71 |
| 22.2 | 9.86 |
| 22.7 | 50.77 |
| 23.4 | 23.46 |
| 23.7 | 42.25 |
| 24.0 | 58.64 |
| 24.3 | 28.46 |
| 25.1 | 42.42 |
| 25.4 | 34.85 |
| 26.3 | 21.03 |
| 26.8 | 12.42 |
| 27.8 | 17.11 |
| 28.4 | 16.14 |
| 29.1 | 17.87 |
| 29.3 | 21.76 |
| 30.1 | 12.70 |
| 30.6 | 15.11 |
| 31.6 | 10.78 |
| 32.3 | 7.26 |
| 34.1 | 4.62 |
| 35.9 | 8.73 |
| 36.8 | 4.44 |
| 38.0 | 5.81 |

In some embodiments, compound 15 is characterized in that it has one or more peaks in its X-ray powder diffraction pattern selected from those at about 17.0, about 19.4 and about 20.9 degrees 2-theta. In some embodiments, compound 15 is characterized in that it has two or more peaks in its X-ray powder diffraction pattern selected from those at about 17.0, about 19.4 and about 20.9 degrees 2-theta. In some embodiments, compound 15 is characterized in that it has all three peaks in its X-ray powder diffraction pattern selected from those at about 17.0, about 19.4 and about 20.9 degrees 2-theta.

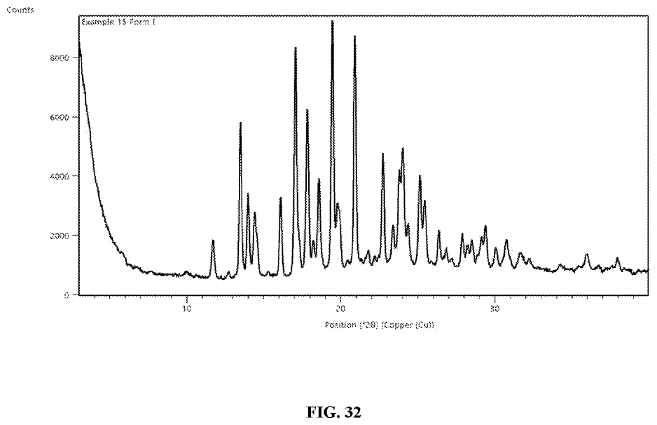

In certain embodiments, the X-ray powder diffraction pattern is substantially similar to the XRPD provided in FIG. 32.

Methods for preparing compound 15 are described infra.

In some embodiments, the present invention provides compound 15, wherein said compound is crystalline.

In some embodiments, the present invention provides compound 15, wherein said compound is a crystalline solid substantially free of amorphous compound A.

In some embodiments, the present invention provides compound 15, wherein said compound is substantially free of impurities.

In some embodiments, the present invention provides a composition comprising compound 15 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 15 or composition thereof.

In some embodiments, the present invention provides a method of treating cancer in a patient by blocking the activity of kinases, comprising administering to said patient compound 15 or composition thereof wherein compound 15 is a crystal form as described herein.

### Compound 16-Form II of Compound A

According to one embodiment, the present invention provides free base Form II of compound A, represented by compound 16.

In some embodiments, the present invention provides compound 16 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 16. In certain embodiments, at least about 95% by weight of compound 16 is present in a composition. In still other embodiments of the invention, at least about 99% by weight of compound 16 is present in a composition.

According to one embodiment, compound 16 is present in a composition in an amount of at least about 97, 97.5, 98.0, 98.5, 99, 99.5, 99.8 weight percent where the percentages are based on the total weight of the composition. According to another embodiment, compound 16 contains no more than about 3.0 area percent HPLC of total organic impurities and, in certain embodiments, no more than about 1.5 area percent HPLC total organic impurities relative to the total area of the HPLC chromatogram. In other embodiments, compound 16 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 16 is also meant to include all tautomeric forms and isomeric forms of compound 16.

It has been found that compound 16 can exist in a variety of solid forms. Exemplary such forms include polymorphs such as those described herein.

In certain embodiments, compound 16 is crystalline solid. In other embodiments, compound 16 is a crystalline solid substantially free of amorphous compound A. As used herein, the term “substantially free of amorphous compound A” means that the compound contains no significant amount of amorphous compound A. In certain embodiments, at least about 95% by weight of crystalline compound 16 is present. In still other embodiments of the invention, at least about 99% by weight of crystalline compound 16 is present.

In some embodiments, compound 16 has at least 1, 2, 3, 4 or 5 spectral peak(s) selected from the peaks listed in Table 32 below.

TABLE 32
XRPD Peak Positions for Compound 16

| Pos. [°2θ] | Rel. Int. [%] |
|---|---|
| 5.1 | 11.34 |
| 5.9 | 0.61 |
| 8.8 | 1.49 |
| 10.0 | 43.56 |
| 11.4 | 0.94 |
| 14.9 | 21.97 |
| 15.6 | 24.00 |
| 17.3 | 29.08 |
| 18.4 | 12.35 |
| 19.3 | 100.00 |
| 22.2 | 56.93 |
| 23.5 | 12.57 |
| 24.2 | 13.82 |
| 24.8 | 33.63 |
| 25.9 | 11.06 |
| 28.2 | 12.48 |
| 29.1 | 10.81 |
| 29.9 | 9.73 |
| 31.4 | 10.23 |
| 33.4 | 6.63 |
| 34.8 | 6.71 |
| 35.6 | 4.70 |
| 36.9 | 4.33 |
| 38.9 | 4.82 |

In some embodiments, compound 16 is characterized in that it has one or more peaks in its X-ray powder diffraction pattern selected from those at about 10.0, about 19.3 and about 22.2 degrees 2-theta. In some embodiments, compound 16 is characterized in that it has two or more peaks in its X-ray powder diffraction pattern selected from those at about 10.0, about 19.3 and about 22.2 degrees 2-theta. In some embodiments, compound 16 is characterized in that it has all three peaks in its X-ray powder diffraction pattern selected from those at about 10.0, about 19.3 and about 22.2 degrees 2-theta.

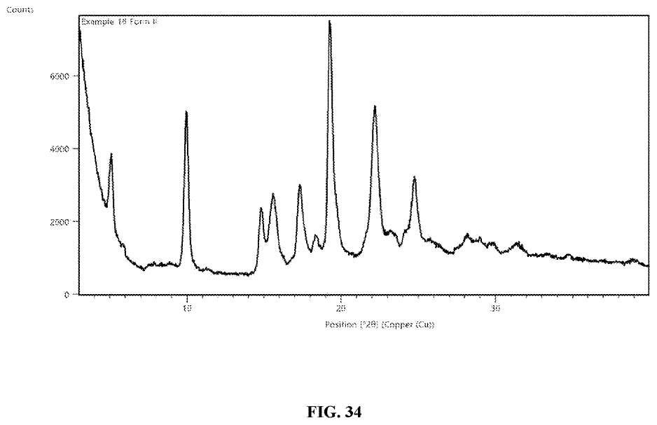

In certain embodiments, the X-ray powder diffraction pattern is substantially similar to the XRPD provided in FIG. 34.

Methods for preparing compound 16 are described infra.

In some embodiments, the present invention provides compound 16, wherein said compound is crystalline.

In some embodiments, the present invention provides compound 16, wherein said compound is a crystalline solid substantially free of amorphous compound A.

In some embodiments, the present invention provides compound 16, wherein said compound is substantially free of impurities.

In some embodiments, the present invention provides a composition comprising compound 16 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 16 or composition thereof.

In some embodiments, the present invention provides a method of treating cancer in a patient by blocking the activity of kinases, comprising administering to said patient compound 16 or composition thereof wherein compound 16 is a crystal form as described herein.

### Compound 17-Form III of Compound A

According to one embodiment, the present invention provides free base Form III of compound A, represented by compound 17.

In some embodiments, the present invention provides compound 17 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 17. In certain embodiments, at least about 95% by weight of compound 17 is present in a composition. In still other embodiments of the invention, at least about 99% by weight of compound 17 is present in a composition.

According to one embodiment, compound 17 is present in a composition in an amount of at least about 97, 97.5, 98.0, 98.5, 99, 99.5, 99.8 weight percent where the percentages are based on the total weight of the composition. According to another embodiment, compound 17 contains no more than about 3.0 area percent HPLC of total organic impurities and, in certain embodiments, no more than about 1.5 area percent HPLC total organic impurities relative to the total area of the HPLC chromatogram. In other embodiments, compound 17 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 17 is also meant to include all tautomeric forms and isomeric forms of compound 17.

It has been found that compound 17 can exist in a variety of solid forms. Exemplary such forms include polymorphs such as those described herein.

In certain embodiments, compound 17 is crystalline solid. In other embodiments, compound 17 is a crystalline solid substantially free of amorphous compound A. As used herein, the term “substantially free of amorphous compound A” means that the compound contains no significant amount of amorphous compound A. In certain embodiments, at least about 95% by weight of crystalline compound 17 is present. In still other embodiments of the invention, at least about 99% by weight of crystalline compound 17 is present.

In some embodiments, compound 17 has at least 1, 2, 3, 4 or 5 spectral peak(s) selected from the peaks listed in Table 33 below.

TABLE 33
XRPD Peak Positions for Compound 17

| Pos. [°2θ] | Rel. Int. [%] |
|---|---|
| 5.9 | 13.29 |
| 9.9 | 7.75 |
| 10.8 | 13.62 |
| 11.3 | 13.88 |
| 12.6 | 7.04 |
| 12.8 | 6.96 |
| 14.2 | 25.75 |
| 15.3 | 7.08 |
| 15.8 | 2.23 |
| 17.0 | 15.81 |
| 18.2 | 12.87 |
| 18.9 | 100.00 |
| 20.1 | 10.46 |
| 21.1 | 51.47 |
| 21.6 | 28.16 |
| 22.1 | 24.85 |
| 22.5 | 63.19 |
| 23.5 | 38.80 |
| 24.6 | 21.24 |
| 25.6 | 18.87 |
| 26.7 | 21.84 |
| 27.1 | 18.43 |
| 28.3 | 10.52 |
| 28.6 | 10.31 |
| 30.0 | 7.03 |
| 30.6 | 8.06 |
| 32.4 | 11.43 |
| 32.8 | 8.32 |
| 33.4 | 10.28 |
| 33.7 | 12.11 |
| 34.6 | 5.02 |
| 35.7 | 5.30 |
| 37.9 | 3.23 |

In some embodiments, compound 17 is characterized in that it has one or more peaks in its X-ray powder diffraction pattern selected from those at about 18.9, about 21.1 and about 22.5 degrees 2-theta. In some embodiments, compound 17 is characterized in that it has two or more peaks in its X-ray powder diffraction pattern selected from those at about 18.9, about 21.1 and about 22.5 degrees 2-theta. In some embodiments, compound 17 is characterized in that it has all three peaks in its X-ray powder diffraction pattern selected from those at about 18.9, about 21.1 and about 22.5 degrees 2-theta.

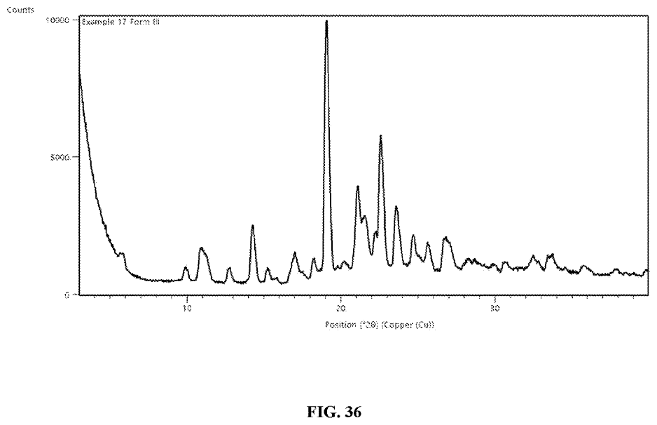

In certain embodiments, the X-ray powder diffraction pattern is substantially similar to the XRPD provided in FIG. 36.

Methods for preparing compound 17 are described infra.

In some embodiments, the present invention provides compound 17, wherein said compound is crystalline.

In some embodiments, the present invention provides compound 17, wherein said compound is a crystalline solid substantially free of amorphous compound A.

In some embodiments, the present invention provides compound 17, wherein said compound is substantially free of impurities.

In some embodiments, the present invention provides a composition comprising compound 17 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 17 or composition thereof.

In some embodiments, the present invention provides a method of treating cancer in a patient by blocking the activity of kinases, comprising administering to said patient compound 17 or composition thereof wherein compound 17 is a crystal form as described herein.

### Compound 18-Form IV of Compound A

According to one embodiment, the present invention provides free base Form IV of compound A, represented by compound 18.

In some embodiments, the present invention provides compound 18 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 18. In certain embodiments, at least about 95% by weight of compound 18 is present in a composition. In still other embodiments of the invention, at least about 99% by weight of compound 18 is present in a composition.

According to one embodiment, compound 18 is present in a composition in an amount of at least about 97, 97.5, 98.0, 98.5, 99, 99.5, 99.8 weight percent where the percentages are based on the total weight of the composition. According to another embodiment, compound 18 contains no more than about 3.0 area percent HPLC of total organic impurities and, in certain embodiments, no more than about 1.5 area percent HPLC total organic impurities relative to the total area of the HPLC chromatogram. In other embodiments, compound 18 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 18 is also meant to include all tautomeric forms and isomeric forms of compound 18.

It has been found that compound 18 can exist in a variety of solid forms. Exemplary such forms include polymorphs such as those described herein.

In certain embodiments, compound 18 is crystalline solid. In other embodiments, compound 18 is a crystalline solid substantially free of amorphous compound A. As used herein, the term “substantially free of amorphous compound A” means that the compound contains no significant amount of amorphous compound A. In certain embodiments, at least about 95% by weight of crystalline compound 18 is present. In still other embodiments of the invention, at least about 99% by weight of crystalline compound 18 is present.

In some embodiments, compound 18 has at least 1, 2, 3, 4 or 5 spectral peak(s) selected from the peaks listed in Table 34 below.

TABLE 34
XRPD Peak Positions for Compound 18

| Pos. [°2θ] | Rel. Int. [%] |
|---|---|
| 7.2 | 0.95 |
| 9.9 | 52.62 |
| 11.5 | 19.31 |
| 12.7 | 10.11 |
| 13.1 | 7.15 |
| 13.5 | 24.48 |
| 14.0 | 16.65 |
| 14.8 | 53.58 |
| 15.2 | 2.61 |
| 15.7 | 29.31 |
| 16.1 | 1.59 |
| 17.1 | 26.19 |
| 17.6 | 14.04 |
| 18.2 | 6.23 |
| 18.7 | 100.00 |
| 19.2 | 37.20 |
| 19.9 | 48.10 |
| 20.2 | 83.54 |
| 21.0 | 2.21 |
| 21.5 | 19.20 |
| 21.9 | 9.05 |
| 22.2 | 20.33 |
| 22.4 | 77.43 |
| 22.8 | 5.02 |
| 23.1 | 14.64 |
| 23.7 | 2.19 |
| 24.3 | 25.61 |
| 24.9 | 13.16 |
| 25.5 | 22.24 |
| 25.8 | 25.13 |
| 26.3 | 14.24 |
| 26.7 | 4.18 |
| 27.0 | 6.66 |
| 27.3 | 3.21 |
| 27.6 | 4.50 |
| 28.6 | 3.49 |
| 29.2 | 6.69 |
| 29.8 | 7.87 |
| 30.1 | 6.84 |
| 30.5 | 2.61 |
| 30.7 | 6.83 |
| 31.4 | 5.24 |
| 31.7 | 3.70 |
| 32.3 | 7.83 |
| 32.6 | 2.35 |
| 32.9 | 2.42 |
| 33.2 | 2.70 |
| 34.1 | 4.50 |
| 34.7 | 4.67 |
| 35.0 | 2.65 |
| 35.4 | 2.89 |
| 35.6 | 3.90 |
| 36.1 | 6.72 |
| 36.3 | 5.48 |
| 36.8 | 2.27 |
| 37.5 | 5.10 |
| 37.7 | 1.74 |
| 38.3 | 1.05 |
| 39.0 | 4.20 |
| 39.6 | 6.25 |

In some embodiments, compound 18 is characterized in that it has one or more peaks in its X-ray powder diffraction pattern selected from those at about 18.7, about 20.2 and about 22.4 degrees 2-theta. In some embodiments, compound 18 is characterized in that it has two or more peaks in its X-ray powder diffraction pattern selected from those at about 18.7, about 20.2 and about 22.4 degrees 2-theta. In some embodiments, compound 18 is characterized in that it has all three peaks in its X-ray powder diffraction pattern selected from those at about 18.7, about 20.2 and about 22.4 degrees 2-theta.

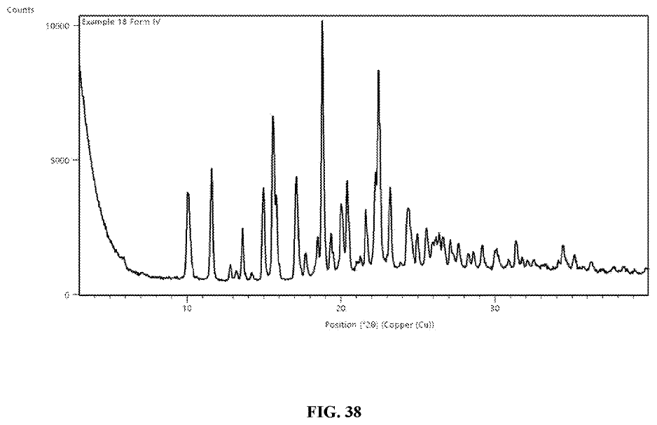

In certain embodiments, the X-ray powder diffraction pattern is substantially similar to the XRPD provided in FIG. 38.

Methods for preparing compound 18 are described infra.

In some embodiments, the present invention provides compound 18, wherein said compound is crystalline.

In some embodiments, the present invention provides compound 18, wherein said compound is a crystalline solid substantially free of amorphous compound A.

In some embodiments, the present invention provides compound 18, wherein said compound is substantially free of impurities.

In some embodiments, the present invention provides a composition comprising compound 18 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 18 or composition thereof.

In some embodiments, the present invention provides a method of treating cancer in a patient by blocking the activity of kinases, comprising administering to said patient compound 18 or composition thereof wherein compound 18 is a crystal form as described herein.

### Compound 19-Form V of Compound A

According to one embodiment, the present invention provides free base Form V of compound A, represented by compound 19.

In some embodiments, the present invention provides compound 19 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 19. In certain embodiments, at least about 95% by weight of compound 19 is present in a composition. In still other embodiments of the invention, at least about 99% by weight of compound 19 is present in a composition.

According to one embodiment, compound 19 is present in a composition in an amount of at least about 97, 97.5, 98.0, 98.5, 99, 99.5, 99.8 weight percent where the percentages are based on the total weight of the composition. According to another embodiment, compound 19 contains no more than about 3.0 area percent HPLC of total organic impurities and, in certain embodiments, no more than about 1.5 area percent HPLC total organic impurities relative to the total area of the HPLC chromatogram. In other embodiments, compound 19 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 19 is also meant to include all tautomeric forms and isomeric forms of compound 19.

It has been found that compound 19 can exist in a variety of solid forms. Exemplary such forms include polymorphs such as those described herein.

In certain embodiments, compound 19 is crystalline solid. In other embodiments, compound 19 is a crystalline solid substantially free of amorphous compound A. As used herein, the term “substantially free of amorphous compound A” means that the compound contains no significant amount of amorphous compound A. In certain embodiments, at least about 95% by weight of crystalline compound 19 is present. In still other embodiments of the invention, at least about 99% by weight of crystalline compound 19 is present.

In some embodiments, compound 19 has at least 1, 2, 3, 4 or 5 spectral peak(s) selected from the peaks listed in Table 35 below.

TABLE 35
XRPD Peak Positions for Compound 19

| Pos. [°2θ] | Rel. Int. [%] |
|---|---|
| 6.3 | 0.83 |
| 9.7 | 0.27 |
| 10.1 | 0.13 |
| 10.6 | 2.93 |
| 12.4 | 4.93 |
| 13.0 | 12.68 |
| 13.7 | 6.29 |
| 15.2 | 13.49 |
| 15.5 | 2.77 |
| 16.2 | 100.00 |
| 17.3 | 9.90 |
| 17.8 | 65.83 |
| 18.4 | 3.24 |
| 18.8 | 15.78 |
| 20.0 | 39.00 |
| 20.7 | 34.90 |
| 21.3 | 18.50 |
| 22.0 | 25.54 |
| 23.4 | 3.88 |
| 23.7 | 20.99 |
| 24.5 | 3.14 |
| 25.0 | 11.25 |
| 25.6 | 17.26 |
| 25.9 | 15.83 |
| 26.7 | 3.33 |
| 27.4 | 6.62 |
| 27.6 | 2.03 |
| 28.1 | 8.09 |
| 29.0 | 6.19 |
| 29.4 | 6.68 |
| 30.1 | 8.55 |
| 30.7 | 5.96 |
| 31.3 | 0.95 |
| 31.8 | 5.29 |
| 32.3 | 11.81 |
| 32.9 | 1.49 |
| 33.4 | 0.80 |
| 33.8 | 1.17 |
| 34.3 | 5.37 |
| 34.6 | 1.47 |
| 35.3 | 4.37 |
| 36.2 | 5.08 |
| 36.8 | 1.45 |
| 37.1 | 3.92 |
| 37.7 | 1.70 |
| 38.3 | 1.40 |
| 39.2 | 1.76 |

In some embodiments, compound 19 is characterized in that it has one or more peaks in its X-ray powder diffraction pattern selected from those at about 16.2, about 17.8 and about 20.0 degrees 2-theta. In some embodiments, compound 19 is characterized in that it has two or more peaks in its X-ray powder diffraction pattern selected from those at about 16.2, about 17.8 and about 20.0 degrees 2-theta. In some embodiments, compound 19 is characterized in that it has all three peaks in its X-ray powder diffraction pattern selected from those at about 16.2, about 17.8 and about 20.0 degrees 2-theta.

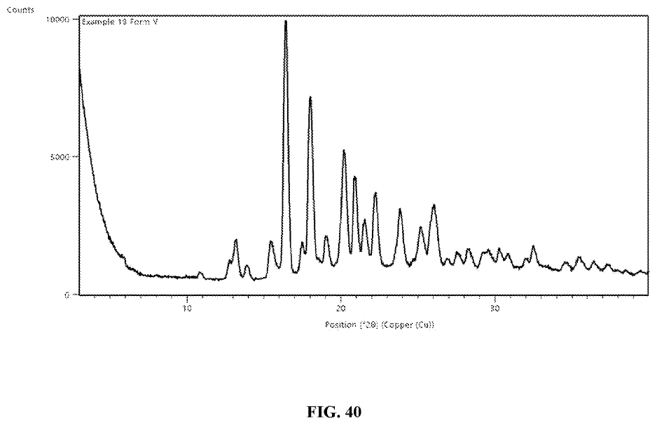

In certain embodiments, the X-ray powder diffraction pattern is substantially similar to the XRPD provided in FIG. 40.

Methods for preparing compound 19 are described infra.

In some embodiments, the present invention provides compound 19, wherein said compound is crystalline.

In some embodiments, the present invention provides compound 19, wherein said compound is a crystalline solid substantially free of amorphous compound A.

In some embodiments, the present invention provides compound 19, wherein said compound is substantially free of impurities.

In some embodiments, the present invention provides a composition comprising compound 19 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 19 or composition thereof.

In some embodiments, the present invention provides a method of treating cancer in a patient by blocking the activity of kinases, comprising administering to said patient compound 19 or composition thereof wherein compound 19 is a crystal form as described herein.

### Compound 20-Form VI of Compound A

According to one embodiment, the present invention provides free base Form VI of compound A, represented by compound 20.

In some embodiments, the present invention provides compound 20 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 20. In certain embodiments, at least about 95% by weight of compound 20 is present in a composition. In still other embodiments of the invention, at least about 99% by weight of compound 20 is present in a composition.

According to one embodiment, compound 20 is present in a composition in an amount of at least about 97, 97.5, 98.0, 98.5, 99, 99.5, 99.8 weight percent where the percentages are based on the total weight of the composition. According to another embodiment, compound 20 contains no more than about 3.0 area percent HPLC of total organic impurities and, in certain embodiments, no more than about 1.5 area percent HPLC total organic impurities relative to the total area of the HPLC chromatogram. In other embodiments, compound 20 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 20 is also meant to include all tautomeric forms and isomeric forms of compound 20.

It has been found that compound 20 can exist in a variety of solid forms. Exemplary such forms include polymorphs such as those described herein.

In certain embodiments, compound 20 is crystalline solid. In other embodiments, compound 20 is a crystalline solid substantially free of amorphous compound A. As used herein, the term “substantially free of amorphous compound A” means that the compound contains no significant amount of amorphous compound A. In certain embodiments, at least about 95% by weight of crystalline compound 20 is present. In still other embodiments of the invention, at least about 99% by weight of crystalline compound 20 is present.

In some embodiments, compound 20 has at least 1, 2, 3, 4 or 5 spectral peak(s) selected from the peaks listed in Table 36 below.

TABLE 36
XRPD Peak Positions for Compound 20

| Pos. [°2θ] | Rel. Int. [%] |
|---|---|
| 5.1 | 8.30 |
| 5.9 | 1.78 |
| 7.0 | 3.33 |
| 7.5 | 4.58 |
| 8.6 | 1.15 |
| 10.0 | 26.72 |
| 14.0 | 3.02 |
| 14.9 | 33.84 |
| 15.4 | 35.82 |
| 16.0 | 17.95 |
| 16.8 | 16.14 |
| 18.2 | 100.00 |
| 18.7 | 52.01 |
| 19.4 | 20.78 |
| 19.8 | 19.09 |
| 20.8 | 61.12 |
| 22.8 | 79.51 |
| 24.0 | 35.04 |
| 24.8 | 27.83 |
| 27.6 | 16.14 |
| 28.7 | 19.93 |
| 30.0 | 13.72 |
| 30.7 | 10.63 |
| 32.0 | 7.44 |
| 32.9 | 7.00 |
| 35.1 | 7.37 |
| 36.5 | 4.15 |
| 37.4 | 4.02 |

In some embodiments, compound 20 is characterized in that it has one or more peaks in its X-ray powder diffraction pattern selected from those at about 18.2, about 18.7 and about 20.8 degrees 2-theta. In some embodiments, compound 20 is characterized in that it has two or more peaks in its X-ray powder diffraction pattern selected from those at about 18.2, about 18.7 and about 20.8 degrees 2-theta. In some embodiments, compound 20 is characterized in that it has all three peaks in its X-ray powder diffraction pattern selected from those at about 18.2, about 18.7 and about 20.8 degrees 2-theta.

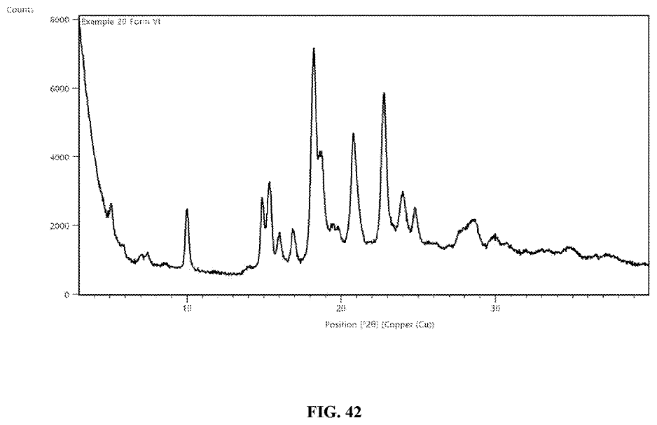

In certain embodiments, the X-ray powder diffraction pattern is substantially similar to the XRPD provided in FIG. 42.

Methods for preparing compound 20 are described infra.

In some embodiments, the present invention provides compound 20, wherein said compound is crystalline.

In some embodiments, the present invention provides compound 20, wherein said compound is a crystalline solid substantially free of amorphous compound A.

In some embodiments, the present invention provides compound 20, wherein said compound is substantially free of impurities.

In some embodiments, the present invention provides a composition comprising compound 20 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 20 or composition thereof.

In some embodiments, the present invention provides a method of treating cancer in a patient by blocking the activity of kinases, comprising administering to said patient compound 20 or composition thereof wherein compound 20 is a crystal form as described herein.

### Compound 21-Form VII of Compound A

According to one embodiment, the present invention provides free base Form VII of compound A, represented by compound 21.

In some embodiments, the present invention provides compound 21 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 21. In certain embodiments, at least about 95% by weight of compound 21 is present in a composition. In still other embodiments of the invention, at least about 99% by weight of compound 21 is present in a composition.

According to one embodiment, compound 21 is present in a composition in an amount of at least about 97, 97.5, 98.0, 98.5, 99, 99.5, 99.8 weight percent where the percentages are based on the total weight of the composition. According to another embodiment, compound 21 contains no more than about 3.0 area percent HPLC of total organic impurities and, in certain embodiments, no more than about 1.5 area percent HPLC total organic impurities relative to the total area of the HPLC chromatogram. In other embodiments, compound 21 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 21 is also meant to include all tautomeric forms and isomeric forms of compound 21.

It has been found that compound 21 can exist in a variety of solid forms. Exemplary such forms include polymorphs such as those described herein.

In certain embodiments, compound 21 is crystalline solid. In other embodiments, compound 21 is a crystalline solid substantially free of amorphous compound A. As used herein, the term “substantially free of amorphous compound A” means that the compound contains no significant amount of amorphous compound A. In certain embodiments, at least about 95% by weight of crystalline compound 21 is present. In still other embodiments of the invention, at least about 99% by weight of crystalline compound 21 is present.

In some embodiments, compound 21 has at least 1, 2, 3, 4 or 5 spectral peak(s) selected from the peaks listed in Table 37 below.

TABLE 37
XRPD Peak Positions for Compound 21

| Pos. [°2θ] | Rel. Int. [%] |
|---|---|
| 6.0 | 3.64 |
| 6.9 | 31.03 |
| 9.3 | 32.32 |
| 11.8 | 38.64 |
| 13.7 | 4.16 |
| 14.4 | 92.61 |
| 15.1 | 24.31 |
| 15.4 | 27.21 |
| 16.8 | 20.54 |
| 17.6 | 31.54 |
| 18.0 | 100.00 |
| 19.9 | 91.65 |
| 20.4 | 15.49 |
| 20.8 | 16.12 |
| 21.2 | 8.12 |
| 22.2 | 77.22 |
| 22.6 | 58.08 |
| 23.1 | 10.59 |
| 23.7 | 15.86 |
| 24.1 | 52.70 |
| 24.7 | 24.46 |
| 25.5 | 50.89 |
| 26.1 | 10.01 |
| 27.1 | 35.79 |
| 28.3 | 22.29 |
| 28.7 | 20.17 |
| 29.4 | 22.75 |
| 29.5 | 20.69 |
| 30.1 | 16.00 |
| 31.5 | 11.72 |
| 32.8 | 10.98 |
| 33.6 | 13.31 |
| 34.5 | 11.43 |
| 35.2 | 6.76 |
| 36.1 | 6.54 |
| 36.7 | 7.56 |
| 37.7 | 9.77 |
| 38.1 | 5.96 |
| 39.0 | 6.21 |

In some embodiments, compound 21 is characterized in that it has one or more peaks in its X-ray powder diffraction pattern selected from those at about 14.4, about 18.0 and about 19.9 degrees 2-theta. In some embodiments, compound 21 is characterized in that it has two or more peaks in its X-ray powder diffraction pattern selected from those at about 14.4, about 18.0 and about 19.9 degrees 2-theta. In some embodiments, compound 21 is characterized in that it has all three peaks in its X-ray powder diffraction pattern selected from those at about 14.4, about 18.0 and about 19.9 degrees 2-theta.

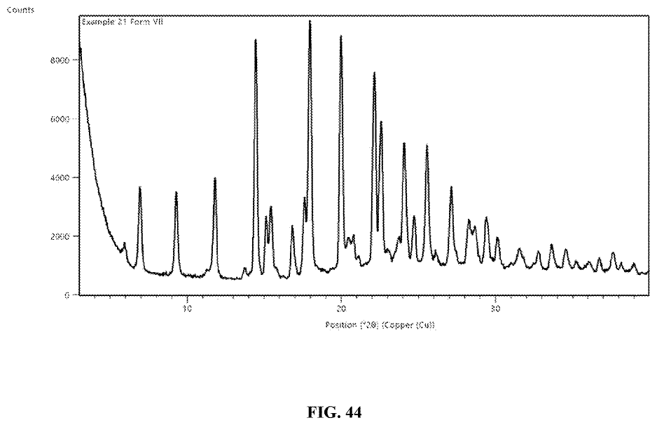

In certain embodiments, the X-ray powder diffraction pattern is substantially similar to the XRPD provided in FIG. 44.

Methods for preparing compound 21 are described infra.

In some embodiments, the present invention provides compound 21, wherein said compound is crystalline.

In some embodiments, the present invention provides compound 21, wherein said compound is a crystalline solid substantially free of amorphous compound A.

In some embodiments, the present invention provides compound 21, wherein said compound is substantially free of impurities.

In some embodiments, the present invention provides a composition comprising compound 21 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 21 or composition thereof.

In some embodiments, the present invention provides a method of treating cancer in a patient by blocking the activity of kinases, comprising administering to said patient compound 21 or composition thereof wherein compound 21 is a crystal form as described herein.

### Compound 22-Form VIII of Compound A

According to one embodiment, the present invention provides free base Form VIII of compound A, represented by compound 22.

In some embodiments, the present invention provides compound 22 substantially free of impurities. As used herein, the term “substantially free of impurities” means that the compound contains no significant amount of extraneous matter. Such extraneous matter may include excess benzenesulfonic acid, excess synthetic reagents, excess compound A, residual solvents, or any other impurities that may result from the preparation of, and/or isolation of, compound 22. In certain embodiments, at least about 95% by weight of compound 22 is present in a composition. In still other embodiments of the invention, at least about 99% by weight of compound 22 is present in a composition.

According to one embodiment, compound 22 is present in a composition in an amount of at least about 97, 97.5, 98.0, 98.5, 99, 99.5, 99.8 weight percent where the percentages are based on the total weight of the composition. According to another embodiment, compound 22 contains no more than about 3.0 area percent HPLC of total organic impurities and, in certain embodiments, no more than about 1.5 area percent HPLC total organic impurities relative to the total area of the HPLC chromatogram. In other embodiments, compound 22 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 22 is also meant to include all tautomeric forms and isomeric forms of compound 22.

It has been found that compound 22 can exist in a variety of solid forms. Exemplary such forms include polymorphs such as those described herein.

In certain embodiments, compound 22 is crystalline solid. In other embodiments, compound 22 is a crystalline solid substantially free of amorphous compound A. As used herein, the term “substantially free of amorphous compound A” means that the compound contains no significant amount of amorphous compound A. In certain embodiments, at least about 95% by weight of crystalline compound 22 is present. In still other embodiments of the invention, at least about 99% by weight of crystalline compound 22 is present.

In some embodiments, compound 22 has at least 1, 2, 3, 4 or 5 spectral peak(s) selected from the peaks listed in Table 38 below.

TABLE 38
XRPD Peak Positions for Compound 22

| Pos. [°2θ] | Rel. Int. [%] |
|---|---|
| 5.2 | 53.77 |
| 5.3 | 45.94 |
| 7.3 | 12.88 |
| 9.5 | 2.93 |
| 10.2 | 65.82 |
| 11.8 | 3.63 |
| 13.7 | 10.89 |
| 14.1 | 11.04 |
| 14.4 | 16.86 |
| 14.6 | 13.48 |
| 15.3 | 40.63 |
| 16.1 | 21.74 |
| 16.6 | 10.77 |
| 17.4 | 43.65 |
| 18.2 | 55.22 |
| 18.7 | 93.74 |
| 19.7 | 50.04 |
| 20.2 | 32.82 |
| 20.9 | 44.61 |
| 21.2 | 77.25 |
| 22.8 | 66.29 |
| 23.0 | 100.00 |
| 24.3 | 48.43 |
| 25.2 | 26.65 |
| 25.7 | 20.22 |
| 26.5 | 10.81 |
| 26.7 | 10.11 |
| 28.5 | 21.38 |
| 28.9 | 17.01 |
| 29.5 | 20.65 |
| 31.9 | 6.56 |
| 35.3 | 5.49 |
| 35.8 | 10.04 |
| 38.0 | 2.53 |

In some embodiments, compound 22 is characterized in that it has one or more peaks in its X-ray powder diffraction pattern selected from those at about 18.7, about 21.2 and about 23.0 degrees 2-theta. In some embodiments, compound 22 is characterized in that it has two or more peaks in its X-ray powder diffraction pattern selected from those at about 18.7, about 21.2 and about 23.0 degrees 2-theta. In some embodiments, compound 22 is characterized in that it has all three peaks in its X-ray powder diffraction pattern selected from those at about 18.7, about 21.2 and about 23.0 degrees 2-theta.

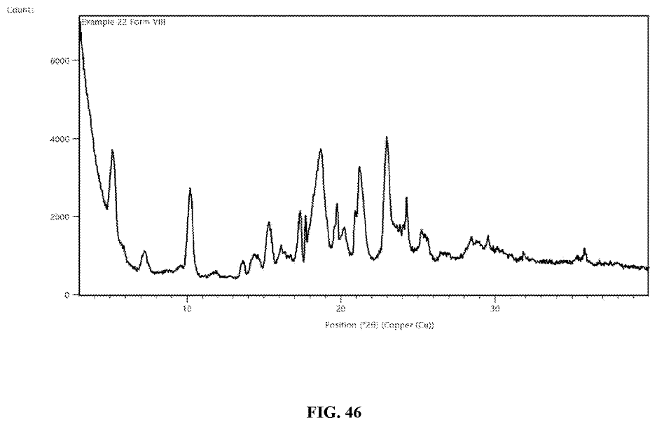

In certain embodiments, the X-ray powder diffraction pattern is substantially similar to the XRPD provided in FIG. 46.

Methods for preparing compound 22 are described infra.

In some embodiments, the present invention provides compound 22, wherein said compound is crystalline.

In some embodiments, the present invention provides compound 22, wherein said compound is a crystalline solid substantially free of amorphous compound A.

In some embodiments, the present invention provides compound 22, wherein said compound is substantially free of impurities.

In some embodiments, the present invention provides a composition comprising compound 22 and a pharmaceutically acceptable carrier or excipient.

In some embodiments, the present invention provides a method of treating, preventing, and/or reducing a risk of a disease, disorder, or condition in a kinase-mediated disorder in a patient comprising administering to said patient compound 22 or composition thereof.

In some embodiments, the present invention provides a method of treating cancer in a patient by blocking the activity of kinases, comprising administering to said patient compound 22 or composition thereof wherein compound 22 is a crystal form as described herein.

In some embodiments, the present invention provides a compound selected from: compound 1, Form I; compound 2, Form I; compound 2, Form II; compound 2, Form III; compound 2, Form IV; compound 2, Form V; compound 2, Form VI; compound 2, Form VII; compound 2, Form VIII; compound 2, Form IX; compound 2, Form X; compound 2, Form XI; compound 2, Form XII; compound 2, Form XIII; compound 3, Form I; compound 4, Form I; compound 5, Form I; compound 6, Form I; compound 7, Form I; compound 8, Form I; compound 9; compound 10, Form I; compound 11, Form I; compound 12, Form I; compound 13; compound 14; compound 15; compound 16; compound 17; compound 18; compound 19; compound 20; compound 21; compound 22; compound 23, Form I; compound 24, Form I; compound 25, Form I; and compound 26, Form I.

In some such embodiments, the present invention provides a composition comprising one of the above compound forms and a pharmaceutically acceptable carrier or excipient. In some such embodiments, the present invention provides a method of treating one or more of the diseases, disorders, or conditions described herein.

Aspects of this disclosure are directed to other salt forms of compound A, including compound A citric acid salt; compound A oxalic acid salt; compound A malonic acid salt; compound A succinic acid salt; compound A fumaric acid salt; compound A L(+)-tartaric acid salt; compound A D(−)-tartaric acid salt; compound A benzoic acid salt; compound A ascorbic acid salt; compound A phosphoric acid salt; compound A L-aspartic acid salt; compound A ketoglutaric acid salt; compound A gluconic acid salt; compound A lactic acid salt; compound A acetic acid salt; and compound A pamoic acid salt.

### General Methods of Providing a Salt Compound

Compound A is prepared according to the methods described in detail in the '500 publication, the entirety of which is hereby incorporated herein by reference. Salt compounds of general formula X, which formula encompasses, inter alia, salt compounds 1-12 and 23-26, and/or particular forms thereof, are prepared from compound A, according to the general Scheme below.

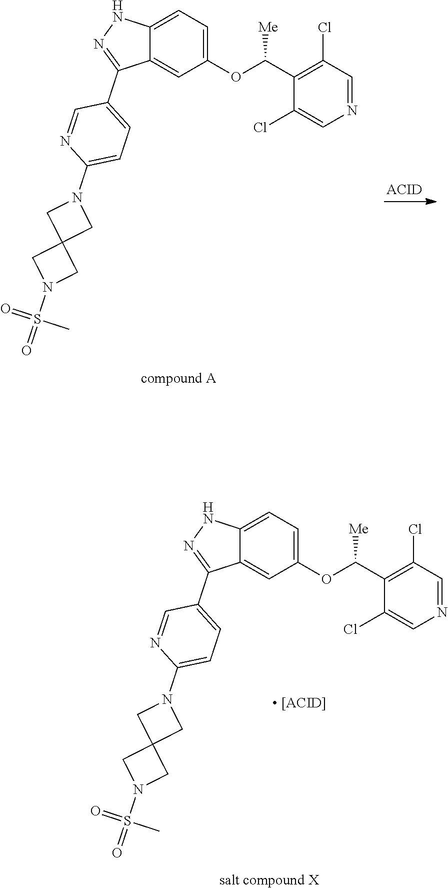

For instance, each of compounds 1-12 and 23-26, and forms thereof, are prepared from compound A by combining compound A with an appropriate acid to form a salt of that acid. Thus, another aspect of the present invention provides a method for preparing compounds 1-12 and 23-26, and forms thereof.

As described generally above, in some embodiments, the present invention provides a method for preparing a salt compound of the general formula X:

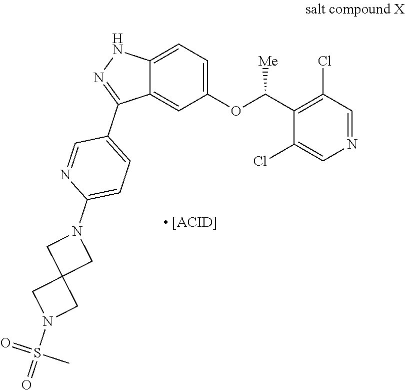

comprising steps of:
combining compound A:

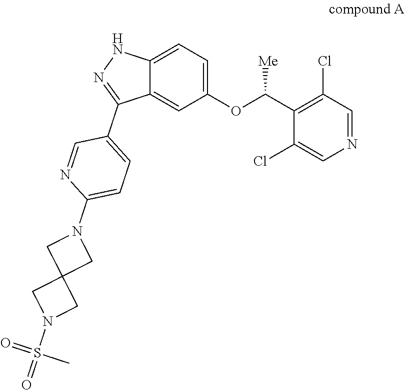 [SMILES: C[C@@H](Oc1ccc2[nH]nc(-c3ccc(N4CC5(C4)CN(S(C)(=O)=O)C5)nc3)c2c1)c1c(Cl)cncc1Cl]

with a suitable acid and optionally a suitable solvent under conditions suitable for forming a salt compound of general formula X.

In some embodiments, a suitable acid is hydrochloric acid. In some embodiments, the present invention provides a method of making a hydrochloride salt of compound A. In certain embodiments, the hydrochloride salt of compound A is compound 1. In certain embodiments, the hydrochloride salt of compound A is Form I of compound 1.

In some embodiments, a suitable acid is benzenesulfonic acid. In some embodiments, the present invention provides a method of making a besylate salt of compound A. In certain embodiments, the besylate salt of compound A is compound 2. In certain embodiments, the besylate salt of compound A is Form I of compound 2. In certain embodiments, the besylate salt of compound A is Form II of compound 2. In certain embodiments, the besylate salt of compound A is Form III of compound 2. In certain embodiments, the besylate salt of compound A is Form IV of compound 2. In certain embodiments, the besylate salt of compound A is Form V of compound 2. In certain embodiments, the besylate salt of compound A is Form VI of compound 2. In certain embodiments, the besylate salt of compound A is Form VII of compound 2. In certain embodiments, the besylate salt of compound A is Form VIII of compound 2. In certain embodiments, the besylate salt of compound A is Form IX of compound 2. In certain embodiments, the besylate salt of compound A is Form X of compound 2. In certain embodiments, the besylate salt of compound A is Form XI of compound 2. In certain embodiments, the besylate salt of compound A is Form XII of compound 2. In certain embodiments, the besylate salt of compound A is Form XIII of compound 2.

In some embodiments, a suitable acid is maleic acid. In some embodiments, the present invention provides a method of making a maleate salt of compound A. In certain embodiments, the maleate salt of compound A is compound 3. In certain embodiments, the maleate salt of compound A is Form I of compound 3.

In some embodiments, a suitable acid is p-toluenesulfonic acid. In some embodiments, the present invention provides a method of making a tosylate salt of compound A. In certain embodiments, the tosylate salt of compound A is compound 4.

In some embodiments, a suitable acid is sulfuric acid. In some embodiments, the present invention provides a method of making a sulfate salt of compound A. In certain embodiments, the sulfate salt of compound A is compound 5.

In some embodiments, a suitable acid is sulfonic acid. In some embodiments, the present invention provides a method of making a sulfonate salt of compound A. In certain embodiments, the sulfonate salt of compound A is compound 6.

In some embodiments, a suitable acid is ethane sulfonic acid. In some embodiments, the present invention provides a method of making an esylate salt of compound A. In certain embodiments, the esylate salt of compound A is compound 7.

In some embodiments, a suitable acid is methane sulfonic acid. In some embodiments, the present invention provides a method of making a mesylate salt of compound A. In certain embodiments, the mesylate salt of compound A is compound 8.

In some embodiments, a suitable acid is methane sulfonic acid. In some embodiments, the present invention provides a method of making a di-mesylate salt of compound A. In certain embodiments, the di-mesylate salt of compound A is compound 9.

In some embodiments, a suitable acid is R-camphor sulfonic acid. In some embodiments, the present invention provides a method of making an R-camsylate salt of compound A. In certain embodiments, the R-camsylate salt of compound A is compound 10. In certain embodiments, the R-camsylate salt of compound A is Form I of compound 10.

In some embodiments, a suitable acid is S-camphor sulfonic acid. In some embodiments, the present invention provides a method of making an S-camsylate salt of compound A. In certain embodiments, the S-camsylate salt of compound A is compound 11.

In some embodiments, a suitable acid is hydrobromic acid. In some embodiments, the present invention provides a method of making a bromate salt of compound A. In certain embodiments, the bromate salt of compound A is compound 12. In certain embodiments, the bromate salt of compound A is Form I of compound 12.

In some embodiments, a suitable acid is phosphoric acid. In some embodiments, the present invention provides a method of making a phosphate salt of compound A. In certain embodiments, the phosphate salt of compound A is compound 23. In certain embodiments, the phosphate salt of compound A is Form I of compound 23.

In some embodiments, a suitable acid is citric acid. In some embodiments, the present invention provides a method of making a citrate salt of compound A. In certain embodiments, the citrate salt of compound A is compound 24. In certain embodiments, the citrate salt of compound A is Form I of compound 24.

In some embodiments, a suitable acid is L-tartaric acid. In some embodiments, the present invention provides a method of making an L-tartrate salt of compound A. In certain embodiments, the L-tartrate salt of compound A is compound 25. In certain embodiments, the L-tartrate salt of compound A is Form I of compound 25.

In some embodiments, a suitable acid is L-malonic acid. In some embodiments, the present invention provides a method of making a malonate salt of compound A. In certain embodiments, the malonate salt of compound A is compound 26. In certain embodiments, the malonate salt of compound A is Form I of compound 26.

A suitable solvent may be any solvent system (e.g., one solvent or a mixture of solvents) in which compound A and/or an acid are soluble or are at least partially soluble.

Examples of suitable solvents useful in the present invention include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, the solvent is one or more organic alcohols. In some embodiments, the solvent is chlorinated. In some embodiments, the solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethylformamide, dimethylsulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, the suitable solvent is MTBE.

In some embodiments, a suitable solvent is ethyl acetate. In some embodiments, a suitable solvent is a mixture of methanol and methylene chloride. In some embodiments, a suitable solvent is a mixture of acetonitrile and water. In certain embodiments, a suitable solvent is methyl acetate, isopropyl acetate, acetone, or tetrahydrofuran. In certain embodiments, a suitable solvent is diethylether. In certain embodiments, a suitable solvent is water. In certain embodiments, a suitable solvent is methyl ethyl ketone. In certain embodiments, a suitable solvent is toluene.

In some embodiments, the present invention provides a method for preparing a salt compound of the general formula X, comprising one or more steps of removing a solvent and adding a solvent. In some embodiments, an added solvent is the same as the solvent removed. In some embodiments, an added solvent is different from the solvent removed. Means of solvent removal are known in the synthetic and chemical arts and include, but are not limited to, any of those described herein and in the Exemplification.

In some embodiments, a method for preparing a salt compound of the general formula X comprises one or more steps of heating or cooling a preparation.

In some embodiments, a method for preparing a salt compound of the general formula X comprises one or more steps of agitating or stirring a preparation.

In some embodiments, a method for preparing a salt compound of the general formula X comprises a step of adding a suitable acid to a solution or slurry of compound A.

In some embodiments, a method for preparing a salt compound of the general formula X comprises a step of heating.

In certain embodiments, a salt compound of formula X precipitates from the mixture. In another embodiment, a salt compound of formula X crystallizes from the mixture. In other embodiments, a salt compound of formula X crystallizes from solution following seeding of the solution (i.e., adding crystals of a salt compound of formula X to the solution).

A salt compound of formula X can precipitate out of the reaction mixture or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a salt compound of formula X is optionally isolated. It will be appreciated that a salt compound of formula X may be isolated by any suitable physical means known to one of ordinary skill in the art. In certain embodiments, precipitated solid salt compound of formula X is separated from the supernatant by filtration. In other embodiments, precipitated solid salt compound of formula X is separated from the supernatant by decanting the supernatant.

In certain embodiments, a salt compound of formula X is separated from the supernatant by filtration.

In certain embodiments, an isolated salt compound of formula X is dried in air. In other embodiments, isolated salt compound of formula X is dried under reduced pressure, optionally at elevated temperature.

### Uses of Compounds and Pharmaceutically Acceptable Compositions Thereof

The FGFR receptors (FGFR1, FGFR2, FGFR3, and FGFR4) share several structural features in common, including three extracellular immunoglobulin-like (Ig) domains, a hydrophobic transmembrane domain, and an intracellular tyrosine kinase domain split by a kinase insert domain, followed by a cytoplasmic c-terminal tail (Johnson et al., Adv. Cancer Res. 60:1-40, 1993; and Wilkie et al., Curr. Biol. 5:500-507, 1995). In FGFR1, the kinase insert domain spans positions 582 to 595 of the alpha Al isoform of FGFR1. In FGFR2, the kinase insert domain spans positions 585 to 598 of the FGFR2 Ille isoform. In FGFR3, the kinase insert domain spans positions 576 to 589 of the FGFR3 Ille isoform. In FGFR4, the kinase insert domain spans positions 571 to 584 of FGFR4 isoForm I. The c-terminal tail of FGFRs begins following the end of the tyrosine kinase domain and extends to the c-terminus of the protein. Several isoforms of each FGFR have been identified and are the result of alternative splicing of their mRNAs (Johnson et al., Mol. Cell. Biol. 11:4627-4634, 1995; and Chellaiah et al., J. Biol. Chem. 269:11620-11627, 1994).

A few of the receptor variants that result from this alternative splicing have different ligand binding specificities and affinities (Zimmer et al., J. Biol. Chem. 268:7899-7903, 1993; Cheon et al., Proc. Natl. Acad. Sci. U.S.A. 91:989-993, 1994; and Miki et al., Proc. Natl. Acad. Sci. U.S.A. 89:246-250, 1992). Protein sequences for FGFR proteins and nucleic acids encoding FGFR proteins are known in the art. Signaling by FGFRs regulates key biological processes including cell proliferation, survival, migration, and differentiation. Dysregulation of a FGFR gene, a FGFR protein, or expression or activity, or level of the same, has been associated with many types of cancer. For example, dysregulation of FGFRs can occur by multiple mechanisms, such as FGFR gene overexpression, FGFR gene amplification, activating mutations (e.g., point mutations or truncations), and chromosomal rearrangements that lead to FGFR fusion proteins. Dysregulation of a FGFR gene, a FGFR protein, or expression or activity, or level of the same, can result in (or cause in part) the development of a variety of different FGFR-associated cancers.

FGFR fusion proteins are known in the art. See, e.g., Baroy et al., PloS One; 11(9):e0163859. doi: 10.1371/journal.pone.0163859, 2016; Ren et al., Int. J. Cancer, 139(4):836-40, 2016; Marchwicka et al., Cell Biosci., 6:7. doi: 10.1186/s13578-016-0075-9, 2016; PCT Patent Application Publication No. WO 2014/071419A2; U.S. Patent Application Publication No. 2015/0366866A1; PCT Patent Application Publication No. WO 2016/084883A1; PCT Patent Application Publication No. WO 2016/030509A1; PCT Patent Application Publication No. WO 2015/150900A2; PCT Patent Application Publication No. WO 2015/120094A2; Kasaian et al., BMC Cancer., 15:984, 2015; Vakil et al., Neuro-Oncology, 18: Supp. Supplement 3, pp. iii93. Abstract Number: LG-64, 17^{th} International Symposium on Pediatric Neuro-Oncology, Liverpool, United Kingdom, 2016; Astsaturov et al., Journal of Clinical Oncology, 34: Supp. Supplement 15, Abstract Number: 11504, 2016 Annual Meeting of the American Society of Clinical Oncology, Chicago, IL; Heinrich et al., Journal of Clinical Oncology, 34: Supp. Supplement 15, Abstract Number: 11012, 2016 Annual Meeting of the American Society of Clinical Oncology, Chicago, IL; Hall et al., Molecular Cancer Therapeutics, Vol. 14, No. 12, Supp. 2, Abstract Number: B151, AACR-NCI-EORTC International Conference: Molecular Targets and Cancer Therapeutics, 2015; Reuther et al., Journal of Molecular Diagnostics, Vol. 17, No. 6, pp. 813, Abstract Number: ST02, 2015 Annual Meeting of the Association for Molecular Pathology, Austin, TX; Moeini et al., Clin. Cancer. Res., 22(2):291-300, 2016; Schrock et al, J Thorac. Oneal. pii S1556-0864(18)30674-9, 2018. doi: 10.1016/j.jtho.2018.05.027; Pekmezci et al, Acta Nurotaphol. Commun. 6(1):47. doi: 10.1186/s40478-018-0551-z; Lowery et al. Clin Cancer Res. pii: clincanres.0078.2018. doi: 10.1158/1078-0432.CCR-18-0078; Ryland et al. J Clin Pathol pii: jclinpath-2018-205195, 2018. doi: 10.1136/jclinpath-2018-205195; Ferguson et al. J Neuropathol Exp Neural 77(6):437-442, 2018. doi: 10.1093/jnen/nly022; Wu et al, BMC Cancer 18(1):343, 2018. doi: 10.1186/s12885-018-4236-6; Shibata et al, Cancer Sci 109(5):1282-1291, 2018. doi: 10.1111/cas. 13582; Papdopoulos et al, Br J Cancer, 1117(11):1592-1599, 2017. doi: 10.1038/bjc.2017.330; Hall et al, PLoS One, 11(9):e1062594, 2016. doi: 10.1371/journal.pone.0162594; Johnson et al, Oncologist, 22(12):1478-1490, 2017. doi: 10.1634/theoncologist.2017-0242; Yang et al, Am J Hum Genet, 98(5):843-856, 2016. doi: 10.1016/j.ajhg.2016.03.017; U.S. Patent Application Publication No. 2013/009621; Babina and Turner, Nat Rev Cancer 17(5):318-332, 2017. doi: 10.1038/nrc.2017.8; Ryland et al, J Clin Pathol., 2018 May 14. pii: jclinpath-2018-205195. doi: 10.1136/jclinpath-2018-205195; Kumar et al, Am J Clin Pathol. 143(5):738-748, 2015. doi: 10.1309/AJCPUD6WIJLQQMNA; Grand et al, Genes Chromosomes Cancer 40(1):78-83, 2004. doi: 10.1002/gcc.20023; Reeser, et al, J Mol Diagn, 19(5):682-696, 2017. doi: 10.1016/j.jmoldx.2017.05.006; Basturk, et al, Mod Pathol, 30(12):1760-1772, 2017. doi: 10.1038/modpathol.2017.60; Wang, et al, Cancer 123(20):3916-3924, 2017. doi: 10.1002/cncr.30837; Kim, et al, Oncotarget, 8(9):15014-15022, 2017. doi: 10.18632/oncotarget. 14788; Busse, et al, Genes Chromosomes Cancer, 56(10):730-749, 2017. doi: 10.1002/gcc.22477; Shi, et al, J Transl Med., 14(1):339, 2016. doi: 10.1186/s12967-016-1075-6, each of which is incorporated by reference herein.

FGFR point mutations are known in the art. See, e.g., UniParc entry UPI00000534B8; UniParc entry UPI0000001COF; UniParc entry UPI000002A99A; UniParc entry UPI000012A72A; UniParc entry UPI000059D1C2; UniParc entry UPI000002A9AC; Uniparc entry UPI000012A72C; Uniparc entry UPI000012A72D; Uniparc entry UPI000013EOB8; Uniparc entry UPI0001CE06A3; Gen bank entry BAD92868.1; Ang et al., Diagn. Mol. Pathol. Feb. 24, 2014; U.S. Patent Application Publication No. 2011/0008347; Gallo et al., Cytokine Growth Factor Rev. 26:425-449, 2015; Davies et al., J. Cancer Res. 65:7591, 2005; Kelleher et al., Carcinogenesis 34:2198, 2013; Cazier et al., Nat. Commun. 5:3756, 2014; Liu et al., Genet. Mol. Res. 13:1109, 2014; Trudel et al., Blood 107:4039, 2006; Gallo et al., Cytokine Growth Factor Rev. 26:425, 2015; Liao et al., Cancer Res. 73:5195-5205, 2013; Martincorena et al., Science 348:880 (2015); U.S. Patent Application Publication No. US2016/0235744A1; U.S. Pat. No. 9,254,288B2; U.S. Pat. No. 9,267,176B2; U.S. Patent Application Publication No. S2016/0215350A1; European Patent Application Publication No. EP3023101A1; PCT Patent Application Publication No. WO2016105503A1; Rivera et al., Acta. Neuropathol., 131(6):847-63, 2016; Lo Iacono et al., Oncotarget., 7(12):14394-404, 2016; Deeken et al., Journal of Clinical Oncology, 34: Supp. Supplement 15, pp. iii93. Abstract Number: el 7520, 2016 Annual Meeting of the American Society of Clinical Oncology, Chicago, IL; Sullivan et al., Journal of Clinical Oncology, 34: Supp. Supplement 15, pp. iii93. Abstract Number: 11596, 2016 Annual Meeting of the American Society of Clinical Oncology, Chicago, IL; Nguyen et al., Molecular Cancer Therapeutics, Vol. 14, No. 12, Supp. 2, Abstract Number: C199, AACR-NCI-EORTC International Conference: Molecular Targets and Cancer Therapeutics, 2015; Li et al., Hum. Pathol., 55:143-50, 2016; European Patent No. EP2203449B1; Yoza et al., Genes Cells, (10):1049-1058, 2016; U.S. Pat. No. 9,254,288B2; European Patent Application Publication No. 3023101A1; PCT Application Publication No. WO 2015/099127A1; European Patent No. EP2203449B1; Yoza et al., Genes Cells, (10):1049-1058, 2016; Bunney et al., EbioMedicine, 2(3):194-204, 2015; Byron et al., Neoplasia, 15(8):975-88, 2013; European Patent Application Publication No. EP3023101A1; PCT Application Publication No. WO 2015/099127A1; Thussbas et al., J. Clin. Oneal., 24(23):3747-55, 2006; Chell et al., Oncogene, 32(25):3059-70, 2013; Tanizaki et al, Cancer Res. 75(15):3149-3146 doi: 10.1158/0008-5472.CAN-14-3771; Yang et al, EBioMedicine pii S2352-3964(18)30218-4. doi: 10.1016/j.ebiom.2018.06.011; Jakobsen, et al Oncotarget 9(40):26195-26208, 2018. doi: 10.18632/oncotarget.25490; Stone, et al Acta Neuropathol 135(1):115-129, 2017. doi: 10.1007/s00401-017-1773-z; Pekmezci et al, Acta Nurotaphol. Commun. 6(1):47. doi: 10.1186/s40478-018-0551-z; De Mattos-Arruda et al, Oncotarget 9(29):20617-20630, 2018. doi: 10.18632/oncotarget.25041; Oliveira et al, J Exp Clin Cancer Res 37(1):84, 2018. doi: 10.1186/s13046-018-0746-y; Cha et al, Mol Oneal 12(7):993-1003, 2018. doi: 10.1002/1878-0261.12194; Ikeda et al, Oncologist, 23(5):586-593, 2018. doi: 10.1634/theoncologist.2017-0479; Pelaez-Garda et al, PLoS One, 8(5):e63695, 2013. doi: 10.1371/journal.pone.0063695; Shimada et al, Oncotarget, 8(55):93567-93579, 2017. doi: 10.18632/oncotarget.20510; Welander et al, World J Surg, 42(2):482-489, 2018. doi: 10.1007/s00268-01 7-4320-0; Chandrani et al, Ann Oneal, 28(3):597-603, 2017. doi: 10.1093/annonc/mdw636; Dalin et al, Nat Commun, 8(1):1197, 2017. doi: 10.1038/s41467-017-01178-z; Taurin et al, Intl Gynecol Cancer, 28(1):152-160, 2018. doi: 10.1097/IGC.0000000000001129; Haugh et al, J Invest Dermatol 138(2):384-393, 2018. doi: 10.1016/j.jid.2017.08.022; Babina and Turner, Nat Rev Cancer 17(5):318-332, 2017. doi: 10.1038/nrc.2017.8; Greenman et al, Nature 446(7132):153-158, 2007. doi: 10.1038/nature05610; Helsten et al, Clin Cancer Res, 22(1):259-267, 2016. doi: 10.1158/1078-0432.CCR-14-3212; Kim et al, BMC Urol, 18:68, 2018. doi: 10.1186/s12894-018-0380-1; Goyal et al, Cancer Discov, 7(3):252-263, 2017. doi: 10.1158/2159-8290.CD-16-1000; Premov et al, Oncogene, 36(22):3168-3177, 2017. doi: 10.1038/one.2016.464; Geelvink et al, Int J Mol Sci. 19(9): pii: E2548, 2018. doi: 10.3390/ijms19092548; Lee et al, Exp Ther Med. 16(2):1343-1349, 2018. doi: 10.3892/etm.2018.6323; Kas et al, Cancer Res, 78(19):5668-5679, 2018. doi: 10.1158/0008-5472.CAN-18-0757; Chesi et al, Blood, 97(3):729-736, 2001. PMID: 11157491. Note that the deletion of FGFR3 isoform Ille residues 795-808 also deletes the stop codon, elongating the protein by 99 amino acids (ATGPQQCEGSLAAHPAAGAQPLPGMRLSADGETATQSFGLCVCVCVCVCVCTSACACVRAH LASRCRGTLGVPAA VQRSPDWCCSTEGPLFWGDPVONVSGPTRWDPVGQGAGPDMARPLPLHHGTSQGALGPSH TQS); Ge, et al, Am J Cancer Res. 7(7):1540-1553, 2017. PMID: 28744403; Jiao et al, Nat Genet, 45(12):1470-1473, 2013. doi: 10.1038/ng.2813; Jusakul et al, Cancer Discov. 7(10):1116-1135, 2017. doi: 10.1158/2159-8290.CD-17-0368; Guyard et al, Respir Res., 18(1):120, 2018. doi: 10.1186/s12931-017-0605-y; Paik et al, Clin Cancer Res., 23(18):5366-5373, 2017. doi: 10.1158/1078-0432.CCR-17-0645; Roy et al, Mod Pathol., 30(8):1133-1143, 2017. doi: 10.1038/modpathol.2017.33; Chakrabarty et al, Br J Cancer, 117(1):136-143, 2017. doi: 10.1038/bjc.2017.148; Hoang et al, Sci Transl Med., 5(197):197ra102. doi: 10.1126/scitranslmed.3006200; Kim et al, Ann Oneal., 28(6):1250-1259. doi: 10.1093/annonc/mdx098, each of which is incorporated by reference herein.

Compounds of the disclosure have been found to inhibit FGFR1, FGFR2, FGFR3, and/or FGFR4 and are therefore believed to be useful for treating diseases and disorders which can be treated with an inhibitor of FGFR1, FGFR2, FGFR3 and/or FGFR4. For example, compounds of the disclosure can be useful in treating FGFR-associated diseases and disorders, e.g., proliferative disorders such as cancers, including hematological cancers and solid tumor, and angiogenesis-related disorders. Compounds of the disclosure may also be useful in treating disorders arising from autosomal dominant mutations in FGFR, e.g., FGFR3, including, for example, developmental disorders. Developmental disorders to be treated with compounds of the disclosure include Achondroplasia (Ach) and related chondrodysplasia syndromes, including Hypochondroplasia (Hch), Severe Achondroplasia with Developmental Delay and Acanthosis Nigricans (SADDAN), and Thanatophoric dysplasia (TD).

Non-limiting examples of FGFR-associated diseases and disorders include Acanthosis nigricans, Achondroplasia, Apert syndrome, Beare-Stevenson syndrome (BSS), Camptodactyly, tall stature, and hearing loss syndrome (CATSHL) syndrome, cleft lip and palate, congenital heart disease (e.g., associated with ambiguous genitalia), craniosynostosis, Crouzon syndrome, ectrodactyly, encephalocraniocutaneous lipomatosis, Hartsfield syndrome, hypochondroplasia, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, lethal pulmonary acinar dysplasia, microphthalmia, Muenke coronal craniosynostosis, osteoglophonic dysplasia, Pfeiffer syndrome, seborrheic keratosis, syndactyly, thanatophoric dysplasia (e.g., type I or type II), trigonocephaly 1 (also called metopic craniosynostosis), and tumor-induced osteomalacia.

Non-limiting examples of FGFR1 associated diseases and disorders include congenital heart disease (e.g., associated with ambiguous genitalia), craniosynostosis, encephalocraniocutaneous lipomatosis, Hartsfield syndrome, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, osteoglophonic dysplasia, Pfeiffer syndrome, trigonocephaly 1 (also called metopic craniosynostosis), and tumor-induced osteomalacia.

Non-limiting examples of FGFR2-associated diseases and disorders include Apert syndrome, Beare-Stevenson syndrome (BSS), Crouzon syndrome, ectrodactyly, Jackson-Weiss syndrome, lethal pulmonary acinar dysplasia, Pfeiffer syndrome, and syndactyly. Non-limiting examples of FGFR3-associated diseases and disorders include acanthosis nigricans, achondroplasia, Camptodactyly, tall stature, and hearing loss syndrome (CATSHL) syndrome, cleft lip and palate, craniosynostosis, hypochondroplasia, microphthalmia, Muenke coronal craniosynostosis, seborrheic keratosis, and thanatophoric dysplasia (e.g., type I or type II). See also, See UniParc entry UPI00000534B8; UniParc entry UPI0000001COF; Uni Pare entry UPI000002A99A; UniParc entry UPI000012A72A; Yong-Xing et al., Hum. Mol. Genet. 9(13):2001-2008, 2000; Eeva-Maria Laitinen et al., PLoS One 7(6):e39450, 2012; Hart et al., Oncogene 19(29):3309-3320, 2000; Shiang et al., Cell 76:335-342, 1994; Rosseau et al., Nature 371:252-254, 1994; Tavormina et al., Nature Genet. 9:321-328, 1995; Bellus et al., Nature Genet. 10:357-359, 1995; Muenke et al., Nature Genet. 8:269-274, 1994; Rutland et al., Nature Genet. 9:173-176, 1995; Reardon et al., Nature Genet. 8:98-103, 1994; Wilkie et al., Nature Genet. 9:165-172, 1995; Jabs et al., Nature Genet. 8:275-279, 1994; Japanese Patent No. JP05868992B2; Ye et al., Plast. Reconstr. Surg., 137(3):952-61, 2016; U.S. Pat. No. 9,447,098B2; Bellus et al., Am. J. Med. Genet. 85(1):53-65, 1999; PCT Patent Application Publication No. WO2016139227A1; Australian Patent Application Publication No. AU2014362227A1; Chinese Patent No. CN102741256B; Ohishi et al., Am. J. Med. Genet. A., doi: 10.1002/ajmg.a.37992, 2016; Nagahara et al., Clin. Pediatr. Endocrinol., 25(3): 103-106, 2016; Hibberd et al., Am. J. Med. Genet. A., doi: 10.1002/ajmg.a.37862, 2016; Dias et al., Exp. Mol. Pathol., 101(1):116-23, 2016; Lin et al., Mol. Med. Rep., 14(3):1941-6, 2016; Barnett et al., Hum. Mutat., 37(9):955-63, 2016; Krstevska-Konstantinova et al., Med. Arch., 70(2):148-50, 2016; Kuentz et al., Br. J. Dermatol., doi: 10.1111/bjd.14681, 2016; Ron et al., Am. J. Case Rep., 15; 17:254-8, 2016; Fernandes et al., Am. J. Med. Genet. A., 170(6):1532-7, 2016; Lindy et al., Am. J. Med. Genet. A., 170(6):1573-9, 2016; Bennett et al., Am. J. Hum. Genet., 98(3):579-87, 2016; Ichiyama et al., J. Eur. Acad. Dermatol. Venereal., 30(3):442-5, 2016; Zhao et al., Int. J. Clin. Exp. Med., 8(10):19241-9, 2015; Hasegawa et al., Am. J. Med. Genet. A., 170A (5):1370-2, 2016; Legeai-Mallet, Endocr. Dev., 30:98-105, 2016; Takagi, Am. J. Med. Genet. A., 167A(ll):2851-4, 2015; Goncalves, Fertil. Steril., 104(5):1261-7.el, 2015; Miller et al., Journal of Clinical Oncology, 34: Supp. Supplement 15, pp. iii93. AbstractNumber: e22500, 2016 Annual Meeting of the American Society of Clinical Oncology, Chicago, IL; Sarabipour et al., J. Mol. Biol., 428(20):3903-3910, 2016; Escobar et al., Am. J. Med. Genet. A., 170(7):1908-11, 2016; Mazen et al., Sex Dev., 10(1):16-22, 2016; Taylan et al., J Allergy Clin Immunol, 136(2):507-9, 2015. doi: 10.1016/j.jaci.2015.02.010; Kant et al, Euro Journ Endocrinol, 172(6):763-770, 2015. doi: 10.1530/EJE-14-0945; Gonzalez-Del Angel et al, Am J med Genet A, 176(1):161-166, 2018. doi: 10.1002/ajmg.a.38526; Lei and Deng, Int J Biol Sci 13(9):1163:1171, 2017. doi: 10.7150/ijbs.20792; Lajeunie et al, Eur J Hum Genet, 14(3):289-298, 2006. doi: 10.1038/sj.ejhg.5201558; Karadimas et al, Prenat Diagn, 26(3):258-261, 2006. doi: 10.1002/pd. 1392; Ibrahimi et al, Hum Mol Genet 13(19):2313-2324, 2004. doi: 10.1093/hmg/ddh235; Trarbach et al, J Clin Endocrinol Metab., 91(10):4006-4012, 2006. doi: 10.1210/jc.2005-2793; Dode et al, Nat Genet, 33(4):463-465, 2003. doi: 10.1038/ng1122, each of which is incorporated by reference herein.

The term “angiogenesis-related disorder” means a disease characterized in part by an increased number or size of blood vessels in a tissue in a subject or patient, as compared to a similar tissue from a subject not having the disease. Non-limiting examples of angiogenesis-related disorders include: cancer (e.g., any of the exemplary cancers described herein, such as prostate cancer, lung cancer, breast cancer, bladder cancer, renal cancer, colon cancer, gastric cancer, pancreatic cancer, ovarian cancer, melanoma, hepatoma, sarcoma, and lymphoma), exudative macular degeneration, proliferative diabetic retinopathy, ischemic retinopathy, retinopathy of prematurity, neovascular glaucoma, iritis rubeosis, corneal neovascularization, cyclitis, sickle cell retinopathy, and pterygium.

Compounds of the disclosure inhibit wild-type FGFR1, FGFR2, FGFR3, and/or FGFR4. In other aspects, compounds of the disclosure inhibit a mutated FGFR1, FGFR2, FGFR3, and/or FGFR4. In other aspects, compounds of the disclosure inhibit FGFR1, FGFR2, FGFR3, and/or FGFR4 that includes an FGFR kinase inhibitor mutation.

In some embodiments of any of the methods or uses described herein, the cancer (e.g., FGFR-associated cancer) is a hematological cancer. In some embodiments of any of the methods or uses described herein, the cancer (e.g., FGFR-associated cancer) is a solid tumor.

In some embodiments of any of the methods or uses described herein, the cancer (e.g., FGFR-associated cancer) is a lung cancer (e.g., small cell lung carcinoma, non-small cell lung carcinoma, squamous cell carcinoma, lung adenocarcinoma, large cell carcinoma, mesothelioma, lung neuroendocrine carcinoma, smoking-associated lung cancer), prostate cancer, colorectal cancer (e.g., rectal adenocarcinoma), endometrial cancer (e.g., endometrioid endometrial cancer, endometrial adenocarcinoma), breast cancer (e.g., hormone-receptor-positive breast cancer, triple-negative breast cancer, neuroendodrine carcinoma of the breast), skin cancer (e.g., melanoma, cutaneous squamous cell carcinoma, basal cell carcinoma, large squamous cell carcinoma), gallbladder cancer, liposarcoma (e.g., dedifferentiated liposarcoma, myxoid liposarcoma), pheochromocytoma, myoepithelial carcinoma, urothelial carcinoma, spermatocytic seminoma, stomach cancer, head and neck cancer (e.g., head and neck (squamous) carcinoma, head and neck adenoid cystic adenocarcinoma), brain cancer (e.g., glialneural tumors, glioma, neuroblastoma, glioblastoma, pilocytic astrocytoma, Rosette forming glioneural tumor, dysembryoplastic neuroepithelial tumor, anaplastic astrocytoma, medulloblastoma, ganglio-glioma, oligodendroglioma), malignant peripheral nerve sheath tumor, sarcoma (e.g., soft tissue sarcoma (e.g., leiomyosarcoma), osteosarcoma), esophageal cancer (e.g., esophageal adenocarcinoma), lymphoma, bladder cancer (e.g., bladder urothelial (transition cell) carcinoma), cervical cancer (e.g., cervical squamous cell carcinoma, cervical adenocarcinoma), fallopian tube cancer (e.g., fallopian tube carcinoma), ovarian cancer (e.g., ovarian serous cancer, ovarian mucinous carcinoma), cholangiocarcinoma, adenoid cystic carcinoma, pancreatic cancer (e.g., pancreatic exocrine carcinoma, pancreatic ductal adenocarcinoma, pancreatic cancer intraepithelial neoplasia), salivary gland cancer (e.g., pleomorphic salivary gland adenocarcinoma, salivary adenoid cystic cancer), oral cancer (e.g., oral squamous cell carcinoma), uterine cancer, gastric or stomach cancer (e.g., gastric adenocarcinoma), gastrointestinal stromal tumors, myeloma (e.g., multiple myeloma), lymphoepithelioma, anal cancer (e.g., anal squamous cell carcinoma), prostate cancer (e.g., prostate adenocarcinoma), renal cell carcinoma, thymic cancer, gastroesophogeal junction adenocarcinoma, testicular cancer, rhabdomyosarcoma (e.g., alveolar rhabdomyosarcoma, embryonic rhabomyosarcoma), renal papillary carcinoma, liver cancer (e.g., hepatocellular carcinoma, intrahepatic cholangiocarcinoma), carcinoid, myeloid proliferative disorders (also called myeloid proliferative neoplasms (MPN); e.g., 8pll myeloproliferative syndrome (EMS, also called stem cell leukemia/lymphoma), acute myeloid leukemia (AML), chronic myeloid leukemia (CML)), lymphoma (e.g., T-cell lymphoma, T-lymphoblastic lymphoma, acute lymphoblastic leukemia (ALL), B-cell lymphoma), myeloid and lymphoid neoplasms, chronic neutrophilic leukemia, phosphaturic mesenchymal tumor, thyroid cancer (e.g. anaplastic thyroid carcinoma), or biliary duct cancer.

In some embodiments of any of the methods or uses described herein, the cancer (e.g., FGFR-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and para nasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, para nasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, retinoblastoma, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, and Wilms' tumor.

In some embodiments, a hematological cancer (e.g., hematological cancers that are FGFR associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CML), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult Tcell ALL, AML with trilineage myelodysplasia (AML/TMDS), mixed lineage leukemia (MLL), myelodysplastic syndromes (MDSs), myeloproliferative disorders (MPD), and multiple myeloma (MM).

Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a FGFR-associated cancer) is AML or CMML.

In some embodiments, the cancer (e.g., the FGFR-associated cancer) is a solid tumor. Examples of solid tumors (e.g., solid tumors that are FGFR-associated cancers) include, for example, lung cancer (e.g., lung adenocarcinoma, non-small-cell lung carcinoma, squamous cell lung cancer), bladder cancer, colorectal cancer, brain cancer, testicular cancer, bile duct cancer cervical cancer, prostate cancer, and sparmatocytic seminomas. See, for example, Turner and Grose, Nat. Rev. Cancer, 10(2):116-129, 2010.

In some embodiments, the cancer is selected from the group consisting of bladder cancer, brain cancer, breast cancer, cholangiocarcinoma, head and neck cancer, lung cancer, multiple myeloma, rhabdomyosarcoma, urethral cancer, and uterine cancer. In some embodiments, the cancer is selected from the group consisting of lung cancer, breast cancer, and brain cancer.

In some embodiments, a FGFR1-associated cancer is selected from the group consisting of lung cancer, breast cancer, and brain cancer.

In some embodiments, the cancer is selected from the group consisting of breast cancer, uterine cancer, cholangiocarcinoma, and lung cancer.

In some embodiments, a FGFR2-associated cancer is selected from the group consisting of breast cancer, uterine cancer, cholangiocarcinoma, and lung cancer. In some embodiments, the cancer is selected from the group consisting of lung cancer, bladder cancer, urethral cancer, multiple myeloma, and head and neck cancer.

In some embodiments, a FGFR3-associated cancer is selected from the group consisting of lung cancer, bladder cancer, urethral cancer, multiple myeloma, and head and neck cancer.

In some embodiments, the cancer is selected from lung cancer, rhabdomyosarcoma, and breast cancer.

In some embodiments, a FGFR4-associated cancer is selected from lung cancer, rhabdomyosarcoma, and breast cancer.

In some aspects, the compounds of the disclosure are useful in treating cancers associated with amplification or overexpression of FGFR1, for example, Breast cancer or carcinoma (e.g., hormone receptor-positive breast cancer, ductal carcinoma in situ (breast)), pancreatic ductal adenocarcinoma, pancreatic exocrine carcinoma, smoking-associated lung cancer, small cell lung cancer, lung adenocarcinoma, non-small cell lung cancer, squamous cell lung cancer or carcinoma, prostate cancer or carcinoma, ovarian cancer, fallopian tube carcinoma, bladder cancer, rhabdomyosarcoma, head and neck carcinoma (e.g., head and neck squamous cell carcinoma), esophageal cancer (e.g., esophageal squamous cell carcinoma), sarcoma (e.g., osteosarcoma), hepatocellular carcinoma, renal cell carcinoma, colorectal cancer (e.g., colorectal adenocarcinoma), prostate cancer, salivary gland tumors, glioblastoma multiforme, urinary bladder cancer, urothelial carcinoma, carcinoma of unknown primary, squamous non-lung tumors, gastric cancer, gastroesophageal junction carcinoma, adenoid cystic carcinoma, anal squamous cell carcinoma, oral squamous cell carcinoma, cholangiocarcinoma, hemangioendothelioma, leiomyosarcoma, melanoma, neuroendocrine carcinoma, squamous cell carcinoma, uterine carcinosarcoma.

In some aspects, the compounds of the disclosure are useful in treating cancers associated with amplification of FGFR2, for example, Gastric cancer, gastroesophageal junction adenocarcinoma, breast cancer (e.g., triple negative breast cancer), colon cancer, colorectal cancer (e.g., colorectal adenocarcinoma), urothelial cancer, bladder adenocarcinoma, carcinoma of unknown primary, cholangiocarcinoma, endometrial adenocarcinoma, esophageal adenocarcinoma, gallbladder carcinoma, ovarian cancer, fallopian tube carcinoma, pancreatic exocrine carcinoma, sarcoma, squamous cell carcinoma.

In some aspects, the compounds of the disclosure are useful in treating cancers associated with overexpression of FGFR2, for example, Myxoid lipocarcinoma, rectal cancer, renal cell carcinoma, breast cancer.

In some aspects, the compounds of the disclosure are useful in treating cancers associated with upregulation of activity of FGFR3, for example, Colorectal cancer, hepatocellular carcinoma, pancreatic exocrine carcinoma. In some aspects, the compounds of the disclosure are useful in treating cancers associated with overexpression of activity of FGFR3, for example, Multiple myeloma, thyroid carcinoma. In some aspects, the compounds of the disclosure are useful in treating cancers associated with amplification of activity of FGFR3, for example, Bladder cancer and salivary adenoid cystic cancer, urothelial cancer, breast cancer, carcinoid, carcinoma of unknown primary, colorectal cancer (e.g., colorectal adenocarcinoma), gallbladder carcinoma, gastric cancer, gastroesophageal junction adenocarcinoma, glioma, mesothelioma, non-small cell lung carcinoma, small cell lung cancer, ovarian cancer, fallopian tube carcinoma, pancreatic exocrine carcinoma.

In some aspects, the compounds of the disclosure are useful in treating cancers associated with amplification of FGFR4, for example, Rhabdomyosarcoma, prostate cancer or carcinoma, breast cancer, urothelial cancer, carcinoid, carcinoma of unknown primary, esophageal adenocarcinoma, head and neck carcinoma, hepatocellular carcinoma, non-small cell lung carcinoma, ovarian cancer, fallopian tube carcinoma, peritoneal carcinoma, renal cell carcinoma.

In some aspects, the compounds of the disclosure are useful in treating cancers associated with upregulation of activity of FGFR4, for example, Colorectal cancer, hepatocellular carcinoma, adrenal carcinoma, breast cancer.

In some aspects, the compounds of the disclosure are useful in treating cancers associated with overexpression of activity of FGFR4, for example, Pancreatic intraepithelial neoplasia, and pancreatic ductal adenocarcinoma.

In some aspects, the compounds of the disclosure are more selective for one FGFR than for another. As used herein, the “selectivity” of a compound for a first target over a second target means that the compound has more potent activity at the first target than the second target. A fold selectivity can be calculated by any method known in the art. For example, a fold selectivity can be calculated by dividing the IC50 value (or Kd value) of a compound for the second target (e.g., FGFR1) by the IC50 value of the same compound for the first target (e.g., FGFR2 or FGFR3). An IC50 value can be determined by any method known in the art. In some embodiments, a compound is first determined to have an activity of less than 500 nM for the first target. In some embodiments, a compound is first determined to have an activity of less than 500 nM for the second target.

For example, in some aspects, the compounds of the disclosure are more selective for FGFR3 than for FGFR1. In some aspects, the compounds are at least 3-fold more selective for FGFR3 than for FGFR1. In some aspects, the compounds are 4, 5, 6, 7, 8, 9, 10, 15, 20, 30, 40, 50, 75, 100, 200, 500, or 1000 fold more selective for FGFR3 than for FGFR1.

In some aspects, the compounds of the disclosure are more selective for FGFR2 than for FGFR1. In some aspects, the compounds are at least 3-fold more selective for FGFR2 than for FGFR1. In some aspects, the compounds are 4, 5, 6, 7, 8, 9, 10, 15, 20, 30, 40, 50, 75, 100, 200, 500, or 1000 fold more selective for FGFR2 than for FGFR1.

In some aspects, the compounds of the disclosure are more selective for a first FGFR family member (e.g., FGFR2 or FGFR3) over a second FGFR family member (e.g., FGFR1 or FGFR4). In some aspects, the compounds of the disclosure are at least 3-fold more selective for a first FGFR family member over a second FGFR family member. In some aspects, the compounds are at least 10, 20, 30, 40, 50, 60, 70, 80, 90, 200, 300, 400, 500, 600, 700, 800, 900, or at least 1000 fold more selective for a first FGFR family member over a second FGFR family member.

In some aspects, the compounds of the disclosure are more selective for an FGFR kinase over another kinase that is not an FGFR kinase. For example, the compounds of the disclosure are at least 3-fold more selective for an FGFR kinase over another kinase that is not an FGFR kinase. In some aspects, the compounds of the disclosure are at least 10, 20, 30, 40, 50, 60, 70, 80, 90, 200, 300, 400, 500, 600, 700, 800, 900, or at least 1000 fold more selective for an FGFR kinase over another kinase that is not an FGFR kinase. Kinases that are not FGFR kinases include, for example, KDR kinase and Aurora B kinase.

In some embodiments, the compounds of the disclosure exhibit brain and/or central nervous system (CNS) penetrance. Such compounds are capable of crossing the blood brain barrier and inhibiting a FGFR kinase in the brain and/or other CNS structures. In some embodiments, the compounds provided herein are capable of crossing the blood brain barrier in a therapeutically effective amount. For example, treatment of a subject with cancer (e.g., a FGFR-associated cancer such as a FGFR-associated brain or CNS cancer) can include administration (e.g., oral administration) of the compound to the subject. In some such embodiments, the compounds provided herein are useful for treating a primary brain tumor or metastatic brain tumor. For example, a FGFR-associated primary brain tumor or metastatic brain tumor.

In some embodiments, the compounds of the disclosure, exhibit one or more of high GI absorption, low clearance, and low potential for drug-drug interactions.

In some aspects, compounds of the disclosure can be used for treating a subject diagnosed with (or identified as having) a FGFR-associated disease or disorder (e.g., a FGFR-associated cancer) that include administering to the subject a therapeutically effective amount of a compound of the disclosure. Also provided herein are methods for treating a subject identified or diagnosed as having a FGFR-associated disease or disorder (e.g., a FGFR-associated cancer) that include administering to the subject a therapeutically effective amount of a compound of the disclosure. In some embodiments, the subject that has been identified or diagnosed as having a FGFR-associated disease or disorder (e.g., a FGFR-associated cancer) through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the FGFR-associated disease or disorder is a FGFR-associated cancer. For example, the FGFR-associated cancer can be a cancer that includes one or more FGFR inhibitor resistance mutations.

Also provided are methods for treating a disease or disorder in a subject in need thereof, the method comprising: (a) detecting a FGFR-associated disease or disorder in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy. In some embodiments, the subject was previously treated with a first FGFR inhibitor or previously treated with another treatment. In some embodiments, the subject is determined to have a FGFR-associated disease or disorder through the use of a regulatory agency-approved, e.g., FDA approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a FGFR-associated cancer in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy). In some embodiments, the subject was previously treated with a first FGFR inhibitor or previously treated with another anticancer treatment, e.g., at least partial resection of the tumor or radiation therapy. In some embodiments, the subject is determined to have a FGFR-associated cancer through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is a FGFR associated cancer. For example, the FGFR-associated cancer can be a cancer that includes one or more FGFR inhibitor resistance mutations. In some embodiments, the cancer is a FGFR associated cancer. For example, the FGFR-associated cancer can be a cancer that includes one or more FGFR activating mutations.

Also provided are methods of treating a subject that include performing an assay on a sample obtained from the subject to determine whether the subject has a dysregulation of a FGFR gene, a FGFR kinase, or expression or activity or level of any of the same, and administering (e.g., specifically or selectively administering) a therapeutically effective amount of a compound of the disclosure or pharmaceutically acceptable salt or solvate thereof to the subject determined to have a dysregulation of a FGFR gene, a FGFR kinase, or expression or activity or level of any of the same. Some embodiments of these methods further include administering to the subject an additional therapy or therapeutic agent (e.g., a second FGFR inhibitor, a second compound of the disclosure, or immunotherapy). In some embodiments of these methods, the subject was previously treated with a first FGFR inhibitor or previously treated with another anticancer treatment, e.g., at least partial resection of a tumor or radiation therapy. In some embodiments, the subject is a subject suspected of having a FGFR-associated disease or disorder (e.g., a FGFR-associated cancer), a subject presenting with one or more symptoms of a FGFR-associated disease or disorder (e.g., a FGFR-associated cancer), or a subject having an elevated risk of developing a FGFR-associated disease or disorder (e.g., a FGFR-associated cancer). In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of a FGFR gene, a FGFR kinase, or expression or activity or level of any of the same includes one or more FGFR inhibitor resistance mutations.

Also provided herein are methods of selecting a treatment for a subject, wherein the methods include a step of performing an assay on a sample obtained from the subject to determine whether the subject has a dysregulation of a FGFR gene, a FGFR kinase, or expression or activity or level of any of the same (e.g., one or more FGFR inhibitor resistance mutations), and identifying or diagnosing a subject determined to have a dysregulation of a FGFR gene, a FGFR kinase, or expression or activity or level of any of the same, as having a FGFR-associated cancer. Some embodiments further include administering the selected treatment to the subject identified or diagnosed as having a FGFR-associated cancer. For example, in some embodiments, the selected treatment can include administration of a therapeutically effective amount of a compound of the disclosure to the subject identified or diagnosed as having a FGFR-associated cancer. In some embodiments, the assay is an in vitro assay. For example, an assay that utilizes the next generation sequencing, immunohistochemistry, or break apart FISH analysis. In some embodiments, the assay is a regulatory agency-approved, e.g., FDA-approved, kit. In some embodiments, the assay is a liquid biopsy.

Also provided herein are methods of treating a FGFR-associated cancer in a subject that include (a) administering one or more (e.g., two or more, three or more, four or more, five or more, or ten or more) doses of a first FGFR kinase inhibitor to a subject identified or diagnosed as having a FGFR associated cancer (e.g., any of the types of FGFR-associated cancers described herein) (e.g., identified or diagnosed as having a FGFR-associated cancer using any of the exemplary methods described herein or known in the art); (b) after step (a), determining a level of circulating tumor DNA in a biological sample (e.g., a biological sample comprising blood, serum, or plasma) obtained from the subject; (c) administering a therapeutically effective amount of a second FGFR inhibitor or a compound of the disclosure as a monotherapy or in conjunction with an additional therapy or therapeutic agent to a subject identified as having about the same or an elevated level of circulating tumor DNA as compared to a reference level of circulating tumor DNA (e.g., any of the reference levels of circulating tumor DNA described herein). In some examples of these methods, the reference level of circulating tumor DNA is a level of circulating tumor DNA in a biological sample obtained from the subject prior to step (a). Some embodiments of these methods further include determining the level of circulating tumor DNA in the biological sample obtained from the subject prior to step (a). In some examples of these methods, the reference level of circulating tumor DNA is a threshold level of circulating tumor DNA (e.g., an average level of circulating tumor DNA in a population of subjects having a similar FGFR-associated cancer and having a similar stage of the FGFR-associated cancer, but receiving a non-effective treatment or a placebo, or not yet receiving therapeutic treatment, or a level of circulating tumor DNA in a subject having a similar FGFR-associated cancer and having a similar stage of the FGFR-associated cancer, but receiving a non-effective treatment or a placebo, or not yet receiving therapeutic treatment). In some examples of these methods, the first FGFR inhibitor is: ARQ-087, ASP5878, AZD4547, B-701, BAY1179470, BAY1187982, BGJ398, brivanib, Debio 1347, dovitinib, E7090, erdafitinib, FPA144, HMPL-453, INCB054828, lenvatinib, lucitanib, LY3076226, MAX-40279, nintedanib, orantinib, pemigatinib, ponatinib, PRN1371, rogaratinib, sulfatinib, TAS-120 or RLY-4008.

### Combination Therapies

Depending upon the particular condition, or disease, to be treated, additional therapeutic agents, which are normally administered to treat that condition, may be administered.

Compounds of the disclosure can also be administered with additional therapy or therapeutic agents. In some aspects, the additional therapy or therapeutic agent includes one or more of radiation therapy, a chemotherapeutic agent (e.g., any of the exemplary chemotherapeutic agents described herein or known in the art), a checkpoint inhibitor (e.g., any of the exemplary checkpoint inhibitors described herein or known in the art), surgery (e.g., at least partial resection of the tumor), and one or more other kinase inhibitors (e.g., any of the kinase inhibitors described herein or known in the art).

Compounds of the disclosure may also be useful as adjuvants to cancer treatment, that is, they can be used in combination with one or more additional therapies or therapeutic agents, for example, a chemotherapeutic agent that works by the same or by a different mechanism of action. In some embodiments, a compound of the disclosure can be used prior to administration of an additional therapeutic agent or additional therapy. For example, a subject in need thereof can be administered one or more doses of a compound of the disclosure for a period of time and then undergo at least partial resection of the tumor. In some embodiments, the treatment with one or more doses of a compound of the disclosure reduces the size of the tumor (e.g., the tumor burden) prior to the at least partial resection of the tumor. In some embodiments, a subject has a cancer (e.g., a locally advanced or metastatic tumor) that is refractory or intolerant to standard therapy (e.g., administration of a chemotherapeutic agent, such as a first FGFR inhibitor or a multikinase inhibitor, immunotherapy, radiation, or a platinum-based agent (e.g., cisplatin)). In some embodiments, a subject has a cancer (e.g., a locally advanced or metastatic tumor) that is refractory or intolerant to prior therapy (e.g., administration of a chemotherapeutic agent, such as a first FGFR inhibitor or a multikinase inhibitor, immunotherapy, radiation, or a platinum-based agent (e.g., cisplatin)).

In some embodiments of any the methods described herein, the compound of the disclosure is administered in combination with a therapeutically effective amount of at least one additional therapeutic agent selected from one or more additional therapies or therapeutic (e.g., chemotherapeutic) agents. Non-limiting examples of additional therapeutic agents include: other FGFR-targeted therapeutic agents (i.e. a first or second FGFR kinase inhibitor), other kinase inhibitors (e.g., receptor tyrosine kinase targeted therapeutic agents (e.g., Trk inhibitors or EGFR inhibitors)), signal transduction pathway inhibitors, checkpoint inhibitors, modulators of the apoptosis pathway (e.g., obatoclax); cytotoxic chemotherapeutics, angiogenesis-targeted therapies, immune-targeted agents, including immunotherapy, and radiotherapy.

Also provided herein are methods of treating a disease or disorder, comprising administering to a subject in need thereof a pharmaceutical combination for treating the disease or disorder which comprises (a) a compound of the disclosure, (b) an additional therapeutic agent, and (c) optionally at least one pharmaceutically acceptable carrier for simultaneous, separate or sequential use for the treatment of the disease or disorder, wherein the amounts of the compound of the disclosure and the additional therapeutic agent are together effective in treating the disease or disorder. In some embodiments, the compound of the disclosure, and the additional therapeutic agent are administered simultaneously as separate dosages. In some embodiments, the compound of the disclosure, and the additional therapeutic agent are administered as separate dosages sequentially in any order, in jointly therapeutically effective amounts, e.g. in daily or intermittently dosages. In some embodiments, the compound of the disclosure, and the additional therapeutic agent are administered simultaneously as a combined dosage. In some embodiments, the disease or disorder is a FGFR-associated disease or disorder. In some embodiments, the subject has been administered one or more doses of a compound of the disclosure, prior to administration of the pharmaceutical composition.

In some embodiments, the treatment period is at least 7 days (e.g., at least or about 8 days, at least or about 9 days, at least or about 10 days, at least or about 11 days, at least or about 12 days, at least or about 13 days, at least or about 14 days, at least or about 15 days, at least or about 16 days, at least or about 17 days, at least or about 18 days, at least or about 19 days, at least or about 20 days, at least or about 21 days, at least or about 22 days, at least or about 23 days, at least or about 24 days, at least or about 25 days, at least or about 26 days, at least or about 27 days, at least or about 28 days, at least or about 29 days, or at least or about 30 days).

In some embodiments, the treatment period is at least 21 days (e.g., at least or about 22 days, at least or about 23 days, at least or about 24 days, at least or about 25 days, at least or about 26 days, at least or about 27 days, at least or about 28 days, at least or about 29 days, at least or about 30 days, at least or about 31 days, at least or about 32 days, at least or about 33 days, at least or about 34 days, at least or about 35 days, at least or about 36 days, at least or about 37 days, at least or about 38 days, at least or about 39 days, or at least or about 40 days).

Also provided herein are pharmaceutical compositions that contain, as the active ingredient, a compound of the disclosure, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is formulated as a tablet or capsule.

The compositions comprising a compound of the disclosure can be formulated in a unit dosage form, each dosage containing from about 5 to about 1,000 mg (1 g), more usually about 100 mg to about 500 mg of the active ingredient. The term “unit dosage form” refers to physically discrete units for human subjects and other subjects, each unit containing a predetermined quantity of active material (i.e., a compound of the disclosure) to produce the desired therapeutic effect, with a suitable pharmaceutical excipient.

In some embodiments, the compositions provided herein contain from about 5 mg to about 50 mg of the active ingredient, i.e., the compound of the disclosure. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient. In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

### Pharmaceutically Acceptable Compositions

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. In some embodiments, the pharmaceutical compositions contain a compound of the present disclosure or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent (such as in solvates) or counterions (such as in pharmaceutically acceptable salts).

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, microcrystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidyl-choline, phosphatidylethanolamine, phosphatidylglycerol, phosphatidic acid, phosphatidylserine, lysophosphatidylcholine, lysophosphatidylethanolamine, lysophosphatidylglycerol, lyso-phosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, tetradecyl sulfate, docusate, lauroyl carnitines, palmitoyl carnitines, myristoyl carnitines, and salts and mixtures thereof.

Hydrophilic non-ionic surfactants may include, but are not limited to, alkyl-glucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%0, 50%), 100% o, or up to about 200%>by weight, based on the combined weight of the drug, and other excipients. If desired, very small amounts of solubilizer may also be used, such as 5%>, 2%>, 1%) or even less. Typically, the solubilizer may be present in an amount of about 1%>to about 100%, more typically about 5%>to about 25%>by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylene-diamine, triethanolamine, triethylamine, triisopropanolamine, trimethylamine, tris(hydroxy-methyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol mono-laurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### General Procedures

X-Ray Powder Diffraction (XRPD) Data Collection Strategy: XRPD patterns of samples were recorded at room temperature on Aeris X-ray diffractometer (Almelo, The Netherlands) using Cu Kα radiation (λ=1.54 Å) at 40 kV, 15 mA passing through a Ni monochromater. Data were collected in a continuous scan mode with a step size of 0.02° and dwell time of 149 s over an angular range of 3° to 40° 2θ. The sample was loaded on a zero-background holder and gently pressed by a clean glass slide to ensure coplanarity of the sample surface with the surface of the holder. Obtained diffractograms were analysed and plotted with HighScore Plus software (V 5.0; Almelo, The Netherlands).

Thermogravimetric Analysis (TGA): TGA was performed using a Discovery TGA 5500 (TA® Instruments, New Castle, Delaware, USA) instrument operating with TRIOS software (Version 5.0). The sample was placed in an aluminum pan. The sample cell was purged with dry nitrogen at a flow rate of 15 mL/min. A heating rate of 10° C./min from 25-350° C. was used in all the experiments.

Differential Scanning calorimetry (DSC): Conventional DSC experiments were performed by using Q100 (TA® Instruments, New Castle, Delaware, USA) instrument equipped with a refrigerated cooling system (RCS90). The sample cell was purged with dry nitrogen at a flow rate of 50 mL/min. Accurately weighed samples (2-5 mg) placed in TZero pans with a pin hole were scanned at a heating rate of 10° C./min over a desired temperature range.

Dynamic Vapor Sorption (DVS): Moisture sorption/desorption data were collected on a DVS-intrinsic vapor sorption analyser (Surface Measurement Systems NA, Allentown, PA, USA) and operating with DVS-intrinsic control software (Version 1.0.5.1). Samples were not dried prior to analysis. Sorption and desorption data were collected over a range from 5% to 98% relative humidity (RH) via 10% RH increments under a nitrogen purge. The equilibrium criterion used for analysis was less than 0.005% weight change in 10 min with a maximum equilibration time of 3 h.

### Example A. General Preparation of Compound A

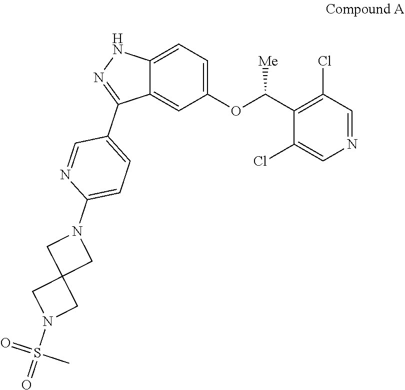 [SMILES: C[C@@H](Oc1ccc2[nH]nc(-c3ccc(N4CC5(C4)CN(S(C)(=O)=O)C5)nc3)c2c1)c1c(Cl)cncc1Cl]

Compound A was prepared according to the steps and intermediates (e.g., Scheme 1) described below.

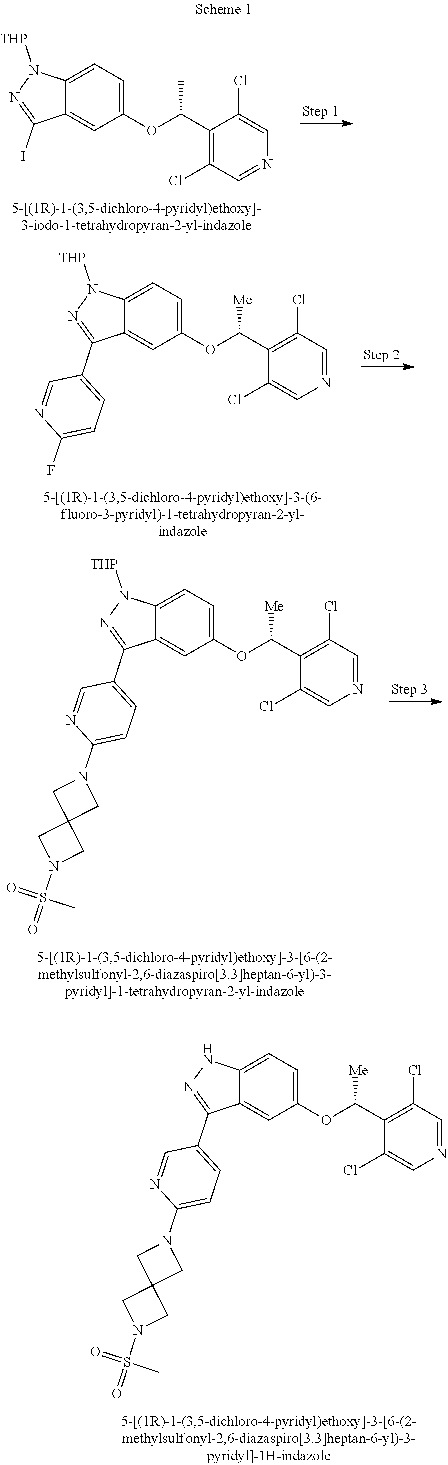

### Step 1. (R)-5-(1-(3,5-Dichloropyridin-4-yl) ethoxy)-3-(6-fluoropyridin-3-yl)-1H-indazole

A mixture of 5-((R)-1-(3,5-dichloropyridin-4-yl) ethoxy)-3-iodo-1-(tetrahydro-2H-pyran-2-yl)-1H-indazole (25.0 g, 48.35 mmol, 1.0 equiv), 2-fluoro-5-(4,4,5,5-tetramethyl-1,3,2-dioxaborolan-2-yl)pyridine (12.94 g, 58.02 mmol, 1.2 equiv), potassium carbonate (13.34 g, 96.70 mmol, 2.0 equiv) and [1,1′-bis(diphenylphosphino) ferrocene]-dichloropalladium (II) (3.56 g, 4.84 mmol, 0.1 equiv) in 1,4-dioxane (250 mL) and water (25 mL) was sparged with nitrogen for 15 minutes and then heated at 100° C. for 4 hours. After cooling to room temperature, the reaction mixture was filtered over a Celite bed (250 g). The Celite bed was washed with ethyl acetate (100 mL) and the combined filtrate was concentrated under reduced pressure. The residue was dissolved in dichloromethane (50 mL) and purified over a silica plug (500 g), eluting with 20% ethyl acetate in heptanes. The fractions containing product were combined and concentrated to dryness under reduced pressure. The resulting light brown solid was triturated in acetonitrile (50 mL) at room temperature for 1 hour. The solids were vacuum filtered to give a white solid (16.77 g, 71% yield). Additional 5-((R)-1-(3,5-dichloropyridin-4-yl) ethoxy)-3-iodo-1-(tetra-hydro-2H-pyran-2-yl)-1H-indazole (25.0 g) was processed as described above to give compound a white solid (16.25 g, 71% yield). The filtrates from both batches were combined and evaporated to dryness under reduced pressure. The resulting residue was purified on a Büchi automated chromatography system (Sorbtech 40 g silica gel column), eluting with a gradient of 0 to 30% ethyl acetate in heptanes to give a white solid (10.2 g). the isolated white solids were combined to give 43.2 g (90% yield). Analysis: LCMS: m/z=487 (M+H).

### Step 2. 5-((R)-1-(3,5-dichloropyridin-4-yl) ethoxy)-3-(6-(6-(methylsulfonyl)-2,6-diazaspiro[3.3]heptan-2-yl)pyridin-3-yl)-1-(tetrahydro-2H-pyran-2-yl)-1H-indazole

A mixture of compound (R)-5-(1-(3,5-dichloropyridin-4-yl) ethoxy)-3-(6-fluoro-pyridin-3-yl)-1H-indazole (5 g, 10.28 mmol, 1.0 equiv), 2-(methylsulfonyl)-2,6-diazaspiro[3.3]heptane dimesylate (4.55 g, 12.34 mmol, 1.2 equiv) and potassium carbonate (4.25 g, 30.84 mmol, 3.0 equiv) in 1-methyl-2-pyrrolidone (70 mL) was heated at 120° C. for 16 hours. The reaction mixture was cooled to room temperature, followed by the addition of water (150 mL). The resulting solids were collected and dried under vacuum at room temperature for 16 hours. The solids were further purified on a Büchi automated chromatography system (Sorbtech 80 g silica gel column), eluting with a gradient of 0 to 10% methanol in ethyl acetate to give a white solid (4.42 g, 67%). LCMS: m/z=643.1 (M+H).

### Step 3. (R)-5-(1-(3,5-Dichloropyridin-4-yl) ethoxy)-3-(6-(6-(methylsulfonyl)-2,6-diazaspiro[3.3]heptan-2-yl)pyridin-3-yl)-1H-indazole

A solution of 5-((R)-1-(3,5-dichloropyridin-4-yl) ethoxy)-3-(6-(6-(methylsulfonyl)-2,6-diazaspiro[3.3]heptan-2-yl)pyridin-3-yl)-1-(tetrahydro-2H-pyran-2-yl)-1H-indazole (4.42 g, 6.88 mmol, 1.0 equiv) in dichloromethane (45 mL) was treated with trifluoroacetic acid (45 mL, 588.15 mmol, 85.0 equiv). After stirring at room temperature for 4 hours, the volatiles were removed under reduced pressure to give a brown oil. The residue was diluted with dichloromethane (100 mL) and poured over crushed ice (35.0 g). The mixture was treated with sodium bicarbonate (25.0 g) in several portions to adjust the pH to 8. The resulting suspension was filtered and the filtrate was transferred to a separatory funnel. The mixture was washed with saturated sodium bicarbonate (50 mL). The aqueous layer was extracted with dichloromethane (2×50 mL). The combined organic layers were concentrated to dryness under reduced pressure. The resulting crude solids (5.0 g) were triturated with methanol (40 mL) at room temperature for 1 hour. The solids were filtered and dried under vacuum at room temperature for 16 hours to give an off-white solid (2.0 g, 55% yield).

LCMS: m/z=559.1 (M+H); ¹H NMR (400 MHz, DMSO-d6) δ=12.99 (s, 1H), 8.59 (s, 2H), 8.52 (d, J=1.7 Hz, 1H), 7.87 (dd, J=2.3, 8.7 Hz, 1H), 7.45 (d, J=8.9 Hz, 1H), 7.17 (d, J=2.1 Hz, 1H), 7.09 (dd, J=2.3, 9.0 Hz, 1H), 6.54 (d, J=8.2 Hz, 1H), 6.11 (q, J=6.6 Hz, 1H), 4.17 (s, 4H), 4.12 (s, 4H), 3.03 (s, 3H), 1.76 (d, J=6.6 Hz, 3H).

### Example 1. Forms I and II of Compound 1

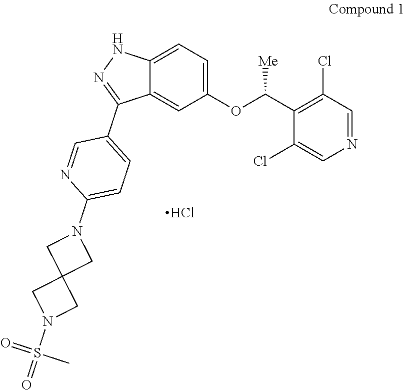 [SMILES: C[C@@H](Oc1ccc2[nH]nc(-c3ccc(N4CC5(C4)CN(S(C)(=O)=O)C5)nc3)c2c1)c1c(Cl)cncc1Cl ; Cl]

### Form I of Compound 1

Form I of compound 1 was prepared as follows: to a suspension of compound A (210 mg, 0.375 mmol) in ethanol (25 mL), cooled in an ice bath, was added a solution of 37% aqueous hydrochloric acid (1.1 equiv) in ethanol (1 mL). The mixture was stirred at 0° C. for 1 hour then warmed to room temperature and stirred overnight. The suspension was diluted with diethyl ether (10 mL), stirred for 15 minutes and the solids were collected by filtration and washed with diethyl ether (40 mL). The solids were dried on the Büchner funnel for 10 minutes, then were dried under vacuum at 40° C. for 5 hours to give a white solid (243 mg, 87% yield).

LCMS: m/z=559.1 (M+H); ¹H NMR (400 MHz, DMSO-d6) δ=13.32 (br s, 1H), 8.59 (s, 2H), 8.31 (s, 1H), 8.26 (br d, J=8.8 Hz, 1H), 7.51 (d, J=9.0 Hz, 1H), 7.20 (d, J=2.1 Hz, 1H), 7.13 (dd, J=2.3, 9.0 Hz, 1H), 6.95 (br d, J=8.6 Hz, 1H), 6.14 (q, J=6.6 Hz, 1H), 4.45 (s, 4H), 4.15 (s, 4H), 3.04 (s, 3H), 1.77 (d, J=6.6 Hz, 3H); m.p.=181.4-215.7° C. (decomposition).

Table 1, supra, is reproduced below and sets forth the X-ray diffraction peaks observed for Form I of compound 1.

TABLE 1
XRPD Peak Positions for Form I of Compound 1

| Pos. [°2θ] | Int. I. (cps) |
|---|---|
| 5.7 | 755(22) |
| 6.9 | 8142(41) |
| 8.0 | 2212(37) |
| 9.7 | 405(26) |
| 11.3 | 839(33) |
| 12.1 | 5541(56) |
| 12.5 | 7326(64) |
| 14.1 | 2819(380) |
| 14.4 | 2541(349) |
| 14.6 | 2308(322) |
| 14.8 | 4143(558) |
| 15.8 | 735(64) |
| 17.2 | 7155(190) |
| 19.9 | 57429(453) |
| 20.6 | 1087(105) |
| 21.9 | 43194(384) |
| 22.7 | 4138(167) |
| 24.1 | 2206(188) |
| 24.5 | 8768(217) |
| 25.2 | 9361(798) |
| 25.7 | 13503(768) |
| 27.1 | 1559(171) |
| 27.4 | 1485(193) |
| 28.2 | 7364(113) |
| 28.7 | 474(177) |
| 29.0 | 940(134) |
| 29.5 | 585(44) |
| 30.3 | 2820(133) |
| 32.5 | 2300(262) |
| 32.9 | 4461(256) |
| 33.8 | 4766(188) |
| 34.1 | 1047(158) |
| 34.6 | 1533(116) |
| 35.0 | 801(129) |
| 36.3 | 4470(62) |
| 37.0 | 211(31) |
| 38.8 | 352(48) |
| 39.2 | 171(33) |
| 39.9 | 713(52) |
| 40.5 | 949(47) |
| 41.4 | 1053(78) |
| 41.9 | 1141(91) |
| 43.6 | 353(64) |
| 44.2 | 1006(210) |
| 44.7 | 1156(268) |
| 45.2 | 1002(204) |
| 47.5 | 354(36) |
| 48.6 | 157(30) |
| 49.2 | 1938(79) |
| 50.2 | 212(28) |
| 56.2 | 1609(85) |

FIG. 1 depicts an XRPD pattern of Form I of compound 1.

### Form II of Compound 1

A reaction between compound A (THE solution) and HCl (ethyl acetate solution) yielded crystalline material. About 3.58% weight loss, from room temperature to 160° C., was observed in the TGA data. The DSC analyses exhibit an exothermic peak at 168.92° C. (peak temperature) followed by two endothermic events between 22° and 270° C. Vapor sorption data (not shown) suggests a moderately hygroscopic material. XRPD data analyses on the material that was recovered from DVS experiment (not shown) suggests stability of the material.

Table 2, supra, is reproduced below and sets forth the X-ray diffraction peaks observed for Form II of compound 1.

TABLE 2
XRPD Peak Positions for Form II of Compound 1

| Pos. [°2θ] | Rel. Int. [%] |
|---|---|
| 5.2 | 1.94 |
| 6.0 | 6.17 |
| 7.3 | 19.96 |
| 8.5 | 2.70 |
| 10.1 | 4.27 |
| 11.7 | 11.14 |
| 12.5 | 17.94 |
| 12.9 | 61.38 |
| 14.4 | 14.18 |
| 14.8 | 55.41 |
| 15.4 | 20.16 |
| 16.3 | 5.08 |
| 17.6 | 40.45 |
| 19.6 | 23.90 |
| 20.3 | 100.00 |
| 22.2 | 56.42 |
| 23.0 | 22.46 |
| 24.5 | 12.18 |
| 25.0 | 47.66 |
| 25.6 | 45.13 |
| 25.9 | 32.96 |
| 26.4 | 29.31 |
| 27.5 | 20.12 |
| 28.6 | 18.85 |
| 30.0 | 10.49 |
| 30.7 | 12.02 |
| 31.7 | 2.87 |
| 33.2 | 5.69 |
| 34.5 | 10.31 |
| 35.1 | 7.15 |
| 36.6 | 5.74 |

FIG. 56 depicts an XRPD pattern of Form II of compound 1.

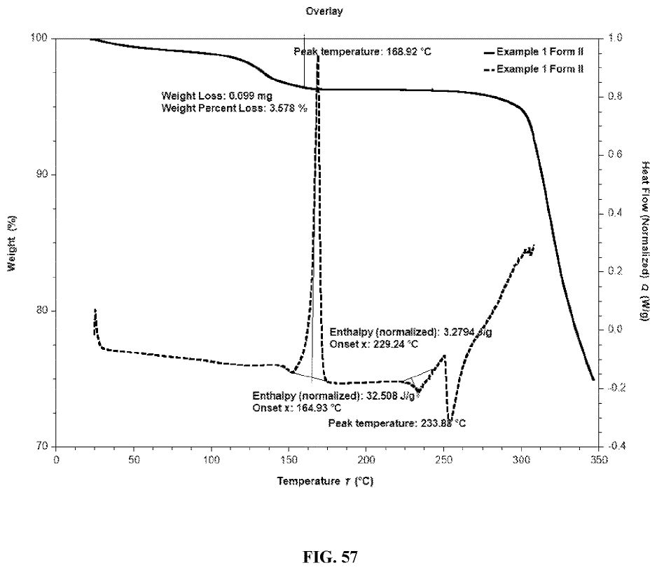

FIG. 57 depicts a DSC thermogram and TGA trace of Form II of compound 2.

### Example 2. Forms I-XIII of Compound 2

### General Synthesis of Solid Forms of Compound 2

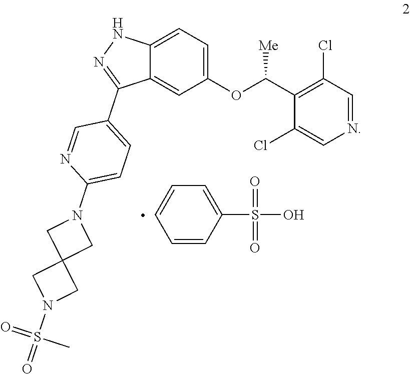

Solid forms of compound 2 were prepared according to either of Method A or Method B described below.

### Method A

A suspension of compound A (5.0 g, 8.94 mmol, 1 equiv.) in acetone (180 mL) was treated with a solution of benzenesulfonic acid (1.63 g, 10.32 mmol, 1.15 equiv) in acetone (17 mL) at room temperature. After 16 hours, the precipitate was filtered, washed with a 1 to 1 mixture of acetone and ethyl ether (3×20 mL) and dried at 50° C. for 5 hours and at room temperature for 2.5 days under vacuum to give an off-white solid (6.28 g, 98%). A second batch was repeated using compound A (4.05 g, 7.25 mmol, 1 equiv) in acetone (145 mL) and benzenesulfonic acid (1.32 g, 8.35 mmol, 1.15 equiv) in acetone (15 mL) at room temperature. After 16 hours, the precipitate was filtered, washed with a 1 to 1 mixture of acetone and ethyl ether (2×12 mL) and dried to give an off-white solid (4.37 g, 84%). A suspension of the combined besylate salts (10.03 g, 13.98 mmol) in ethyl alcohol (1 L) was heated with stirring at 67° C. under a nitrogen atmosphere. After 16 hours, the suspension was cooled to room temperature without stirring for 30 minutes, filtered, washed with cold ethyl alcohol (3×120 mL) and dried at 50° C. for 5 hours and overnight at room temperature under vacuum to give a white solid (8.35 g, 83% yield, 99.5% purity).

LCMS: m/z=559.1 (M+H); ¹H NMR (400 MHz, DMSO-d6) δ=13.27 (br s, 1H), 8.58 (s, 2H), 8.36 (d, J=1.7 Hz, 1H), 8.24 (br d, J=9.2 Hz, 1H), 7.65-7.56 (m, 2H), 7.51 (d, J=9.2 Hz, 1H), 7.36-7.24 (m, 3H), 7.21 (d, J=2.2 Hz, 1H), 7.13 (dd, J=2.3, 9.0 Hz, 1H), 6.94 (br d, J=8.8 Hz, 1H), 6.14 (q, J=6.7 Hz, 1H), 4.42 (s, 4H), 4.15 (s, 4H), 3.04 (s, 3H), 1.77 (d, J=6.7 Hz, 3H); m.p.=186.6-191.4° C. (decomposed).

### Method B

A suspension of compound A (0.200 g, 0.357 mmol) in acetonitrile (9 mL) was treated with benzenesulfonic acid (0.065 g. 0.441, 1.15 equiv) at room temperature. After 3 hours, diethyl ether (6 mL) was added and the mixture stirred for 5 minutes. The resulting precipitate was filtered, washed with diethyl ether (2×3 mL), dried under vacuum at room temperature to give a white solid (0.218 g, 85% yield).

LCMS: m/z=559.1 (M+H); ¹H NMR (400 MHz, DMSO-d6) δ=13.33 (br s, 1H), 8.58 (s, 2H), 8.34 (s, 1H), 8.31 (br d, J=9.2 Hz, 1H), 7.69-7.56 (m, 2H), 7.53 (d, J=9.0 Hz, 1H), 7.34-7.25 (m, 3H), 7.23 (d, J=2.0 Hz, 1H), 7.14 (dd, J=2.2, 9.0 Hz, 1H), 7.01 (d, J=9.3 Hz, 1H), 6.14 (q, J=6.6 Hz, 1H), 4.47 (s, 4H), 4.15 (s, 4H), 3.04 (s, 3H), 1.77 (d, J=6.7 Hz, 3H); m.p.=194.3-201.3° C.

### Synthesis of Forms I-XIII of Compound 2

Forms I-XIII of compound 2 were prepared according to one of the crystallization methods described below, e.g., fast crystallization; reactive crystallization; slurry crystallization or anti-solvent crystallization.

Fast Crystallization Method: In a typical experiment, compound 2 (ca. 10 mg) was dissolved in minimum quantity the stated solvent while heating to 80° C. The resulted solution was allowed to cool down to room temperature. The solutions those that did not yield precipitate were allowed to evaporate slowly at room temperature (RT). The results of the fast crystallization studies are summarized below in Table 39.

TABLE 39
Fast Crystallization Studies of Compound 2

| Solvent | Fast Crystallization Result |
|---|---|
| MeOH | Form-V |
| EtOH | Form-I |
| IPA | Form-I |
| ACN | Form-I |
| THF | Form-I |
| Acetone | Form-II (SE*) |
| AcOH | Form-IV (SE*) |
| DCM | Form-III |
| 14DOX | — |
| 1,2 propdiol | Form-VI |
| Et Gly | Form-VI |
| CHCl3 | Form-I |
| 4-Me-2-Pentanone | — |
| DMSO | — |
| EtOAc | — |
| DMF | — |

(SE*): Solids were not obtained through fast crystallization experiments and the resultant solutions were allowed to evaporate at room temperature.

Reactive Crystallization: Benzene sulfonic acid (BSA) was dissolved in THF to prepare a stock solution and used for all the reactive crystallization experiments. Compound A free base (ca. 30 mg) was dissolved in minimum quantity the stated solvent while heating to 80° C. The resultant compound A free base solutions were allowed to cooldown to RT and added one equivalent of BSA stock solution. It was observed that most of the reactions yielded precipitate. Reactions those did not yielded precipitate were allowed to evaporate slowly at RT. The results of the reactive crystallization studies are summarized below in Table 40.

TABLE 40
Reactive Crystallization Studies of Compound 2

| Solvent | Reactive Crystallization Result |
|---|---|
| MeOH | Form-V (SE*) |
| EtOH | Form-I |
| Acetone | — |
| DMF | Form-I (SE*) |
| ACN | Form-I |
| IPA | Form-VIII |
| THF | Form-I |
| 4-Me-2-Pentanone | Form-IX |
| DMSO | Form-VII(SE*) |
| AcOH | Form-I(SE*) |
| DCM | Form-III |
| CHCl3 | Form-I |
| 1,2 propdiol | — |
| Et Gly | — |
| EtOAc | Form-I |
| 14DOX | Form-I |

(SE*): Solids were not obtained through fast crystallization experiments and the resultant solutions were allowed to evaporate at room temperature.

Slurry Crystallization at 40° C. using Form I of compound 2: In a typical experiment, compound 2 (ca. 100 mg) was suspended in minimum quantity the stated solvent while heating to 40° C. The resulted slurries were allowed to stir at 40° C. for three days. The results of the slurry crystallization studies are summarized below in Table 41.

TABLE 41
Slurry Crystallization Studies of Compound 2

| Solvent | Slurry Crystallization Result |
|---|---|
| MeOH | Form-V |
| EtOH | Form-I |
| IPA | Form-I |
| ACN | Form-XIII |
| THF | Form-I |
| Acetone | Form-XII |
| AcOH | Form-XV |
| DCM | Form-XIV |
| 14DOX | Form-XII |
| 1,2-propane diol | Form-XVI |
| Ethylene glycol | Form-XVII |
| CHCl₃ | Form-I |

Anti-Solvent Crystallization: In a typical experiment, compound 2 (ca. 100 mg) was dissolved in solubilizing solvent. To that solution 200 μL anti-solvent was added slowly. These iterations were continued until the precipitation observed or maximum solvent quantity of ~15 mL. The results of the anti-solvent crystallization studies are summarized below in Table 42.

TABLE 42
Anti-Solvent Crystallization Studies of Compound 2

| | | Anti-Solvent |
|---|---|---|
| Solvent | Anti-solvent | Crystallization Result |
| 1,2 Prop Diol | EtOAc | Form-XI |
| 1,2 Prop Diol | T-butyl-methyl-ether | Form-XI |
| 1,2 Prop Diol | DEE | Form-XI |
| 1,2 Prop Diol | 4-Me-2-Pentanone | Form-X |

### Form I of Compound 2

Table 3, supra, is reproduced below and sets forth the X-ray diffraction peaks observed for Form II of compound 2.

TABLE 3
XRPD Peak Positions for Form I of Compound 2

| Pos. [°2θ] | Int. I. (cps) |
|---|---|
| 4.9 | 4508(29) |
| 6.7 | 10424(54) |
| 7.6 | 1046(31) |
| 10.8 | 1587(35) |
| 11.2 | 639(30) |
| 12.3 | 1389(43) |
| 12.7 | 4231(71) |
| 13.3 | 339(31) |
| 14.5 | 4359(133) |
| 15.0 | 8811(282) |
| 15.2 | 8395(304) |
| 15.8 | 2471(80) |
| 16.4 | 2315(107) |
| 17.7 | 7387(187) |
| 18.3 | 19860(311) |
| 18.6 | 1771(182) |
| 19.5 | 7297(166) |
| 20.0 | 32751(798) |
| 20.2 | 9954(1452) |
| 20.4 | 9283(869) |
| 21.0 | 5422(192) |
| 21.6 | 16308(223) |
| 23.1 | 5128(91) |
| 23.7 | 4317(96) |
| 24.4 | 1779(71) |
| 24.8 | 6104(164) |
| 25.2 | 4179(200) |
| 25.7 | 6236(138) |
| 26.4 | 1252(111) |
| 26.6 | 1595(141) |
| 27.1 | 12460(176) |
| 28.3 | 1394(525) |
| 28.5 | 1942(590) |
| 29.3 | 3612(162) |
| 29.8 | 4004(156) |
| 30.5 | 631(94) |
| 31.3 | 637(101) |
| 32.8 | 2547(88) |
| 33.5 | 533(61) |
| 33.9 | 3214(91) |
| 34.7 | 1408(64) |
| 35.1 | 660(64) |
| 35.9 | 1637(62) |
| 36.6 | 1378(72) |
| 39.2 | 960(98) |
| 40.5 | 715(66) |
| 41.1 | 2482(98) |
| 42.6 | 817(58) |
| 44.3 | 441(79) |
| 46.3 | 623(56) |
| 47.7 | 228(32) |
| 48.1 | 1604(47) |
| 49.5 | 1109(58) |
| 51.1 | 946(64) |
| 56.2 | 451(65) |

FIG. 2 depicts an XRPD pattern of Form I of compound 2.

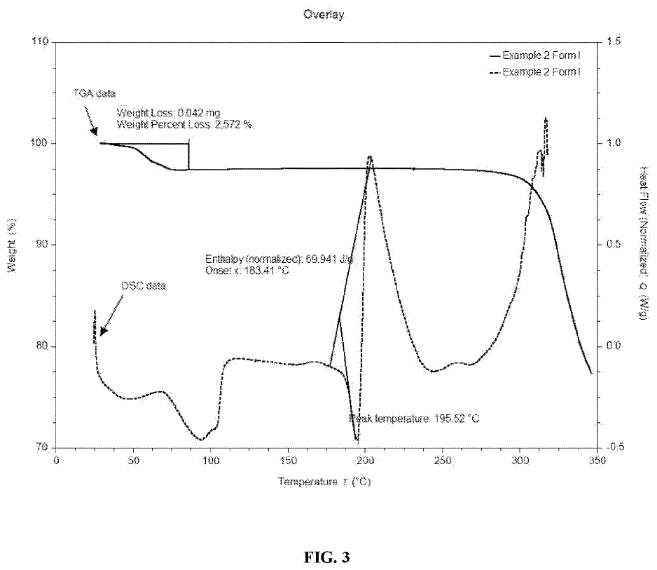

FIG. 3 depicts a DSC thermogram and TGA trace of Form I of compound 2.

### Form II of Compound 2

Table 4, supra, is reproduced below and sets forth the X-ray diffraction peaks observed for Form II of compound 2.

TABLE 4
XRPD Peak Positions for Form II of Compound 2

| Pos. [°2θ] | Int. I. (cps) |
|---|---|
| 5.9 | 39498(195) |
| 8.5 | 181(16) |
| 9.0 | 353(25) |
| 9.7 | 251(22) |
| 10.3 | 270(26) |
| 11.4 | 975(41) |
| 11.9 | 5702(71) |
| 12.3 | 3042(74) |
| 13.4 | 5035(44) |
| 14.1 | 592(51) |
| 15.0 | 4888(73) |
| 15.8 | 4747(49) |
| 16.3 | 238(30) |
| 16.5 | 658(35) |
| 17.0 | 2625(120) |
| 17.2 | 10641(150) |
| 18.0 | 9441(134) |
| 18.3 | 2191(116) |
| 18.6 | 2866(88) |
| 19.2 | 1066(89) |
| 19.9 | 18727(155) |
| 20.4 | 2275(170) |
| 20.8 | 2205(182) |
| 21.7 | 10648(166) |
| 22.2 | 1366(38) |
| 22.7 | 3181(170) |
| 23.0 | 6765(203) |
| 23.3 | 3703(220) |
| 23.8 | 253(52) |
| 24.3 | 3114(58) |
| 24.8 | 2040(47) |
| 25.3 | 799(54) |
| 25.7 | 2350(107) |
| 26.0 | 585(67) |
| 26.7 | 532(559) |
| 26.8 | 837(581) |
| 27.9 | 1956(390) |
| 28.1 | 4487(581) |
| 28.5 | 2880(1108) |
| 28.6 | 2757(1380) |
| 29.0 | 189(18) |
| 29.4 | 794(73) |
| 29.7 | 5478(90) |
| 30.2 | 4964(76) |
| 30.9 | 425(38) |
| 31.4 | 266(46) |
| 31.9 | 3067(130) |
| 33.4 | 424(35) |
| 33.8 | 486(63) |
| 34.1 | 807(70) |
| 34.6 | 1041(78) |
| 35.5 | 1184(67) |
| 36.6 | 1815(85) |
| 37.6 | 5204(130) |
| 38.5 | 288(24) |
| 39.5 | 1325(64) |
| 40.0 | 231(31) |
| 40.6 | 280(42) |
| 40.9 | 256(43) |
| 42.4 | 146(72) |
| 42.6 | 1123(146) |
| 43.2 | 561(134) |
| 44.1 | 452(31) |
| 45.5 | 982(68) |
| 47.7 | 225(21) |
| 48.3 | 131(18) |
| 48.9 | 104(26) |
| 49.3 | 973(52) |
| 51.1 | 677(66) |
| 52.2 | 789(63) |
| 53.5 | 261(22) |
| 58.0 | 801(128) |
| 58.7 | 446(121) |

FIG. 4 depicts an XRPD pattern of Form II of compound 2.

### Form III of Compound 2

Table 5, supra, is reproduced below and sets forth the X-ray diffraction peaks observed for Form III of compound 2.

TABLE 5
XRPD Peak Positions for Form III of Compound 2

| Pos. [°2θ] | Rel. Int. [%] |
|---|---|
| 5.3 | 2.25 |
| 5.9 | 1.18 |
| 6.8 | 100.00 |
| 9.7 | 0.25 |
| 11.4 | 0.46 |
| 12.4 | 0.41 |
| 13.5 | 1.06 |
| 16.5 | 3.86 |
| 18.9 | 22.55 |
| 20.3 | 34.74 |
| 21.1 | 4.51 |
| 23.1 | 2.84 |
| 25.5 | 2.39 |
| 27.1 | 8.12 |
| 29.9 | 0.92 |
| 32.4 | 0.90 |
| 34.3 | 0.71 |
| 38.0 | 0.33 |

FIG. 5 depicts an XRPD pattern of Form III of compound 2.

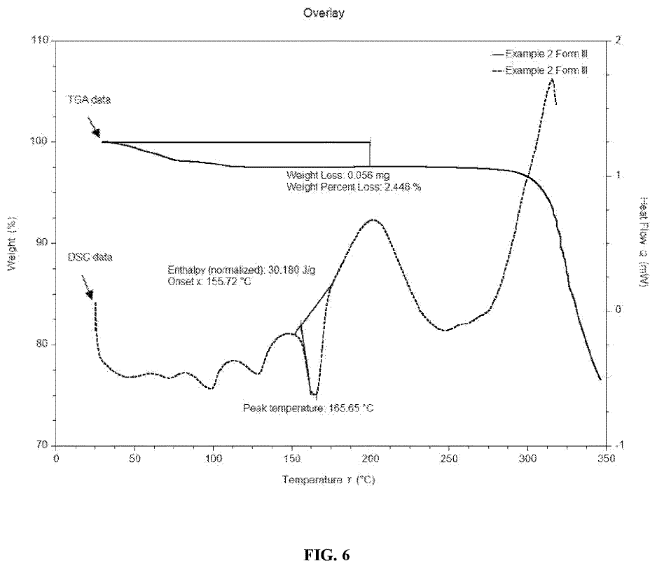

FIG. 6 depicts a DSC thermogram and TGA trace of Form III of compound 2.

### Form IV of Compound 2

Table 6, supra, is reproduced below and sets forth the X-ray diffraction peaks observed for Form IV of compound 2.

TABLE 6
XRPD Peak Positions for Form IV of Compound 2

| Pos. [°2θ] | Rel. Int. [%] |
|---|---|
| 4.9 | 74.87 |
| 5.0 | 100.00 |
| 5.9 | 5.03 |
| 6.5 | 37.18 |
| 6.7 | 30.87 |
| 7.6 | 9.33 |
| 10.0 | 4.23 |
| 11.1 | 16.81 |
| 12.7 | 27.20 |
| 14.6 | 9.90 |
| 15.1 | 28.28 |
| 15.2 | 39.66 |
| 15.4 | 38.95 |
| 16.4 | 6.02 |
| 17.2 | 19.66 |
| 17.6 | 6.62 |
| 18.3 | 10.37 |
| 18.7 | 16.70 |
| 19.2 | 8.12 |
| 19.4 | 22.51 |
| 19.6 | 31.54 |
| 20.0 | 33.63 |
| 21.0 | 10.69 |
| 21.7 | 12.80 |
| 22.0 | 7.71 |
| 22.6 | 9.65 |
| 24.8 | 39.13 |
| 25.0 | 40.45 |
| 26.2 | 13.76 |
| 26.6 | 8.43 |
| 27.4 | 3.30 |
| 30.3 | 2.97 |
| 34.1 | 2.57 |
| 34.9 | 1.73 |

FIG. 7 depicts an XRPD pattern of Form IV of compound 2.

### Form V of Compound 2

Table 7, supra, is reproduced below and sets forth the X-ray diffraction peaks observed for Form V of compound 2.

### Table 7—XRPD Peak Positions for Form V of Compound 2

TABLE 7
XRPD Peak Positions for Form V of Compound 2

| Pos. [°2θ] | Rel. Int. [%] |
|---|---|
| 5.1 | 31.78 |
| 6.0 | 5.46 |
| 7.0 | 55.46 |
| 8.2 | 4.80 |
| 9.9 | 2.26 |
| 11.5 | 12.97 |
| 12.6 | 43.76 |
| 14.2 | 25.26 |
| 15.0 | 48.37 |
| 15.7 | 56.56 |
| 16.2 | 58.98 |
| 17.1 | 8.40 |
| 18.0 | 66.45 |
| 18.7 | 58.86 |
| 19.1 | 47.03 |
| 19.7 | 52.49 |
| 20.7 | 100.00 |
| 21.5 | 49.43 |
| 23.9 | 23.93 |
| 25.3 | 45.23 |
| 25.9 | 63.11 |
| 26.7 | 37.64 |
| 27.4 | 31.90 |
| 28.3 | 14.15 |
| 29.7 | 23.55 |
| 30.9 | 7.44 |
| 31.6 | 8.29 |
| 33.2 | 5.61 |
| 34.5 | 8.31 |
| 38.8 | 2.87 |

FIG. 8 depicts an XRPD pattern of Form V of compound 2.

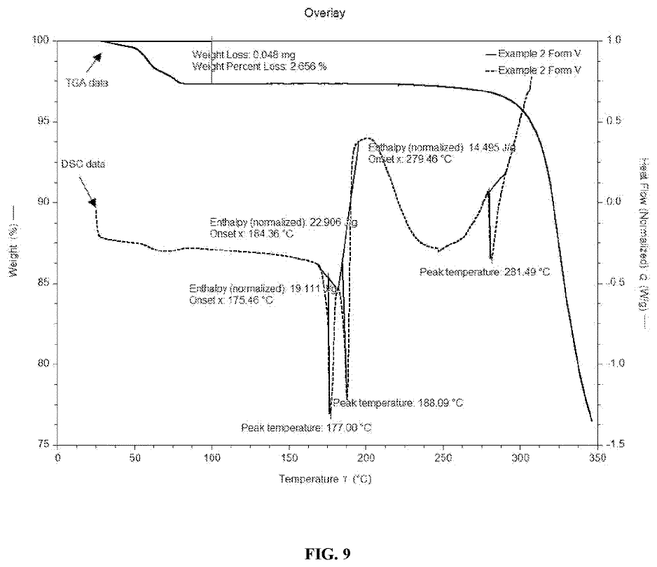

FIG. 9 depicts a DSC thermogram and TGA trace of Form V of compound 2.

### Form VI of Compound 2

Table 8, supra, is reproduced below and sets forth the X-ray diffraction peaks observed for Form VI of compound 2.

TABLE 8
XRPD Peak Positions for Form VI of Compound 2

| Pos. [°2θ] | Rel. Int. [%] |
|---|---|
| 6.5 | 100.00 |
| 12.9 | 13.43 |
| 14.4 | 6.06 |
| 18.3 | 41.70 |
| 19.2 | 70.88 |
| 20.2 | 64.82 |
| 21.3 | 51.71 |
| 32.3 | 4.33 |

FIG. 10 depicts an XRPD pattern of Form VI of compound 2.

### Form VII of Compound 2

Table 9, supra, is reproduced below and sets forth the X-ray diffraction peaks observed for Form VII of compound 2.

TABLE 9
XRPD Peak Positions for Form VII of Compound 2

| Pos. [°2θ] | Rel. Int. [%] |
|---|---|
| 5.0 | 11.63 |
| 6.0 | 3.23 |
| 6.6 | 8.48 |
| 8.2 | 0.33 |
| 9.9 | 2.94 |
| 10.4 | 32.26 |
| 11.9 | 0.21 |
| 13.0 | 1.84 |
| 13.9 | 1.84 |
| 14.4 | 1.15 |
| 14.8 | 100.00 |
| 15.2 | 7.47 |
| 16.2 | 0.92 |
| 16.3 | 1.02 |
| 17.0 | 0.39 |
| 17.7 | 0.78 |
| 19.0 | 1.42 |
| 19.5 | 13.27 |
| 20.2 | 5.25 |
| 20.8 | 5.25 |
| 22.0 | 11.31 |
| 24.7 | 9.21 |
| 24.9 | 71.35 |
| 25.6 | 3.27 |
| 26.1 | 2.85 |
| 26.6 | 4.77 |
| 28.0 | 1.15 |
| 29.7 | 6.68 |
| 29.9 | 6.08 |
| 30.3 | 2.35 |
| 31.4 | 1.56 |
| 32.6 | 3.64 |
| 33.1 | 1.41 |
| 34.0 | 0.80 |
| 34.8 | 3.22 |
| 35.4 | 1.13 |
| 35.7 | 1.14 |
| 36.2 | 1.38 |
| 37.3 | 0.81 |
| 38.6 | 1.41 |
| 39.6 | 0.50 |

FIG. 11 depicts an XRPD pattern of Form VII of compound 2.

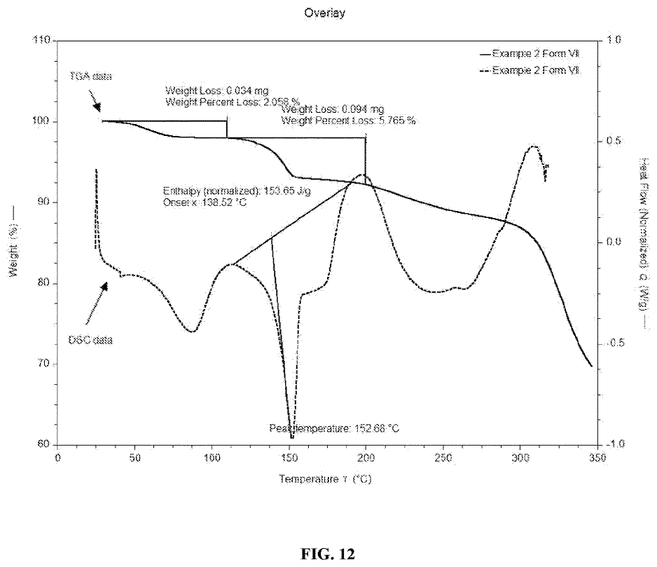

FIG. 12 depicts a DSC thermogram and TGA trace of Form VII of compound 2.

### Form VIII of Compound 2

XRPD data indicated Form VIII of compound 2 is an amorphous form. It was observed that Form VIII of compound 2 transformed to Form I of compound 2 during storage at RT for about one month.

FIG. 13 depicts an XRPD pattern of Form VIII of compound 2.

### Form IX of Compound 2

Table 10, supra, is reproduced below and sets forth the X-ray diffraction peaks observed for Form IX of compound 2.

TABLE 10
XRPD Peak Positions for Form IX of Compound 2

| Pos. [°2θ] | Rel. Int. [%] |
|---|---|
| 6.3 | 100.00 |
| 6.7 | 24.97 |
| 12.6 | 5.78 |
| 14.6 | 5.25 |
| 15.0 | 12.61 |
| 17.8 | 10.38 |
| 18.3 | 18.10 |
| 18.9 | 45.02 |
| 19.9 | 23.15 |
| 21.1 | 9.47 |
| 25.1 | 5.04 |
| 31.7 | 3.22 |

FIG. 14 depicts an XRPD pattern of Form IX of compound 2.

### Form X of Compound 2

Table 11, supra, is reproduced below and sets forth the X-ray diffraction peaks observed for Form X of compound 2.

TABLE 11
XRPD Peak Positions for Form X of Compound 2

| Pos. [°2θ] | Rel. Int. [%] |
|---|---|
| 5.3 | 24.25 |
| 6.6 | 100.00 |
| 7.0 | 41.87 |
| 9.6 | 15.85 |
| 10.3 | 18.42 |
| 12.2 | 3.06 |
| 13.0 | 18.74 |
| 13.8 | 26.18 |
| 14.7 | 37.68 |
| 15.2 | 26.06 |
| 16.0 | 12.47 |
| 16.5 | 23.68 |
| 17.6 | 21.19 |
| 18.2 | 92.96 |
| 19.2 | 70.67 |
| 19.8 | 85.36 |
| 20.3 | 38.87 |
| 21.1 | 29.23 |
| 21.5 | 39.52 |
| 22.6 | 27.01 |
| 23.1 | 18.23 |
| 24.0 | 16.42 |
| 25.3 | 31.31 |
| 26.5 | 11.77 |
| 27.3 | 9.87 |
| 28.2 | 8.29 |
| 30.2 | 6.03 |
| 30.6 | 5.09 |
| 31.8 | 6.76 |
| 34.9 | 2.82 |
| 36.7 | 3.09 |

FIG. 15 depicts an XRPD pattern of Form X of compound 2.

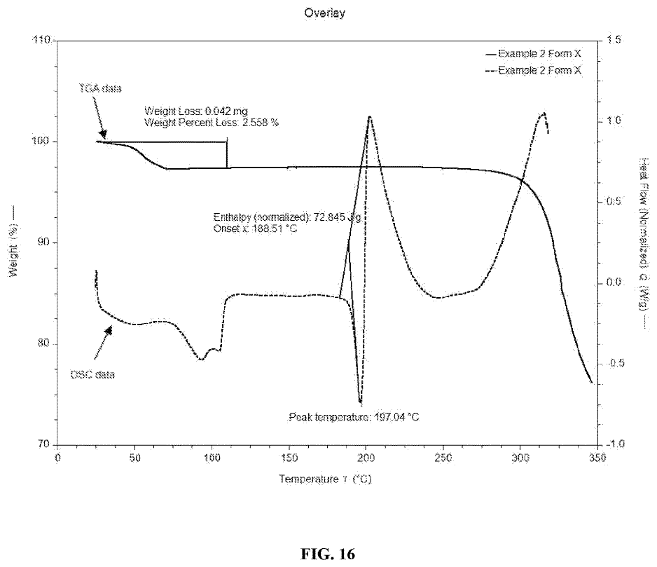

FIG. 16 depicts a DSC thermogram and TGA trace of Form X of compound 2.

### Form XI of Compound 2

Table 12, supra, is reproduced below and sets forth the X-ray diffraction peaks observed for Form XI of compound 2.

TABLE 12
XRPD Peak Positions for Form XI of Compound 2

| Pos. [°2θ] | Rel. Int. [%] |
|---|---|
| 5.2 | 7.06 |
| 7.0 | 45.43 |
| 7.8 | 1.72 |
| 11.0 | 2.44 |
| 13.1 | 12.60 |
| 13.6 | 9.76 |
| 15.2 | 27.47 |
| 16.6 | 1.48 |
| 18.4 | 7.96 |
| 20.0 | 100.00 |
| 20.3 | 41.62 |
| 21.3 | 19.61 |
| 22.1 | 18.70 |
| 23.3 | 6.64 |
| 24.2 | 8.18 |
| 25.3 | 5.47 |
| 26.0 | 5.08 |
| 27.0 | 17.56 |
| 28.4 | 3.67 |
| 29.7 | 5.14 |
| 30.9 | 3.68 |
| 31.9 | 1.72 |
| 33.3 | 3.52 |
| 34.1 | 3.13 |
| 35.0 | 1.64 |
| 37.1 | 1.59 |

FIG. 17 depicts an XRPD pattern of Form XI of compound 2.

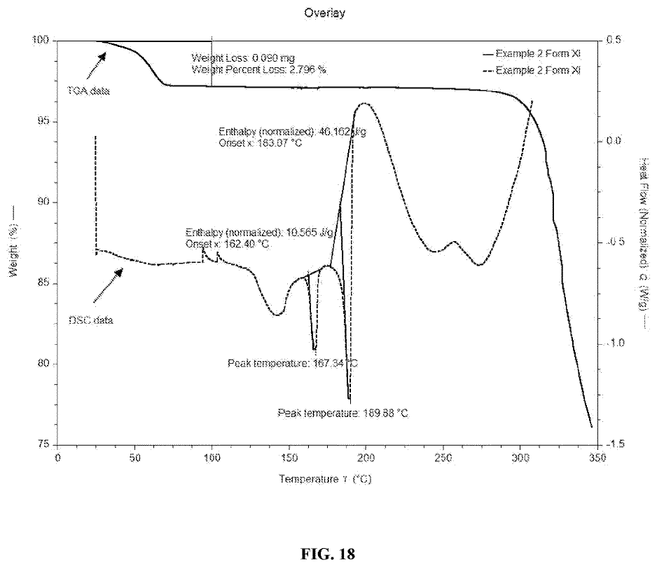

FIG. 18 depicts a DSC thermogram and TGA trace of Form XI of compound 2.

### Form XII of Compound 2

Table 13, supra, is reproduced below and sets forth the X-ray diffraction peaks observed for Form XII of compound 2.

TABLE 13
XRPD Peak Positions for Form XII of Compound 2

| Pos. [°2θ] | Rel. Int. [%] |
|---|---|
| 5.8 | 100.00 |
| 5.9 | 69.25 |
| 8.6 | 1.17 |
| 9.0 | 0.97 |
| 10.3 | 1.23 |
| 11.5 | 9.82 |
| 12.2 | 2.11 |
| 12.5 | 5.65 |
| 13.4 | 10.91 |
| 14.2 | 6.82 |
| 15.0 | 14.07 |
| 15.3 | 6.94 |
| 15.8 | 17.30 |
| 16.0 | 11.92 |
| 16.4 | 5.13 |
| 16.7 | 11.16 |
| 16.8 | 14.16 |
| 17.1 | 18.55 |
| 17.4 | 11.14 |
| 18.1 | 22.70 |
| 18.6 | 11.84 |
| 18.9 | 7.03 |
| 19.3 | 22.76 |
| 20.1 | 17.67 |
| 20.6 | 17.20 |
| 21.4 | 9.99 |
| 21.6 | 12.63 |
| 21.8 | 13.31 |
| 22.2 | 18.68 |
| 22.4 | 14.59 |
| 23.0 | 16.52 |
| 23.3 | 17.08 |
| 23.7 | 14.68 |
| 24.4 | 6.94 |
| 25.0 | 4.77 |
| 25.6 | 5.06 |
| 26.2 | 6.37 |
| 26.5 | 3.93 |
| 27.1 | 5.46 |
| 27.8 | 8.51 |
| 28.4 | 11.99 |
| 29.1 | 8.09 |
| 29.6 | 6.24 |
| 29.9 | 2.79 |
| 30.2 | 4.58 |
| 31.0 | 4.31 |
| 31.9 | 3.42 |
| 33.0 | 1.91 |
| 34.4 | 1.83 |
| 36.5 | 1.09 |
| 37.1 | 1.94 |

FIG. 19 depicts an XRPD pattern of Form XII of compound 2.

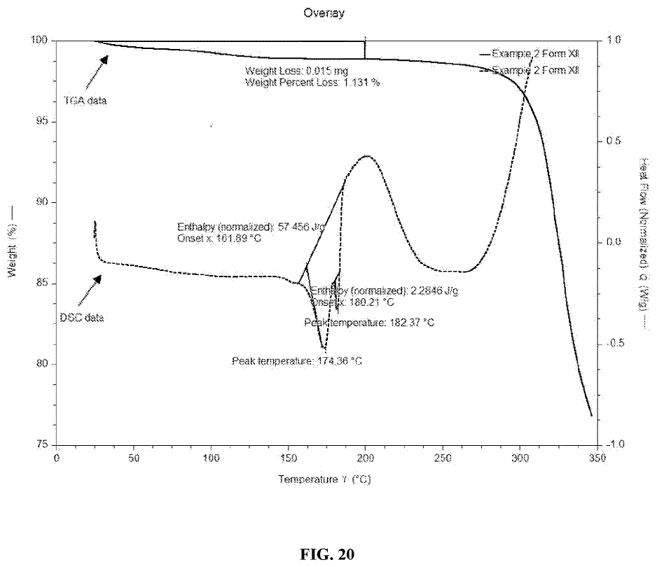

FIG. 20 depicts a DSC thermogram and TGA trace of Form XII of compound 2.

### Form XIII of Compound 2

Table 14, supra, is reproduced below and sets forth the X-ray diffraction peaks observed for Form XIII of compound 2.

TABLE 14
XRPD Peak Positions for Form XIII of Compound 2

| Pos. [°2θ] | Rel. Int. [%] |
|---|---|
| 6.1 | 100.00 |
| 9.8 | 2.20 |
| 11.6 | 4.94 |
| 12.3 | 5.16 |
| 13.2 | 6.01 |
| 14.2 | 7.59 |
| 15.0 | 2.94 |
| 15.7 | 8.58 |
| 15.9 | 10.93 |
| 16.3 | 0.66 |
| 17.2 | 11.74 |
| 17.8 | 8.25 |
| 18.4 | 15.70 |
| 19.1 | 5.29 |
| 19.6 | 10.12 |
| 20.3 | 9.65 |
| 20.9 | 9.86 |
| 21.8 | 18.01 |
| 22.8 | 13.65 |
| 23.2 | 12.51 |
| 24.7 | 6.83 |
| 26.7 | 5.37 |
| 28.4 | 6.93 |
| 31.0 | 2.50 |

FIG. 21 depicts an XRPD pattern of Form XIII of compound 2.

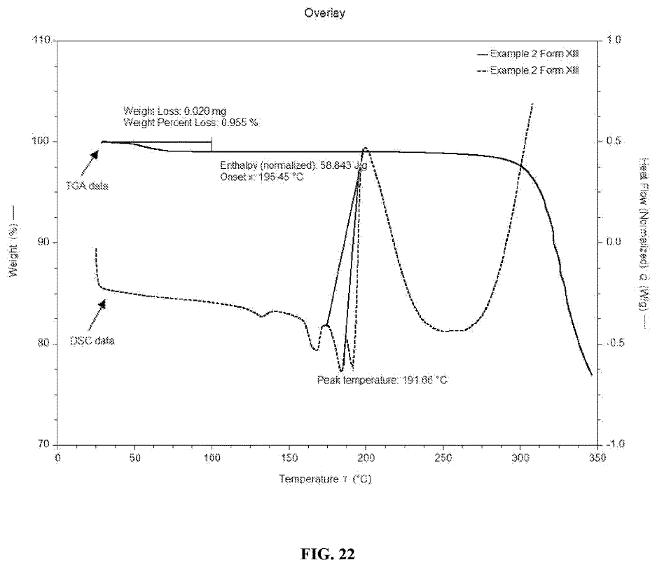

FIG. 22 depicts a DSC thermogram and TGA trace of Form XIII of compound 2.

### Example 3. Forms I and II of Compound 3

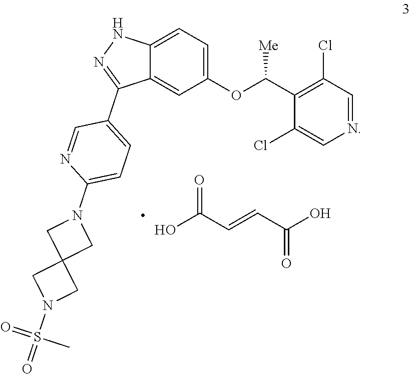 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=C(O)/C=C/C(=O)O]

### Form I of Compound 3

Form I of compound 3 was prepared as follows: compound A (192 mg) was treated with maleic acid (1.00 equiv) in acetonitrile (25 mL) at room temperature for 2 hours and collected to give a white solid (207 mg, 89% yield, 99.4% purity).

LCMS: m/z=559.1 (M+H); ¹H NMR (400 MHz, DMSO-d6) δ=13.07 (br s, 3H), 8.58 (s, 2H), 8.49 (d, J=1.8 Hz, 1H), 7.93 (dd, J=2.3, 8.7 Hz, 1H), 7.47 (d, J=9.0 Hz, 1H), 7.17 (d, J=2.1 Hz, 1H), 7.10 (dd, J=2.3, 9.0 Hz, 1H), 6.61 (d, J=8.6 Hz, 1H), 6.24 (s, 2H), 6.11 (q, J=6.6 Hz, 1H), 4.22 (s, 4H), 4.12 (s, 4H), 3.03 (s, 3H), 1.76 (d, J=6.6 Hz, 3H; m.p.=181.3-184.5° C.

Table 15, supra, is reproduced below and sets forth the X-ray diffraction peaks observed for Form I of compound 3.

TABLE 15
XRPD Peak Positions for Form I of Compound 3

| Pos. [°2θ] | Int. I. (cps) |
|---|---|
| 4.9 | 284(18) |
| 6.2 | 6356(70) |
| 7.3 | 8657(47) |
| 9.9 | 682(19) |
| 12.5 | 2706(116) |
| 13.6 | 218(42) |
| 14.4 | 5298(76) |
| 14.9 | 5278(125) |
| 15.2 | 9250(169) |
| 15.8 | 910(269) |
| 16.1 | 5740(230) |
| 16.9 | 2080(50) |
| 17.1 | 3987(70) |
| 17.9 | 2194(47) |
| 18.4 | 4591(126) |
| 18.8 | 27152(1704) |
| 18.9 | 23725(1657) |
| 19.1 | 3249(523) |
| 19.8 | 2272(104) |
| 20.3 | 700(36) |
| 21.0 | 1233(26) |
| 21.4 | 1064(30) |
| 21.7 | 1599(49) |
| 21.9 | 967(93) |
| 22.1 | 617(79) |
| 22.6 | 6119(51) |
| 23.7 | 2378(81) |
| 24.1 | 11676(113) |
| 24.7 | 4255(225) |
| 25.1 | 5960(373) |
| 25.3 | 4847(267) |
| 25.5 | 19272(611) |
| 26.0 | 2754(71) |
| 26.5 | 4946(434) |
| 26.7 | 15174(569) |
| 27.0 | 1977(242) |
| 27.6 | 3722(49) |
| 29.1 | 3876(298) |
| 29.2 | 6614(309) |
| 29.7 | 3994(51) |
| 30.3 | 6155(59) |
| 30.8 | 3684(58) |
| 31.1 | 191(37) |
| 31.6 | 2371(40) |
| 32.9 | 200(27) |
| 33.2 | 763(29) |
| 33.5 | 1947(68) |
| 34.2 | 6405(95) |
| 34.6 | 2308(91) |
| 35.3 | 5280(80) |
| 36.6 | 825(74) |
| 36.9 | 1463(91) |
| 37.5 | 1832(63) |
| 38.4 | 3245(73) |
| 39.6 | 516(27) |
| 40.8 | 422(36) |
| 41.0 | 1123(55) |
| 41.7 | 120(20) |
| 42.2 | 1045(34) |
| 43.0 | 1097(38) |
| 43.9 | 1616(38) |
| 45.8 | 584(52) |
| 46.2 | 560(51) |
| 46.8 | 1434(65) |
| 47.7 | 158(22) |
| 48.2 | 145(33) |
| 48.5 | 892(39) |
| 49.0 | 837(71) |
| 49.4 | 1676(86) |
| 50.5 | 575(46) |
| 51.3 | 576(52) |
| 52.2 | 355(49) |
| 53.4 | 875(38) |
| 54.6 | 2339(82) |
| 57.5 | 914(69) |
| 58.5 | 188(29) |

FIG. 23 depicts an XRPD pattern of Form I of compound 3.

### Form II of Compound 3

A reaction between compound A (THE solution or acetone solution) and maleic acid (MeOH solutions; 1 and 2 equivalents of acid with respect to TYRA-300) yielded crystalline material. No weight loss, from room temperature to 150° C., was observed in the TGA data which suggests an anhydrous form. DSC data exhibited an endothermic peak at 185.57° C. (peak temperature) correspond to the melting point. Vapor sorption data (not shown) suggests a moderately hygroscopic material with stability.

Table 16, supra, is reproduced below and sets forth the X-ray diffraction peaks observed for Form II of compound 3.

TABLE 16
XRPD Peak Positions for Form II of Compound 3

| Pos. [°2θ] | Rel. Int. [%] |
|---|---|
| 5.9 | 6.62 |
| 7.2 | 3.30 |
| 7.7 | 1.16 |
| 11.5 | 8.29 |
| 13.6 | 2.43 |
| 14.5 | 5.10 |
| 15.2 | 100.00 |
| 16.7 | 10.50 |
| 18.6 | 91.20 |
| 19.4 | 12.89 |
| 20.3 | 15.85 |
| 20.9 | 38.76 |
| 21.6 | 17.12 |
| 22.2 | 12.86 |
| 22.8 | 15.59 |
| 23.6 | 4.81 |
| 24.4 | 7.97 |
| 24.9 | 8.75 |
| 25.8 | 10.48 |
| 26.5 | 5.49 |
| 27.1 | 13.06 |
| 27.7 | 21.02 |
| 28.4 | 20.89 |
| 28.7 | 5.57 |
| 29.7 | 7.00 |
| 29.9 | 6.89 |
| 30.3 | 7.08 |
| 30.5 | 8.96 |
| 30.9 | 9.98 |
| 32.8 | 22.21 |
| 34.4 | 5.09 |
| 35.1 | 4.96 |
| 35.5 | 4.46 |
| 36.4 | 10.29 |
| 37.7 | 4.34 |
| 38.2 | 13.47 |
| 39.3 | 3.41 |

FIG. 58 depicts an XRPD pattern of Form II of compound 3.

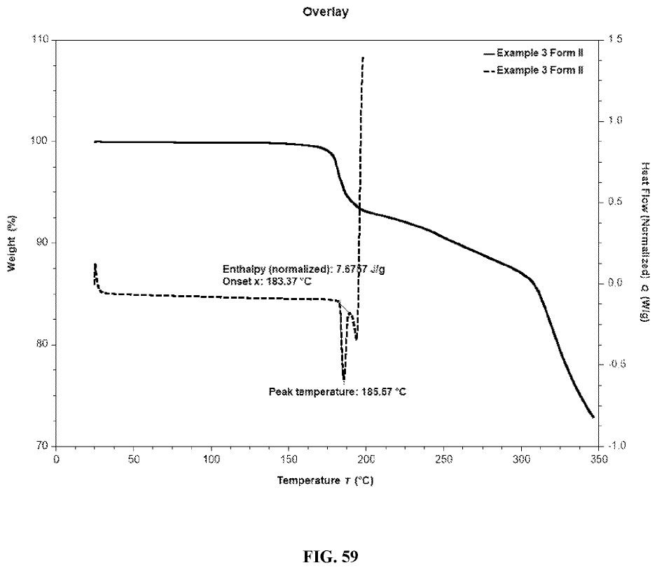

FIG. 59 depicts a DSC thermogram and TGA trace of Form II of compound 3.

### Example 4. Form I of Compound 4

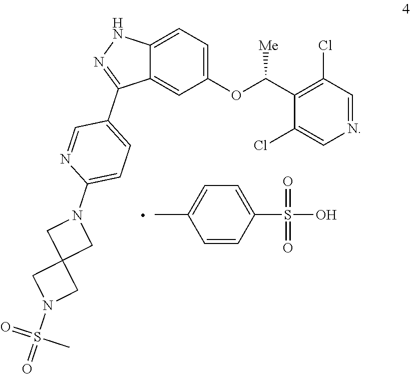

Form I of Compound 4 was synthesized using the method described in Example 3.

LCMS: m/z=559.1 (M+H); ¹H NMR (400 MHz, DMSO-d6) δ=13.27 (br s, 1H), 8.58 (s, 2H), 8.36 (d, J=1.5 Hz, 1H), 8.24 (br d, J=8.3 Hz, 1H), 7.55-7.44 (m, 3H), 7.21 (d, J=2.1 Hz, 1H), 7.15-7.08 (m, 3H), 6.95 (br d, J=9.0 Hz, 1H), 6.14 (q, J=6.6 Hz, 1H), 4.43 (s, 4H), 4.15 (s, 4H), 3.04 (s, 3H), 2.28 (s, 3H), 1.77 (d, J=6.6 Hz, 3H); m.p.>160° C. decompose.

Table 17, supra, is reproduced below and sets forth the X-ray diffraction peaks observed for Form I of compound 4.

TABLE 17
XRPD Peak Positions for Form I of Compound 4

| Pos. [°2θ] | Int. I. (cps) |
|---|---|
| 4.8 | 4690(227) |
| 6.2 | 5431(253) |
| 8.2 | 450(54) |
| 14.3 | 18673(927) |
| 17.8 | 35277(922) |
| 19.5 | 72093(411) |
| 25.7 | 38317(380) |
| 27.8 | 24015(382) |
| 31.3 | 899(132) |
| 51.4 | 2203(116) |

FIG. 24 depicts an XRPD pattern of Form I of compound 4.

### Example 5. Forms I and II of Compound 5

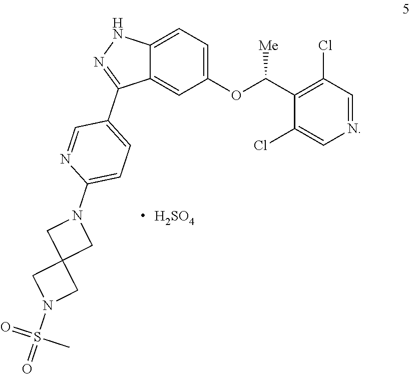

### Form I of Compound 5

Form I of Compound 5 was synthesized using the method described in Example 3.

LCMS: m/z=559.1 (M+H); ¹H NMR (400 MHz, DMSO-d6) δ=13.27 (br s, 1H), 8.58 (s, 2H), 8.36 (d, J=1.7 Hz, 1H), 8.23 (br d, J=9.0 Hz, 1H), 7.51 (d, J=9.0 Hz, 1H), 7.21 (d, J=2.1 Hz, 1H), 7.13 (dd, J=2.3, 9.0 Hz, 1H), 6.93 (br d, J=8.7 Hz, 1H), 6.14 (q, J=6.7 Hz, 1H), 4.42 (s, 4H), 4.15 (s, 4H), 3.04 (s, 3H), 1.77 (d, J=6.6 Hz, 3H); m.p.=210.6-241.6° C.

Table 18, supra, is reproduced below and sets forth the X-ray diffraction peaks observed for Form I of compound 5.

TABLE 18
XRPD Peak Positions for Form I of Compound 5

| Pos. [°2θ] | Int. I. (cps) |
|---|---|
| 5.5 | 6513(57) |
| 7.3 | 251(21) |
| 12.0 | 90(30) |
| 13.4 | 4945(41) |
| 14.2 | 8543(67) |
| 14.7 | 8586(65) |
| 15.7 | 1640(36) |
| 16.4 | 7024(73) |
| 16.8 | 4885(126) |
| 17.2 | 3627(121) |
| 19.1 | 5061(104) |
| 20.0 | 27700(225) |
| 20.6 | 32930(226) |
| 21.6 | 2912(81) |
| 22.0 | 719(65) |
| 22.7 | 6423(189) |
| 23.3 | 2953(126) |
| 24.5 | 10203(133) |
| 25.2 | 7983(221) |
| 25.5 | 4630(388) |
| 25.9 | 9491(299) |
| 26.4 | 626(82) |
| 26.9 | 1085(79) |
| 27.3 | 5244(95) |
| 28.0 | 903(70) |
| 28.4 | 2010(107) |
| 28.9 | 1910(87) |
| 31.1 | 1903(131) |
| 32.3 | 767(94) |
| 33.2 | 1653(608) |
| 33.6 | 2411(606) |
| 34.4 | 1219(60) |
| 36.2 | 1405(75) |
| 36.8 | 934(57) |
| 37.8 | 982(178) |
| 38.4 | 2898(222) |
| 38.9 | 899(242) |
| 40.0 | 879(98) |
| 41.0 | 1197(73) |
| 42.3 | 356(76) |
| 44.3 | 633(45) |
| 45.0 | 1050(52) |
| 46.1 | 1698(307) |
| 46.6 | 904(311) |
| 47.7 | 213(18) |
| 50.4 | 642(138) |
| 51.1 | 2936(155) |
| 53.2 | 1117(66) |
| 55.4 | 391(49) |
| 58.4 | 646(55) |
| 59.1 | 143(43) |

FIG. 25 depicts an XRPD pattern of Form I of compound 5.

### Form II of Compound 5

A reaction between compound A (THF solution) and H₂SO₄ (87% aqueous) yielded crystalline material. About 2.76% weight loss, from room temperature to 120° C., was observed in the TGA data. The DSC data exhibited an endothermic peak at 140.26° C. (peak temperature) followed by an exothermic event at 238.01° C. Vapor sorption data (not shown) suggest a moderately hygroscopic material. XRPD data analyses on the material that was recovered from DVS experiment (not shown) suggests stability.

Table 19, supra, is reproduced below and sets forth the X-ray diffraction peaks observed for Form II of compound 5.

TABLE 19
XRPD Peak Positions for Form II of Compound 5

| Pos. [°2θ] | Rel. Int. [%] |
|---|---|
| 5.8 | 12.69 |
| 8.7 | 0.80 |
| 12.4 | 0.91 |
| 13.7 | 17.45 |
| 14.5 | 28.06 |
| 15.1 | 21.74 |
| 16.0 | 8.07 |
| 16.7 | 28.78 |
| 17.2 | 22.90 |
| 17.5 | 15.24 |
| 18.9 | 15.59 |
| 19.4 | 26.63 |
| 20.4 | 100.00 |
| 21.1 | 71.79 |
| 22.0 | 22.25 |
| 23.1 | 31.80 |
| 23.9 | 16.43 |
| 24.8 | 36.99 |
| 25.5 | 37.70 |
| 26.2 | 38.46 |
| 27.8 | 27.73 |
| 28.3 | 16.40 |
| 29.3 | 18.26 |
| 30.1 | 9.80 |
| 31.4 | 8.83 |
| 32.6 | 8.74 |
| 34.0 | 12.25 |
| 34.8 | 8.63 |
| 36.6 | 7.80 |
| 37.2 | 6.67 |
| 38.7 | 7.13 |

FIG. 60 depicts an XRPD pattern of Form II of compound 5.

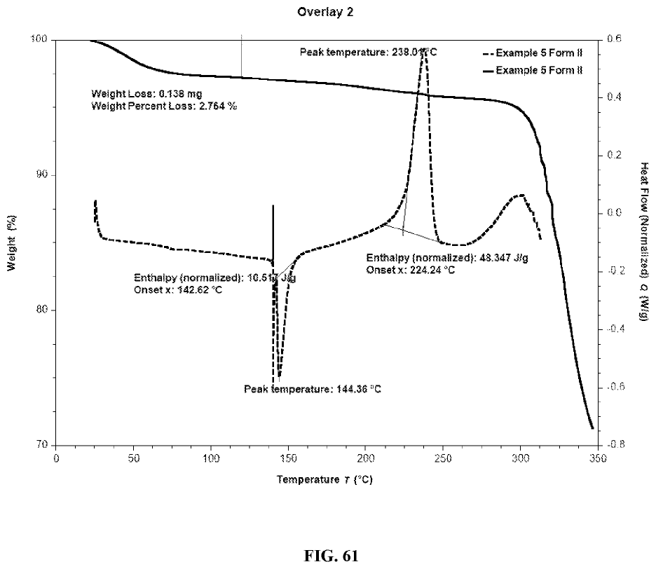

FIG. 61 depicts a DSC thermogram and TGA trace of Form II of compound 5.

### Example 6. Form I of Compound 6

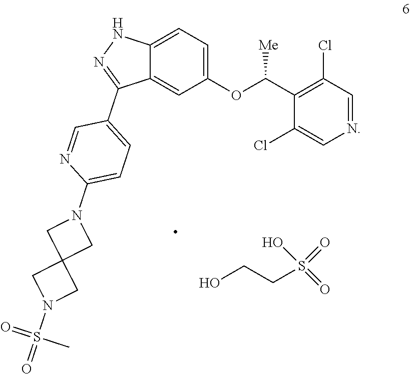 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=S(=O)(O)CCO]

Form I of Compound 6 was synthesized using the method described in Example 3.

LCMS: m/z=559.1 (M+H); ¹H NMR (400 MHz, DMSO-d6) δ=13.26 (br s, 1H), 8.58 (s, 2H), 8.37 (d, J=1.6 Hz, 1H), 8.23 (br d, J=8.7 Hz, 1H), 7.51 (d, J=9.0 Hz, 1H), 7.21 (d, J=2.1 Hz, 1H), 7.13 (dd, J=2.3, 9.0 Hz, 1H), 6.93 (br d, J=8.4 Hz, 1H), 6.14 (q, J=6.6 Hz, 1H), 4.42 (s, 4H), 4.15 (s, 4H), 3.63 (t, J=6.7 Hz, 3H), 3.04 (s, 3H), 2.61 (t, J=6.7 Hz, 2H), 1.77 (d, J=6.6 Hz, 3H); m.p.=176.3-187.5° C.

Table 20, supra, is reproduced below and sets forth the X-ray diffraction peaks observed for Form I of compound 6.

TABLE 20
XRPD Peak Positions for Form I of Compound 6

| Pos. [°2θ] | Int. I. (cps) |
|---|---|
| 4.7 | 2276(155) |
| 4.9 | 1117(154) |
| 5.8 | 659(24) |
| 6.6 | 3628(31) |
| 10.0 | 472(56) |
| 11.6 | 1980(48) |
| 12.6 | 247(42) |
| 13.5 | 863(71) |
| 13.9 | 1113(80) |
| 14.7 | 2879(87) |
| 15.8 | 4362(101) |
| 16.4 | 1854(74) |
| 16.9 | 302(45) |
| 17.4 | 1343(45) |
| 18.5 | 915(107) |
| 18.8 | 1386(230) |
| 19.4 | 3378(170) |
| 20.1 | 11319(156) |
| 20.7 | 12797(258) |
| 21.7 | 1150(82) |
| 24.8 | 4223(268) |
| 25.6 | 7663(311) |
| 26.0 | 1192(167) |
| 26.7 | 1664(165) |
| 27.0 | 2340(190) |
| 28.0 | 163(43) |
| 28.4 | 1210(139) |
| 28.9 | 342(98) |
| 33.2 | 471(64) |
| 34.1 | 1628(83) |
| 35.5 | 558(48) |
| 37.6 | 451(62) |
| 38.7 | 629(53) |
| 41.0 | 1242(73) |

FIG. 26 depicts an XRPD pattern of Form I of compound 6.

### Example 7. Form I of Compound 7

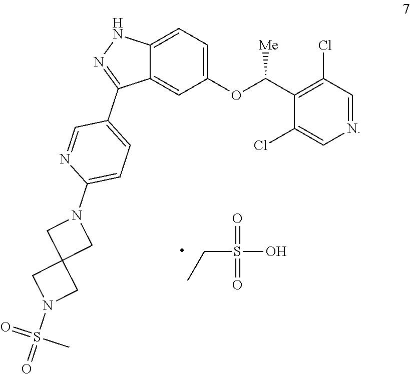

Form I of Compound 7 was synthesized using the method described in Example 3.

LCMS: m/z=559.1 (M+H); ¹H NMR (400 MHz, DMSO-d₆) δ=13.25 (br s, 1H), 8.58 (s, 2H), 8.38 (d, J=1.6 Hz, 1H), 8.21 (br d, J=7.9 Hz, 1H), 7.51 (d, J=9.0 Hz, 1H), 7.21 (d, J=2.1 Hz, 1H), 7.13 (dd, J=2.3, 9.0 Hz, 1H), 6.91 (br d, J=8.6 Hz, 1H), 6.14 (q, J=6.6 Hz, 1H), 4.41 (s, 4H), 4.15 (s, 4H), 3.04 (s, 3H), 2.39 (q, J=7.4 Hz, 2H), 1.77 (d, J=6.6 Hz, 3H), 1.06 (t, J=7.5 Hz, 3H); m.p.=161.4-172.6° C.

Table 21, supra, is reproduced below and sets forth the X-ray diffraction peaks observed for Form I of compound 7.

TABLE 21
XRPD Peak Positions for Form I of Compound 7

| Pos. [°2θ] | Int. I. (cps) |
|---|---|
| 4.8 | 183(32) |
| 5.2 | 868(44) |
| 6.6 | 3587(45) |
| 10.0 | 1023(40) |
| 12.3 | 173(29) |
| 13.2 | 1038(66) |
| 14.4 | 5426(82) |
| 15.8 | 414(76) |
| 16.2 | 1847(112) |
| 17.5 | 1265(130) |
| 17.8 | 1029(218) |
| 18.1 | 1564(215) |
| 18.7 | 6577(152) |
| 19.2 | 3040(100) |
| 20.1 | 1607(61) |
| 20.6 | 716(65) |
| 21.7 | 1380(85) |
| 23.6 | 996(77) |
| 24.6 | 1820(233) |
| 25.4 | 11781(378) |
| 26.4 | 5524(239) |
| 27.0 | 4023(165) |
| 29.2 | 777(69) |
| 32.0 | 635(38) |
| 33.9 | 259(41) |
| 34.5 | 1089(76) |
| 36.0 | 154(35) |
| 40.6 | 143(34) |
| 41.6 | 636(59) |
| 44.3 | 171(26) |
| 46.1 | 156(37) |
| 47.7 | 146(30) |

FIG. 27 depicts an XRPD pattern of Form I of compound 7.

### Example 8. Form I of Compound 8

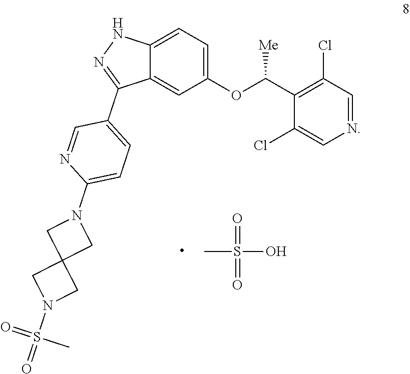

Form I of Compound 8 was synthesized using the method described in Example 3.

LCMS: m/z=559.1 (M+H); ¹H NMR (400 MHz, DMSO-d₆) δ=13.27 (br s, 1H), 8.58 (s, 2H), 8.37 (d, J=1.7 Hz, 1H), 8.23 (br d, J=8.4 Hz, 1H), 7.51 (d, J=9.0 Hz, 1H), 7.21 (d, J=2.1 Hz, 1H), 7.13 (dd, J=2.3, 9.0 Hz, 1H), 6.93 (br d, J=9.3 Hz, 1H), 6.14 (q, J=6.8 Hz, 1H), 4.42 (s, 4H), 4.15 (s, 4H), 3.04 (s, 3H), 2.32 (s, 3H), 1.77 (d, J=6.6 Hz, 3H); m.p. 147.1-185 (decompose).

Table 22, supra, is reproduced below and sets forth the X-ray diffraction peaks observed for Form I of compound 8.

TABLE 22
XRPD Peak Positions for Form I of Compound 8

| Pos. [°2θ] | Int. I. (cps) |
|---|---|
| 5.0 | 2567(46) |
| 6.9 | 3400(45) |
| 10.0 | 518(56) |
| 11.7 | 484(47) |
| 14.2 | 2223(146) |
| 16.1 | 1174(149) |
| 20.0 | 13723(880) |
| 21.2 | 4446(856) |
| 25.0 | 5231(219) |
| 25.6 | 6827(209) |
| 26.8 | 3362(89) |
| 27.5 | 494(86) |
| 28.3 | 1948(85) |
| 32.0 | 387(44) |
| 33.2 | 313(43) |
| 34.3 | 758(64) |

FIG. 28 depicts an XRPD pattern of Form I of compound 8.

### Example 9. Form I of Compound 9

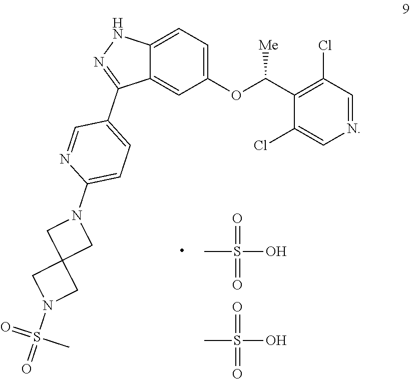

Form I of Compound 9 was synthesized using the method described in Example 3.

LCMS: m/z=559.1 (M+H); ¹H NMR (400 MHz, DMSO-d6) δ=13.31 (br s, 1H), 8.58 (s, 2H), 8.35 (d, J=1.8 Hz, 1H), 8.29 (br d, J=8.8 Hz, 1H), 7.52 (d, J=9.0 Hz, 1H), 7.22 (d, J=2.2 Hz, 1H), 7.14 (dd, J=2.3, 9.0 Hz, 1H), 6.99 (br d, J=8.9 Hz, 1H), 6.14 (q, J=6.7 Hz, 1H), 4.46 (s, 4H), 4.15 (s, 4H), 3.04 (s, 3H), 2.33 (s, 6H), 1.77 (d, J=6.7 Hz, 3H).

### Example 10. Form I of Compound 10

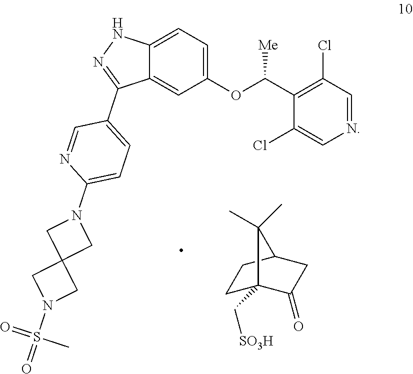

Form I of compound 10 was synthesized using the method described in Example 3.

LCMS: m/z=559.1 (M+H); ¹H NMR (400 MHz, DMSO-d6) δ=13.25 (br s, 1H), 8.58 (s, 2H), 8.38 (d, J=1.7 Hz, 1H), 8.23 (br d, J=8.8 Hz, 1H), 7.51 (d, J=9.0 Hz, 1H), 7.21 (d, J=2.1 Hz, 1H), 7.13 (dd, J=2.3, 9.0 Hz, 1H), 6.93 (br d, J=8.4 Hz, 1H), 6.14 (q, J=6.7 Hz, 1H), 4.42 (s, 4H), 4.15 (s, 4H), 3.04 (s, 3H), 2.87 (d, J=14.7 Hz, 1H), 2.73-2.64 (m, 1H), 2.38 (d, J=14.7 Hz, 1H), 2.23 (td, J=3.9, 17.9 Hz, 1H), 1.93 (t, J=4.5 Hz, 1H), 1.89-1.74 (m, 5H), 1.32-1.23 (m, 2H), 1.05 (s, 3H), 0.74 (s, 3H); m.p.=192.7-201.1° C.

Table 23, supra, is reproduced below and sets forth the X-ray diffraction peaks observed for Form I of compound 10.

TABLE 23
XRPD Peak Positions for Form I of Compound 10

| Pos. [°2θ] | Int. I. (cps) |
|---|---|
| 3.9 | 2000(60) |
| 4.4 | 3761(63) |
| 5.4 | 3038(37) |
| 6.4 | 1584(71) |
| 6.8 | 2431(102) |
| 7.3 | 710(79) |
| 12.7 | 6433(96) |
| 14.0 | 7612(247) |
| 14.6 | 12020(246) |
| 15.7 | 3748(115) |
| 16.7 | 1606(86) |
| 18.4 | 2590(55) |
| 19.5 | 2956(68) |
| 20.4 | 5956(98) |
| 22.2 | 2847(88) |
| 22.8 | 339(60) |
| 24.8 | 5782(210) |
| 25.4 | 13235(804) |
| 26.5 | 12678(768) |
| 28.1 | 7260(139) |
| 29.8 | 3756(109) |
| 32.2 | 424(47) |
| 33.4 | 1000(87) |
| 35.4 | 272(46) |
| 39.4 | 1643(91) |
| 52.0 | 1425(114) |

FIG. 29 depicts an XRPD pattern of Form I of compound 10.

### Example 11. Form I of Compound 11

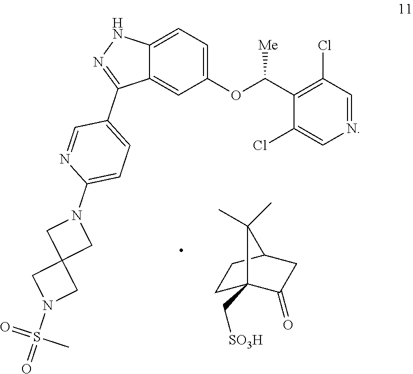 [SMILES: C[C@@H](Oc1ccc2[nH]nc(-c3ccc(N4CC5(C4)CN(S(C)(=O)=O)C5)nc3)c2c1)c1c(Cl)cncc1Cl ; CC1(C)C2CC[C@@]1(CS(=O)(=O)O)C(=O)C2]

Form I of Compound 11 was synthesized using the method described in Example 3.

LCMS: m/z=559.1 (M+H); ¹H NMR (400 MHz, DMSO-d6) δ=13.25 (br s, 1H), 8.58 (s, 2H), 8.38 (d, J=1.7 Hz, 1H), 8.23 (br d, J=8.8 Hz, 1H), 7.51 (d, J=9.0 Hz, 1H), 7.21 (d, J=2.1 Hz, 1H), 7.13 (dd, J=2.3, 9.0 Hz, 1H), 6.93 (br d, J=8.4 Hz, 1H), 6.14 (q, J=6.7 Hz, 1H), 4.42 (s, 4H), 4.15 (s, 4H), 3.04 (s, 3H), 2.87 (d, J=14.7 Hz, 1H), 2.73-2.64 (m, 1H), 2.38 (d, J=14.7 Hz, 1H), 2.23 (td, J=3.9, 17.9 Hz, 1H), 1.93 (t, J=4.5 Hz, 1H), 1.89-1.74 (m, 5H), 1.32-1.23 (m, 2H), 1.05 (s, 3H), 0.74 (s, 3H); m.p.=179.8-198.9° C.

Table 24, supra, is reproduced below and sets forth the X-ray diffraction peaks observed for Form I of compound 11.

TABLE 24
XRPD Peak Positions for Form I of Compound 11

| Pos. [°2θ] | Int. I. (cps) |
|---|---|
| 5.0 | 1450(99) |
| 5.3 | 6923(102) |
| 5.8 | 8981(100) |
| 8.2 | 587(129) |
| 8.9 | 1127(142) |
| 10.6 | 1755(43) |
| 11.9 | 1049(39) |
| 12.7 | 2026(73) |
| 14.0 | 1434(59) |
| 14.4 | 1199(47) |
| 15.4 | 9720(212) |
| 15.9 | 4663(196) |
| 17.5 | 12649(116) |
| 19.4 | 2088(218) |
| 20.1 | 3132(354) |
| 21.1 | 4287(280) |
| 21.3 | 5897(355) |
| 25.1 | 4377(194) |
| 26.0 | 12841(530) |
| 26.9 | 2181(449) |
| 29.3 | 958(78) |
| 32.0 | 616(314) |
| 33.0 | 662(187) |
| 34.1 | 995(125) |

FIG. 30 depicts an XRPD pattern of Form I of compound 11.

### Example 12. Forms I and II of Compound 12

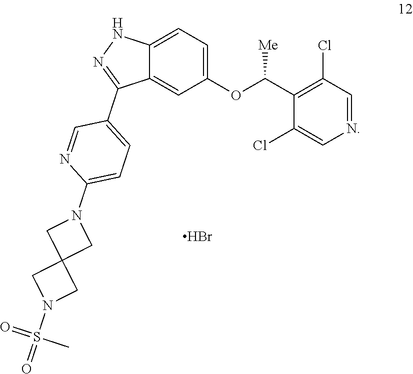 [SMILES: C[C@@H](Oc1ccc2[nH]nc(-c3ccc(N4CC5(C4)CN(S(C)(=O)=O)C5)nc3)c2c1)c1c(Cl)cncc1Cl ; Br]

### Form I of Compound 12

Form I of compound 12 was synthesized using the method described in Example 3.

LCMS: m/z=559.1 (M+H); ¹H NMR (400 MHz, DMSO-d₆) δ=13.28 (br s, 1H), 8.59 (s, 2H), 8.35 (s, 1H), 8.24 (br d, J=8.4 Hz, 1H), 7.52 (d, J=9.0 Hz, 1H), 7.21 (d, J=2.2 Hz, 1H), 7.13 (dd, J=2.3, 9.0 Hz, 1H), 6.94 (br d, J=8.6 Hz, 1H), 6.14 (q, J=6.7 Hz, 1H), 4.43 (s, 4H), 4.15 (s, 4H), 3.05 (s, 3H), 1.77 (d, J=6.6 Hz, 3H), m.p.=186.9-215.1° C.

Table 25, supra, is reproduced below and sets forth the X-ray diffraction peaks observed for Form I of compound 12.

TABLE 25
XRPD Peak Positions for Form I of Compound 12

| Pos. [°2θ] | Rel. Int. [%] |
|---|---|
| 5.5 | 45.68 |
| 7.2 | 6.85 |
| 8.0 | 3.49 |
| 11.0 | 7.77 |
| 11.8 | 2.52 |
| 13.5 | 18.64 |
| 14.3 | 56.73 |
| 15.8 | 6.40 |
| 16.6 | 97.97 |
| 19.4 | 58.58 |
| 20.2 | 100.00 |
| 21.9 | 26.17 |
| 23.1 | 43.14 |
| 24.8 | 34.94 |
| 26.0 | 46.19 |
| 27.6 | 28.68 |
| 28.5 | 5.84 |
| 29.0 | 8.52 |
| 29.9 | 22.73 |
| 31.2 | 3.19 |
| 31.6 | 4.03 |
| 32.4 | 6.71 |
| 34.4 | 7.80 |
| 34.8 | 10.82 |
| 35.9 | 9.16 |
| 36.4 | 11.10 |
| 38.1 | 6.82 |
| 39.2 | 10.54 |

FIG. 25 depicts an XRPD pattern of Form I of compound 12.

### Form II of Compound 12

A reaction between compound A (THE solution) and HBr (48% aqueous) yielded crystalline material. About 2.88% weight loss, from room temperature to 150° C., was observed in the TGA data. The DSC data exhibited an exothermic peak at 165.34° C. (peak temperature) followed by an endothermic event at 259.54° C. Vapor sorption data (not shown) suggests a moderately hygroscopic material. XRPD data analyses on the material that was recovered from DVS experiment (not shown) suggests stability.

Table 26, supra, is reproduced below and sets forth the X-ray diffraction peaks observed for Form II of compound 12.

TABLE 26
XRPD Peak Positions for Form II of Compound 12

| Pos. [°2θ] | Rel. Int. [%] |
|---|---|
| 5.8 | 18.67 |
| 7.4 | 1.87 |
| 8.3 | 2.05 |
| 11.3 | 7.23 |
| 12.1 | 3.97 |
| 13.8 | 22.01 |
| 14.6 | 55.91 |
| 15.0 | 22.25 |
| 16.1 | 14.58 |
| 16.9 | 95.33 |
| 17.4 | 36.88 |
| 19.6 | 68.30 |
| 20.5 | 100.00 |
| 22.0 | 38.60 |
| 23.4 | 56.80 |
| 24.5 | 25.08 |
| 25.0 | 56.87 |
| 26.0 | 48.43 |
| 26.4 | 49.57 |
| 27.0 | 38.00 |
| 27.5 | 38.71 |
| 27.9 | 37.61 |
| 28.6 | 19.31 |
| 29.2 | 22.73 |
| 30.2 | 28.81 |
| 31.4 | 13.05 |
| 32.6 | 15.94 |
| 33.8 | 15.01 |
| 34.5 | 17.10 |
| 35.1 | 14.35 |
| 36.0 | 14.08 |
| 36.5 | 16.51 |
| 38.3 | 11.73 |
| 39.6 | 13.58 |

FIG. 62 depicts an XRPD pattern of Form II of compound 12.

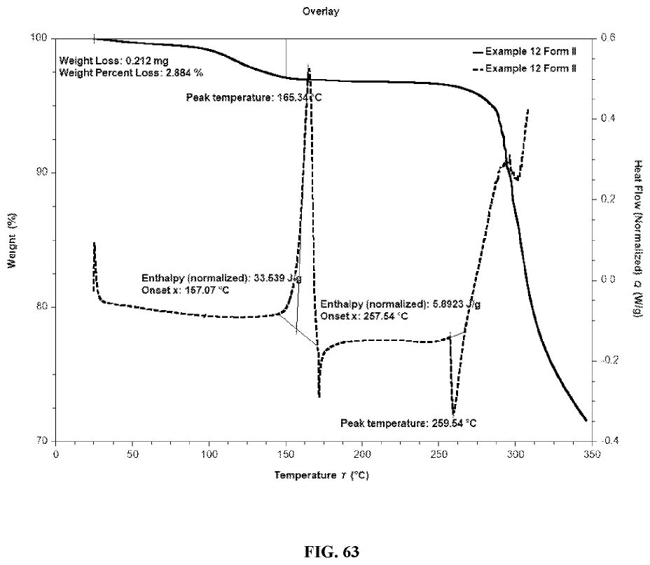

FIG. 63 depicts a DSC thermogram and TGA trace of Form II of compound 1.

### Example 13. Compound 13

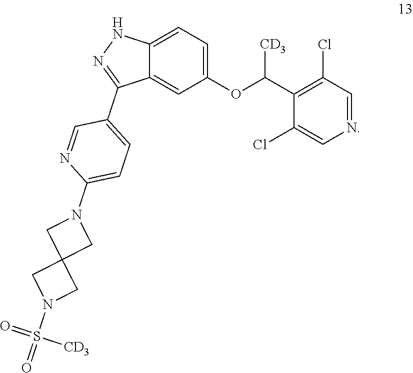 [SMILES: [2H]C([2H])([2H])C(Oc1ccc2[nH]nc(-c3ccc(N4CC5(C4)CN(S(=O)(=O)C([2H])([2H])[2H])C5)nc3)c2c1)c1c(Cl)cncc1Cl]

Compound 13 was prepared according to Scheme 2 and the following description.

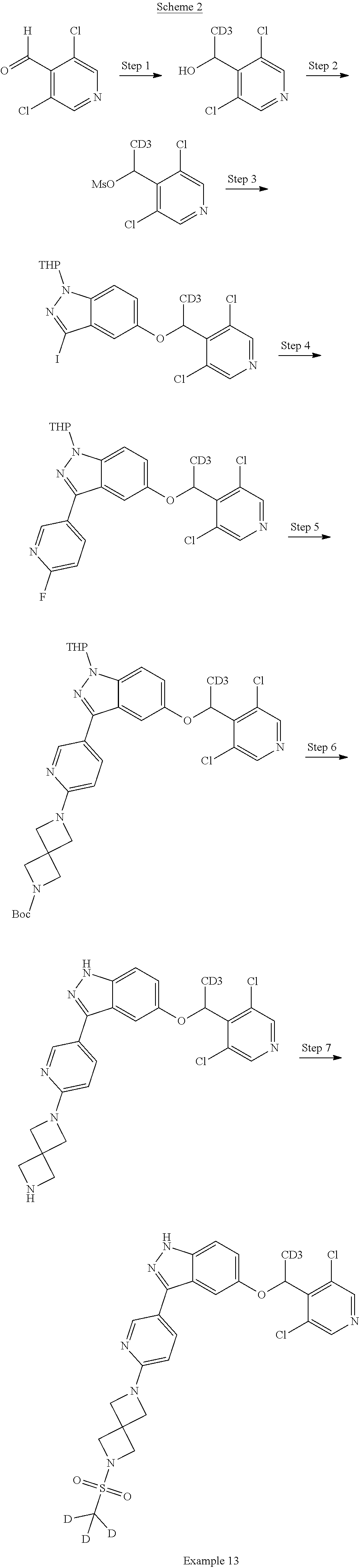

### Step 1. 1-(3,5-Dichloropyridin-4-yl) ethan-2,2,2-d3-1-ol

A suspension of magnesium turnings (0.82 g, 33.7 mmol, 1.85 equiv) in ether (68 mL) at room temperature was treated with iodine (23 mg, 0.09 mmol, 0.005 equiv) for 5 minutes then was cooled to −10° C. in an acetone ice bath. A solution of iodomethane-d3 (4.0 g, 27.6 mmol, 1.5 equiv) in ether (4 mL) was added to the reaction and external cooling was maintained for 30 minutes then the reaction was stirred at room temperature. The mixture changed from an initial orange suspension to a yellow suspension then to a grey-white suspension. After 2 hours, the Grignard reagent mixture was cooled in an acetone-dry ice bath then was treated with a solution of 3,5-dichloropyridine-4-carbaldehyde (3.2 g, 18.18 mmol, 1 equiv) in ether (32 mL) added over 5 minutes. The reaction was maintained at −78° C. for 20 minutes, then was stirred at room temperature for 1 hour. The reaction was cooled in an ice-water bath, quenched with water (5 mL) then treated with saturated ammonium chloride (50 mL) and was stirred for 30 minutes. The mixture was separated, and the aqueous layer was extracted with ethyl acetate (25 mL). The combined organic layers were washed with saturated brine (25 mL), dried over sodium sulfate, filtered and concentrated under reduced pressure, azeotroping with heptanes (10 mL) to give an orange oil (3.43 g, 92%). LCMS m/z=195 (M+H).

### Step 2. 1-(3,5-Dichloropyridin-4-yl)ethyl-2,2,2-d3 methanesulfonate

Triethylamine (7.0 mL, 50.3 mmol, 2.9 equiv) was added to a solution 1-(3,5-dichloropyridin-4-yl) ethan-2,2,2-d3-1-ol (3.43 g, 17.5 mmol) in dichloromethane (50 mL) at 0° C. Methanesulfonyl chloride (2.0 mL, 25.8 mmol, 1.5 equiv) was then added dropwise over 2 minutes. After 2 hours, the mixture was diluted with water (100 mL), the layers were separated and the aqueous layer was extracted with dichloromethane (50 mL). The combined organic layers were diluted with ethyl acetate (100 mL), dried over sodium sulfate and filtered through silica gel (2 cm high x 6 cm wide) in a sintered glass funnel, washing the filter pad with ethyl acetate (200 mL). The filtrate was concentrated under reduced pressure and the residue was dried at 35° C. under vacuum for 90 minutes to give an orange solid (4.22 g, 88%). LCMS m/z=273 (M+H).

### Step 3. 5-(1-(3,5-Dichloropyridin-4-yl) ethoxy-2,2,2-d3)-3-iodo-1-(tetrahydro-2H-pyran-2-yl)-1H-indazole

1-(3,5-Dichloropyridin-4-yl)ethyl-2,2,2-d3 methanesulfonate (4.22 g, 15.4 mmol, 1 equiv), 3-iodo-1-tetrahydropyran-2-yl-indazol-5-ol (5.32 g, 15.4 mmol, 1 equiv) and cesium carbonate (10.0 g, 30.6 mmol, 2 equiv) in acetonitrile (100 mL) were heated at 60° C. for 16 hours. The mixture was cooled to room temperature then diluted with ethyl acetate (150 mL) and water (150 mL). The layers were separated and the aqueous layer was extracted with ethyl acetate (50 mL). The combined organic extracts were diluted with heptanes (10 mL), washed with water (50 mL), saturated brine (50 mL), dried over sodium sulfate, filtered and concentrated onto Celite (20 g). The residue was purified on a Büchi automated chromatography system (Sorbtech, 120 g silica gel column), eluting with a gradient of 0 to 40% ethyl acetate in heptanes to give a white solid (6.65 g, 82% yield) after drying under vacuum at 30° C. overnight. LCMS m/z=521 (M+H).

### Step 4. 5-(1-(3,5-Dichloropyridin-4-yl) ethoxy-2,2, 2-d3)-3-(6-fluoropyridin-3-yl)-1-(tetrahydro-2H-pyran-2-yl)-1H-indazole

5-(1-(3,5-Dichloropyridin-4-yl) ethoxy-2,2,2-d3)-3-iodo-1-(tetrahydro-2H-pyran-2-yl)-1H-indazole (6.64 g, 12.7 mmol), 2-fluoro-5-(4,4,5,5-tetramethyl-1,3,2-dioxaborolan-2-yl)pyridine (3.45 g, 15.4 mmol, 1.2 equiv), potassium carbonate (3.50 g, 25.3 mmol, 2 equiv) and [1,l′-bis(diphenylphosphino) ferrocene]dichloropalladium (II) (0.93 g, 1.27 mmol, 0.10 equiv) in a mixture of 1,4-dioxane (120 mL) and water (15 mL) was sparged with nitrogen for 15 minutes then heated at 90° C. for 23 hours. The mixture was cooled to room temperature, filtered through Celite (20 g), washing the filter pad with ethyl acetate (250 mL). The filtrate was washed with water (100 mL) and the aqueous wash was extracted with ethyl acetate (50 mL). The combined organic extracts were dried over sodium sulfate, filtered and concentrated under reduced pressure onto Celite (70 g). The residue was purified on a Büchi automated chromatography system (Sorbtech, 220 g silica gel column), eluting with a gradient of 0 to 40% ethyl acetate in heptanes to give a white solid (5.56 g, 89%) after drying under vacuum for 6 hours at 30° C. LCMS m/z=490 (M+H).

### Step 5. tert-Butyl 6-(5-(5-(1-(3,5-dichloropyridin-4-yl) ethoxy-2,2,2-d3)-1-(tetrahydro-2H-pyran-2-yl)-1H-indazol-3-yl)pyridin-2-yl)-2,6-diazaspiro[3.3]heptane-2-carboxylate

5-(1-(3,5-Dichloropyridin-4-yl) ethoxy-2,2,2-d3)-3-(6-fluoropyridin-3-yl)-1-(tetrahydro-2H-pyran-2-yl)-1H-indazole (4.47 g, 9.12 mmol), tert-butyl 2,6-diazaspiro[3.3] heptane-2-carboxylate hemi-oxalic acid (2.77 g, 11.4 mmol, 1.25 equiv) and potassium carbonate (3.78 g, 27.4 mmol, 3 equiv) in 1-methyl-2-pyrrolidone (100 mL) were heated at 120° C. for 24 hours, Additional compound 189-2 (0.54 g, 1.1 mmol, 0.24 equiv) and potassium carbonate (1.3 g, 9.1 mmol, 1 equiv) were added and the mixture was heated overnight. The reaction was cooled to room temperature and poured into stirred water (1 L). The resulting solids were filtered. Filtration was exceedingly slow. The filtrate (~500 mL) was extracted with methyl tert-butyl ether (500 mL) and ethyl acetate (2×500 mL). The combined organic extracts, filtered solids and aqueous layer were combined. After mixing for 15 minutes, the layers were separated and the aqueous was extracted with ethyl acetate (500 mL). The combined organic extracts were dried over sodium sulfate, filtered and concentrated under reduced pressure onto Celite (40 g). The residue was purified on a Büchi automated chromatography system (Sorbtech, 80 g silica gel column), eluting with a gradient of 5 to 100% methyl tert-butyl ether in heptanes to give a white solid (5.32 g, 87%) after drying under vacuum at 40° C. overnight. LCMS m/z=668 (M+H).

### Step 6. 3-(6-(2,6-Diazaspiro[3.3]heptan-2-yl)pyridin-3-yl)-5-(1-(3,5-dichloropyridin-4-yl) ethoxy-2,2,2-d3)-1H-indazole

tert-Butyl 6-(5-(5-(1-(3,5-dichloropyridin-4-yl) ethoxy-2,2,2-d3)-1-(tetrahydro-2H-pyran-2-yl)-1H-indazol-3-yl)pyridin-2-yl)-2,6-diazaspiro[3.3]heptane-2-carboxylate (2.98 g, 4.4 mmol, with residual 1-methyl-2-pyrrolidone) in dichloromethane (30 mL) was treated with trifluoroacetic acid (30 mL) at room temperature for 5 hours. The reaction was concentrated under reduced pressure, the residue was dissolved in dichloromethane (50 mL) and reconcentrated under reduced pressure. The residue was dissolved in dichloromethane (60 mL), cooled in an ice/water bath then treated with 6M aqueous sodium hydroxide (10 mL) dropwise. A precipitate formed as the base was added. After stirring for 2 hours, the suspended solids were dissolved after the addition of methanol (10 mL) and the mixture was stirred at room temperature overnight. The layers were separated and the aqueous layer was saturated with solid sodium chloride then was extracted with dichloromethane (50 mL). The aqueous layer was decanted from the sodium chloride solids, the solids were washed with dichloromethane (20 mL) which was then added to the aqueous layer. The layers were separated and the combined organic extracts were dried over sodium sulfate, filtered, and concentrated under reduced pressure to give an orange solid (2.24 g, >100% yield) after drying under vacuum at 40° C. overnight. LCMS m/z=484 (M+H).

### Step 7. 5-(1-(3,5-Dichloropyridin-4-yl) ethoxy-2,2, 2-d3)-3-(6-(6-((methyl-d3) sulfonyl)-2,6-diazaspiro[3.3]heptan-2-yl)pyridin-3-yl)-1H-indazole

Sodium bicarbonate (2.0 g, 23.8 mmol, 5.4 equiv) in water (30 mL) was added to a suspension of product step 6 (2.24 g, 4.4 mmol) in dichloromethane (100 mL). After cooling to 0° C., a solution of methane-d3 sulfonyl chloride (0.67 g, 5.69 mmol, 1.3 equiv) in dichloromethane (2 mL) was added over 2 minutes. The mixture was stirred and warmed to room temperature. After 90 minutes, acetonitrile (20 mL) was added to assist in dissolution of insoluble matter. After an additional 90 minutes, the layers were separated, decanted away from insoluble matter. The layers were separated and the aqueous layer was extracted with dichloromethane (50 mL). The combined organic extracts were dried over sodium sulfate, filtered and concentrated under reduced pressure. The residue was dissolved in dichloromethane (10 mL) with minimal methanol, adsorbed onto Celite (20 g) then purified on a Büchi automated chromatography system (Sorbtech, 80 g silica gel column), eluting with a gradient of 0 to 10% methanol in ethyl acetate to give an off-white solid (0.71 g, 29% yield two steps) after drying at 40° C. overnight under vacuum.

LCMS m/z=565 (M+H); 1H NMR (400 MHz, DMSO-d₆) δ=12.99 (s, 1H), 8.59 (s, 2H), 8.52 (d, J=1.7 Hz, 1H), 7.87 (dd, J=2.3, 8.6 Hz, 1H), 7.45 (d, J=9.0 Hz, 1H), 7.17 (d, J=2.0 Hz, 1H), 7.09 (dd, J=2.3, 9.0 Hz, 1H), 6.55 (d, J=8.2 Hz, 1H), 6.10 (s, 1H), 4.17 (s, 4H), 4.12 (s, 4H).

### Example 14. Compound 14

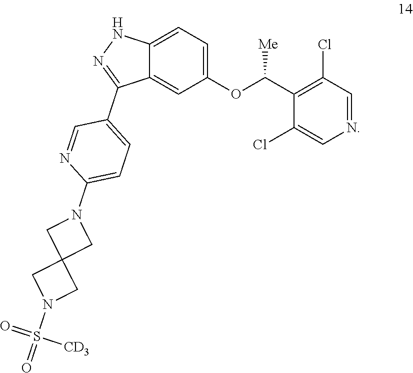 [SMILES: [2H]C([2H])([2H])S(=O)(=O)N1CC2(CN(c3ccc(-c4n[nH]c5ccc(O[C@H](C)c6c(Cl)cncc6Cl)cc45)cn3)C2)C1]

Compound 14 was prepared according to Scheme 3 and the following description.

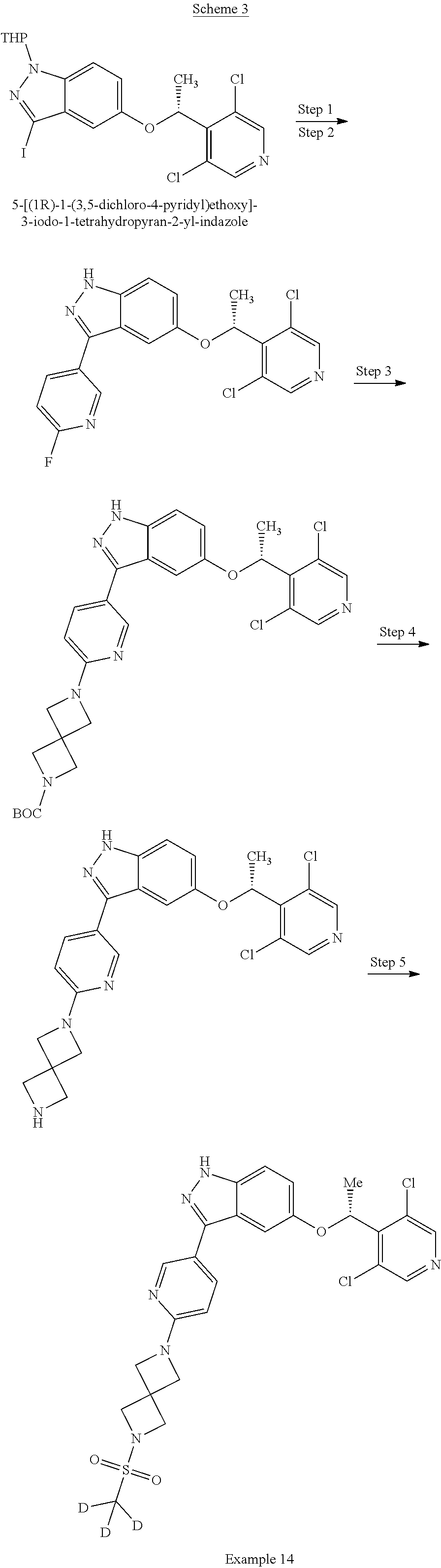

### Step 1. (R)-5-(1-(3,5-Dichloropyridin-4-yl) ethoxy)-3-iodo-1H-indazole

A mixture of 5-[(1R)-1-(3,5-dichloro-4-pyridyl) ethoxy]-3-iodo-1-tetrahydropyran-2-yl-indazole (1.0 g, 1.93 mmol, 1 equiv) in dichloromethane (10 mL) was treated with trifluoroacetic acid (3 mL, 39.2 mmol, 20 equiv) at room temperature for 24 hours. The mixture was concentrated under reduced pressure. The residue was dissolved in dichloromethane (100 mL), washed with saturated sodium bicarbonate (100 mL), dried over sodium sulfate, filtered and concentrated under reduced pressure. The product was dried at room temperature under vacuum overnight to give an orange solid (1.05 g), which was used subsequently. LCMS m/z=434 (M+H); 1H NMR (400 MHz, DMSO-d6) δ 13.37 (br s, 1H), 8.60 (s, 2H), 7.46 (d, J=8.9 Hz, 1H), 7.13 (dd, J=2.4, 9.0 Hz, 1H), 6.59 (d, J=2.3 Hz, 1H), 6.07 (q, J=6.6 Hz, 1H), 1.75 (d, J=6.6 Hz, 3H).

### Step 2. (R)-5-(1-(3,5-Dichloropyridin-4-yl) ethoxy)-3-(6-fluoropyridin-3-yl)-1H-indazole

A solution of product step 1 (1.0 g, 2.3 mmol, 1 equiv) and 2-fluoro-5-(4,4,5,5-tetramethyl-1,3,2-dioxaborolan-2-yl)pyridine (0.67 g, 2.995 mmol, 1.3 equiv) in 20 to 1 mixture of 1,4-dioxane and water (21 mL) was sparged with nitrogen for 15 minutes. Potassium carbonate (0.825 g, 5.98 mmol, 2.6 equiv) and (1,l′-bis(diphenylphosphino) ferrocene) palladium (II) dichloride (168 mg, 0.23 mmol, 0.01 equiv) were added and the reaction mixture was sparged with nitrogen for an additional 5 minutes. The reaction was heated at 90° C. for 2 hours. After cooling to room temperature, the reaction was concentrated under reduced pressure and diluted with saturated brine (30 mL) and dichloromethane (30 mL). The layers were separated and the organic layer was dried over sodium sulfate, filtered and concentrated under reduced pressure. The residue was absorbed onto silica gel (2 g) and purified on an Interchim automated chromatography system (Sorbtech 40 g silica gel cartridge), eluting with a gradient of 20 to 80% ethyl acetate in heptanes to give a yellow solid (0.42 g, 45% yield). LCMS m/z=403.1 (M+H); 1H NMR (400 MHz, CDCl3) δ 10.55 (br s, 1H), 8.68 (d, J=2.1 Hz, 1H), 8.43 (s, 2H), 8.24 (dt, J=2.4, 8.1 Hz, 1H), 7.39 (dd, J=0.8, 8.7 Hz, 1H), 7.20-7.14 (m, 2H), 7.06 (dd, J=2.8, 8.4 Hz, 1H), 6.06 (q, J=6.7 Hz, 1H), 1.82 (d, J=6.6 Hz, 3H).

### Step 3. tert-Butyl (R)-6-(5-(5-(1-(3,5-dichloropyridin-4-yl) ethoxy)-1H-indazol-3-yl)pyridin-2-yl)-2,6-diazaspiro[3.3]heptane-2-carboxylate

A mixture of product step 2 (0.295 g, 0.732 mmol, 1 equiv), tert-butyl 2,6-diazaspiro[3.3]heptane-2-carboxylate (0.29 g, 1.464 mmol, 2 equiv), and potassium carbonate (0.4 g, 2.93 mmol, 4 equiv) in anhydrous N-methylpyrrolidone was heated at 120° C. for 16 hours. The reaction mixture was filtered through a syringe filter and the filtrate was pre-absorbed on Celite (5 g). The material was purified on an Interchim automated chromatography system (RediSep Rf Gold HP C18, 15.5 g cartridge), eluting with a gradient of 0 to 100% acetonitrile in water. The fractions containing product were collected and lyophilized to give a yellowish solid (0.3 g, 71% yield). LCMS m/z=581 (M+H).

### Step 4. 3-[6-(2,6-diazaspiro[3.3]heptan-2-yl)-3-pyridyl]-5-[(1R)-1-(3,5-dichloro-4-pyridyl) ethoxy]-1H-indazole

A solution of step 1 product (50 mg, 0.086 mmol, 1 equiv) in anhydrous dichloromethane (2 mL) was treated with trifluoroacetic acid (0.53 mL, 6.88 mmol, 80 equiv) at room temperature for 16 hours. Additional trifluoroacetic acid (0.23 mL, 3.01 mmol, 35 equiv) was added and stirred for 1 hour. The reaction mixture was concentrated under reduced pressure to dryness. The residue was dissolved in methanol (10 mL), treated with MP-carbonate resin (3.2 mmol/g, 1 g), stirred for 30 minutes, filtered and concentrated under reduced pressure. The residue was absorbed onto Celite (1 g) and purified on an Interchim automated chromatography system (RediSep Rf Gold HP C18, 15.5 g cartridge), eluting with a gradient of 0 to 100% acetonitrile in water. The fractions containing product were collected and lyophilized to give a white solid (30 mg, 73% yield). LCMS: m/z=481.1 (M+H); 1H NMR (400 MHz, DMSO-d6) δ 13.00 (br s, 1H), 8.59 (s, 2H), 8.50 (d, J=1.8 Hz, 1H), 7.84 (dd, J=2.3, 8.7 Hz, 1H), 7.45 (d, J=9.0 Hz, 1H), 7.16 (d, J=2.1 Hz, 1H), 7.08 (dd, J=2.3, 9.0 Hz, 1H), 6.51 (d, J=8.4 Hz, 1H), 6.10 (q, J=6.6 Hz, 1H), 4.20-3.82 (m, 5H), 3.66 (br s, 4H), 1.76 (d, J=6.6 Hz, 3H).

### Step 5. (R)-5-(1-(3,5-Dichloropyridin-4-yl) ethoxy)-3-(6-(6-((methyl-d3) sulfonyl)-2,6-diazaspiro[3.3]heptan-2-yl)pyridin-3-yl)-1H-indazole

Triethylamine (0.23 mL, 1.68 mmol, 4 equiv) was added to a solution of 3-[6-(2,6-diazaspiro[3.3]heptan-2-yl)-3-pyridyl]-5-[(1R)-1-(3,5-dichloro-4-pyridyl) ethoxy]-1H-indazole (0.25 g, 0.42 mmol, 1 equiv) at 0° C. After stirring for 10 minutes, methanesulfonyl chloride-d3 (46.9 mg, 0.399 mmol, 0.95 equiv) was added and the mixture was stirred at 0 to 5° C. for 1 hour and then at room temperature for 1 hour. LCMS analysis indicated that a 1 to 2 mixture of starting material and product was formed. Additional methanesulfonyl chloride-d3 (30 mg) was added in 2 batches and the mixture was stirred at room temperature overnight. Water (10 mL) was added to quench the reaction and the mixture was extracted with ethyl acetate (3×30 mL). The combined organic layers were washed with saturated brine (30 mL), dried over sodium sulfate, filtered and concentrated under reduced pressure. The residue was purified on an Interchim automated chromatography system (Sorbtech 25 g silica gel column), eluting with a gradient of 0 to 10% methanol in dichloromethane to give a white solid. (0.122 g, 52% yield)

LCMS m/z=562.2 (M+H); 1H NMR (400 MHz, DMSO-d₆) δ=13.00 (s, 1H), 8.59 (s, 2H), 8.52 (d, J=1.8 Hz, 1H), 7.87 (dd, J=2.3, 8.6 Hz, 1H), 7.46 (d, J=8.9 Hz, 1H), 7.19-7.14 (m, 1H), 7.09 (dd, J=2.3, 9.0 Hz, 1H), 6.55 (d, J=8.2 Hz, 1H), 6.11 (q, J=6.7 Hz, 1H), 4.18 (s, 4H), 4.12 (s, 4H), 1.76 (d, J=6.7 Hz, 3H).

### Examples 15-22

Examples 15-22 are free base forms I, II, III, IV, V, VI, VII and VIII, respectively, of compound A and were produced by the following methods.

I. Fast Evaporative Crystallization Method (FE): compound A (ca. 30 mg) was dissolved in minimum quantity the stated solvent while heating at 80° C. The resulted solutions were filtered using a 0.45 μm PTFE syringe filter and the filtrate was collected. The filtrate was evaporated using a rotavap.

II. Slow Evaporative Crystallization Method (SE): compound A (ca. 30 mg) was dissolved in minimum quantity the stated solvent while heating at 80° C. The resulted solutions were filtered using a 0.45 μm PTFE syringe filter and the filtrate was collected. The filtrate was covered with perforated aluminum foil and left to evaporate at ambient temperature.

III. Slurry Crystallization at 25° C. using Form I of compound A: compound A (ca. 100 mg) was suspended in minimum quantity the stated solvent. The resulted slurries were allowed to stir at 25° C. for three days.

IV. Anti-Solvent Addition Crystallization: compound A (ca. 50 mg) was dissolved in solubilizing solvent. To that solution 200 μL anti-solvent was added slowly. These iterations were continued until the precipitation observed or maximum solvent quantity of ~25 mL. Many solvents were used as the solubilizing solvent in these experiments. Selection of anti-solvent for each solubilizing solvent is based on the miscibility between them. When anti-solvent addition did not yield immediate precipitation, the vials were sealed and the resultant solutions were kept in undisturbed laboratory bench and observed periodically to see if any crystals were grown during that time.

V. Vapor Diffusion Crystallization: compound A (ca. 30 mg) was dissolved in solubilizing solvent and kept in a small (8 mL) vial. These vials were kept in a larger vial (40 mL) that had 15 mL anti-solvents and closed the vial cap to allow vapor diffusion between them. Many solvents were used as the solubilizing solvent in these experiments. Selection of anti-solvent for each solubilizing solvent is based on the miscibility between them. All the experiments were kept in undisturbed laboratory bench and observed periodically to see if any crystals were grown during that time.

### Results—Slurry Crystallization Studies.

Compound A was slurred in respective solvents and stirred at 25° C. for three days. The resultant solids obtained from these experiments were analysed by XRPD and summarized in Table 43. A total of five unique XRPD patterns were identified and designated as Form I, Form II, Form III, Form IV, and Form V.

TABLE 43
Results of Slurry Crystallization Studies

| S. No. | Solvent | Slurry |
|---|---|---|
| 1 | MeOH | Form II |
| 2 | EtOH | Form I |
| 3 | IPA | Form I |
| 4 | ACN | Form III |
| 5 | EtOAc | Form I |
| 6 | THF | Form III |
| 7 | Acetone | Form II |
| 8 | DCM | Form II |
| 9 | H₂O | Form I |
| 10 | 14DOX | Form VI |
| 11 | 1,2-Propdiol | Form I |
| 12 | Eth. Gly | Form I |
| 13 | T-ButylMethEther | Form I |
| 14 | DMF | Form VII |
| 15 | DEE | Form I |
| 16 | 4-Me 2-Pentanone | Form I |
| 17 | CHCl₃ | Form III |

### Results—Slow Evaporation Studies.

Compound A was dissolved in a suitable solvent while heating up to 80° C. and the resultant solutions were allowed to slowly evaporate at room temperature (25° C.). The resultant solids obtained from these experiments were analysed by XRPD and summarized in Table 44.

TABLE 44
Results of Slurry Crystallization Studies

| | | Results from |
|---|---|---|
| | | Slow Evaporation |
| | | Crystallization |
| S. No. | Solvent | Experiments |
| 1 | MeOH | Form II |
| 2 | EtOH | Form IV |
| 3 | IPA | Form IV |
| 4 | ACN | Form IV |
| 5 | EtOAc | Form IV |
| 6 | THF | Form IV |
| 7 | Acetone | Form IV and 5 |
| 8 | DMSO | Form IV |
| 9 | DCM | Form IV |
| 10 | H₂O | Form I |
| 11 | 14DOX | — |
| 12 | 1,2-Propdiol | Form IV |
| 13 | Eth. Gly | Form IV |
| 14 | T-ButylMethEther | Form I |
| 15 | DMF | Form IV |
| 16 | DEE | Form I |
| 17 | 4-Me 2-Pentanone | Form I |
| 18 | CHCl₃ | — |

### Results—Fast Crystallization Studies.

The resultant solids obtained from these experiments were analyzed by XRPD data and summarized in Table 45. The experiments which did not yield solids through fast crystallization were further allowed to evaporate the resultant solution by slow evaporation.

TABLE 45
Results of Fast Crystallization Studies

| S. No. | Solvent | FE |
|---|---|---|
| 1 | MeOH | Form IV |
| 2 | EtOH | Form IV |
| 3 | IPA | Form IV |
| 4 | ACN | Form II |
| 5 | EtOAc | Form VIII |
| 6 | THF | Clear film |
| 7 | Acetone | Form II |
| 8 | DMSO | — |
| 9 | DCM | — |
| 10 | H₂O | Not Dissolved |
| 11 | 14DOX | — |
| 12 | 1,2-propane diol | — |
| 13 | Ethylene glycol | — |
| 14 | tert-butyl methyl ether | Not Dissolved |
| 15 | DMF | — |
| 16 | DEE | Not Dissolved |
| 17 | 4-Me 2-Pentanone | Not Dissolved |
| 18 | CHCl₃ | Clear film |

### Results—Anti-Solvent Addition Studies.

Compound A was dissolved in suitable solvents. To those solutions anti-solvents were added to obtain the solids. The solvent and anti-solvent selection was based on the solubility of compound A and also miscibility between them. The resultant solids obtained from these experiments were analyzed by XRPD experiments and summarized in Table 46.

TABLE 46
Results of Anti-Solvent Addition Studies

| S. No. | Solvent | Anti-Solvent | Results |
|---|---|---|---|
| 1 | MeOH | Water | Form II |
| | | T-Butyl Methyl Ether | Form II |
| | | DEE | Form II |
| | | 4-Me-2Pentanone | No XRPD - |
| | | | Gummy film |
| 2 | EtOH | Water | Form IV + |
| | | | Some extra peaks |
| | | T-Butyl Methyl Ether | Form IV |
| | | DEE | Form IV |
| | | 4-Me-2Pentanone | No XRPD - |
| | | | Gummy film |
| 3 | THF | Water | Form V |
| | | T-Butyl Methyl Ether | Form I |
| | | DEE | Form II |
| | | 4-Me-2Pentanone | Form I |
| 4 | 14DOX | Water | Form V |
| | | T-Butyl Methyl Ether | Form I |
| | | DEE | Form II |
| | | 4-Me-2Pentanone | No XRPD - |
| | | | Gummy film |
| 5 | Acetone | Water | Form V |
| | | T-Butyl Methyl Ether | Form I |
| | | DEE | Form II |
| | | 4-Me-2Pentanone | No XRPD - |
| | | | Gummy film |
| 6 | EtOAc | Not Miscible with | — |
| | | Water | |
| | | T-Butyl Methyl Ether | Form I |
| | | DEE | Form IV |
| | | 4-Me-2Pentanone | Form IV + |
| | | | Some extra peaks |
| 7 | ACN | Water | Form V + |
| | | | some extra peaks |
| | | T-Butyl Methyl Ether | Form IV |
| | | DEE | No XRPD - |
| | | | Gummy film |
| | | 4-Me-2Pentanone | No XRPD - |
| | | | Gummy film |
| 8 | DMSO | Water | Amorphous |
| | | T-Butyl Methyl Ether | Form IV |
| | | Not Miscible with | — |
| | | DEE | |
| | | 4-Me-2Pentanone | — |
| 9 | DCM | Not Miscible with | — |
| | | Water | |
| | | T-Butyl Methyl Ether | Form I |
| | | DEE | Form II |
| | | 4-Me-2Pentanone | Form IV |
| 10 | CHCl₃ | Not Miscible with | — |
| | | Water | |
| | | T-Butyl Methyl Ether | Form I |
| | | DEE | Form I |
| | | 4-Me-2Pentanone | Form II |
| 11 | DMF | Water | Form II |
| | | T-Butyl Methyl Ether | Form II |
| | | DEE | Form VIII |
| | | 4-Me-2Pentanone | No XRPD - |
| | | | Gummy film |
| — | 1,2-Propdiol | Not Miscible with all | — |
| | | 4 Anti-solvents | |
| 12 | IPA | Water | Form IV + |
| | | | Some extra peaks |
| | | T-Butyl Methyl Ether | Form IV |
| | | DEE | Form IV |
| | | 4-Me-2Pentanone | No XRPD - |
| | | | Gummy film |

### Results—Vapor Diffusion Studies.

The resultant solids obtained from these experiments were analyzed by XRPD data and summarized in Table 47.

TABLE 47
Results of Vapor Diffusion Studies

| S. No. | Solvent | Anti-Solvent | Results |
|---|---|---|---|
| 1 | MeOH | Water | Form II |
| | | T-Butyl Methyl Ether | Form II |
| | | DEE | — |
| | | 4-Me-2Pentanone | No XRPD - |
| | | | Gummy film |
| 2 | EtOH | Water | Form IV + |
| | | | Some extra peaks |
| | | T-Butyl Methyl Ether | Form IV |
| | | DEE | Form IV |
| | | 4-Me-2Pentanone | No XRPD - |
| | | | Gummy film |
| 3 | THF | Water | Form V |
| | | T-Butyl Methyl Ether | Form VIII |
| | | DEE | Form IV |
| | | 4-Me-2Pentanone | Form II |
| 4 | 14DOX | Water | — |
| | | T-Butyl Methyl Ether | Form VIII |
| | | DEE | Form VIII |
| | | 4-Me-2Pentanone | — |
| 5 | Acetone | Water | — |
| | | T-Butyl Methyl Ether | — |
| | | DEE | — |
| | | 4-Me-2Pentanone | — |
| 6 | EtOAc | Not Miscible with Water | — |
| | | T-Butyl Methyl Ether | Form IV |
| | | DEE | Form IV |
| | | 4-Me-2Pentanone | Form IV |
| 7 | ACN | Water | — |
| | | T-Butyl Methyl Ether | — |
| | | DEE | — |
| | | 4-Me-2Pentanone | — |
| 8 | DMSO | Water | Form IV + |
| | | | extra peaks |
| | | T-Butyl Methyl Ether | — |
| | | Not Miscible with DEE | — |
| | | 4-Me-2Pentanone | — |
| 9 | DCM | Not Miscible with Water | — |
| | | T-Butyl Methyl Ether | Form I + |
| | | | extra peaks |
| | | DEE | Form IV |
| | | 4-Me-2Pentanone | Form I + |
| | | | extra peaks |
| 10 | CHCl₃ | Not Miscible with Water | — |
| | | T-Butyl Methyl Ether | Form I |
| | | DEE | Form VIII |
| | | 4-Me-2Pentanone | Form I + |
| | | | Form IV |
| 11 | DMF | Water | — |
| | | T-Butyl Methyl Ether | — |
| | | DEE | — |
| | | 4-Me-2Pentanone | — |
| — | 1,2-Propdiol | Not Miscible with all | — |
| | | 4 Anti-solvents | |
| 12 | IPA | Water | — |
| | | T-Butyl Methyl Ether | Form IV + |
| | | | Some extra peaks |
| | | DEE | Form IV |
| | | 4-Me-2Pentanone | Form IV |

### Example 15. Compound 15 (Form I of Compound A)

Table 31, supra, is reproduced below and sets forth the X-ray diffraction peaks observed for compound 15.

TABLE 31
XRPD Peak Positions for Compound 15

| Pos. [°2θ] | Rel. Int. [%] |
|---|---|
| 10.0 | 1.71 |
| 11.6 | 16.08 |
| 12.7 | 1.91 |
| 13.4 | 59.80 |
| 13.9 | 32.74 |
| 14.4 | 27.13 |
| 15.3 | 1.94 |
| 16.0 | 32.05 |
| 17.0 | 92.98 |
| 17.8 | 71.79 |
| 18.2 | 17.05 |
| 18.5 | 42.00 |
| 19.4 | 100.00 |
| 19.9 | 34.39 |
| 20.9 | 98.58 |
| 21.7 | 12.71 |
| 22.2 | 9.86 |
| 22.7 | 50.77 |
| 23.4 | 23.46 |
| 23.7 | 42.25 |
| 24.0 | 58.64 |
| 24.3 | 28.46 |
| 25.1 | 42.42 |
| 25.4 | 34.85 |
| 26.3 | 21.03 |
| 26.8 | 12.42 |
| 27.8 | 17.11 |
| 28.4 | 16.14 |
| 29.1 | 17.87 |
| 29.3 | 21.76 |
| 30.1 | 12.70 |
| 30.6 | 15.11 |
| 31.6 | 10.78 |
| 32.3 | 7.26 |
| 34.1 | 4.62 |
| 35.9 | 8.73 |
| 36.8 | 4.44 |
| 38.0 | 5.81 |

FIG. 32 depicts an XRPD pattern of compound 15.

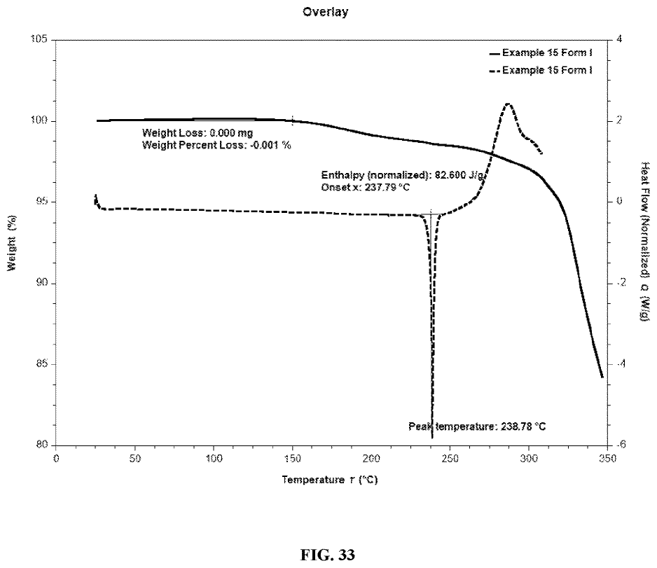

FIG. 33 depicts a DSC thermogram and TGA trace of compound 15.

### Example 16. Compound 16 (Form II of Compound A)

Table 32, supra, is reproduced below and sets forth the X-ray diffraction peaks observed for compound 16.

TABLE 32
XRPD Peak Positions for Compound 16

| Pos. [°2θ] | Rel. Int. [%] |
|---|---|
| 5.1 | 11.34 |
| 5.9 | 0.61 |
| 8.8 | 1.49 |
| 10.0 | 43.56 |
| 11.4 | 0.94 |
| 14.9 | 21.97 |
| 15.6 | 24.00 |
| 17.3 | 29.08 |
| 18.4 | 12.35 |
| 19.3 | 100.00 |
| 22.2 | 56.93 |
| 23.5 | 12.57 |
| 24.2 | 13.82 |
| 24.8 | 33.63 |
| 25.9 | 11.06 |
| 28.2 | 12.48 |
| 29.1 | 10.81 |
| 29.9 | 9.73 |
| 31.4 | 10.23 |
| 33.4 | 6.63 |
| 34.8 | 6.71 |
| 35.6 | 4.70 |
| 36.9 | 4.33 |
| 38.9 | 4.82 |

FIG. 34 depicts an XRPD pattern of compound 16.

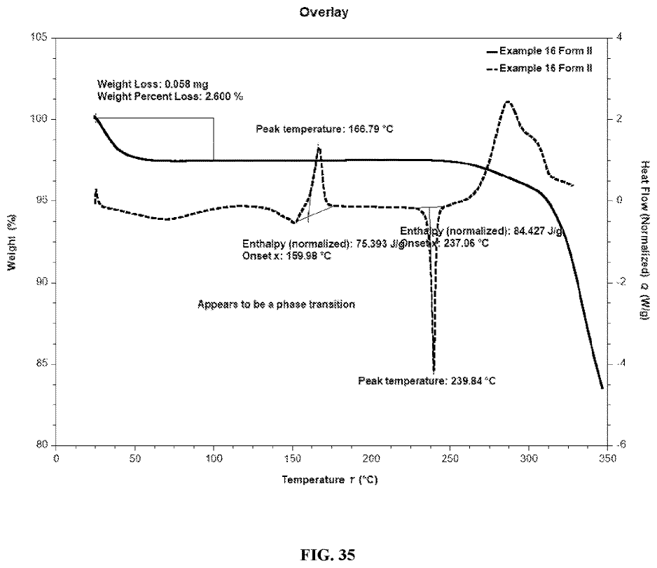

FIG. 35 depicts a DSC thermogram and TGA trace of compound 16.

### Example 17. Compound 17 (Form III of Compound A)

Table 33, supra, is reproduced below and sets forth the X-ray diffraction peaks observed for compound 17.

TABLE 33
XRPD Peak Positions for Compound 17

| Pos. [°2θ] | Rel. Int. [%] |
|---|---|
| 5.9 | 13.29 |
| 9.9 | 7.75 |
| 10.8 | 13.62 |
| 11.3 | 13.88 |
| 12.6 | 7.04 |
| 12.8 | 6.96 |
| 14.2 | 25.75 |
| 15.3 | 7.08 |
| 15.8 | 2.23 |
| 17.0 | 15.81 |
| 18.2 | 12.87 |
| 18.9 | 100.00 |
| 20.1 | 10.46 |
| 21.1 | 51.47 |
| 21.6 | 28.16 |
| 22.1 | 24.85 |
| 22.5 | 63.19 |
| 23.5 | 38.80 |
| 24.6 | 21.24 |
| 25.6 | 18.87 |
| 26.7 | 21.84 |
| 27.1 | 18.43 |
| 28.3 | 10.52 |
| 28.6 | 10.31 |
| 30.0 | 7.03 |
| 30.6 | 8.06 |
| 32.4 | 11.43 |
| 32.8 | 8.32 |
| 33.4 | 10.28 |
| 33.7 | 12.11 |
| 34.6 | 5.02 |
| 35.7 | 5.30 |
| 37.9 | 3.23 |

FIG. 36 depicts an XRPD pattern of compound 17.

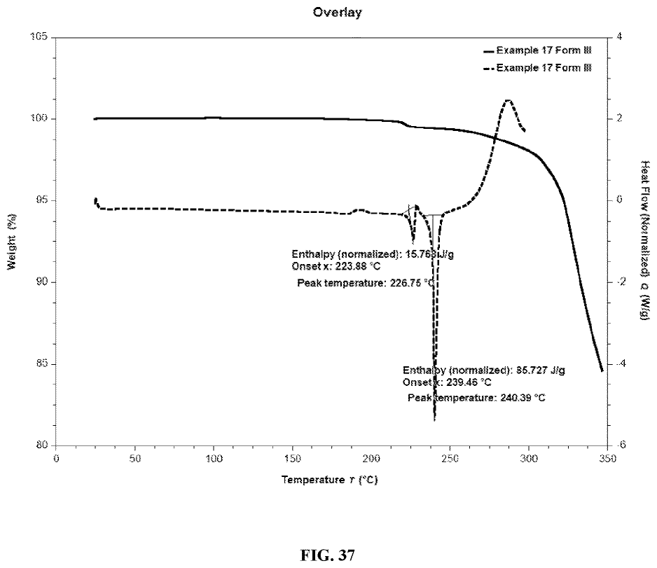

FIG. 37 depicts a DSC thermogram and TGA trace of compound 17.

### Example 18. Compound 18 (Form IV of Compound A)

Table 34, supra, is reproduced below and sets forth the X-ray diffraction peaks observed for compound 18.

TABLE 34
XRPD Peak Positions for Compound 18

| Pos. [°2θ] | Rel. Int. [%] |
|---|---|
| 7.2 | 0.95 |
| 9.9 | 52.62 |
| 11.5 | 19.31 |
| 12.7 | 10.11 |
| 13.1 | 7.15 |
| 13.5 | 24.48 |
| 14.0 | 16.65 |
| 14.8 | 53.58 |
| 15.2 | 2.61 |
| 15.7 | 29.31 |
| 16.1 | 1.59 |
| 17.1 | 26.19 |
| 17.6 | 14.04 |
| 18.2 | 6.23 |
| 18.7 | 100.00 |
| 19.2 | 37.20 |
| 19.9 | 48.10 |
| 20.2 | 83.54 |
| 21.0 | 2.21 |
| 21.5 | 19.20 |
| 21.9 | 9.05 |
| 22.2 | 20.33 |
| 22.4 | 77.43 |
| 22.8 | 5.02 |
| 23.1 | 14.64 |
| 23.7 | 2.19 |
| 24.3 | 25.61 |
| 24.9 | 13.16 |
| 25.5 | 22.24 |
| 25.8 | 25.13 |
| 26.3 | 14.24 |
| 26.7 | 4.18 |
| 27.0 | 6.66 |
| 27.3 | 3.21 |
| 27.6 | 4.50 |
| 28.6 | 3.49 |
| 29.2 | 6.69 |
| 29.8 | 7.87 |
| 30.1 | 6.84 |
| 30.5 | 2.61 |
| 30.7 | 6.83 |
| 31.4 | 5.24 |
| 31.7 | 3.70 |
| 32.3 | 7.83 |
| 32.6 | 2.35 |
| 32.9 | 2.42 |
| 33.2 | 2.70 |
| 34.1 | 4.50 |
| 34.7 | 4.67 |
| 35.0 | 2.65 |
| 35.4 | 2.89 |
| 35.6 | 3.90 |
| 36.1 | 6.72 |
| 36.3 | 5.48 |
| 36.8 | 2.27 |
| 37.5 | 5.10 |
| 37.7 | 1.74 |
| 38.3 | 1.05 |
| 39.0 | 4.20 |
| 39.6 | 6.25 |

FIG. 38 depicts an XRPD pattern of compound 18.

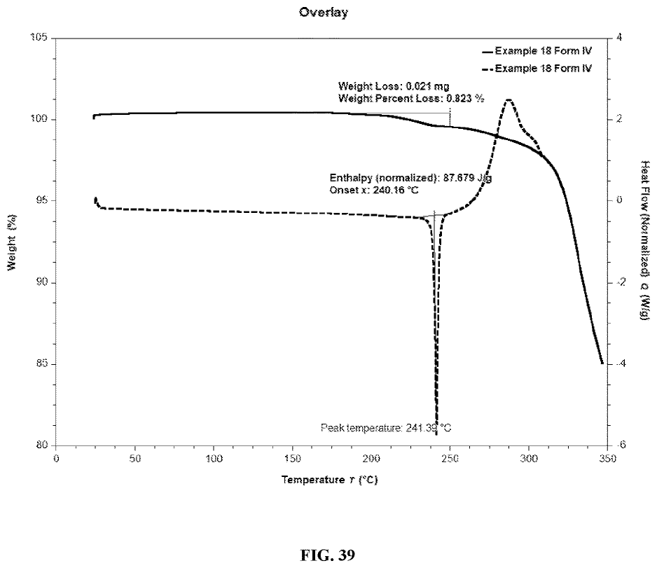

FIG. 39 depicts a DSC thermogram and TGA trace of compound 18.

### Example 19. Compound 19 (Form V of Compound A)

Table 35, supra, is reproduced below and sets forth the X-ray diffraction peaks observed for compound 19.

TABLE 35
XRPD Peak Positions for Compound 19

| Pos. [°2θ] | Rel. Int. [%] |
|---|---|
| 6.3 | 0.83 |
| 9.7 | 0.27 |
| 10.1 | 0.13 |
| 10.6 | 2.93 |
| 12.4 | 4.93 |
| 13.0 | 12.68 |
| 13.7 | 6.29 |
| 15.2 | 13.49 |
| 15.5 | 2.77 |
| 16.2 | 100.00 |
| 17.3 | 9.90 |
| 17.8 | 65.83 |
| 18.4 | 3.24 |
| 18.8 | 15.78 |
| 20.0 | 39.00 |
| 20.7 | 34.90 |
| 21.3 | 18.50 |
| 22.0 | 25.54 |
| 23.4 | 3.88 |
| 23.7 | 20.99 |
| 24.5 | 3.14 |
| 25.0 | 11.25 |
| 25.6 | 17.26 |
| 25.9 | 15.83 |
| 26.7 | 3.33 |
| 27.4 | 6.62 |
| 27.6 | 2.03 |
| 28.1 | 8.09 |
| 29.0 | 6.19 |
| 29.4 | 6.68 |
| 30.1 | 8.55 |
| 30.7 | 5.96 |
| 31.3 | 0.95 |
| 31.8 | 5.29 |
| 32.3 | 11.81 |
| 32.9 | 1.49 |
| 33.4 | 0.80 |
| 33.8 | 1.17 |
| 34.3 | 5.37 |
| 34.6 | 1.47 |
| 35.3 | 4.37 |
| 36.2 | 5.08 |
| 36.8 | 1.45 |
| 37.1 | 3.92 |
| 37.7 | 1.70 |
| 38.3 | 1.40 |
| 39.2 | 1.76 |

FIG. 40 depicts an XRPD pattern of compound 19.

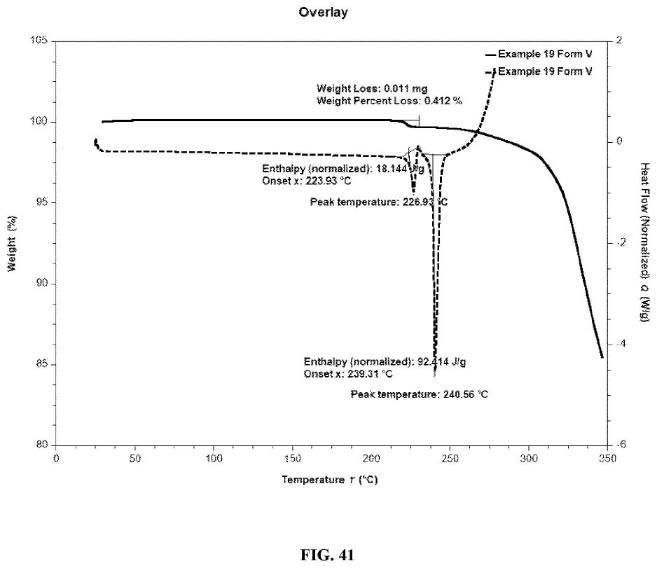

FIG. 41 depicts a DSC thermogram and TGA trace of compound 19.

### Example 20. Compound 20 (Form VI of Compound A)

Table 36, supra, is reproduced below and sets forth the X-ray diffraction peaks observed for compound 20.

TABLE 36
XRPD Peak Positions for Compound 20

| Pos. [°2θ] | Rel. Int. [%] |
|---|---|
| 5.1 | 8.30 |
| 5.9 | 1.78 |
| 7.0 | 3.33 |
| 7.5 | 4.58 |
| 8.6 | 1.15 |
| 10.0 | 26.72 |
| 14.0 | 3.02 |
| 14.9 | 33.84 |
| 15.4 | 35.82 |
| 16.0 | 17.95 |
| 16.8 | 16.14 |
| 18.2 | 100.00 |
| 18.7 | 52.01 |
| 19.4 | 20.78 |
| 19.8 | 19.09 |
| 20.8 | 61.12 |
| 22.8 | 79.51 |
| 24.0 | 35.04 |
| 24.8 | 27.83 |
| 27.6 | 16.14 |
| 28.7 | 19.93 |
| 30.0 | 13.72 |
| 30.7 | 10.63 |
| 32.0 | 7.44 |
| 32.9 | 7.00 |
| 35.1 | 7.37 |
| 36.5 | 4.15 |
| 37.4 | 4.02 |

FIG. 42 depicts an XRPD pattern of compound 20.

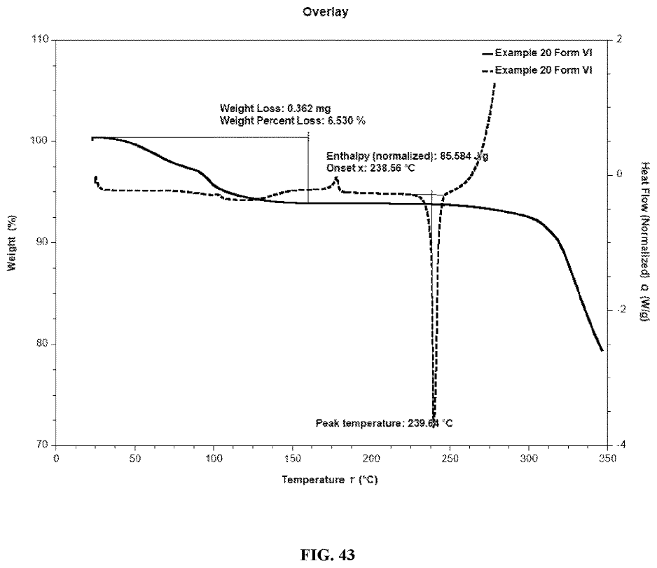

FIG. 43 depicts a DSC thermogram and TGA trace of compound 20.

### Example 21. Compound 21 (Form VII of Compound A)

Table 37, supra, is reproduced below and sets forth the X-ray diffraction peaks observed for compound 21.

TABLE 37
XRPD Peak Positions for Compound 21

| Pos. [°2θ] | Rel. Int. [%] |
|---|---|
| 6.0 | 3.64 |
| 6.9 | 31.03 |
| 9.3 | 32.32 |
| 11.8 | 38.64 |
| 13.7 | 4.16 |
| 14.4 | 92.61 |
| 15.1 | 24.31 |
| 15.4 | 27.21 |
| 16.8 | 20.54 |
| 17.6 | 31.54 |
| 18.0 | 100.00 |
| 19.9 | 91.65 |
| 20.4 | 15.49 |
| 20.8 | 16.12 |
| 21.2 | 8.12 |
| 22.2 | 77.22 |
| 22.6 | 58.08 |
| 23.1 | 10.59 |
| 23.7 | 15.86 |
| 24.1 | 52.70 |
| 24.7 | 24.46 |
| 25.5 | 50.89 |
| 26.1 | 10.01 |
| 27.1 | 35.79 |
| 28.3 | 22.29 |
| 28.7 | 20.17 |
| 29.4 | 22.75 |
| 29.5 | 20.69 |
| 30.1 | 16.00 |
| 31.5 | 11.72 |
| 32.8 | 10.98 |
| 33.6 | 13.31 |
| 34.5 | 11.43 |
| 35.2 | 6.76 |
| 36.1 | 6.54 |
| 36.7 | 7.56 |
| 37.7 | 9.77 |
| 38.1 | 5.96 |
| 39.0 | 6.21 |

FIG. 44 depicts an XRPD pattern of compound 21.

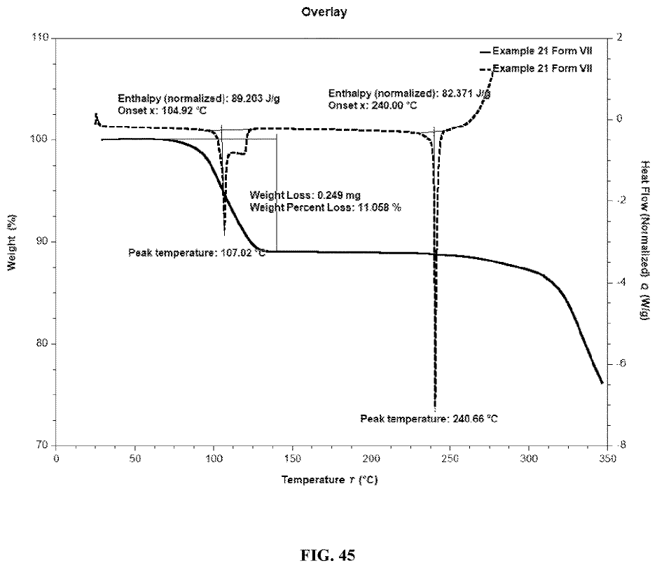

FIG. 45 depicts a DSC thermogram and TGA trace of compound 21.

### Example 22. Compound 22 (Form VIII of Compound A)

Table 38, supra, is reproduced below and sets forth the X-ray diffraction peaks observed for compound 22.

TABLE 38
XRPD Peak Positions for Compound 22

| Pos. [°2θ] | Rel. Int. [%] |
|---|---|
| 5.2 | 53.77 |
| 5.3 | 45.94 |
| 7.3 | 12.88 |
| 9.5 | 2.93 |
| 10.2 | 65.82 |
| 11.8 | 3.63 |
| 13.7 | 10.89 |
| 14.1 | 11.04 |
| 14.4 | 16.86 |
| 14.6 | 13.48 |
| 15.3 | 40.63 |
| 16.1 | 21.74 |
| 16.6 | 10.77 |
| 17.4 | 43.65 |
| 18.2 | 55.22 |
| 18.7 | 93.74 |
| 19.7 | 50.04 |
| 20.2 | 32.82 |
| 20.9 | 44.61 |
| 21.2 | 77.25 |
| 22.8 | 66.29 |
| 23.0 | 100.00 |
| 24.3 | 48.43 |
| 25.2 | 26.65 |
| 25.7 | 20.22 |
| 26.5 | 10.81 |
| 26.7 | 10.11 |
| 28.5 | 21.38 |
| 28.9 | 17.01 |
| 29.5 | 20.65 |
| 31.9 | 6.56 |
| 35.3 | 5.49 |
| 35.8 | 10.04 |
| 38.0 | 2.53 |

FIG. 46 depicts an XRPD pattern of compound 22.

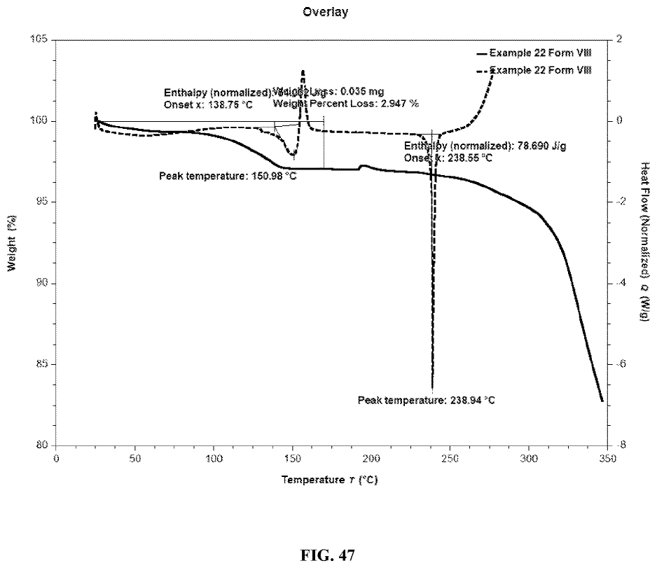

FIG. 47 depicts a DSC thermogram and TGA trace of compound 22.

### Example 23. Form I of Compound 23

A reaction between compound A (THF solution) and H₃PO₄ (85% aqueous) yielded crystalline material. About 1% weight loss, from room temperature to 150° C., was observed in the TGA data. The DSC data exhibited multiple thermal events. Vapor sorption data (not shown) suggests a hygroscopic material.

Table 27, supra, is reproduced below and sets forth the X-ray diffraction peaks observed for compound 23.

TABLE 27
XRPD Peak Positions for Compound 23

| Pos. [°2θ] | Int. I. (cps) |
|---|---|
| 5.5 | 1.80 |
| 5.9 | 2.10 |
| 7.1 | 1.77 |
| 12.2 | 2.75 |
| 12.9 | 2.98 |
| 13.9 | 18.02 |
| 14.8 | 20.69 |
| 15.4 | 17.45 |
| 16.7 | 25.43 |
| 17.8 | 25.53 |
| 18.9 | 25.60 |
| 19.8 | 100.00 |
| 21.6 | 18.15 |
| 22.6 | 18.58 |
| 23.3 | 25.08 |
| 24.3 | 39.32 |
| 25.4 | 78.05 |
| 26.5 | 31.83 |
| 27.2 | 26.75 |
| 27.6 | 21.57 |
| 29.7 | 10.78 |
| 30.4 | 8.60 |
| 34.0 | 3.95 |
| 35.6 | 1.80 |
| 36.9 | 2.36 |

FIG. 48 depicts an XRPD pattern of compound 23.

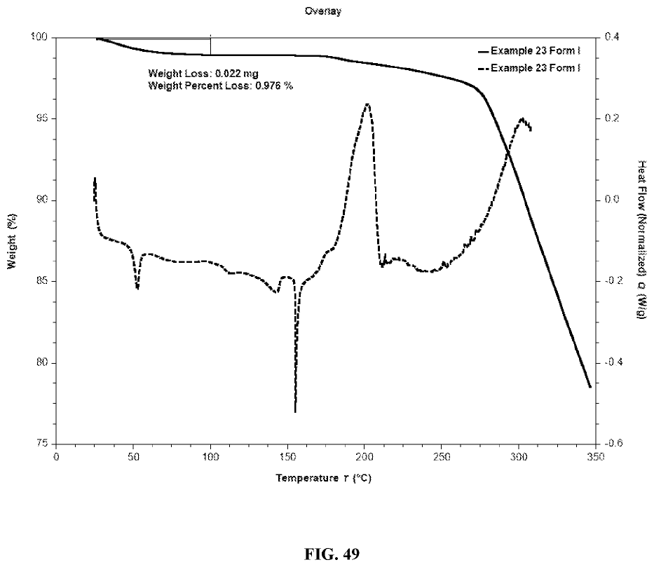

FIG. 49 depicts a DSC thermogram and TGA trace of compound 23.

### Example 24. Form I of Compound 24

A reaction between compound A (THF or acetone solution) and citric acid (MeOH solutions; 1 and 2 equivalents of acid with respect to compound A) yielded crystalline material. About 2.70% weight loss, from room temperature to 70° C., was observed in the TGA data. An endothermic peak at 142.74° C. which corresponds to the melting point observed in the DSC data was observed. Vapor sorption data (not shown) suggests a moderately hygroscopic material. Weight loss of about 3.27% was observed in the DVS data (not shown) and suggests instability.

Table 28, supra, is reproduced below and sets forth the X-ray diffraction peaks observed for compound 24.

TABLE 28
XRPD Peak Positions for Compound 24

| Pos. [°2θ] | Int. I. (cps) |
|---|---|
| 5.1 | 14.23 |
| 6.8 | 22.39 |
| 7.8 | 14.31 |
| 11.1 | 2.81 |
| 12.6 | 12.82 |
| 13.7 | 11.14 |
| 14.6 | 8.54 |
| 15.2 | 34.49 |
| 16.4 | 1.53 |
| 17.3 | 19.91 |
| 18.3 | 8.73 |
| 18.9 | 9.34 |
| 19.8 | 100.00 |
| 20.8 | 10.85 |
| 21.4 | 13.71 |
| 22.5 | 10.42 |
| 22.9 | 7.50 |
| 24.4 | 8.08 |
| 24.9 | 11.67 |
| 25.3 | 14.80 |
| 26.4 | 13.41 |
| 26.9 | 8.29 |
| 27.3 | 7.69 |
| 27.7 | 6.18 |
| 28.3 | 7.09 |
| 29.2 | 6.64 |
| 30.3 | 6.64 |
| 31.0 | 5.14 |
| 33.2 | 3.23 |
| 33.9 | 4.63 |
| 35.1 | 2.82 |
| 36.8 | 1.43 |
| 38.2 | 0.80 |

FIG. 50 depicts an XRPD pattern of compound 24.

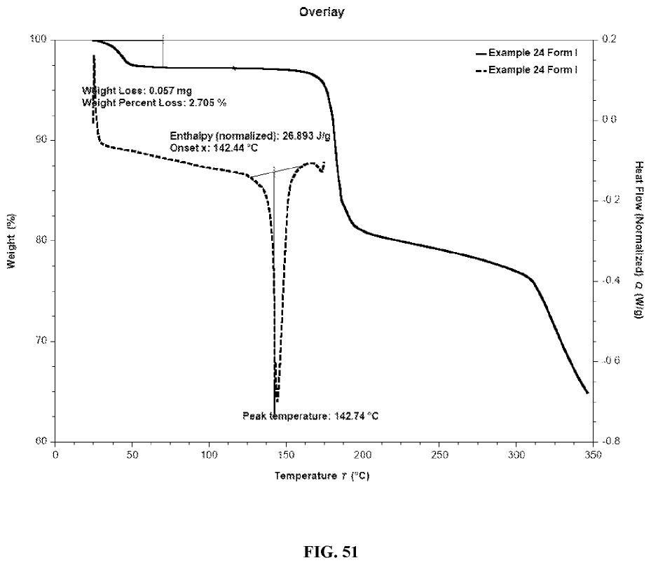

FIG. 51 depicts a DSC thermogram and TGA trace of compound 24.

### Example 25. Form I of Compound 25

A reaction between compound A (THF solution or acetone solution) and L-tartaric acid (MeOH solutions; 1 and 2 equivalents of acid with respect to compound A) yielded crystalline material. About 2.66% weight loss, from room temperature to 120° C., was observed in the TGA data. The DSC data exhibited an endothermic peak at 170.51° C. (peak temperature) which corresponds to the melting point. Vapor sorption data (not shown) suggests a moderately hygroscopic material.

Table 29, supra, is reproduced below and sets forth the X-ray diffraction peaks observed for compound 25.

TABLE 29
XRPD Peak Positions for Compound 25

| Pos. [°2θ] | Int. I. (cps) |
|---|---|
| 6.4 | 9.08 |
| 9.9 | 4.27 |
| 12.1 | 15.87 |
| 14.5 | 55.97 |
| 15.1 | 50.42 |
| 17.1 | 63.56 |
| 17.4 | 72.15 |
| 17.8 | 60.94 |
| 18.9 | 99.72 |
| 21.0 | 38.57 |
| 22.5 | 59.48 |
| 24.0 | 96.86 |
| 24.8 | 71.76 |
| 25.6 | 100.00 |
| 26.5 | 90.07 |
| 27.7 | 52.06 |
| 28.9 | 69.56 |
| 31.5 | 20.26 |
| 34.4 | 24.23 |
| 37.2 | 5.79 |

FIG. 52 depicts an XRPD pattern of compound 25.

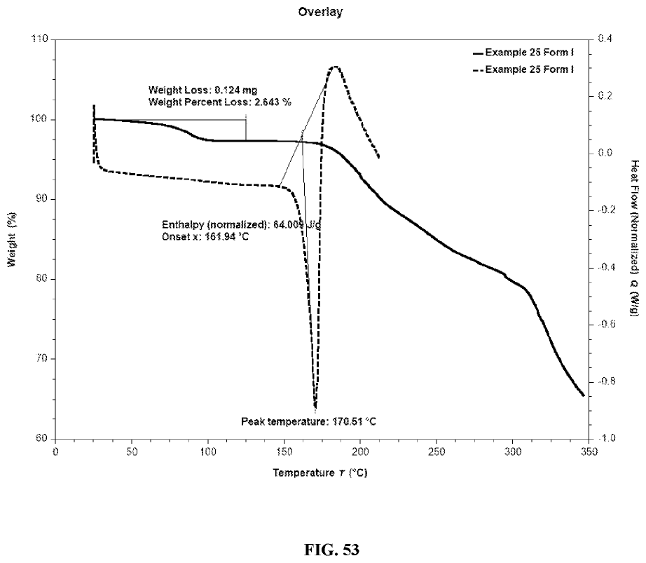

FIG. 53 depicts a DSC thermogram and TGA trace of compound 25.

### Example 26. Form I of Compound 26

A reaction between compound A (THF solution or acetone solution) and maleic acid (MeOH solutions; 1 and 2 equivalents of acid with respect to compound A) yielded crystalline material. About 3.61% weight loss, from room temperature to 140° C., observed in the TGA data indicates that it has volatile components in its crystal lattice. The DSC data exhibit an endothermic peak at 183.38° C. (peak temperature) which corresponds to the melting point. Vapor sorption data (not shown) suggest a moderately hygroscopic material. A weight loss of about 2.1% was observed.

Table 30, supra, is reproduced below and sets forth the X-ray diffraction peaks observed for compound 26.

TABLE 30
XRPD Peak Positions for Compound 26

| Pos. [°2θ] | Int. I. (cps) |
|---|---|
| 5.9 | 33.96 |
| 6.5 | 11.05 |
| 7.7 | 16.88 |
| 12.8 | 58.97 |
| 13.9 | 5.33 |
| 14.6 | 50.56 |
| 15.6 | 5.12 |
| 17.6 | 11.46 |
| 18.5 | 30.50 |
| 19.1 | 100.00 |
| 20.0 | 25.43 |
| 21.0 | 26.11 |
| 21.3 | 36.29 |
| 22.8 | 13.22 |
| 24.8 | 42.86 |
| 25.6 | 50.48 |
| 26.0 | 43.14 |
| 26.7 | 17.93 |
| 27.6 | 21.22 |
| 28.0 | 15.76 |
| 29.3 | 16.11 |
| 30.5 | 10.95 |
| 31.6 | 13.49 |
| 33.8 | 8.23 |
| 34.5 | 8.30 |
| 35.3 | 6.85 |
| 37.7 | 4.60 |
| 38.8 | 5.59 |

FIG. 54 depicts an XRPD pattern of compound 26.

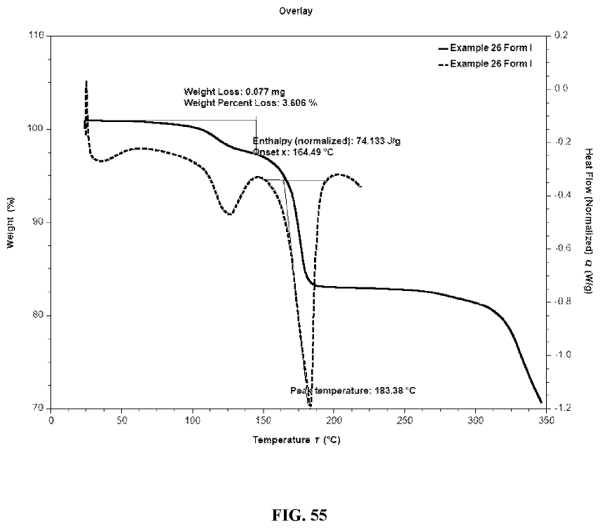

FIG. 55 depicts a DSC thermogram and TGA trace of compound 26.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. Compound 2:

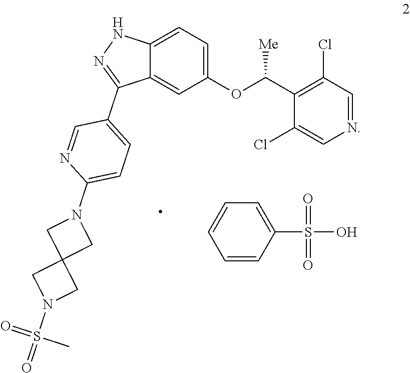 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=S(=O)(O)c1ccccc1]

2. The compound according to claim 1, wherein said compound is crystalline.

3. The compound according to claim 1, wherein said compound is a crystalline solid substantially free of amorphous compound 2.

4. The compound according to claim 1, wherein said compound is substantially free of impurities.

5. The compound according to claim 1, having one or more peaks in its XRPD selected from those at about 18.3, about 20.0 and about 21.6 degrees 2-theta.

6. (canceled)

7. The compound according to claim 5, wherein said compound is of Form I as disclosed herein.

8. (canceled)

9. The compound according to claim 1, having one or more peaks in its XRPD selected from those at about 5.9, about 19.9 and about 21.7 degrees 2-theta.

10. (canceled)

11. The compound according to claim 9, wherein said compound is of Form II as disclosed herein.

12. (canceled)

13. The compound according to claim 1, having one or more peaks in its XRPD selected from those at about 6.8, about 18.9, and about 20.3 degrees 2-theta.

14. (canceled)

15. The compound according to claim 13, wherein said compound is of Form III as disclosed herein.

16. (canceled)

17. The compound according to claim 1, having one or more peaks in its XRPD selected from those at about 4.9, about 5.0, and about 25.0 degrees 2-theta.

18. (canceled)

19. The compound according to claim 17, wherein said compound is of Form IV as disclosed herein.

20. (canceled)

21. The compound according to claim 1, having one or more peaks in its XRPD selected from those at about 18.0, about 20.7, and about 25.9 degrees 2-theta.

22. (canceled)

23. The compound according to claim 21, wherein said compound is of Form V as disclosed herein.

24. (canceled)

25. The compound according to claim 1, having one or more peaks in its XRPD selected from those at about 6.5, about 19.2, and about 20.2 degrees 2-theta.

26. (canceled)

27. The compound according to claim 25, wherein said compound is of Form VI as disclosed herein.

28. (canceled)

29. The compound according to claim 1, having one or more peaks in its XRPD selected from those at about 10.4, about 14.8, and about 24.9 degrees 2-theta.

30. (canceled)

31. The compound according to claim 29, wherein said compound is of Form VII as disclosed herein.

32. (canceled)

33. The compound according to claim 1, wherein said compound is of Form VIII.

34. (canceled)

35. The compound according to claim 1, having one or more peaks in its XRPD selected from those at about 6.3, about 6.7, and about 18.9 degrees 2-theta.

36. (canceled)

37. The compound according to claim 35, wherein said compound is of Form IX as disclosed herein.

38. (canceled)

39. The compound according to claim 1, having one or more peaks in its XRPD selected from those at about 6.6, about 18.2, and about 19.8 degrees 2-theta.

40. (canceled)

41. The compound according to claim 39, wherein said compound is of Form X as disclosed herein.

42. (canceled)

43. The compound according to claim 1, having one or more peaks in its XRPD selected from those at about 7.0, about 20.0, and about 20.3 degrees 2-theta.

44. (canceled)

45. The compound according to claim 43, wherein said compound is of Form XI as disclosed herein.

46. (canceled)

47. The compound according to claim 1, having one or more peaks in its XRPD selected from those at about 5.8, about 5.9, and about 19.3 degrees 2-theta.

48. (canceled)

49. The compound according to claim 47, wherein said compound is of Form XII.

50. (canceled)

51. The compound according to claim 1, having one or more peaks in its XRPD selected from those at about 6.1, about 21.8, and about 22.8 degrees 2-theta.

52. (canceled)

53. The compound according to claim 51, wherein said compound is of Form XIII as disclosed herein.

54. (canceled)

55. A pharmaceutically acceptable composition comprising the compound according to claim 1 and a pharmaceutically acceptable carrier, excipient, or vehicle.

56. A method of treating cancer in a patient comprising administering to said patient the pharmaceutical composition of claim 55.

57. The method according to claim 56, further comprising administering to the patient an additional therapeutic agent.

58. A compound selected from:
Compound 1: 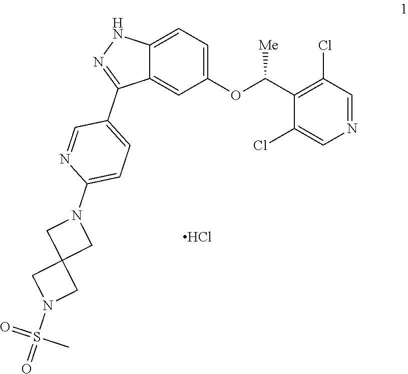 [SMILES: C[C@@H](Oc1ccc2[nH]nc(-c3ccc(N4CC5(C4)CN(S(C)(=O)=O)C5)nc3)c2c1)c1c(Cl)cncc1Cl ; Cl]
or a crystalline form thereof;
Compound 3: 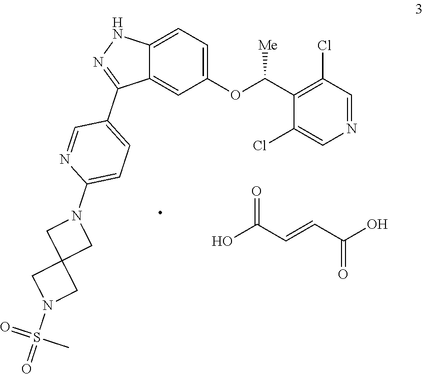
or a crystalline form thereof;
Compound 4: 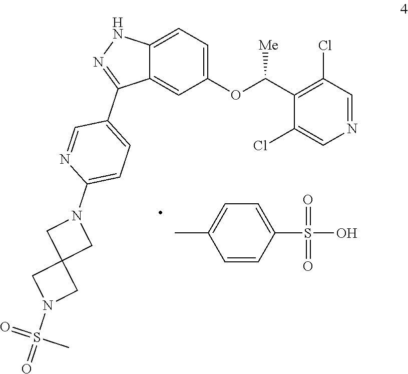 [SMILES: C[C@@H](Oc1ccc2[nH]nc(-c3ccc(N4CC5(C4)CN(S(C)(=O)=O)C5)nc3)c2c1)c1c(Cl)cncc1Cl ; Cc1ccc(S(=O)(=O)O)cc1]
or a crystalline form thereof;
Compound 5: 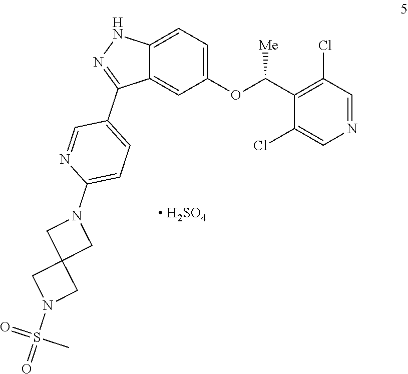
or a crystalline form thereof;
Compound 6: 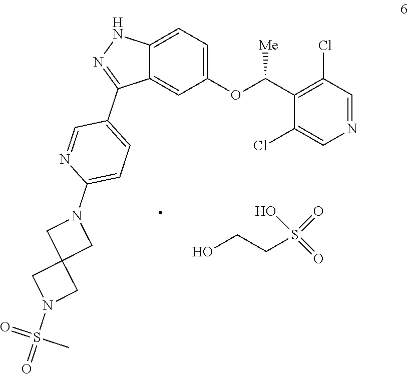 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=S(=O)(O)CCO]
or a crystalline form thereof;
Compound 7: 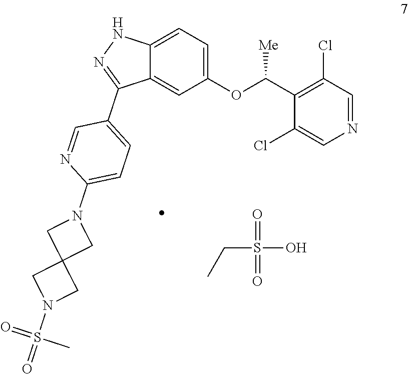
or a crystalline form thereof;
Compound 8: 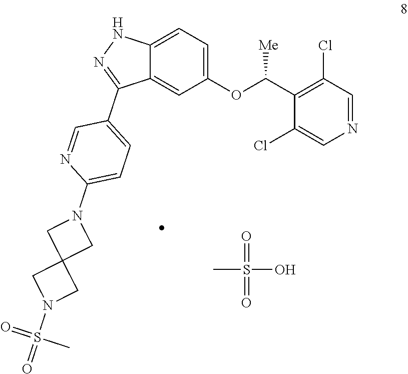
or a crystalline form thereof;
Compound 9: 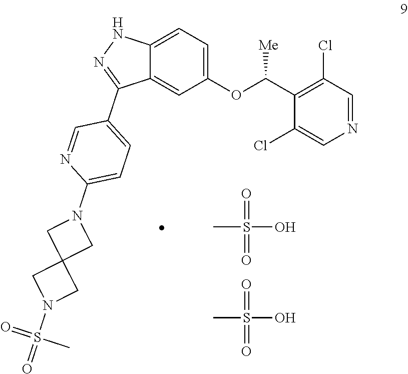
or a crystalline form thereof;
Compound 10: 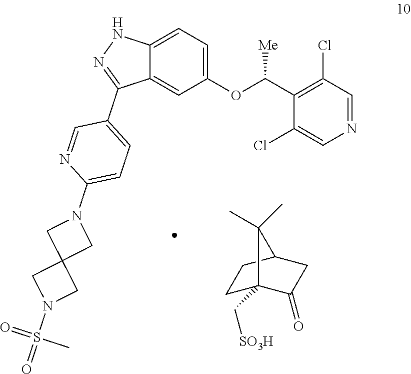
or a crystalline form thereof;
Compound 11: 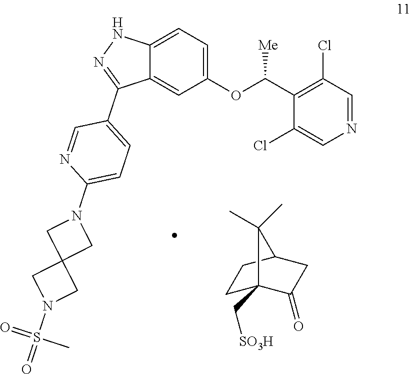 [SMILES: C[C@@H](Oc1ccc2[nH]nc(-c3ccc(N4CC5(C4)CN(S(C)(=O)=O)C5)nc3)c2c1)c1c(Cl)cncc1Cl ; CC1(C)C2CC[C@@]1(CS(=O)(=O)O)C(=O)C2]
or a crystalline form thereof;
Compound 12: 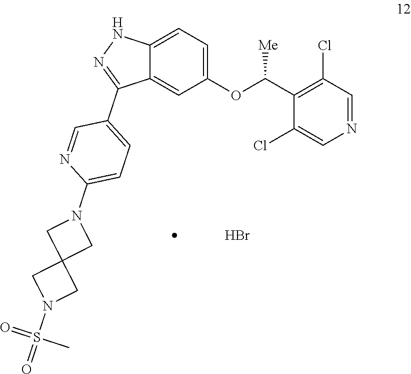 [SMILES: C[C@@H](Oc1ccc2[nH]nc(-c3ccc(N4CC5(C4)CN(S(C)(=O)=O)C5)nc3)c2c1)c1c(Cl)cncc1Cl ; Br]
or a crystalline form thereof;
Compound 13: 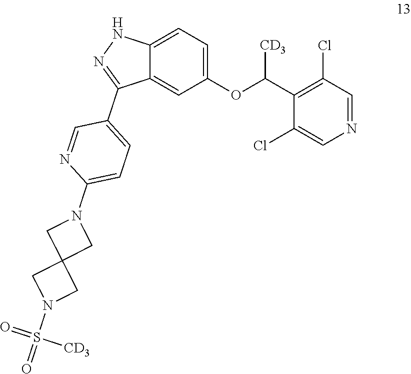 [SMILES: [2H]C([2H])([2H])C(Oc1ccc2[nH]nc(-c3ccc(N4CC5(C4)CN(S(=O)(=O)C([2H])([2H])[2H])C5)nc3)c2c1)c1c(Cl)cncc1Cl]
or a crystalline form thereof; or
Compound 14: 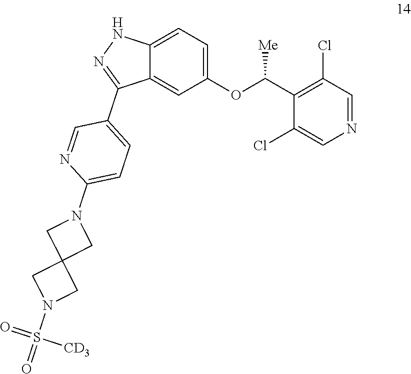 [SMILES: [2H]C([2H])([2H])S(=O)(=O)N1CC2(CN(c3ccc(-c4n[nH]c5ccc(O[C@H](C)c6c(Cl)cncc6Cl)cc45)cn3)C2)C1]
or a crystalline form thereof; or
Compound A: 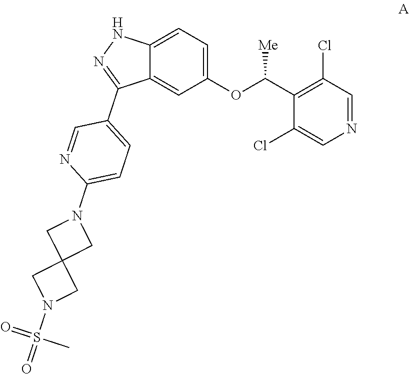 [SMILES: C[C@@H](Oc1ccc2[nH]nc(-c3ccc(N4CC5(C4)CN(S(C)(=O)=O)C5)nc3)c2c1)c1c(Cl)cncc1Cl]
of crystalline Form I, II, III, IV, V, VI, VII or VIII; or
Compound 23: 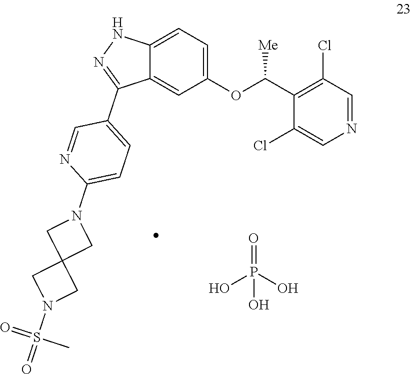 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=P(O)(O)O]
or a crystalline form thereof; or
Compound 24: 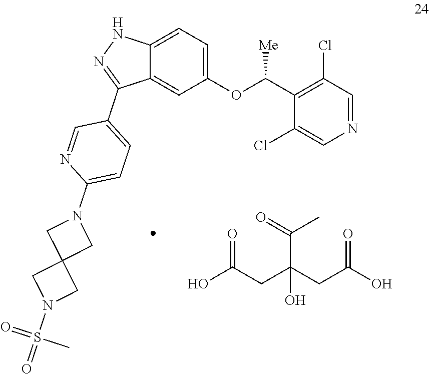 [SMILES: C[C@@H](Oc1ccc2[nH]nc(-c3ccc(N4CC5(C4)CN(S(C)(=O)=O)C5)nc3)c2c1)c1c(Cl)cncc1Cl ; "·" ; CC(=O)C(O)(CC(=O)O)CC(=O)O]
or a crystalline form thereof; or
Compound 25: 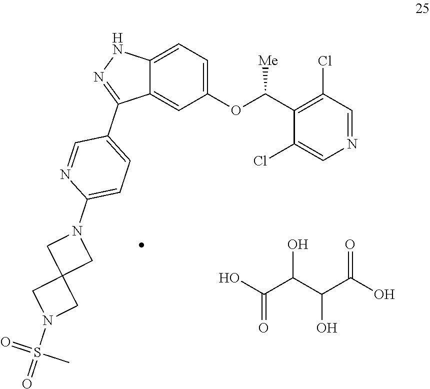 [SMILES: C[C@@H](Oc1ccc2[nH]nc(-c3ccc(N4CC5(C4)CN(S(C)(=O)=O)C5)nc3)c2c1)c1c(Cl)cncc1Cl ; "·" ; O=C(O)C(O)C(O)C(=O)O]
or a crystalline form thereof; or
Compound 26: 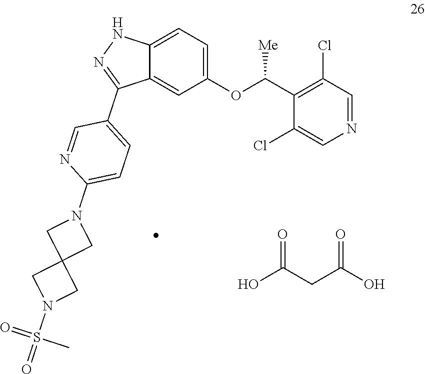 [SMILES: C[C@@H](Oc1ccc2[nH]nc(-c3ccc(N4CC5(C4)CN(S(C)(=O)=O)C5)nc3)c2c1)c1c(Cl)cncc1Cl ; O=C(O)CC(=O)O]
or a crystalline form thereof.

59. A pharmaceutically acceptable composition comprising a compound according to claim 58, and a pharmaceutically acceptable carrier, excipient, or vehicle.

60. A method of treating cancer in a patient comprising administering to said patient a composition according to claim 59.

61. The method according to claim 60, further comprising administering to the patient an additional therapeutic agent.

62. A method for preparing a salt compound of the formula X: 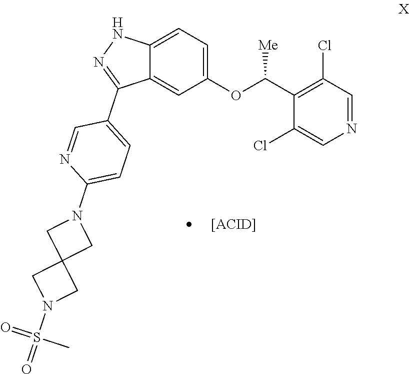 [SMILES: C[C@@H](Oc1ccc2[nH]nc(-c3ccc(N4CC5(C4)CN(S(C)(=O)=O)C5)nc3)c2c1)c1c(Cl)cncc1Cl]
comprising steps of:
combining A: 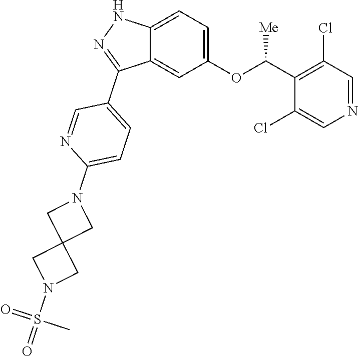 [SMILES: C[C@@H](Oc1ccc2[nH]nc(-c3ccc(N4CC5(C4)CN(S(C)(=O)=O)C5)nc3)c2c1)c1c(Cl)cncc1Cl]
with a suitable acid and optionally a solvent under conditions suitable for producing the compound of formula X.

63. The method of claim 62, wherein:
(a) the suitable acid is hydrochloric acid, and the compound of formula X is the hydrochloride salt of compound A; and optionally crystallizing said hydrochloride salt to hydrochloride salt Form I or Form II as disclosed herein;
(b) the suitable acid is benzenesulfonic acid and the compound of formula X is the besylate salt of compound A; and optionally crystallizing said besylate salt to form besylate salt of compound A Form I, besylate salt of compound A Form II, besylate salt of compound A Form III, besylate salt of compound A Form IV, besylate salt of compound A Form V, besylate salt of compound A Form VI, besylate salt of compound A Form VII, besylate salt of compound A Form IX, besylate salt of compound A Form X, besylate salt of compound A Form XI, besylate salt of compound A Form XII or besylate salt of compound A Form XIII, each as described herein;
(c) the suitable acid is maleic acid, and the compound of formula X is the maleate salt of compound A; and optionally crystallizing said maleate salt to form maleate salt of compound A Form I or Form II as described herein;
(d) the suitable acid is p-toluenesulfonic acid, and the compound of formula X is the tosylate salt of compound A and optionally recrystallizing the tosylate salt to form Form I as described herein;
(e) the suitable acid is sulfuric acid, and the compound of formula X is the sulfate salt of compound A and optionally recrystallizing the sulfate salt to form Form I or Form II as described herein;
(f) the suitable acid is sulfonic acid, and the compound of formula X is the sulfonate salt of compound A and optionally recrystallizing the sulfonate salt to form Form I as described herein;
(g) the suitable acid is ethane sulfonic acid, and the compound of formula X is the esylate salt of compound A and optionally recrystallizing the esylate salt to form Form I as described herein;
(h) the suitable acid is methane sulfonic acid, and the compound of formula X is the mesylate salt of compound A and optionally recrystallizing the mesylate salt to form Form I as described herein;
(i) the suitable acid is methane sulfonic acid, and the compound of formula X is the di-mesylate salt of compound A and optionally recrystallizing the di-mesylate salt to form Form I as described herein;
(j) the suitable acid is R-camphor sulfonic acid, and the compound of formula X is the R-camsylate salt of compound A and optionally crystallizing said R-camsylate salt to form Form I as described herein;
(k) the suitable acid is S-camphor sulfonic acid, and the compound of formula X is the S-camsylate salt of compound A and optionally crystallizing said S-camsylate salt to form Form I as described herein; or
(l) the suitable acid is hydrobromic acid, and the compound of formula X is the bromate salt of compound A and optionally recrystallizing the bromate salt to form Form I or Form II as described herein; or
(m) the suitable acid is phosphoric acid, and the compound of formula X is the phosphate salt of compound A and optionally recrystallizing the phosphate salt to form Form I as described herein; or
(n) the suitable acid is citric acid, and the compound of formula X is the citrate salt of compound A and optionally recrystallizing the citrate salt to form Form I as described herein; or
(o) the suitable acid is L-tartaric acid, and the compound of formula X is the L-tartrate salt of compound A and optionally recrystallizing the L-tartrate salt to form Form I as described herein; or
(p) the suitable acid is malonic acid, and the compound of formula X is the malonate salt of compound A and optionally recrystallizing the malonate salt to form Form I as described herein.
(p) the suitable acid is malonic acid, and the compound of formula X is the malonate salt of compound A and optionally recrystallizing the malonate salt to form Form I as described herein.
